(19) United States
(12) Patent Application Publication
(10) Pub. No.: US 2011/0136801 A1
(43) Pub. Date: Jun. 9, 2011

(54) Novel Compounds

(75) Inventors: Yoshiaki Isobe, Osaka-shi (JP); Mai Kasai, Osaka-shi (JP); Tomoaki Nakamura, Osaka-shi (JP); Shingo Tojo, Osaka-shi (JP); Hirotaka Kurebayashi, Osaka-shi (JP)

(73) Assignees: Dainippon Sumitomo Pharma Co. Ltd., Osaka (JP); AstraZeneca AB, Södertälje (SE)

(21) Appl. No.: 12/958,846

(22) Filed: Dec. 2, 2010

Related U.S. Application Data
(60) Provisional application No. 61/282,017, filed on Dec. 3, 2009.

(51) Int. Cl. A61K 31/4745; C07D 471/04; A61K 31/496; A61K 31/5377; A61P 35/00; A61P 31/04; A61P 31/12; A61P 11/06; A61P 17/00; A61P 31/18; A61P 31/20; A61P 31/14

(52) U.S. Cl. 514/232.8; 546/82; 514/293; 544/361; 514/253.03; 544/126

(57) ABSTRACT

The present invention provides compounds of formula (I):

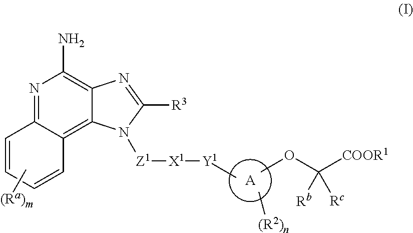

wherein R^{a}, R^{b}, R^{c}, R¹, R², R³, X¹, Y¹, Z¹, A, n and m are as defined in the specification,
   and pharmaceutically acceptable salts thereof, as well as processes for their preparation, pharmaceutical compositions containing them and their use in therapy.

### TECHNICAL FIELD

The present invention relates to imidazoquinoline derivatives, pharmaceutical compositions containing them and their use in therapy.

### BACKGROUND ART

The immune system is comprised of innate and acquired immunity, both of which work cooperatively to protect the host from microbial infections. It has been shown that innate immunity can recognize conserved pathogen-associated molecular patterns through toll-like receptors (TLRs) expressed on the cell surface of immune cells. Recognition of invading pathogens then triggers cytokine production (including interferon alpha (IFNα)) and upregulation of co-stimulatory molecules on phagocytes, leading to modulation of T cell function. Thus, innate immunity is closely linked to acquired immunity and can influence the development and regulation of an acquired response.

TLRs are a family of type I transmembrane receptors characterized by an NH₂-terminal extracellular leucine-rich repeat domain (LRR) and a COOH-terminal intracellular tail containing a conserved region called the Toll/IL1 receptor (TIR) homology domain. The extracellular domain contains a varying number of LRR, which are thought to be involved in ligand binding. Eleven TLRs have been described to date in humans and mice. They differ from each other in ligand specificities, expression patterns, and in the target genes they can induce.

Ligands which act via TLRs (also known as immune response modifiers (IRMS)) have been developed, for example, the imidazoquinoline derivatives described in U.S. Pat. No. 4,689,338 which include the product Imiquimod for treating genital warts, and the adenine derivatives described in WO 98/01448 and WO 99/28321.

### DISCLOSURE OF INVENTION

This patent application describes a class of imidazoquinoline compounds having immuno-modulating properties which act via TLR7 that are useful in the treatment of viral or allergic diseases and cancers.

In accordance with the present invention, there is therefore provided a compound of formula (I):

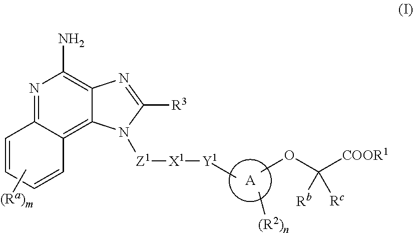

wherein
R¹ represents C₁-C₈ alkyl group, C₃₋₈ cycloalkyl group, or a 3- to 8-membered saturated heterocyclic ring group comprising a O atom, wherein each of said groups is optionally substituted by one or more substituents independently selected from halogen, cyano, hydroxyl and C₁-C₃ alkoxy;
Z¹ represents a C₂-C₆ alkylene, wherein a carbon atom in Z¹ which is not adjacent to a nitrogen atom may be replaced with an oxygen atom;
X¹ represents NR⁵, >N—COR⁵, >N—CONR⁵R^{5a}, CONR⁵, NR⁵CO, NR⁵CONR⁶ or NR⁶CONR⁵;
Y¹ represents a single bond or C₁-C₆ alkylene;
each R² is independently selected from halogen, cyano, hydroxy, thiol, C₁-C₃ alkyl, C₁-C₃ hydroxyalkyl, C₁-C₃ haloalkyl, C₁-C₃ alkoxy, C₁-C₃ haloalkoxy,
C₁₋₃ alkylthio, C₁₋₃ alkylsulfonyl and C₁₋₃ alkylsulfinyl;
R³ represents C₁₋₆ alkyl optionally substituted by C₁₋₆ alkoxy;
each R^{a} is independently selected from halogen, cyano, hydroxy, thiol, C₁-C₃ alkyl, C₁-C₃ hydroxyalkyl, C₁-C₃ haloalkyl, C₁-C₃ alkoxy, C₁-C₃ haloalkoxy, C₁₋₃ alkylthio, C₁₋₃ alkylsulfonyl and C₁₋₃ alkylsulfinyl;
R⁵ and R^{5a} each independently represents hydrogen, a 3- to 8-membered saturated heterocyclic ring comprising a ring group O, S(O)ₚ or NR¹⁰, a C₁-C₆ alkyl group or C₃-C₆ cycloalkyl group, the latter two groups being optionally substituted by one or more substituents independently selected from NR⁷R⁸ or R⁹;
R⁷ and R⁸ each independently represent hydrogen, a 3- to 8-membered saturated heterocyclic ring comprising a ring group O, S(O)ₚ or NR^{10a}, C₁-C₆ alkyl or C₃-C₆ cycloalkyl, the latter two groups being optionally substituted by one or more groups independently selected from halogen, cyano, S(O)_{q}R¹¹, OR¹², CO₂R¹², OC(O)R¹², SO₂NR¹²R¹³, CONR¹²R¹³, NR¹²R¹³, NR¹²SO₂R¹⁴, NR¹²COR¹³, or a 3- to 8-membered saturated heterocyclic ring comprising a ring group O, S(O)ₚ or NR^{10b},
or R⁷ and R⁸ together with the nitrogen atom to which they are attached form a 3- to 8-membered saturated heterocyclic ring comprising a ring nitrogen atom and optionally one or more further heteroatoms independently selected from nitrogen, oxygen, sulphur and sulphonyl, the heterocyclic ring being optionally substituted by one or more substituents independently selected from halogen, cyano, S(O)_{q}R¹⁵, OR¹⁵, CO₂R¹⁵, COR¹⁵, OC(O)R¹⁵, SO₂NR¹⁵R¹⁶, CONR¹⁵R¹⁶, NR¹⁵R¹⁶, NR¹⁵SO₂R¹⁷, NR¹⁵COR¹⁶, NR¹⁵CO₂R¹⁶, heteroaryl, C₁-C₆ haloalkyl, C₃-C₈ cycloalkyl and C₁-C₆ alkyl, the latter two groups being optionally substituted by one or more groups independently selected from cyano, S(O)_{q}R¹⁸, OR¹⁸, CO₂R¹⁸, SO₂NR¹⁸R¹⁹, CONR¹⁸R¹⁹ or NR¹⁸R¹⁹;
R⁹ represents halogen, cyano, CO₂R²⁰, S(O)_{q}R²⁰, OR²⁰, SO₂NR²OR²², CONR²OR²², NR²⁰SO₂R²¹, NR²⁰CO₂R²¹, NR²⁰COR²² or a 3- to 8-membered saturated heterocyclic ring comprising a ring group NR^{10c};
R¹⁰, R^{10a}, R^{10b} and R^{10c} independently represent hydrogen, CO₂R²³, S(O)_{q}R²³, COR²⁴, or a C₁-C₆ alkyl, C₂-C₆ alkenyl, C₂-C₆ alkynyl or C₃-C₈ cycloalkyl group, each of which may be optionally substituted by one or more substituents independently selected from halogen, cyano, OR²⁸ or NR²⁵R²⁶;
R⁶, R¹¹, R¹², R¹³, R¹⁵, R¹⁶, R¹⁸, R¹⁹, R²⁰, R²², R²⁴, R²⁵ and R²⁶ each independently represent hydrogen, C₁-C₆ alkyl or C₃-C₆ cycloalkyl;
R¹⁴, R¹⁷, R²¹ and R²³ each independently represent C₁-C₆ alkyl or C₃-C₆ cycloalkyl;
m, n, p and q each independently represent an integer 0, 1 or 2; and
A represents a monocyclic or bicyclic C₆-C₁₀ aryl or a monocyclic or bicyclic C₅-C₁₂ heteroaryl group containing 1-3 heteroatoms;
R^{b} and R^{c} each independently represent hydrogen or C₁-C₆ alkyl, or R^{b}
and R^{c} combine together to form C₃-C₈ cycloalkyl;
   or a pharmaceutically acceptable salt thereof.

In the context of the present specification, unless otherwise stated, an alkyl substituent group or an alkyl moiety in a substituent group may be linear or branched. They may for example contain from 1 to 8 carbon atoms. Examples of C₁-C₈ alkyl groups/moieties include methyl, ethyl, n-propyl, isopropyl, n-butyl, isobutyl, tert-butyl, n-pentyl, n-hexyl, n-heptyl or n-octyl. Similarly, an alkylene group/moiety may be linear or branched. Examples of C₁-C₆ alkylene groups/moieties include methylene, ethylene, n-propylene, n-butylene, n-pentylene, n-hexylene, 1-m ethylethylene, 2-methylethylene, 1,2-dimethylethylene, 1-ethylethylene, 2-ethylethylene, 1-, 2- or 3-methylpropylene and 1-, 2- or 3-ethylpropylene. An alkenyl or alkynyl group is an unsaturated linear or branched group, containing for example from 2 to 6 carbon atoms. It should be appreciated that, in formula (I), if more than one substituent contains a group or moiety S(O)ₚ or S(O)_{q} or if a substituent contains two or more S(O)ₚ or S(O)_{q}, then each “p” or each “q” independently represents an integer 0, 1 or 2. For example, if R⁷ represents a C₃-C₆ cycloalkyl group substituted by two groups S(O)_{q}R¹¹, then each “q” may be the same or different. In the same way, each group “R¹¹”, where there is more than one such group, may be the same or different.

Cycloalkyl or carbocycle groups are rings containing, for example, from 3 to 8 carbon atoms and are saturated.

Heterocyclic groups are rings which may be saturated, partially unsaturated or unsaturated, and contain from 3 to 20 atoms, at least one and suitably from 1 to 4 atoms are heteroatoms selected from oxygen, sulphur and nitrogen. Rings may be monocyclic, fused, bridged, or spiro bicyclic heterocyclic ring system(s). Monocyclic heterocyclic rings contain from about 3 to 12 ring atoms, with from 1 to 5 heteroatoms selected from N, O and S, and suitably from 3 to 7 member atoms, in the ring. Bicyclic heterocycles contain from 7 to 17 member atoms, suitably 7 to 12 member atoms, in the ring. Bicyclic heterocyclic(s) rings may be fused, spiro, or bridged ring systems.

Examples of heterocyclic groups which are saturated or partially saturated include cyclic ethers (oxiranes) such as ethyleneoxide, tetrahydrofuran, dioxane, and substituted cyclic ethers. Heterocycles containing nitrogen include, for example, azetidine, pyrrolidine, piperidine, piperazine, tetrahydrotriazine, tetrahydropyrazole, and the like. Typical sulfur containing heterocycles include tetrahydrothiophene, dihydro-1,3-dithiol-2-yl, and hexahydrothiepin-4-yl. Other heterocycles include dihydro-oxathiol-4-yl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene. A suitable value for a heterocyclyl group which bears 1 or oxo or thioxo substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl.

Heterocyclic groups which are aromatic in nature are referred to as “heteroaryl” groups. These groups are aromatic mono-, bi-, or polycyclic heterocyclic ring incorporating one or more (for example 1-4) heteroatoms selected from N, O, and S. The term heteroaryl includes both monovalent species and divalent species. Examples of heteroaryl groups include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers ring systems wherein at least one ring is an aromatic ring containing 1 or more heteroatoms selected from O, S and N and one or more of the other rings is a non-aromatic, saturated or partially unsaturated ring optionally containing one or more heteroatoms selected from O, S and N, for example 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

A preferred heteroaryl group is a 5-7 member aromatic ring or 6,6- or 6,5-fused bicyclic ring containing one or more ring heteroatoms selected from N, S, O. Examples include pyridine, pyrimidine, thiazole, oxazole, pyrazole, imidazole, furan, isoxazole, pyrrole, isothiazole and azulene, naphthyl, indene, quinoline, isoquinoline, indole, indolizine, benzo[b]furan, benzo[b]thiophene, 1H-indazole, benzimidazole, benzthiazole, benzoxazole, purine, 4H-quinolizine, cinnoline, phthalazine, quinazoline, quinoxaline, 1,8-naphthyridine, pteridine and quinolone.

In one embodiment R¹ represents a straight or branched chain C₁₋₈ alkyl group optionally substituted by C₁₋₃ alkoxy or hydroxy, for example, methyl, ethyl, n-propyl, isopropyl, n-butyl, sec-butyl, isobutyl, t-butyl, methoxymethyl, methoxyethyl or hydroxyethyl. In another embodiment R¹ represents a straight or branched chain C₁₋₄ alkyl group. In a particular embodiment R¹ is methyl, ethyl, propyl, or isopropyl.

In one embodiment R^{b} and R^{c} independently represent hydrogen or C₁-C₃ alkyl, or R^{b} and R^{c} combine together to form C₃-C₆ cycloalkyl. In another embodiment R^{b} and R^{c} each independently represent hydrogen or methyl, or R^{b} and R^{c} combine together to form cyclopropyl, cyclobutyl, cyclopentyl or cyclohexyl.

In one embodiment R¹ represents a straight chain C₁₋₄ alkyl group, and at least one of R^{b} and R^{c} independently represent C₁-C₄ alkyl or R^{b} and R^{c} combine together to form C₃-C₆ cycloalkyl. In another embodiment R¹ represents methyl or ethyl, and R^{b} represents methyl and R^{c} represents hydrogen or methyl, or R^{b} and R^{c} combine together to form cyclopropyl, cyclobutyl, cyclopentyl or cyclohexyl. In another embodiment R¹, R^{b} and R^{c} represent methyl. In another embodiment R¹ represents ethyl, R^{b} represents methyl and R^{c} represents hydrogen.

In another embodiment when R¹ represents branched chain C₃₋₆ alkyl group, C₃₋₆ cycloalkyl or a tetrahydropyranyl, R^{b} and R^{c} represent hydrogen. For example, R¹ represents isopropyl, and R^{b} and R^{c} represent hydrogen.

In a particular embodiment, Z¹ is a C₂₋₆ alkylene, in particular a straight chain C₂₋₆ alkylene group, for example a straight chain C₂₋₄ alkylene group. A particular example of Z¹ is n-propylene. Another particular example of Z¹ is n-butylene.

In a particular embodiment, X¹ represents NR⁵, >N—COR⁵, >NCONR⁵R^{5a}, NR⁵CO, CONR⁵, NR⁵CONR⁶, or NR⁶CONR⁵. (For the avoidance of doubt, within the definition of X¹, the first atom appearing is linked to the Z¹ group. Thus, when X¹ is CONR⁵, the carbon atom is linked to the Z¹ group and the nitrogen atom is linked to the Y¹ group.) As will be understood, when X¹ represents >N—COR⁵, the nitrogen is attached to Z¹ and Y¹. The same applies when X¹ is >NCONR⁵R^{5a}.

In another embodiment, X¹ represents NR⁵, >N—COR⁵, or >N—CONR⁵R^{5a}.

Where R⁶ is present in any group X¹, it is suitably selected from hydrogen or C₁₋₆ alkyl such as methyl.

A particular example of X¹ is a group NR⁵.

Another particular example of an X¹ group is >N—COR⁵.

Another particular example of an X¹ group is >N—CONR⁵R^{5a}.

Particular examples of R⁵ groups include hydrogen or a C₁₋₆alkyl optionally substituted by one or more substituents independently selected from NR⁷R⁸ or R⁹, where R⁷, R⁸ and R⁹ are as defined above.

For instance, R⁵ represents a C₁-C₆ alkyl or C₁-C₄ alkyl optionally substituted by one or more substituents independently selected from NR⁷R⁸ or R⁹, where R⁷, R⁸ and R⁹ are as defined above.

In particular, R⁵ is a C₁-C₆ alkyl, particularly C₁-C₃ alkyl such as methyl, ethyl or n-propyl, optionally substituted by one or more substituents independently selected from NR⁷, R⁸ where R⁷ and R⁸ are as defined above.

In yet a further embodiment, R⁵ is a C₁-C₆ alkylene which may be linked to a carbon atom within a C₂-C₆ alkylene group Z¹ so as to form a saturated 4-7 membered nitrogen containing ring. In particular, R⁵ is linked to a carbon atom in the Z¹ chain so as to form for example, where X¹ is a group NR⁵, a piperidine ring.

In a particular embodiment, Y¹ represents C₁-C₆ alkylene, such as a CH₂ group.

In a further embodiment, where A is a heteroaryl group, it is suitably a monocyclic ring containing six atoms, one or two of which are nitrogen. Thus particular examples of heteroaryl groups A include pyridyl and pyrimidinyl, suitably pyridyl. A particular example of ring A is phenyl.

In one embodiment A is phenyl and the groups Y¹ and O are in the meta- or para-position on A. In one embodiment A is 1,3-phenylene. In another one embodiment A is 1,4-phenylene.

Where present, R² is suitably halogen such as fluoro or chloro, cyano, hydroxy, thiol, C₁-C₃ alkyl such as methyl, C₁-C₃ hydroxyalkyl such as hydroxymethyl, C₁-C₃ haloalkyl such as trifluoromethyl, C₁-C₃ alkoxy such as methoxy or ethoxy, C₁-C₃ haloalkoxy such as trifluoromethoxy, C₁₋₃alkylthio such as methylthio, C₁₋₃ alkylsulfonyl such as methylsulfonyl or C₁₋₃ alkylsulfinyl such as methylsulfinyl.

Preferably however, n is 0.

In a particular embodiment, R³ represents a C₁₋₆ alkyl group optionally substituted by a C₁₋₄ alkoxy group. Examples of alkyl groups include methyl, ethyl, iso-propyl, n-propyl, and n-butyl. A particular example of R³ is n-propyl or n-butyl. Particular examples of an alkoxy substituted alkyl group R³ include a C₁₋₆ alkyl group substituted by a C₁₋₄ alkoxy group such as methoxy, ethoxy or propoxy, for example R³ is ethoxymethyl or 2-methoxyethyl. In one embodiment R³ is 2-methoxyethyl. In another embodiment R³ is ethoxymethyl. In another embodiment R³ is a C₁₋₆ alkyl group substituted by a C₁₋₄ alkoxy group, provided R³ is not 2-methoxyethyl.

Where present, each R^{a} suitably independently represents halogen such as chloro or fluoro, cyano, hydroxy, thiol, C₁-C₃ alkyl such as methyl, C₁-C₃ hydroxyalkyl such as hydroxymethyl, C₁-C₃ haloalkyl such as trifluoromethyl, C₁-C₃ alkoxy such as methoxy or ethoxy, C₁-C₃ haloalkoxy such as trifluoromethoxy, C₁₋₃alkylthio such as methylthio, C₁₋₃ alkylsulfonyl such as methylsulfonyl or C₁₋₃ alkylsulfinyl such as methylsulfinyl.

Suitably however, m is 0.

R⁷ and R⁸ each independently represent hydrogen, a 3- to 8- or 5- to 6-membered saturated heterocyclic ring comprising a ring group O, S(O)ₚ or NR^{10a}, C₁-C₆, or C₁-C₄, or C₁-C₂ alkyl or C₃-C₆ or C₅-C₆ cycloalkyl, the latter two groups being optionally substituted by one or more (e.g. one, two, three or four) groups independently selected from halogen (e.g. fluorine, chlorine, bromine or iodine), cyano, S(O)_{g}R¹¹, OR¹², CO₂R¹², OC(O)R¹², SO₂NR¹²R¹³, CONR¹²R¹³, NR¹²R¹³, NR¹²SO₂R¹⁴, NR¹²COR¹³, or a 3- to 8- or 5- to 6-membered saturated heterocyclic ring comprising a ring group O, S(O)ₚ or NR^{10b},

or R⁷ and R⁸ together with the nitrogen atom to which they are attached form a 3- to 8-membered saturated heterocyclic ring comprising a ring nitrogen atom and optionally one or more (e.g. one, two or three) further heteroatoms independently selected from nitrogen, oxygen, sulphur and sulphonyl (such as piperidinyl, piperazinyl, morpholinyl or pyrrolidinyl), the heterocyclic ring being optionally substituted by one or more (e.g. one, two, three or four) substituents independently selected from halogen (e.g. fluorine, chlorine, bromine or iodine), cyano, S(O)_{q}R¹⁵, OR¹⁵, CO₂R¹⁵, COR¹⁵, OC(O)R¹⁵, SO₂NR¹⁵R¹⁶, CONR¹⁵R¹⁶, NR¹⁵R¹⁶, NR¹⁵SO₂R¹⁷, NR¹⁵COR¹⁶, NR¹⁵CO₂R¹⁶, heteroaryl (particularly pyrimidinyl), C₁-C₆, or C₁-C₄, or C₁-C₂ haloalkyl (e.g. trifluoromethyl, trifluoromethoxy or pentafluoroethyl), C₃-C₈ or C₅-C₆ cycloalkyl and C₁-C₆, or C₁-C₄, or C₁-C₂alkyl, the latter two groups being optionally substituted by one or more (e.g. one, two, three or four) groups independently selected from cyano, S(O)_{q}R¹⁸, OR¹⁸, CO₂R¹⁸, SO₂NR¹⁸R¹⁹, CONR¹⁸R¹⁹ or NR¹⁸R¹⁹.

In one embodiment, R⁷ and R⁸ each independently represent hydrogen, a 5- to 6-membered saturated heterocyclic ring comprising a ring group O or NR^{10a}, or a C₁-C₆, or C₁-C₄, or C₁-C₂ alkyl group optionally substituted by one or more (e.g. one, two, three or four) groups independently selected from halogen (e.g. fluorine, chlorine, bromine or iodine), cyano, S(O)_{q}R¹¹, OR¹², CO₂R¹², OC(O)R¹², SO₂NR¹²R¹³, CONR¹²R¹³, NR¹²R¹³, NR¹²SO₂R¹⁴, NR¹²COR¹³, or a 3- to 8- or 5- to 6-membered saturated heterocyclic ring comprising a ring group O, S(O)ₚ or NR^{10b}.

In one embodiment, R⁷ and R⁸ represent methyl or ethyl.

In one embodiment, R⁷ and R⁸ represent ethyl.

In another embodiment, R⁷ and R⁸ each independently represent hydrogen, a 5- to 6-membered saturated heterocyclic ring comprising a ring group O or NR^{10a}, or a C₁-C₄alkyl group optionally substituted by one or two groups independently selected from halogen (e.g. fluorine, chlorine, bromine or iodine), cyano, S(O)_{q}R¹¹, OR¹², CO₂R¹², OC(O)R¹², SO₂NR¹²R¹³, CONR¹²R¹³, NR¹²R¹³, NR¹²SO₂R¹⁴, NR¹²COR¹³, or a 3- to 8- or 5- to 6-membered saturated heterocyclic ring comprising a ring group O, S(O)ₚ or NR^{10b}.

In a further embodiment, R⁷ and R⁸ each independently represent a 5- to 6-membered saturated heterocyclic ring comprising a ring group O or NR^{10a} (such as tetrahydropyranyl or N-acetylpiperidinyl) or a C₁-C₄ alkyl group optionally substituted by OR¹².

In an alternative embodiment, R⁷ and R⁸ together with the nitrogen atom to which they are attached form a 3- to 8-membered, particularly 4- to 7- or 5- to 6-membered, saturated heterocyclic ring comprising a ring nitrogen atom and optionally one or more further heteroatoms independently selected from nitrogen, oxygen, sulphur and sulphonyl, the heterocyclic ring being optionally substituted by one or more (e.g. one, two, three or four) substituents independently selected from halogen (e.g. fluorine, chlorine, bromine or iodine), cyano, S(O)_{q}R¹⁵, OR¹⁵, CO₂R¹⁵, COR¹⁵, CONR¹⁵R¹⁶, NR¹⁵CO₂R¹⁶, heteroaryl and C₁-C₆, or C₁-C₄, or C₁-C₂ alkyl, the alkyl group being optionally substituted by one or more (e.g. one, two, three or four) groups independently selected from cyano, S(O)_{q}R¹⁸, OR¹⁸, CO₂R¹⁸, SO₂NR¹⁸R¹⁹, CONR¹⁸R¹⁹ or NR¹⁸R¹⁹.

According to a further embodiment, R⁷ and R⁸ together with the nitrogen atom to which they are attached form a 4- to 7-membered saturated heterocyclic ring comprising a ring nitrogen atom and optionally one further heteroatom selected from nitrogen and oxygen, the heterocyclic ring being optionally substituted by one or two substituents independently selected from S(O)_{q}R¹⁵, OR¹⁵, CO₂R¹⁵, COR¹⁵, CONR¹⁵R¹⁶, NR¹⁵CO₂R¹⁶, pyrimidinyl and C₁-C₂ alkyl, the alkyl group being optionally substituted by one or two groups independently selected from OR¹⁸ and CO₂R¹⁸. In another embodiment of the invention X¹ represents >NCOR⁵ wherein R⁵ represents methyl substituted with NR⁷R⁸; and R⁷ and R⁸ represent independently methyl or ethyl. For example in one embodiment R⁷ and R⁸ are both methyl. In another embodiment R⁷ and R⁸ are both ethyl.

In another embodiment of the invention there is provided a compound of the formula (I), or a pharmaceutically acceptable salt thereof wherein:

Z¹ is n-propylene or n-butylene;
Y¹ is methylene;

### A is

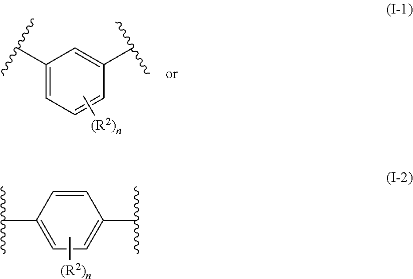

and;
R¹, R², R³, R^{a}, R^{b}, R^{c}, X¹, m and n have any of the values described hereinbefore.

In another embodiment of the invention, there is provided a compound of formula (I), or a pharmaceutically acceptable salt thereof wherein:

Z¹ is n-propylene;
Y¹ is methylene;
X¹ represents >NCOR⁵ wherein R⁵ represents methyl substituted with NR⁷R⁸;
R⁷ and R⁸ represent, independently, methyl or ethyl;
A represents formula (I-1) above;
R¹ represents ⁱPr, and R^{b} and R^{c} represent hydrogen atom, or R¹, R^{b} and R^{c} represent methyl;
R³ represents n-butyl, methoxyethyl or ethoxymethyl; and
m and n represents 0.

In another embodiment of the invention, there is provided a compound of formula (I), or a pharmaceutically acceptable salt thereof wherein:

Z¹ is n-propylene;
Y¹ is methylene;
X¹ represent >NCOR⁵ wherein R⁵ represents methyl substituted with NR⁷R⁸;
R⁷ and R⁸ represent, independently, methyl or ethyl;
A represents formula (I-1) above;
R¹ represents Tr, and R^{b} and R^{c} represent hydrogen atom;
R³ represents ethoxyethyl; and
m and n represents 0.

In another embodiment of the invention, there is provided a compound of formula (I), or a pharmaceutically acceptable salt thereof wherein:

Z¹ is n-propylene;
Y¹ is methylene;
X¹ represents >NCOR⁵ wherein R⁵ represents methyl substituted with NR⁷R⁸;
R⁷ and R⁸ represent, independently, methyl or ethyl;
A represents formula (I-1) above;
R¹, R^{b} and R^{c} represent methyl;
R³ represents methoxyethyl; and
m and n represents 0.

Examples of compounds of the invention include a compound selected from List A:

### List A:

Methyl 2-(3-{[3-(4-amino-2-butyl-1H-imidazo[4,5-c]quinolin-1-yl)propylamino]methyl}phenoxy)acetate
Methyl (3-{[[3-(4-amino-2-butyl-1H-imidazo[4,5-c]quinolin-1-yl)propyl](chloroacetyl)amino]methyl}phenoxy)acetate
Methyl (4-{[[3-(4-amino-2-butyl-1H-imidazo[4,5-c]quinolin-1-yl)propyl]amino]methyl}phenoxy)acetate
Methyl (4-{[[3-(4-amino-2-butyl-1H-imidazo[4,5-c]quinolin-1-yl)propyl](chloroacetyl)amino]methyl}phenoxy)acetate
Methyl (4-{[[3-(4-amino-2-butyl-1H-imidazo[4,5-c]quinolin-1-yl)propyl](N,N-dimethylglycyl)amino]methyl}phenoxy)acetate
Methyl (4-{[[3-(4-amino-2-butyl-1H-imidazo[4,5-c]quinolin-1-yl)propyl](piperidin-1-ylacetyl)amino]methyl}phenoxy)acetate
Methyl [4-({[3-(4-amino-2-butyl-1H-imidazo[4,5-c]quinolin-1-yl)propyl][(4-methylpiperazin-1-yl)acetyl]amino}methyl)phenoxy]acetate
Methyl {4-[([3-(4-amino-2-butyl-1H-imidazo[4,5-c]quinolin-1-yl)propyl]{[4-(2-methoxyethyl)piperazin-1-yl]acetyl}amino)methyl]phenoxy}acetate
Methyl (3-{[[3-(4-amino-2-butyl-1H-imidazo[4,5-c]quinolin-1-yl)propyl](N,N-dimethylglycyl)amino]methyl}phenoxy)acetate
Methyl (3-{[[3-(4-amino-2-butyl-1H-imidazo[4,5-c]quinolin-1-yl)propyl](piperidin-1-ylacetyl)amino]methyl}phenoxy)acetate
Methyl [3-({[3-(4-amino-2-butyl-1H-imidazo[4,5-c]quinolin-1-yl)propyl][(4-methylpiperazin-1-yl)acetyl]amino}methyl)phenoxy]acetate
Methyl {3-[([3-(4-amino-2-butyl-1H-imidazo[4,5-c]quinolin-1-yl)propyl]{[4-(2-methoxyethyl)piperazin-1-yl]acetyl]amino}methyl)phenoxy}acetate
Methyl (3-{[[3-(4-amino-2-butyl-1H-imidazo[4,5-c]quinolin-1-yl)propyl](pyrrolidin-1-ylacetyl)amino]methyl}phenoxy)acetate
Methyl (3-{[[3-(4-amino-2-butyl-1H-imidazo[4,5-c]quinolin-1-yl)propyl](N,N-diethylglycyl)amino]methyl}phenoxy)acetate
Methyl (3-{[[3-(4-amino-2-(2-methoxyethyl)-1H-imidazo[4,5-c]quinolin-1-yl)propyl]amino]methyl}phenoxy)acetate
Methyl (3-{[[3-(4-amino-2-(2-methoxyethyl)-1H-imidazo[4,5-c]quinolin-1-yl)propyl](chloroacetyl)amino]methyl}phenoxy)acetate
Methyl (3-{[[3-(4-amino-2-(2-methoxyethyl)-1H-imidazo[4,5-c]quinolin-1-yl)propyl](N,N-dimethylglycyl)amino]methyl}phenoxy)acetate
Methyl (3-{[[3-(4-amino-2-(2-methoxyethyl)-1H-imidazo[4,5-c]quinolin-1-yl)propyl][(4-methylpiperazin-1-yl)acetyl]amino]methyl}phenoxy)acetate
Methyl (3-{[[3-(4-amino-2-(2-methoxyethyl)-1H-imidazo[4,5-c]quinolin-1-yl)propyl](piperidin-1-ylacetyl)amino]methyl}phenoxy)acetate
Methyl (3-{[[3-(4-amino-2-(2-methoxyethyl)-1H-imidazo[4,5-c]quinolin-1-yl)propyl](N,N-diethylglycyl)amino]methyl}phenoxy)acetate
Methyl (3-{[[3-(4-amino-2-butyl-1H-imidazo[4,5-c]quinolin-1-yl)propyl](3-morpholinopropyl)amino]methyl}phenoxy)acetate
Methyl [4-({[({3-[4-amino-2-(2-methoxyethyl)-1H-imidazo[4,5-c]quinolin-1-yl]propyl}amino)carbonyl][3-(dimethylamino)propyl]amino}methyl)phenoxy]acetate
Ethyl 2-[3-({3-[4-amino-2-(2-methoxyethyl)-1H-imidazo[4,5-c]quinolin-1-yl]propylamino}methyl)phenoxy]acetate
Ethyl 2-{3-[(N-{3-[4-amino-2-(2-methoxyethyl)-1H-imidazo[4,5-c]quinolin-1-yl]propyl}-2-chloroacetamido)methyl)phenoxy]acetate
Ethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate
Propyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate
Isopropyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate
Isobutyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate
2-Methoxyethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate
2-Hydroxyethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate
Ethyl 2-{3-[(N-{3-[4-amino-2-(2-methoxyethyl)-1H-imidazo[4,5-c]quinolin-1-yl]propyl}-2-(pyrrolidin-1-yl)acetamido)methyl]phenoxy}acetate
Ethyl 2-{3-[(N-{3-[4-amino-2-(2-methoxyethyl)-1H-imidazo[4,5-c]quinolin-1-yl]propyl}-2-(piperidin-1-yl)acetamido)methyl]phenoxy}acetate
Ethyl 2-{3-[(N-{3-[4-amino-2-(2-methoxyethyl)-1H-imidazo[4,5-c]quinolin-1-yl]propyl}-2-(dimethylamino)acetamido)methyl]phenoxy}acetate
Methyl 2-[4-({3-[4-amino-2-(2-methoxyethyl)-1H-imidazo[4,5-c]quinolin-1-yl]propylamino}methyl)phenoxy]acetate
Methyl 2-{4-[(N-{3-[4-amino-2-(2-methoxyethyl)-1H-imidazo[4,5-c]quinolin-1-yl]propyl}-2-chloroacetamido)methyl]phenoxy}acetate
Methyl 2-{4-[(N-{3-[4-amino-2-(2-methoxyethyl)-1H-imidazo[4,5-c]quinolin-1-yl]propyl}-2-(diethylamino)acetamido)methyl]phenoxy}acetate
Ethyl 2-{4-[(N-{3-[4-amino-2-(2-methoxyethyl)-1H-imidazo[4,5-c]quinolin-1-yl]propyl}-2-(diethylamino)acetamido)methyl]phenoxy}acetate
Methyl 2-[2-({3-[4-amino-2-(2-methoxyethyl)-1H-imidazo[4,5-c]quinolin-1-yl]propylamino}methyl)phenoxy]acetate
Methyl 2-{2-[(N-{3-[4-amino-2-(2-methoxyethyl)-1H-imidazo[4,5-c]quinolin-1-yl]propyl}-2-chloroacetamido)methyl]phenoxy}acetate
Methyl 2-{2-[(N-{3-[4-amino-2-(2-methoxyethyl)-1H-imidazo[4,5-c]quinolin-1-yl]propyl}-2-(diethylamino)acetamido)methyl]phenoxy}acetate
Ethyl 2-{2-[(N-{3-[4-amino-2-(2-methoxyethyl)-1H-imidazo[4,5-c]quinolin-1-yl]propyl}-2-(diethylamino)acetamido)methyl]phenoxy}acetate
Ethyl 2-[3-({4-[4-amino-2-(2-methoxyethyl)-1H-imidazo[4,5-c]quinolin-1-yl]butylamino}methyl)phenoxy]acetate
Ethyl 2-{3-[(N-{4-[4-amino-2-(2-methoxyethyl)-1H-imidazo[4,5-c]quinolin-1-yl]butyl}-2-chloroacetamido)methyl]phenoxy}acetate
Ethyl 2-{3-[(N-{4-[4-amino-2-(2-methoxyethyl)-1H-imidazo[4,5-c]quinolin-1-yl]butyl}-2-(diethylamino)acetamido)methyl]phenoxy}acetate
Isopropyl 2-{3-[(N-{4-[4-amino-2-(2-methoxyethyl)-1H-imidazo[4,5-c]quinolin-1-yl]butyl}-2-{diethylamino}acetamido)methyl]phenoxy}acetate
tert-Butyl 2-[3-({4-[4-amino-2-(2-methoxyethyl)-1H-imidazo[4,5-c]quinolin-1-yl]butylamino}methyl)phenoxy]acetate
tert-Butyl 2-{3-[(N-{4-[4-amino-2-(2-methoxyethyl)-1H-imidazo[4,5-c]quinolin-1-yl]butyl}-2-chloroacetamido)methyl]phenoxy}acetate
tert-Butyl 2-{3-[(N-{4-[4-amino-2-(2-methoxyethyl)-1H-imidazo[4,5-c]quinolin-1-yl]butyl}-2-{diethylamino}acetamido)methyl]phenoxy}acetate
Methyl 2-[3-({3-[4-amino-2-(2-methoxyethyl)-1H-imidazo[4,5-c]quinolin-1-yl]propylamino}methyl)phenoxy]propanoate
Methyl 2-{3-[(N-{3-[4-amino-2-(2-methoxyethyl)-1H-imidazo[4,5-c]quinolin-1-yl]propyl}-2-chloroacetamido)methyl]phenoxy}propanoate
Methyl

2-{3-[(N-{3-[4-amino-2-(2-methoxyethyl)-1H-imidazo[4,5-c]quinolin-1-yl]propyl}-2-{diethylamino}acetamido)methyl]phenoxy}propanoate
Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}propanoate
Ethyl 2-[3-({3-[4-amino-2-(2-methoxyethyl)-1H-imidazo[4,5-c]quinolin-1-yl]propylamino}methyl)phenoxy]-2-methylpropanoate
Ethyl 2-{3-[(N-{3-[4-amino-2-(2-methoxyethyl)-1H-imidazo[4,5-c]quinolin-1-yl]propyl}-2-chloroacetamido)methyl]phenoxy}-2-methylpropanoate
Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}-2-methylpropanoate
Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}-2-methylpropanoate
Ethyl 1-[3-({3-[4-amino-2-(2-methoxyethyl)-1H-imidazo[4,5-c]quinolin-1-yl]propylamino}methyl)phenoxy]cyclobutanecarboxylate
Ethyl 1-{3-[(N-{3-[4-amino-2-(2-methoxyethyl)-1H-imidazo[4,5-c]quinolin-1-yl]propyl}-2-chloroacetamido)methyl]phenoxy}cyclobutanecarboxylate
Ethyl 1-{3-[(N-{3-[4-amino-2-(2-methoxyethyl)-1H-imidazo[4,5-c]quinolin-1-yl]propyl}-2-{diethylamino}acetamido)methyl]phenoxy}cyclobutanecarboxylate
Ethyl 2-[5-({3-[4-amino-2-(2-methoxyethyl)-1H-imidazo[4,5-c]quinolin-1-yl]propylamino}methyl)-2-methoxyphenoxy]acetate
Ethyl 2-{5-[(N-{3-[4-amino-2-(2-methoxyethyl)-1H-imidazo[4,5-c]quinolin-1-yl]propyl}2-chloroacetamido)methyl]-2-methoxyphenoxy}acetate
Ethyl 2-{5-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate
Methyl 2-{5-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate
Ethyl 2-[5-({3-[4-amino-2-(2-methoxyethyl)-1H-imidazo[4,5-c]quinolin-1-yl]propylamino}methyl)-2-methylphenoxy]acetate
Ethyl 2-{5-[(N-{3-[4-amino-2-(2-methoxyethyl)-1H-imidazo[4,5-c]quinolin-1-yl]propyl}-2-chloroacetamido)methyl]-2-methylphenoxy}acetate
Ethyl 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetate
Isopropyl 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetate
Methyl 2-[3-({3-[4-amino-2-(2-methoxyethyl)-1H-imidazo[4,5-c]quinolin-1-yl]propylamino}methyl)phenoxy]butanoate
Methyl 2-{3-[(N-{3-[4-amino-2-(2-methoxyethyl)-1H-imidazo[4,5-c]quinolin-1-yl]propyl}-2-chloroacetamido)methyl]phenoxy}butanoate
Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}butanoate
Ethyl

2-{3-[(N-{3-[4-amino-2-(2-methoxyethyl)-1H-imidazo[4,5-c]quinolin-1-yl]propyl}-2-{diethylamino}acetamido)methyl]phenoxy}butanoate
Isopropyl 2-{5-[(N-{3-[4-amino-2-(2-methoxyethyl)-1H-imidazo[4,5-c]quinolin-1-yl]propyl}-2-{diethylamino}acetamido)methyl]-2-methoylphenoxy}acetate
Isopropyl 2-[5-({3-[4-amino-2-(2-methoxyethyl)-1H-imidazo[4,5-c]quinolin-1-yl]propylamino}methyl)-2-methoxyphenoxy]acetate
Isopropyl 2-{5-[(N-{3-[4-amino-2-(2-methoxyethyl)-1H-imidazo[4,5-c]quinolin-1-yl]propyl}-2-chloroacetamido)methyl]-2-methoxyphenoxy}acetate
Isopropyl 2-{5-[(N-{3-[4-amino-2-(2-methoxyethyl)-1H-imidazo[4,5-c]quinolin-1-yl]propyl}-2-{dimethylamino}acetamido)methyl]-2-methoxyphenoxy}acetate
Isopropyl 2-{5-[(N-{3-[4-amino-2-(2-methoxyethyl)-1H-imidazo[4,5-c]quinolin-1-yl]propyl}-2-{ethyl(methyl)amino}acetamido)methyl]-2-methoxyphenoxy}acetate
Methyl 1-[3-({3-[4-amino-2-(2-methoxyethyl)-1H-imidazo[4,5-c]quinolin-1-yl]propylamino}methyl)phenoxy]cyclopropanecarboxylate
Methyl 1-{3-[(N-{3-[4-amino-2-(2-methoxyethyl)-1H-imidazo[4,5-c]quinolin-1-yl]propyl}-2-chloroacetamido)methyl]phenoxy}cyclopropanecarboxylate
Methyl 1-{3-[(N-{3-[4-amino-2-(2-methoxyethyl)-1H-imidazo[4,5-c]quinolin-1-yl]propyl}-2-{diethylamino}acetamido)methyl]phenoxy}cyclopropanecarboxylate
Cyclopentyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate
Cyclobutyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate
Tetrahydro-2H-pyran-4-yl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate
Butyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate
tert-Butyl 2-[3-({3-[4-amino-2-(2-methoxyethyl)-1H-imidazo[4,5-c]quinolin-1-yl]propylamino}methyl)phenoxy]acetate
tert-Butyl

2-{3-[(N-{3-[4-amino-2-(2-methoxyethyl)-1H-imidazo[4,5-c]quinolin-1-yl]propyl}-2-chloroacetamido)methyl]phenoxy}acetate
tert-Butyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate
Ethyl 2-[3-({3-[4-amino-2-(2-methoxyethyl)-1H-imidazo[4,5-c]quinolin-1-yl]propylamino}methyl)-2-methoxyphenoxy]acetate
Ethyl 2-{3-[(N-{3-[4-amino-2-(2-methoxyethyl)-1H-imidazo[4,5-c]quinolin-1-yl]propyl}-2-chloroacetamido)methyl]-2-methoxyphenoxy}acetate
Ethyl 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate
Isopropyl 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate
Ethyl 2-[3-({3-[4-amino-2-(2-methoxyethyl)-1H-imidazo[4,5-c]quinolin-1-yl]propylamino}methyl)-2-fluorophenoxy]acetate
Ethyl 2-{3-[(N-{3-[4-amino-2-(2-methoxyethyl)-1H-imidazo[4,5-c]quinolin-1-yl]propyl}-2-chloroacetamido)methyl]-2-fluorophenoxy}acetate
Ethyl 2-{3-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate
Isopropyl 2-{3-[(N-{3-[4-amino-2-(2-methoxyethyl)-1H-imidazo[4,5-c]quinolin-1-yl]propyl}-2-{diethylamino}acetamido)methy]-2-fluorophenoxy}acetate
Ethyl 2-{3-[(N-{3-[4-amino-2-(2-methoxyethyl)-1H-imidazo[4,5-c]quinolin-1-yl]propyl}-2-{dimethylamino}acetamido)methyl]-2-fluorophenoxy}acetate
Isopropyl 2-{3-[(N-{3-[4-amino-2-(2-methoxyethyl)-1H-imidazo[4,5-c]quinolin-1-yl]propyl}-2-{dimethylamino}acetamido)methyl]-2-fluorophenoxy}acetate
Ethyl 2-{3-[(N-{3-[4-amino-2-(2-methoxyethyl)-1H-imidazo[4,5-c]quinolin-1-yl]propyl}-2-{ethyl(methyl)amino}acetamido)methyl]-2-fluorophenoxy}acetate
Isopropyl 2-{3-[(N-{3-[4-amino-2-(2-methoxyethyl)-1H-imidazo[4,5-c]quinolin-1-yl]propyl}-2-{ethyl(methyl)amino}acetamido)methyl]-2-fluorophenoxy}acetate
Ethyl 2-[3-({2-[4-amino-2-(2-methoxyethyl)-1H-imidazo[4,5-c]quinolin-1-yl]ethylamino}methyl)phenoxy]acetate
Ethyl 2-{3-[(N-{2-[4-amino-2-(2-methoxyethyl)-1H-imidazo[4,5-c]quinolin-1-yl]ethyl}-2-chloroacetamido)methyl]phenoxy}acetate
Ethyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate
Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate
Isopropyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate
Ethyl 2-[3-({2-[4-amino-2-(2-methoxyethyl)-1H-imidazo[4,5-c]quinolin-1-yl]ethylamino}methyl)phenoxy]-2-methylpropanoate
Ethyl 2-{3-[(N-{2-[4-amino-2-(2-methoxyethyl)-1H-imidazo[4,5-c]quinolin-1-yl]ethyl}-2-chloroacetamido)methyl]phenoxy}-2-methylpropanoate
Ethyl 2-{3-[(N-{2-[4-amino-2-(2-methoxyethyl)-1H-imidazo[4,5-c]quinolin-1-yl]ethyl}-2-{diethylamino}acetamido)methyl]phenoxy}-2-methylpropanoate
Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}-2-methylpropanoate
Cyclopentyl 2-[3-({3-[4-amino-2-(2-methoxyethyl)-1H-imidazo[4,5-c]quinolin-1-yl]propylamino}methyl)phenoxy]acetate
Cyclopentyl 2-{3-[(N-{3-[4-amino-2-(2-methoxyethyl)-1H-imidazo[4,5-c]quinolin-1-yl]propyl}-2-chloroacetamido)methyl]phenoxy}acetate
Cyclopentyl 2-{3-[(N-{3-[4-amino-2-(2-methoxyethyl)-1H-imidazo[4,5-c]quinolin-1-yl]propyl}-2-{dimethylamino}acetamido)methyl]phenoxy}acetate
Cyclopentyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}acetate
Isopropyl 2-{3-[(N-{3-[4-amino-2-(2-methoxyethyl)-1H-imidazo[4,5-c]quinolin-1-yl]propyl}-2-morpholinoacetamido)methyl]phenoxy}acetate
Isopropyl 2-{3-[(N-{3-[4-amino-2-(2-methoxyethyl)-1H-imidazo[4,5-c]quinolin-1-yl]propyl}-2-{dimethylamino}acetamido)methyl]phenoxy}acetate
Isopropyl 2-{3-[(N-{3-[4-amino-2-(2-methoxyethyl)-1H-imidazo[4,5-c]quinolin-1-yl]propyl}-2-{ethyl(methyl)amino}acetamido]methyl)phenoxy}acetate
Isopropyl 2-{3-[(N-{3-[4-amino-2-(2-methoxyethyl)-1H-imidazo[4,5-c]quinolin-1-yl]propyl}-2-{(2-methoxyethyl)(methyl)amino}acetamido)methyl]phenoxy}acetate
Isopropyl 2-[5-({3-[4-amino-2-(2-methoxyethyl)-1H-imidazo[4,5-c]quinolin-1-yl]propylamino}methyl)-2-fluorophenoxy]acetate
Isopropyl 2-{5-[(N-{3-[4-amino-2-(2-methoxyethyl)-1H-imidazo[4,5-c]quinolin-1-yl]propyl}-2-chloroacetamido)methyl]-2-fluorophenoxy}acetate
Isopropyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate
Ethyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate
Methyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate
Isopropyl 2-[3-({3-[4-amino-2-(2-methoxyethyl)-1H-imidazo[4,5-c]quinolin-1-yl]propylamino}methyl)-5-fluorophenoxy]acetate
Isopropyl 2-{3-[(N-{3-[4-amino-2-(2-methoxyethyl)-1H-imidazo[4,5-c]quinolin-1-yl]propyl}-2-chloroacetamido)methyl]-5-fluorophenoxy}acetate
Isopropyl 2-{3-[(N-{3-[4-amino-2-(2-methoxyethyl)-1H-imidazo[4,5-c]quinolin-1-yl]propyl}-2-{diethylamino}acetamido)methyl]-5-fluorophenoxy]acetate
Ethyl

2-{3-[(N-{3-[4-amino-2-(2-methoxyethyl)-1H-imidazo[4,5-c]quinolin-1-yl]propyl}-2-{diethylamino}acetamido)methyl]-5-fluorophenoxy}acetate
Ethyl

2-{4-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate
Ethyl 2-{3-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate
Ethyl 2-{3-[(1-{4-[4-amino-2-(2-methoxyethyl)-1H-imidazo[4,5-c]quinolin-1-yl]butyl}-3-{2-(dimethylamino)ethyl}ureido)methyl]phenoxy}acetate
Ethyl 2-{3-[(1-{4-[4-amino-2-(2-methoxyethyl)-1H-imidazo[4,5-c]quinolin-1-yl]butyl}-3-{3-(piperidin-1-yl)propyl}ureido)methyl]phenoxy}acetate
Ethyl 2-{3-[(1-{4-[4-amino-2-(2-methoxyethyl)-1H-imidazo[4,5-c]quinolin-1-yl]butyl}-3-{3-(dimethylamino)propyl}ureido)methyl]phenoxy}acetate
Ethyl

2-{3-[(3-{4-[4-amino-2-(2-methoxyethyl)-1H-imidazo[4,5-c]quinolin-1-yl]butyl}-1-{2-(piperidin-1-yl)ethyl}ureido)methyl]phenoxy}acetate
Ethyl 2-{4-[(3-{4-[4-amino-2-(2-methoxyethyl)-1H-imidazo[4,5-c]quinolin-1-yl]butyl}-1-{2-(piperidin-1-yl)ethyl}ureido)methyl]phenoxy}acetate
Isopropyl 2-{3-[({4-[4-amino-2-(2-methoxyethyl)-1H-imidazo[4,5-c]quinolin-1-yl]butyl}{2-[di methylamino]ethyl}amino)methyl]phenoxy}acetate
Isopropyl 2-{3-[({4-[4-amino-2-(2-methoxyethyl)-1H-imidazo[4,5-c]quinolin-1-yl]butyl}{3-morpholinopropyl}amino)methyl]phenoxy}acetate
Ethyl 2-{3-[({4-[4-amino-2-(2-methoxyethyl)-1H-imidazo[4,5-c]quinolin-1-yl]butyl}{2-(di methylamino)ethyl}amino)methyl]phenoxy}-2-methylpropanoate
Methyl 2-{3-[({4-[4-amino-2-(2-methoxyethyl)-1H-imidazo[4,5-c]quinolin-1-yl]butyl}{2-(dimethylamino)ethyl}amino)methyl]phenoxy}-2-methylpropanoate
Ethyl 2-{3-[({4-[4-amino-2-(2-methoxyethyl)-1H-imidazo[4,5-c]quinolin-1-yl]butyl}{3-morpholinopropyl}amino)methyl]phenoxy}-2-methylpropanoate
Methyl 2-{3-[({4-[4-amino-2-(2-methoxyethyl)-1H-imidazo[4,5-c]quinolin-1-yl]butyl}{3-morpholinopropyl}amino)methyl]phenoxy}-2-methylpropanoate
Isopropyl 2-[5-({4-[4-amino-2-(2-methoxyethyl)-1H-imidazo[4,5-c]quinolin-1-yl]butylamino}methyl)-2-fluorophenoxy]acetate
Isopropyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate
Ethyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate
Methyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate
Isopropyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate
Ethyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate
Methyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate
Isopropyl 2-[3-({3-[4-amino-2-(2-methoxyethyl)-1H-imidazo[4,5-c]quinolin-1-yl]propylamino}methyl)-2-methylphenoxy]acetate
Isopropyl 2-{3-[(N-{3-[4-amino-2-(2-methoxyethyl)-1H-imidazo[4,5-c]quinolin-1-yl]propyl}-2-chloroacetamido)methyl]-2-methylphenoxy}acetate
Isopropyl 2-{3-[(N-{3-[4-amino-2-(2-methoxyethyl)-1H-imidazo[4,5-c]quinolin-1-yl]propyl}-2-{diethylamino}acetamido)methyl]-2-methylphenoxy}acetate
Ethyl 2-{3-[(N-{3-[4-amino-2-(2-methoxyethyl)-1H-imidazo[4,5-c]quinolin-1-yl]propyl}-2-{dimethylamino}acetamido)methyl]phenoxy}-2-methylpropanoate
Methyl 2-{3-[(N-{3-[4-amino-2-(2-methoxyethyl)-1H-imidazo[4,5-c]quinolin-1-yl]propyl}-2-{dimethylamino}acetamido)methyl]phenoxy}-2-methylpropanoate
Ethyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}-2-methylpropanoate
Methyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}-2-methylpropanoate
Isopropyl 2-(3-{[1-(2-{2-[4-amino-2-(2-methoxyethyl)-1H-imidazo[4,5-c]quinolin-1-yl]ethoxy}ethyl)-3-{2-(piperidin-1-yl)ethyl}ureido]methyl}phenoxy)acetate
Ethyl 2-[3-({N-[3-(4-amino-2-butyl-1H-imidazo[4,5-c]quinolin-1-yl)propyl]-2-(diethylamino)acetamido}methyl)phenoxy]-2-methylpropanoate
Methyl 2-[3-({N-[3-(4-amino-2-butyl-1H-imidazo[4,5-c]quinolin-1-yl)propyl]-2-(diethylamino)acetamido}methyl)phenoxy]-2-methylpropanoate
Ethyl 2-[3-({N-[3-(4-amino-2-butyl-1H-imidazo[4,5-c]quinolin-1-yl)propyl]-2-(dimethylamino)acetamido}methyl)phenoxy]-2-methylpropanoate
Methyl 2-[3-({N-[3-(4-amino-2-butyl-1H-imidazo[4,5-c]quinolin-1-yl)propyl]-2-(dimethylamino)acetamido}methyl)phenoxy]-2-methylpropanoate
Isopropyl 2-[3-({N-[3-(4-amino-2-butyl-1H-imidazo[4,5-c]quinolin-1-yl)propyl]-2-(diethylamino)acetamido}methyl)phenoxy]acetate
Isopropyl 2-[3-({N-[3-(4-amino-2-butyl-1H-imidazo[4,5-c]quinolin-1-yl)propyl]-2-[ethyl(methyl)amino]acetamido}methyl)phenoxy]acetate
Isopropyl 2-[3-({N-[3-(4-amino-2-butyl-1H-imidazo[4,5-c]quinolin-1-yl)propyl]-2-(dimethylamino)acetamido}methyl)phenoxy]acetate
Isopropyl

2-[3-({N-[3-(4-amino-2-propyl-1H-imidazo[4,5-c]quinolin-1-yl)propyl]-2-(diethylamino)acetamido}methyl)phenoxy]acetate
Isopropyl 2-{3-[(N-{3-[4-amino-2-(ethoxymethyl)-1H-imidazo[4,5-c]quinolin-1-yl]propyl}-2-{diethylamino}acetamido)methyl]phenoxy}acetate
Ethyl 2-{3-[(N-{3-[4-amino-2-(ethoxymethyl)-1H-imidazo[4,5-c]quinolin-1-yl]propyl}-2-{diethylamino}acetamido)methyl]phenoxy}-2-methylpropanoate
Methyl 2-{3-[(N-{3-[4-amino-2-(ethoxymethyl)-1H-imidazo[4,5-c]quinolin-1-yl]propyl}-2-{diethylamino}acetamido)methyl]phenoxy}-2-methylpropanoate
Isopropyl 2-[3-({N-[3-(4-amino-2-butyl-1H-imidazo[4,5-c]quinolin-1-yl)propyl]-2-(diethylamino)acetamido}methyl)-2-fluorophenoxy]acetate
Isopropyl 2-[3-({N-[3-(4-amino-2-butyl-1H-imidazo[4,5-c]quinolin-1-yl)propyl]-2-(dimethylamino)acetamido}methyl)-2-fluorophenoxy]acetate
Isopropyl 2-{3-[(N-{3-[4-amino-2-(ethoxymethyl)-1H-imidazo[4,5-c]quinolin-1-yl]propyl}-2-{diethylamino}acetamido)methyl]-2-fluorophenoxy}acetate
Isopropyl 2-{3-[(N-{3-[4-amino-2-(ethoxymethyl)-1H-imidazo[4,5-c]quinolin-1-yl]propyl}-2-{dimethylamino}acetamido)methyl]-2-fluorophenoxy}acetate
Ethyl 2-[3-({N-[3-(4-amino-2-butyl-1H-imidazo[4,5-c]quinolin-1-yl)propyl]-2-(pyrrolidin-1-yl)acetamido}methyl)phenoxy]-2-methylpropanoate
Methyl 2-[3-({N-[3-(4-amino-2-butyl-1H-imidazo[4,5-c]quinolin-1-yl)propyl]-2-(pyrrolidin-1-yl)acetamido}methyl)phenoxy]-2-methylpropanoate
Ethyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}-2-methylpropanoate
Methyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}-2-methylpropanoate
Isopropyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}acetate,
Ethyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate and
Methyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate,
   or a pharmaceutically acceptable salt thereof.

According to another embodiment of the invention there is provided a compound of the formula (I), or a pharmaceutically acceptable salt thereof as hereinbefore defined, other than any one of the compounds described in List A.

The present invention further provides a process for the preparation of a compound of formula (I) or a pharmaceutically acceptable salt thereof as defined above which comprises either:

(a) where X¹ is a group NR⁵, reacting a compound of formula (II):

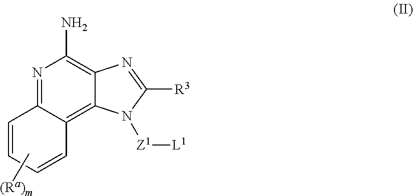

wherein Z¹, R³, R^{a} and m are as defined in formula (I) and L¹ is a leaving group, with a compound of formula (III):

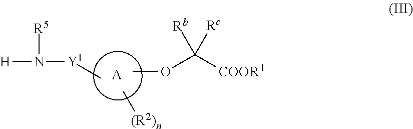

where Y¹, R¹, R², R⁵, A and n are as defined in formula (I); or
(b) where X¹ is a group NR⁵ and Y¹ is C₁-C₆ alkylene, reacting a compound of formula (IV):

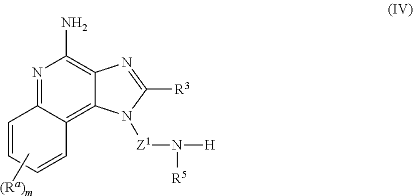

where R^{a}, R³, R⁵, Z¹ and m are as defined in formula (I), with a compound of formula (V):

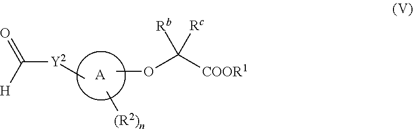

where R¹, R², A and n are as defined in formula (I) and Y² is a bond or a C₁₋₅ alkylene group in the presence of a suitable reducing agent (e.g. sodium triacetoxyborohydride); or
(c) where X¹ is a group NR⁵, reacting a compound of formula (VI):

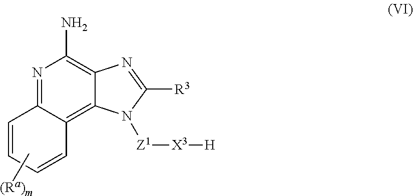

wherein X³ is a group NR⁵, and Z¹, R³, R⁵, R^{a} and m are as defined in formula (I), with a compound of formula (VII):

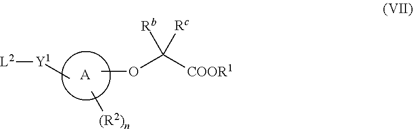

where Y¹, R¹, R², A and n are as defined in formula (I) and L² is a leaving group; or (d) where X¹ is a group NR⁵CO, NR⁵CONR⁶ or NR⁶CONR⁵, reacting a compound of formula (IVA):

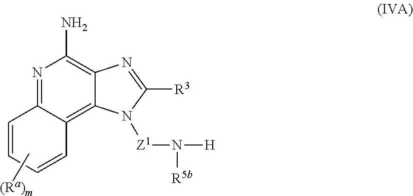

where R^{a}, R³, Z¹ and m are as defined in relation to formula (I) and R^{5b} is a group R⁵ or R⁶ as defined in relation to formula (I),
with a compound of formula (VIII):

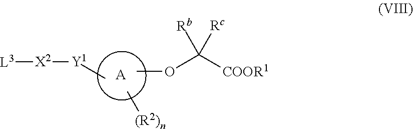

where L³ is a leaving group such as halo, phenoxy or 4-nitrophenoxy, X² is a CO, CONR⁶ or CONR⁵ group respectively, and Y¹, R¹, R², A and n are as defined in relation to formula (I); or
(e) where X¹ is CONR⁵, reacting a compound of formula (IX):

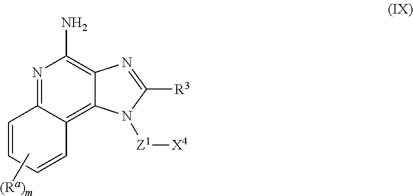

where X⁴ is an activated acid such as an acid chloride, R^{a}, R³, Z¹ and m are as defined in formula (I), with a compound of formula (III) as defined above; or
(f) where X¹ is >N—COR⁵, or >N—CONR⁵R^{5a}, reacting a compound of formula (I) where X¹ is NR⁵ where R⁵ is hydrogen with a compound of formula (X) or (X¹) respectively

L⁴-COR⁵ (X)

L⁴-CONR⁵R^{5a} (XI)

where L⁴ is a leaving group such as halo for instance chloro, and R⁵ is defined in relation to formula (I);
and thereafter, if desired or necessary, carrying out one or more of the following steps:
converting the compound obtained to a further compound of formula (I)
removal of any protecting groups
forming a pharmaceutically acceptable salt of the compound.

In reactions (a) and (c) above, suitable leaving groups L¹ and L² are halogen atoms such as bromine, or chlorine, as well as an activated alcohol such as mesylate or tosylate. The reactions may conveniently be carried out in an organic solvent such as acetonitrile, 1-methyl-2-pyrrolidinone or N,N-dimethylformamide at a temperature, for example, in the range from 0 to 150° C. The reaction may be suitably effected by the presence of a base (e.g. sodium carbonate or potassium carbonate).

In process (b), the reaction may conveniently be carried out in an organic solvent such as 1-methyl-2-pyrrolidinone, 1,2-dichloroethane or tetrahydrofuran at a temperature, for example, in the range from 0 to 100° C.

Compounds of formula (II) may be prepared as illustrated in the reaction scheme

### A:

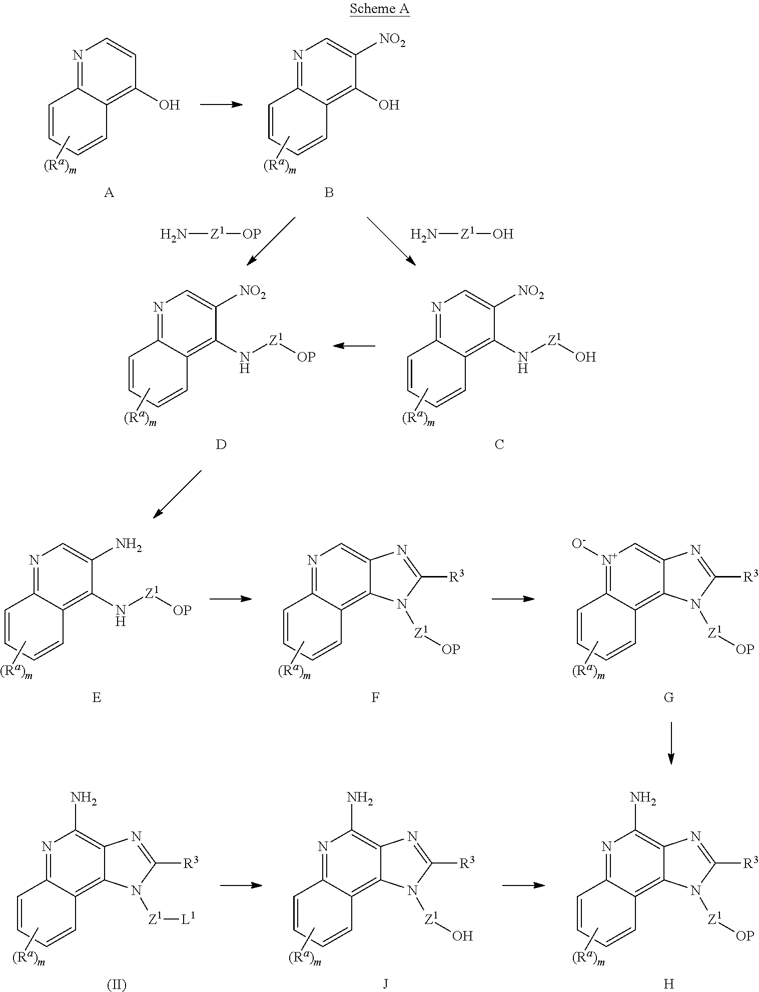

where R^{a}, m, R³ and Z¹ are as defined in relation to formula (I) and P is a protecting group.

The compound of formula (B) is prepared by nitration of a compound of formula (A). Suitable nitrating agents include nitric acid. The reaction is suitably effected in an organic solvent such as an organic acid such as propionic acid. The reaction may be carried out at elevated temperature, for example from room temperature to 150° C.

Compounds of formula (C) may be prepared by reacting the compound of formula (B) with a mixture of thionyl chloride and DMF to give the aryl chloride which can then be displaced with an aminoalkanol. The chlorination is suitably carried out in a solvent such as dichloromethane, preferably at elevated temperature. The displacement of the chloride with an aminoalkanol, is suitably carried out in the presence of a base for example triethylamine or Hunigs base and in an organic solvent such as dichloromethane, at a temperature in the range from 0 to 40° C.

Compounds of formula (D) are prepared by adding a suitable protecting group to the hydroxy terminal group. This can be effected using conventional chemistry as outlined for example in ‘Protective Groups in Organic Synthesis’ by Theodora Green (publisher: John Wiley 85 Sons). A suitable protecting group P for the hydroxy group is, for example, an alkanoyl group such as acetyl, an aroyl group, for example benzoyl, or an arylmethyl group, for example benzyl, or a silyl group for example tert-butyl(dimethyl)silyl.

Compounds of formula (D) may also be prepared by adding a protected aminoalkanol to a compound of formula (B), using the same conditions as above.

The compound of formula (D) is then reduced to form a compound of formula (E). Suitable reducing agents include iron powder in a suitable solvent such as acetic acid or sodium borohydride in the presence of a suitable catalyst such as a 15% of nickel chloride in a suitable solvent such as methanol or hydrogenation. Suitable hydrogenation conditions include the use of hydrogen gas at elevated pressure, for example at 2-5 Bar in the presence of a suitable catalyst such as a 1% platinum on carbon catalyst. The reaction is suitably effected at room temperature.

Compounds of formula (E) are then cyclised to form the compound of formula (F). Suitable cyclisation conditions include reaction with an acid chloride in the presence of a base such as triethylamine in a suitable solvent such as N-methyl pyrrolidinone or an acid in the presence of a coupling reagent such as O-(7-azabenzotriazol-1-yl)-N,N,N′,N′-tetramethyluroniumhexafluorophosphat purum (HATU) in the presence of a base such as triethylamine in a suitable solvent such as N-methylpyrrolidine. Alternatively the compound of formula (F) may be prepared by cyclisation reaction with an orthoester in a suitable solvent such as N-methylpyrrolidinone in the presence of a suitable catalyst such as 10 mol % of toluensulphuric acid. The reaction is suitably effected at elevated temperatures, for example from 30-150° C.

Compounds of formula (F) may be oxidised to compounds of formula (G) by reaction with an oxidising agent such as meta-chloroperoxybenzoic acid or hydrogen peroxide. The reaction is suitably effected in an organic solvent such as dichloromethane or ethanol at reduced temperatures for example in the range of −10° C. to room temperature.

Subsequently, the compound of formula (G) is reacted with p-toluenesulphonyl chloride and aqueous ammonia to convert it to the compound of formula (H). The reaction is suitably effected in an organic solvent such as dichloromethane. Temperatures in the range from 0-40° C. and conveniently at room temperature are suitably employed.

Deprotection of the resultant compound of formula (H) yields a compound of formula (J). The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively a benzyl group may be removed, for example, by hydrogenation over a catalyst such as palladium-on-carbon.

The product of formula (J) is then converted to a compound of formula (II) by formation of a suitable leaving group such as halo, for instance chloro or bromo, or an activated alcohol such as a mesylate or tosylate. For example, the chloride may be formed by reacting the compound of formula (J) with thionyl chloride, preferably in a solvent such as dichloromethane at a temperature between 20-40° C.

Compounds of formulae (IV) and (IVA) may be prepared by an analogous route as illustrated in Scheme B.

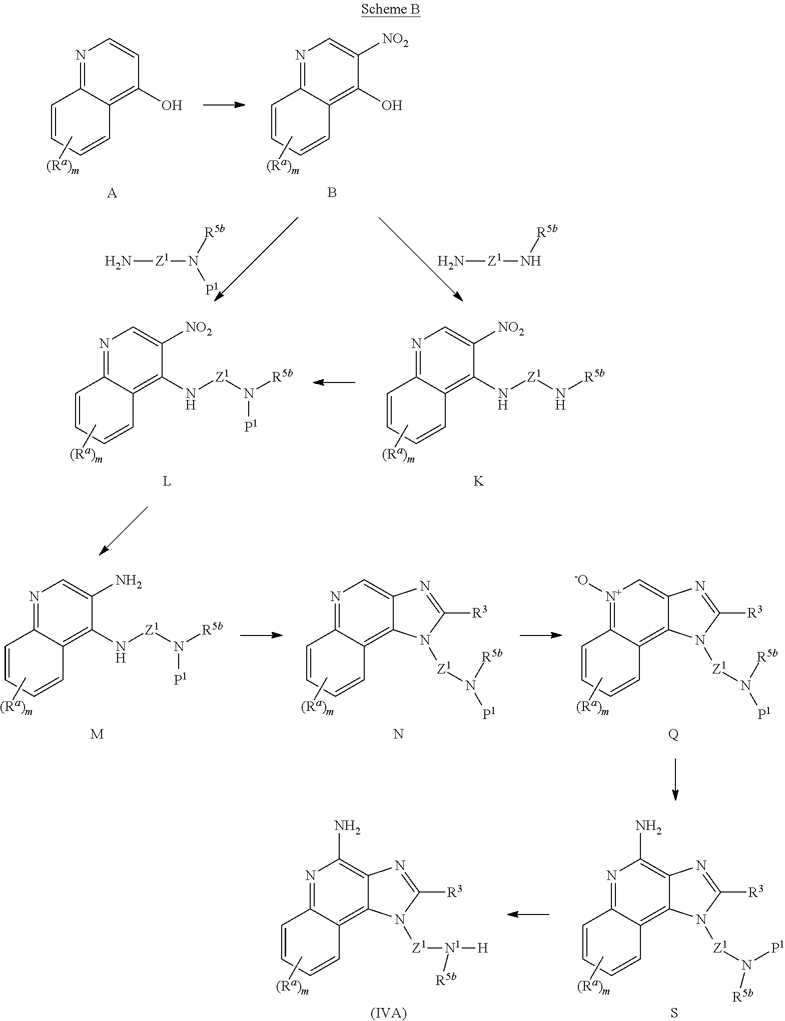

where R^{a}, m, R³ and Z¹ are as defined in relation to formula (I), R^{5b} is as defined in relation to formula (IVA) and P¹ is an amino protecting group.

Compounds of formula (K) or (L) may be prepared by reacting the compound of formula (B) with a mixture of thionyl chloride and DMF to give the aryl chloride which can then be displaced with a di-amino alkane, or a protected form thereof. The chlorination is suitably carried out in a solvent such as dichloromethane, preferably at elevated temperature. The displacement of the chloride with a di-amino alkane, or a protected form thereof, is suitably carried out in the presence of a base for example triethylamine or Hunigs base and in an organic solvent such as dichloromethane, at a temperature in the range from 0 to 40° C.

Where a diaminoalkane is used, a compound of formula (K) is prepared which may be subsequently protected to form a compound of formula (L) using conventional methods.

A suitable protecting group P¹ is for example, a group such as an alkoxycarbonyl group, for example a methoxycarbonyl, ethoxycarbonyl or t-butoxycarbonyl group, an arylmethoxycarbonyl group, for example benzyloxycarbonyl. A suitable alternative protecting group for a primary amino group is, for example, a phthaloyl group.

Reduction of the product of formula (L) using for example analogous conditions to those described above for the reduction of the compound of formula (D), will yield a compound of formula (M). This in turn may be cyclised to a compound of formula (N) using conditions analogous to those described above for the cyclisation of the compound of formula (E), oxidised to a compound of formula (O) using conditions analogous to those described above for the oxidation of the compound of formula (F), and the product reacted with p-toluenesulphonyl chloride and aqueous ammonia to form the compound of formula (S) using for example conditions analogous to those described above for the preparation of the compound of formula (H).

Deprotection of the resultant compound of formula (S) yields a compound of formula (IV). The deprotection conditions for the above protecting groups necessarily vary with the choice of protecting group. Thus, for example, an alkoxycarbonyl group may be removed for example, by hydrolysis with a suitable base such as an alkali metal hydroxide, for example lithium or sodium hydroxide. Alternatively an alkoxycarbonyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A phthaloyl protecting group which be removed by treatment with an alkylamine, for example dimethylaminopropylamine, or with hydrazine.

Suitably in Scheme B, R⁵ is hydrogen, which may be converted to a different R⁵ group later, for example once the compound of formula (IV) has been converted to a compound of formula (I).

Compounds of formula (VI) where X³ is NR⁵ may be prepared by reacting compounds of formula (II) with compounds of formula (XI):

R⁵NH₂ (XI)

Coupling Conditions Will be Similar to Those Described Above for the Reactions (a) and (c).

Compounds of formula (I) may be converted to other compounds of formula (I) using conventional methods. For example, in process (h) above, compounds where R⁵ is hydrogen may be reacted with compounds of formula (X):

L⁴-COR⁵ (X)

where L⁴ is a leaving group such as halo for instance chloro, and R⁵ is defined in relation to formula (I). The reaction is suitably carried out in an organic solvent such as acetonitrile, dimethylformamide and/or dichloromethane optionally in the presence of a base such as triethylamine. Temperatures in the range from 0 to 150° C. are suitably employed.

Similarly, oxidation of compounds of formula (I) during process (d) above can be carried out under conventional conditions, for example by reaction with an oxidising agent such as meta-chloroperoxybenzoic acid or hydrogen peroxide. The reaction is suitably effected in an organic solvent such as dichloromethane or ethanol at temperatures for example in the range of 0-40° C.

Compounds of formula (IX) above where X⁴ is an activated acid such as an acid chloride are suitably prepared by a reaction as set out in Scheme C.

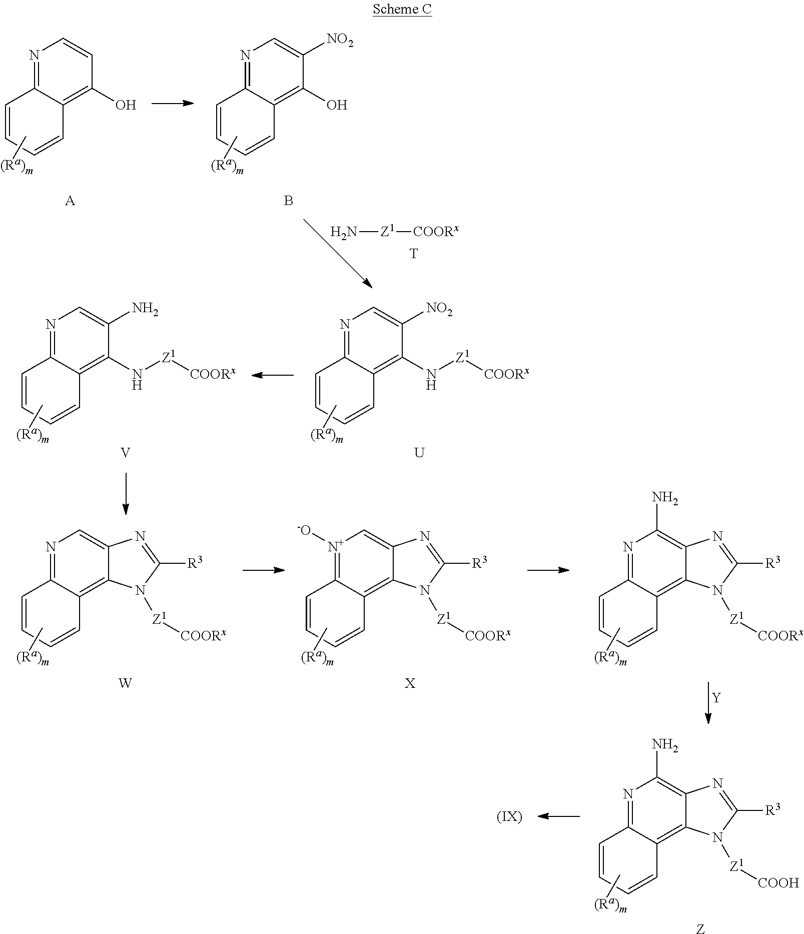

where R^{a}, m, R³ and Z¹ are as defined in relation to formula (I), R^{x} is an alkyl such as methyl or ethyl, or ester protecting group.

Conditions used for the reactions shown in Scheme C are generally similar to those used in analogous steps in Scheme B. A compound of formula Y may be converted to a compound of formula Z with a base such as lithium or sodium hydroxide, in a suitable solvent such as tetrahydrofuran or methanol and water. Alternatively the ester may be hydrolysed under acidic conditions such as aqueous HCl, preferably at elevated temperature. A compound of formula (IX) may be prepared from a compound of formula (Z) by activation of the acid to an acyl halide, such as chloride with a reagent such as thionyl chloride then treated with a compound of formula (III). The formation of the acid chloride may conveniently be carried out neat or in an organic solvent such as dichloromethane at a temperature, for example, in the range from 0 to 80° C. The activated acid is then treated with a compound of formula (III), the reaction may conveniently be carried out in an organic solvent such as tetrahydrofuran or dimethylformamide, with a base such as triethylamine at a temperature, for example, in the range from 0 to 80° C. Alternatively the acid may be activated with a coupling agent such as 1,3-dicyclohexylcarbodiimide or benzotriazol-1-yloxytripyrrolidinophosphonium hexafluorophosphate.

A compound of formula (I) in which X¹ is NR⁵ and R⁵ is hydrogen may be converted to a corresponding compound of formula (I) in which R⁵ is —COCH₂NR⁷R⁸ by reaction with chloroacetyl chloride followed by an amine of formula R⁷R⁸NH where R⁷ and R⁸ are as defined above. The first stage is suitably carried out in an organic solvent such as chloroform, dichloromethane or acetonitrile, with one equivalent of chloroacetyl chloride. Temperatures in the range from 0 to 50° C. are suitably employed. In the second stage the reaction is suitably carried out in an organic solvent such as dichloromethane or acetonitrile, with excess of an amine R⁷R⁸NH. Temperatures in the range from 0° C. to 100° C. are suitably employed.

A compound of formula (I) in which X¹ is NR⁵ and R⁵ is hydrogen may also be converted to a corresponding compound of formula (I) in which R⁵ is a C₁-C₆ alkyl (e.g. propyl) group substituted by NR⁷R⁸ by reaction with a compound of formula (XX), L¹⁰-R⁵, where L¹⁰ is a leaving group such as halo for instance chloro and R⁵ is as defined above. The reaction is suitably carried out in an organic solvent such as dimethylformaldehyde or acetonitrile, with preferably one equivalent of formula (XX) compound optionally in the presence of a base such as triethylamine and a salt such as sodium iodide or potassium iodide. Temperatures in the range from 0° C. to 100° C. are suitably employed.

A compound of formula (I) in which X¹ is NR⁵ and R⁵ is a C₁-C₆ alkyl (e.g. propyl) group substituted by NR⁷R⁵ may also be prepared by reacting a compound of formula (XII):

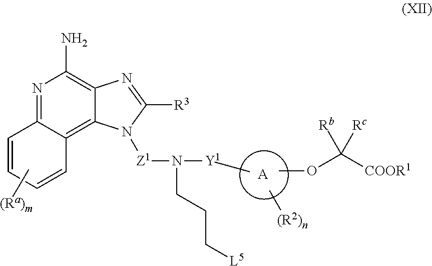

where L⁵ is a leaving group for example chloro or mesylate and m R^{a}, R¹, n, R², R³, A, Z¹ and Y¹ are as defined above, with an amine of formula (XXI), R⁷R⁵NH, where R⁷ and R⁸ are as defined above. The reaction may be carried out using an excess of the amine R⁷R⁵NH in an organic solvent such as DMF or dioxane at a temperature in the range of, for example, 40° C.-150° C. Sodium iodide may be used as an additive in the reaction.

A compound of formula (XII) may be prepared from a corresponding compound of formula (XIII):

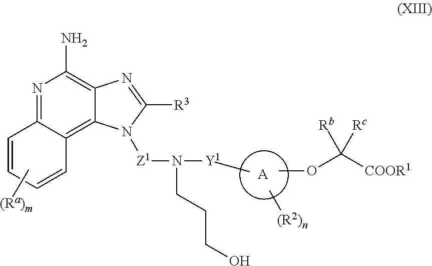

The alcohol may be converted into a leaving group using conventional methods, for example, by reaction with thionyl chloride in an appropriate solvent such as DCM at a temperature from 20-100° C.

A compound of formula (XIII) may be formed using the route in scheme A and the chemistry above.

Compounds of formulae (III), (V), (VII), (VIII), A, (X), (XI), (XX) and (XXI) are known compounds or can be prepared from known compounds by conventional methods.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

In another embodiment of the invention there is provided an intermediate compound of the formula (I′):

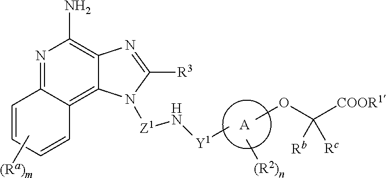

wherein Z¹, Y¹, R^{a}, R^{b}, R^{c}, R², R³, m and n are as defined in claim 1; and R^{1′} represents hydrogen, C₁-C₈ alkyl, C₃-C₈ cycloalkyl, or a 3- to 8-membered saturated heterocyclic ring group comprising a O atom, wherein R^{1′} is optionally substituted by one or more substituents independently selected from halogen, cyano, hydroxyl and C₁-C₃ alkoxy;
or a salt thereof, for synthesis of a compound of formula (I) or its pharmaceutically acceptable salt.

The compounds of formula (I) above may be converted to a pharmaceutically acceptable salt thereof, preferably an acid addition salt such as a hydrochloride, hydrobromide, trifluoroacetate, sulphate, phosphate, acetate, fumarate, maleate, tartrate, lactate, citrate, pyruvate, succinate, oxalate, methanesulphonate or p-toluenesulphonate. Preferred salts include dimethane sulphonic acid, monosaccharin, disaccharin, di-1-hydroxy-2-naphthoic acid (di-xinafoate), dibenzenesulphonic acid (di-besylate), mandelic and fumaric acid salts.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as modulators of toll-like receptor (especially TLR7) activity, and is expected to provide an immuno-modulator effect and thus be useful as a therapeutic and prophylactic agent for diseases associated with an abnormal immune response (e.g. autoimmune diseases and allergic diseases) and various infections and cancers which are required for activation of an immune response. Compound (1), or a pharmaceutically acceptable salt thereof may also be useful as a vaccine adjuvant. For example, Compound (1), or a pharmaceutically acceptable salt thereof, may be administered to a mammal, including man, for the treatment of the following conditions or diseases:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, actinic keratosis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; hemangioma; pre-cancerous skin lesions; basal cell carcinoma, for example superficial basal cell carcinoma, nodular basal cell carcinoma and bowen's disease; cutaneous lymphomas, non-melanoma skin cancer and other dysplastic lesions; drug-induced disorders including fixed drug eruptions, skin scarring, including keloids; cutaneous infections, including viral cutaneous infections; and cosmetic effects including photo-damaged skin;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carnii, cryptosporidiosis, histoplasmosis, toxoplasmosis, trypanosome infection and leishmaniasis.

The compounds of formula (I) and their pharmaceutically acceptable salts have antedrug properties. An antedrug is defined as an active synthetic derivative that is designed to undergo biotransformations to a readily excretable less active form upon entry into the systemic circulation, therefore minimizing systemic side-effects. Thus, on administration, a compound of the invention is rapidly degraded enzymatically to yield a degradation product having a substantially reduced medical effect. A medical effect as defined herein means a pharmacological activity of the compound of the invention, including specifically interferon inducing activity and/or suppression of IL4/IL5 production activity.

The medical effect of the degradation product is preferably 10 times, more preferably 100 times less than that of the compound of the invention (i.e. parent compound).

The pharmacological activity can be measured using methods known in the art, preferably using in vitro evaluation methods such as commercially available ELISA kits or the biological assay described in Example 7 of the present specification.

Furthermore a compound of formula (I) or a pharmaceutically acceptable salt thereof, wherein R¹ represents branched chain alkyl, ⁱPr, or R^{b} and R^{c} represent methyl show good chemical stability.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In particular, the compounds of the invention may be used in the treatment of asthma, COPD, allergic rhinitis, allergic conjunctivitis, cancer, hepatitis B, hepatitis C, HIV, HPV, bacterial infections or a skin condition as listed hereinbefore (for example, atopic dermatitis, actinic keratosis, pre-cancerous skin lesions or cutaneous vial infections). Compound (1), or a pharmaceutically acceptable salt thereof, may also be useful as a vaccine adjuvant.

The anti-cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, hydroxyurea or paclitaxel); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention still further provides a method of treating, or reducing the risk of, an obstructive airways disease or condition (e.g. asthma or COPD) which comprises administering to a patient in need thereof a therapeutically effective amount of a compound of formula (I) or a pharmaceutically acceptable salt thereof as hereinbefore defined.

Accordingly, as a further aspect of the invention there is provided Compound (1),

or a pharmaceutically acceptable salt thereof, for use in the treatment of asthma, COPD or allergic rhinitis.

As a further aspect of the invention, there is provided Compound (1), or a pharmaceutically acceptable salt thereof, for use in the treatment of asthma.

As a further aspect of the invention, there is provided Compound (1), or a pharmaceutically acceptable salt thereof, for use in the treatment of COPD.

As a further aspect of the invention, there is provided Compound (1), or a pharmaceutically acceptable salt thereof, for use in the treatment of allergic rhinitis.

As a further aspect of the invention, there is provided Compound (1), or a pharmaceutically acceptable salt thereof, for use as a vaccine adjuvant.

As a further aspect of the invention, there is provided Compound (1), or a pharmaceutically acceptable salt thereof, for use in the treatment of a skin condition as hereinbefore described (for example atopic dermatitis, actinic keratosis, pre-cancerous lesions or cutaneous vial infections).

As a further aspect of the invention, there is provided the use of Compound (1), or a pharmaceutically acceptable salt thereof, in the manufacture of a medicament for the treatment of asthma, COPD or allergic rhinitis.

As a further aspect of the invention, there is provided the use of Compound (1), or a pharmaceutically acceptable salt thereof, in the manufacture of a medicament for the treatment of asthma.

As a further aspect of the invention, there is provided the use of Compound (1), or a pharmaceutically acceptable salt thereof, in the manufacture of a medicament for the treatment of COPD.

As a further aspect of the invention, there is provided the use of Compound (1), or a pharmaceutically acceptable salt thereof, in the manufacture of a medicament for the treatment of allergic rhinitis.

As a further aspect of the invention, there is provided the use of Compound (1), or a pharmaceutically acceptable salt thereof, in the manufacture of a medicament for the treatment of a skin condition as hereinbefore described (for example atopic dermatitis, actinic keratosis, pre-cancerous lesions or cutaneous vial infections).

As a further aspect of the invention, there is provided the use of Compound (1), or a pharmaceutically acceptable salt thereof, as a vaccine adjuvant, in the manufacture of a vaccine for the treatment of a disease or condition.

The invention therefore provides a method of treating an inflammatory disease in a patient suffering from, or at risk of, said disease, which comprises administering to the patient a therapeutically effective amount of Compound (1), or a pharmaceutically acceptable salt thereof.

The invention also provides a method of treating an airways disease, e.g. a reversible obstructive airways disease such as asthma, in a patient suffering from, or at risk of, said disease, which comprises administering to the patient a therapeutically effective amount of Compound (1), or a pharmaceutically acceptable salt thereof.

The invention still further provides a method of treating, or reducing the risk of, a disease or condition comprising or arising from abnormal cell growth (e.g. a cancer), which method comprises administering to a patient in need thereof a therapeutically effective amount of Compound (1), or a pharmaceutically acceptable salt thereof.

The invention still further provides a method of treating, or reducing the risk of, a skin disease or condition as hereinbefore described (for example atopic dermatitis, actinic keratosis, pre-cancerous lesions or cutaneous vial infections), which method comprises administering to a patient in need thereof a therapeutically effective amount of Compound (1), or a pharmaceutically acceptable salt thereof. The invention still further provides a method of treating, or reducing the risk of, a disease or condition, which method comprises administering to a patient in need thereof a therapeutically effective amount of a vaccine and a salt of Compound (1) defined herein or a solvate of the salt.

The invention still further provides a method of increasing the response to a vaccine in a patient, which method comprises administering to a patient in need thereof a therapeutically effective amount of a vaccine and Compound (1), or a pharmaceutically acceptable salt thereof.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). For example, a dose of about 0.1 to 100 μg/kg such as a dose of about 0.1, 0.5, 1, 2, 5, 10, 20, 50 or 100 μg/kg. Alternatively, if the compound is administered orally, then the daily dosage of the compound of the invention may be in the range from 0.01 micrograms per kilogram body weight (μg/kg) to 100 milligrams per kilogram body weight (mg/kg).

The dosages mentioned herein refer to the dose of Compound (1) as the free base. Accordingly, the equivalent dose of a particular salt will be higher because of the increased molecular weight of the salt compared to the free base.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (including to the skin, eye, buccal cavity, respiratory tract or nasally) in the form, e.g., of creams, solutions, suspensions, heptafluoroalkane (HFA) aerosols and dry powder formulations, for example, formulations administered from a suitable device such as a pressurised metered dose inhaler (pMDI), a dry powder inhaler (DPI) or nebuliser, such as the inhaler device known as the Turbuhaler™; or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the invention (including pharmaceutically acceptable salts) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant. For nasal administration the compound is suitably dissolved in an aqueous medium, which is suitably buffered to maintain the pH at a desired level.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler™ in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

In particular, for the treatment of the inflammatory diseases COPD, asthma and allergic rhinitis the compounds of the invention may be combined with agents such as tumour necrosis factor alpha (TNF-alpha) inhibitors such as anti-TNF monoclonal antibodies (for example Remicade, CDP-870 and adalimumab) and TNF receptor immunoglobulin molecules (such as Enbrel); non-selective cyclo-oxygenase COX-1/COX-2 inhibitors whether applied topically or systemically (such as piroxicam, diclofenac, propionic acids such as naproxen, flubiprofen, fenoprofen, ketoprofen and ibuprofen, fenamates such as mefenamic acid, indomethacin, sulindac, azapropazone, pyrazolones such as phenylbutazone, salicylates such as aspirin), COX-2 inhibitors (such as meloxicam, celecoxib, rofecoxib, valdecoxib, lumarocoxib, parecoxib and etoricoxib); glucocorticosteroids (whether administered by topical, oral, intramuscular, intravenous, or intra-articular routes); methotrexate, lefunomide; hydroxychloroquine, d-penicillamine, auranofin or other parenteral or oral gold preparations.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention and a receptor antagonist for leukotrienes (LT B4, LTC4, LTD4, and LTE4) selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention and an anticholinergic agent including muscarinic receptor (M1, M2, and M3) antagonists such as atropine, hyoscine, glycopyrrolate, ipratropium bromide, tiotropium bromide, oxitropium bromide, pirenzepine or telenzepine.

The present invention still further relates to the combination of a compound of the invention together with a beta-adrenoceptor agonist (including beta receptor subtypes 1-4) such as isoprenaline, salbutamol, formoterol, salmeterol, terbutaline, orciprenaline, bitolterol mesylate, and pirbuterol.

The present invention further relates to the combination of a compound of the invention and a chromone, such as sodium cromoglycate or nedocromil sodium.

The present invention still further relates to the combination of a compound of the invention together with an insulin-like growth factor type I (IGF-1) mimetic.

The present invention still further relates to the combination of a compound of the invention and a glucocorticoid, such as flunisolide, triamcinolone acetonide, beclomethasone dipropionate, budesonide, fluticasone propionate, ciclesonide or mometasone furoate.

The present invention still further relates to the combination of a compound of the invention together with an inhibitor of matrix metallo proteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12.

The present invention still further relates to the combination of a compound of the invention together with modulators of chemokine receptor function such as antagonists of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX3CR1 for the C—X3-C family.

The present invention still further relates to the combination of a compound of the invention together with a cytokine or modulator of cytokine function, including alpha-, beta-, and gamma-interferon; interleukins (IL) including IL1 to 15, and interleukin antagonists or inhibitors, including agents which act on cytokine signalling pathways.

The present invention still further relates to the combination of a compound of the invention together with an immunoglobulin (Ig) or Ig preparation or an antagonist or antibody modulating Ig function such as anti-IgE (omalizumab).

The present invention further relates to the combination of a compound of the invention and another systemic or topically-applied anti-inflammatory agent, such as thalidomide or a derivative thereof, a retinoid, dithranol or calcipotriol.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention will be further explained by reference to the following illustrative examples.

### EXPERIMENTAL

Unless otherwise stated organic solutions were dried over magnesium sulphate. RPHPLC means reversed phase preparative HPLC using Waters Symmetry C8, Xterra, Xbridge or Phenomenex Gemini columns using acetonitrile and either aqueous ammonium acetate, ammonia, formic acid or trifluoroacetic acid as buffer where appropriate. Column chromatography was carried out on silica gel. Treating with SCX means the mixture was absorbed on SCX and eluted with an appropriate solvent such as methanol or acetonitrile then the free base product eluted with aqueous ammonia/methanol.

The following abbreviations are used;
EtOAc ethyl acetate
DCM dichloromethane
NMP N-methylpyrrolidinone
NBS N-bromosuccinimide
DMF N,N-dimethylformamide
DMSO dimethylsulfoxide
THF tetrahydrofuran
MeOH methanol
TFA trifluoroacetic acid
HCl hydrogen chloride
K₂CO₃ potassium carbonate
NaHCO₃ sodium hydrogen carbonate
TEA triethylamine
MeCN acetonitrile
HATU O-(7-azabezotriazol-1-yl)-N,N,N′, N′-tetramethyluronium hexafluorophosphate
EDCI N-(3-dimethylaminopropyl)-N′-ethylcarbodiimide hydrochloride
HOBt 1-hydroxybenzotriazole
rt room temperature
h hours
min minutes
M molar
MS mass spectrometry
PyBop Benzotriazol-1-yloxytripyrrolidinophosphonium hexafluorophosphate
APCI atmospheric chemical ionisation method
ESI electron spray ionisation method
NMR nuclear magnetic resonance

### Example 1

### Methyl 2-(3-{[3-(4-amino-2-butyl-1H-imidazo[4,5-c]quinolin-1-yl)propylamino]methyl}phenoxy)acetate

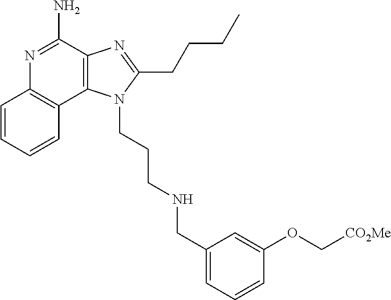

### (i) 3-Nitroquinolin-4-ol

4-Hydroxyquinoline (79.3 g) and propionic acid (790 mL) were combined and heated to 125° C. Nitric acid (79 mL) was added dropwise over 40 minutes. The reaction mixture was stirred at reflux temperature for a further 3 h and cooled to rt. The mixture was diluted with ethanol and the solid was collected by vacuum filtration. The solid was washed with ethanol, water then ethanol. The residue was refluxed in ethanol and the hot mixture was filtered and dried to give the subtitle compound (80.9 g). Yield: 76%

¹H NMR δ (DMSO-d₆) 13.00 (1H, s), 9.19 (1H, s), 8.26-8.23 (1H, m), 7.81-7.77 (1H, m), 7.75-7.71 (1H, m), 7.53-7.49 (1H, m)

### (ii) tert-Butyl {3-[(3-nitroquinolin-4-yl)amino]propyl}carbamate

To a stirred solution of 3-nitroquinolin-4-ol (30 g) in DCM (250 mL) was added DMF (6 mL) and thionyl chloride (13.9 mL) and the reaction mixture was refluxed for 2.5 h when all solids dissolved. The solution was cooled to 0° C. and a solution of (3-aminopropyl)-carbamic acid tert-butyl ester (45.6 g) and Et₃N (67 mL) in DCM (250 mL) was added dropwise. The reaction mixture was stirred overnight and then evaporated. Potassium carbonate solution and MTBE were added to the residue and stirred for 1 h. The product was filtered and washed with water and MTBE and dried to give the subtitle compound (50.7 g). Yield: 94%

¹H NMR δ (CDCl₃) 9.66 (1H, s), 9.36 (1H, s), 8.31-8.29 (1H, m), 7.98-7.95 (1H, m), 7.77-7.72 (1H, m), 7.48-7.44 (1H, m), 4.67 (1H, s), 4.00-3.96 (2H, m), 3.34-3.29 (2H, m), 2.03-1.96 (2H, m), 1.41 (9H, s)

MS: ESI 347 (M+1)

### (iii) tert-Butyl {3-[(3-aminoquinolin-4-yl)amino]propyl}carbamate NiCl₂6H₂O (15.4 g) was dissolved in MeOH (220 ml) and cooled to 5-10° C. After the addition of sodium borohydride (2.4 g), the product from step (ii) (22.4 g) was added. More sodium borohydride (9.8 g) was added slowly under 23° C. and the reaction mixture was stirred for 1 h. The reaction mixture was filtered using celite and the filtrate was poured into sodium bicarbonate solution (300 ml). After removal of the 250 ml of solvent, extracted with chloroform, dried, filtered and evaporated to give the subtitle compound (21.7 g). Yield 85%.

¹H NMR δ (DMSO-d₆) 8.36 (1H, s), 8.00-7.97 (1H, m), 7.72-7.70 (1H, m), 7.36-7.29 (2H, m), 6.87-6.84 (1H, m), 5.00 (2H, s), 4.76 (1H, t, J=6.4 Hz), 3.13-3.09 (2H, m), 3.01-2.97 (2H, m), 1.62-1.58 (2H, m), 1.39 (9H, s)

### (iv) tert-Butyl [3-(2-butyl-1H-imidazo[4,5-c]quinolin-1-yl)propyl]carbamate

The product from step (iii) (49.7 g) was dissolved in NMP (150 mL) and ortho-valeric acid triethyl ester (54.6 mL) and para-toluene sulufonic acid mono hydrate (2.7 g) were added. The reaction mixture was stirred at 80° C. for 1 h. Sodium bicarbonate solution (300 ml), water (500 ml) and diethyl ether (200 ml) were added to the reaction mixture and stirred for 1 h. The solid precipitate was filtered and washed with water and diethyl ether to give subtitle compound (44.8 g).

¹H NMR δ (DMSO-d₆) 9.12 (1H, s), 8.37-8.35 (1H, m), 8.15-8.12 (1H, m), 7.69-7.66 (2H, m), 7.15-7.10 (1H, brs), 4.59 (2H, t, J=7.6 Hz), 3.11-3.07 (2H, m), 2.95 (2H, t, J=7.2 Hz), 1.97-1.92 (2H, m), 1.86-1.81 (2H, m), 1.48-1.37 (11H, m), 0.95 (3H, t, J=7.6 Hz),

MS: ESI 383 (M+1)

### (v) tert-Butyl [3-(2-butyl-5-oxido-1H-imidazo[4,5-c]quinolin-1-yl)propyl]carbamate

The product from step (iv) (42 g) was dissolved in DCM (2000 mL) and cooled to 5° C. 3-Chloroperoxybenzoic acid (38 g) was added and the reaction was allowed to warm to room temperature. The reaction mixture was stirred for 12 h. The reaction mixture was washed with saturated sodium thiosulfate solution and sodium bicarbonate solution, dried, filtered and evaporated to give the subtitle compound (48 g).

MS: ESI 399 (M+1)

### (vi) tert-Butyl [3-(4-amino-2-butyl-1H-imidazo[4,5-c]quinolin-1-yl)propyl]carbamate

p-Toluenesulphonyl chloride (25 g) was added portionwise to a vigorously stirred mixture of the product from step (v) (48 g) in DCM (420 mL) and ammonium hydroxide solution (35%, 2.5 mL) at 0° C. The mixture was allowed to warm to rt over night then partitioned between water/DCM, washed with saturated sodium bicarbonate solution, dried, filtered and the solvent evaporated. The solid product was recrystallized from the mixture of MeOH and acetonitrile to give the subtitle compound (25 g) (yield 57% by 2 steps).

¹H NMR δ (DMSO-d₆) 8.04-8.02 (1H, m), 7.60-7.57 (1H, m), 7.42-7.38 (1H, m), 7.24-7.20 (1H, m), 7.14-7.11 (1H, m), 6.45 (2H, s), 4.48 (2H, t, J=7.6 Hz), 3.11-3.06 (2H, m), 2.91-2.87 (2H, m), 1.93-1.89 (2H, m), 1.82-1.75 (2H, m), 1.47-1.37 (11H, m), 0.97 (3H, t, J=7.6 Hz),

MS: ESI 398 (M+1)

### (vii) 1-(3-Aminopropyl)-2-butyl-1H-imidazo[4,5-c]quinolin-4-amine

The product from step (vi) (124 g) was suspended in EtOH (270 mL) and 6N HCl (270 mL) was added. The reaction mixture was stirred at 50° C. for 1 h. After the removal of the 300 ml of the solvent, the residue was washed with chloroform and then poured into 7% NH₃ solution, extracted with EtOH/CHCl₃ (1/5), dried and evaporated to give the subtitle compound (63 g). Yield 94%.

¹H NMR δ (CDCl₃) 8.12 (1H, d, J=7.2 Hz), 7.60-7.58 (1H, m), 7.41-7.37 (1H, m), 7.25-7.21 (1H, m), 6.43 (2H, s), 4.55 (2H, t, J=7.6 Hz), 2.93 (2H, t, J=7.6 Hz), 2.67 (2H, t, J=7.6 Hz), 1.87-1.75 (4H, m), 1.55-1.41 (4H, m), 0.95 (3H, t, J=7.6 Hz).

MS: ESI 298 (M+1)

### (viii) Methyl 2-(3-{[3-(4-amino-2-butyl-1H-imidazo[4,5-c]quinolin-1-yl)propylamino]methyl}phenoxy)acetate

To a solution of the product from step (vii) (5.01 g, 16.8 mmol) in MeOH (75 ml) were added methyl (3-formylphenoxy)acetate (3.26 g, 16.8 mmol), AcOH (1.94 ml, 33.6 mmol) and NaBH₃CN (2.21 g, 33.7 mmol) at room temperature. After stirring for 26 h at the same temperature, the reaction mixture was concentrated. The residue was washed with 1% NH₃ aq. (100 ml), and extracted with CHCl₃ (100 ml×3). The combined extracts were dried over MgSO₄ and concentrated. The residue was purified by flash column chromatography to afford the subtitle compound (5.38 g, 67%) as colorless amorphous.

¹H NMR δ (CDCl₃) 8.13 (1H, d, J=8.2), 7.60 (1H, d, J=8.2), 7.40 (1H, dd, J=7.2, 7.9), 7.26-7.18 (2H, m), 6.97-6.95 (2H, m), 6.81-6.77 (1H, m), 6.46 (2H, brs), 4.78 (2H, s), 4.58 (2H, brt, J=7.1), 3.68 (3H, s), 3.68 (2H, s), 2.94 (2H, t, J=7.7), 2.62-2.58 (2H, m), 2.38 (1H, brs), 2.00-1.91 (2H, m), 1.79 (2H, tt, J=7.5, 7.7), 1.44 (2H, qt, J=7.3, 7.5), 0.95 (3H, t, J=7.3).

### Example 2

### Methyl (3-{[[3-(4-amino-2-butyl-1H-imidazo[4,5-c]quinolin-1-yl)propyl](chloroacetyl)amino]methyl}phenoxy)acetate hydrochloride

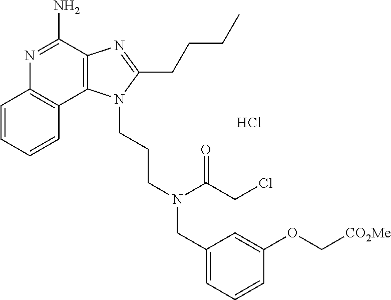

To a solution of the product from example 1 (5.38 g, 11.3 mmol) in CHCl₃ (160 ml) was added chloroacetyl chloride (0.9 ml, 11.3 mmol) at 0° C. After stirring for 2 h at the same temperature, the reaction mixture was quenched by 0.2N HCl (220 ml). The aq. layer was extracted with CHCl₃ (220 ml×3), dried over MgSO₄, and concentrated. The residue was purified by flash column chromatography to give the title compound (6.46 g, 97%) as a white solid.

¹H NMR δ (DMSO-d₆) 13.69 (1H, brs), 8.58 (1H, brs), 8.17 (½H, d, J=8.4), 8.13 (½H, d, J=8.2), 7.83-7.81 (1H, m), 7.71 (½H, d, J=7.5), 7.69 (½H, d, J=8.0), 7.56-7.50 (1H, m), 7.22 (½H, dd, J=7.7, 7.9), 7.17 (½H, dd, J=7.8, 7.9), 6.82-6.73 (3H, m), 4.75 (1H, s), 4.72 (1H, s), 4.64-4.57 (1H, m), 4.58 (1H, s), 4.56-4.47 (1H, m), 4.51 (1H, s), 4.49 (1H, s), 4.42 (1H, s), 3.69 ( 3/2H, s), 3.68 ( 3/2H, s), 3.53 (1H, brt, J=7.6), 3.45 (1H, brt, J=7.6), 2.95 (1H, dd, J=7.7, 7.8), 2.92 (1H, dd, J=7.6, 7.8), 2.11-1.92 (2H, m), 1.83-1.77 (2H, m), 1.50-1.39 (2H, m), 0.96 ( 3/2H, t, J=7.4), 0.95 ( 3/2H, t, J=7.3).

### Example 3

### Methyl (4-{[[3-(4-amino-2-butyl-1H-imidazo[4,5-c]quinolin-1-yl)propyl]amino]methyl}phenoxy)acetate

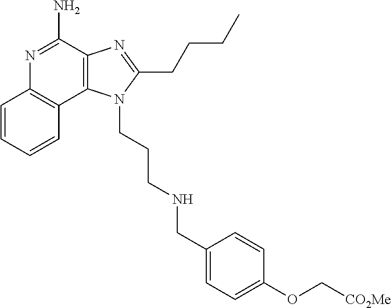

The title compound was prepared by the method of example 1 step (viii) using methyl (4-formylphenoxy)acetate (5.01 g) to afford the title compound, 2.70 g (34%) as colorless amorphous.

¹H NMR δ (CDCl₃) 8.12 (1H, d, J=8.1), 7.16 (1H, d, J=8.2), 7.40 (1H, dd, J=7.2, 8.2), 7.27-7.24 (2H, m), 7.20 (1H, dd, J=7.2, 8.1), 6.88-6.86 (2H, m), 6.45 (2H, brs), 4.78 (2H, s), 4.58 (2H, brt, J=7.2), 3.70 (3H, s), 3.63 (2H, s), 2.93 (2H, dd, J=7.6, 7.9), 2.60-2.55 (2H, m), 2.26 (1H, brs), 2.00-1.91 (2H, m), 1.79 (2H, tt, J=7.5, 7.7), 1.43 (2H, qt, J=7.3, 7.5), 0.95 (3H, t, J=7.3).

### Example 4

### Methyl (4-{[[3-(4-amino-2-butyl-1H-imidazo[4,5-c]quinolin-1-yl)propyl](chloroacetyl)amino]methyl}phenoxy)acetate hydrochloride

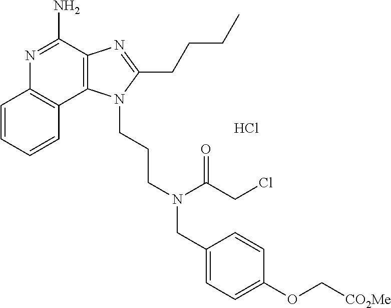

By the method of example 2 using the product of example 3 (2.70 g), there was obtained the title compound, 3.13 g (94%) as a white solid.

¹H NMR δ (DMSO-d₆) 13.82 (1H, brs), 8.60 (1H, brs), 8.21 (½H, d, J=8.2), 8.13 (½H, d, J=8.2), 7.82 (1H, dd, J=3.1, 8.3), 7.73-7.68 (1H, m), 7.57-7.52 (1H, m), 7.15 (1H, d, J=8.6), 7.08 (1H, d, J=8.6), 6.87 (1H, d, J=8.6), 6.81 (1H, d, J=8.6), 4.77 (1H, s), 4.75 (1H, s), 4.65-4.59 (1H, m), 4.55 (1H, s), 4.55-4.49 (1H, m), 4.48 (1H, s), 4.45 (1H, s), 4.43 (1H, s), 3.70 (3H, s), 3.54-3.39 (2H, m), 2.97-2.89 (2H, m), 2.12-1.92 (2H, m), 1.86-1.74 (2H, m), 1.50-1.39 (2H, m), 0.96 ( 3/2H, t, J=7.3), 0.95 ( 3/2H, t, J=7.3).

### Example 5

### Methyl (4-{[[3-(4-amino-2-butyl-1H-imidazo[4,5-c]quinolin-1-yl)propyl](N,N-dimethylglycyl)amino]methyl}phenoxy)acetate

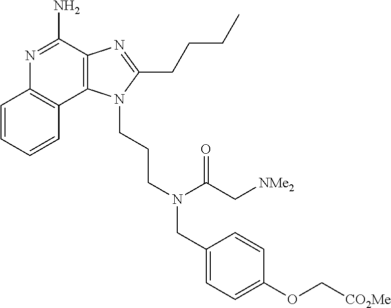

The product from example 4 (206 mg) was dissolved in MeCN (6 ml) and Me₂NH (2.0 M THF solution, 0.93 ml) was added. After stirring for 15 h, the reaction mixture was diluted with EtOAc, washed with water (twice), brine, dried and filtered, and the solvent was evaporated. The residue was purified by silica gel chromatography which afforded 189 mg of the desired product as a colorless gum. Yield 90%.

¹H NMR δ (DMSO-d₆) 8.02 (0.5H, d, J=8.1 Hz), 7.96 (0.5H, d, J=8.3 Hz), 7.63-7.60 (1H, m), 7.47-7.41 (1H, m), 7.27-7.23 (1H, m), 7.13 (1H, d, J=8.6 Hz), 7.09 (1H, d, J=8.6 Hz), 6.88 (1H, d, J=8.6 Hz), 6.83 (1H, d, J=8.6 Hz), 6.47 (2H, brs), 4.76 (1H, s), 4.75 (1H, s), 4.61 (1H, s), 4.52 (1H, t, J=7.4 Hz), 4.44-4.40 (2H, m), 3.70 (3H, s), 3.44-3.36 (2H, m), 3.12 (1H, s), 2.97 (1H, s), 2.88-2.83 (2H, m), 2.20 (2H, s), 2.14-2.06 (1H, m), 2.00 (3H, s), 1.97-1.91 (1H, m), 1.80-1.74 (2H, m), 1.46-1.41 (2H, m), 0.95 (3H, t, J=7.3 Hz).

MS: ESI 561 (M+1)

### Example 6

### Methyl (4-{[[3-(4-amino-2-butyl-1H-imidazo[4,5-c]quinolin-1-yl)propyl](piperidin-1-ylacetyl)amino]methyl}phenoxy)acetate

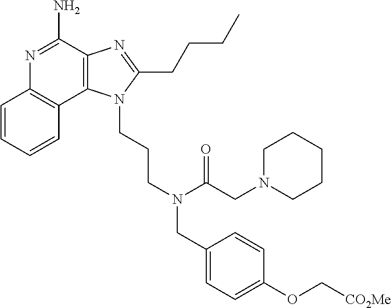

The title compound was prepared by the method of example 5 using the product from example 4 (260 mg) and piperidine, to give a colorless gum (229 mg). Yield 82%.

¹H NMR δ (CDCl₃) 8.01 (½H, d, J=8.2), 7.95 (½H, d, J=8.2), 7.63-7.60 (1H, m), 7.46-7.40 (1H, m), 7.25 (1H, dd, J=7.1, 15.0), 7.15 (1H, d, J=8.6), 7.05 (1H, d J=8.6), 6.88 (1H, d, J=8.6), 6.82 (1H, d, J=8.6), 6.48 (2H, brs), 4.76 (1H, s), 4.75 (1H, s), 4.62 (1H, s), 4.54 (1H, brt, J=7.0), 4.42-4.38 (1H, m), 4.40 (1H, s), 3.70 ( 3/2H, s), 3.69 ( 3/2H, s), 3.47-3.30 (2H, m), 3.14 (1H, s), 2.95 (1H, s), 2.85 (2H, dd, J=7.6, 15.3), 2.40-2.31 (2H, m), 2.23-2.17 (2H, m), 2.17-2.03 (1H, m), 1.99-1.89 (1H, m), 1.82-1.72 (2H, m), 1.48-1.22 (8H, m), 0.94 (3H, t, J=7.4).

### Example 7

### Methyl [4-({[3-(4-amino-2-butyl-1H-imidazo[4,5-c]quinolin-1-yl)propyl][(4-methylpiperazin-1-yl)acetyl]amino}methyl)phenoxy]acetate

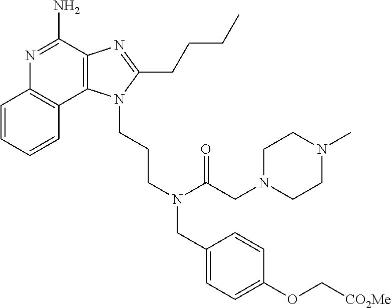

By the method of example 5 using the product from example 4 (300 mg) and 1-methylpiperazine, there was obtained the title compound as a colorless gum (228 mg). Yield 68%.

¹H NMR δ (DMSO-d₆) 8.01 (0.5H, d, J=8.0 Hz), 7.94 (0.5H, d, J=8.0 Hz), 7.62 (0.5H, d, J=7.8 Hz), 7.61 (0.5H, d, J=7.8 Hz), 7.45-7.40 (1H, m), 7.27-7.21 (1H, m), 7.15 (1H, d, J=8.5 Hz), 7.05 (1H, d, J=8.5 Hz), 6.88 (1H, d, J=8.6 Hz), 6.82 (1H, d, J=8.6 Hz), 6.46 (2H, brs), 4.76 (1H, s), 4.75 (1H, s), 4.60 (1H, s), 4.53 (1H, t, J=7.1 Hz), 4.42-4.35 (2H, m), 3.69 (3H, s), 3.43 (1H, t, J=7.0 Hz), 3.37 (1H, t, J=7.0 Hz), 3.16 (1H, s), 2.98 (1H, s), 2.89-2.81 (2H, m), 2.44-2.37 (2H, m), 2.30-2.17 (4H, m), 2.14-2.05 (3H, m), 2.09 (1.5H, s), 2.03 (1.5H, s), 1.97-1.88 (1H, m), 1.83-1.71 (2H, m), 1.45-1.39 (2H, m), 0.94 (3H, t, J=7.3 Hz).

MS: ESI 616 (M+1)

### Example 8

### Methyl

### {4-[([3-(4-amino-2-butyl-1H-imidazo[4,5-c]quinolin-1-yl)propyl]{[4-(2-methoxyeth yl)piperazin-1-yl]acetyl}amino)methyl]phenoxy}acetate

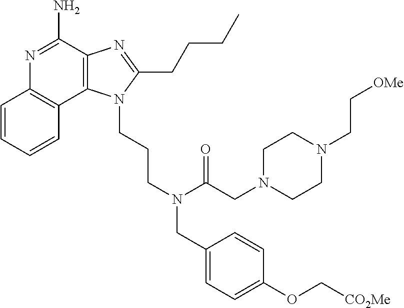

By the method of example 5 using the product from example 4 (300 mg) and 1-(2-methoxyethyl)piperazine, there was obtained the title compound as a white solid (277 mg). Yield 77%.

¹H NMR δ (CDCl₃) 7.83 (2H, m), 7.52 (1H, m), 7.33 (1H, m), 7.02 (2H, d, J=8.6 Hz), 6.79 (2H, d, J=8.6 Hz), 5.57 (2H, brs), 4.61 (2H, s), 4.58 (2H, s), 4.41 (2H, t, J=7.6 Hz), 3.81 (3H, s), 3.49-3.44 (4H, m), 3.33 (3H, s), 3.25 (2H, s), 2.86-2.02 (14H, m), 1.85-1.80 (2H, m), 1.51-1.44 (2H, m), 0.99 (3H, t, J=7.4 Hz).

MS:ESI 660 (M+1)

### Example 9

### Methyl (3-{[[3-(4-amino-2-butyl-1H-imidazo[4,5-c]quinolin-1-yl)propyl](N,N-dimethylglycyl)amino]methyl}phenoxy)acetate

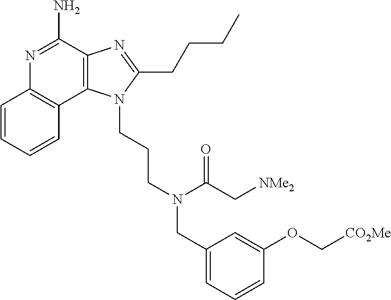

By the method of example 5 using the product from example 2 (304 mg), there was obtained the title compound as a colorless gum (265 mg). Yield 86%.

¹H NMR δ (DMSO-d₆) 8.00 (0.5H, d), 7.96 (0.5H, d), 7.60 (1H, d), 7.42 (1H, dd), 7.27-7.18 (2H, m), 6.83-6.75 (3H, m), 6.46 (2H, brs), 4.76 (1H, s), 4.73 (1H, t), 4.68 (1H, s), 4.51 (1H, t), 4.46 (1H, s), 4.41 (1H, t), 3.68 (1.5H, s), 3.67 (1.5H, s), 3.46 (1H, t), 3.41 (1H, t), 3.09 (1H, s), 2.98 (1H, s), 2.88-2.82 (2H, m), 2.19 (3H, s), 2.15-2.06 (1H, m), 2.00 (3H, s), 2.00-1.92 (1H, m), 1.81-1.72 (2H, m), 1.46-1.38 (2H, m), 0.94 (3H, t).

MS:ESI 561(M+1)

### Example 10

### Methyl (3-{[[3-(4-amino-2-butyl-1H-imidazo[4,5-c]quinolin-1-yl)propyl](piperidin-1-ylacetyl)amino]methyl}phenoxy)acetate

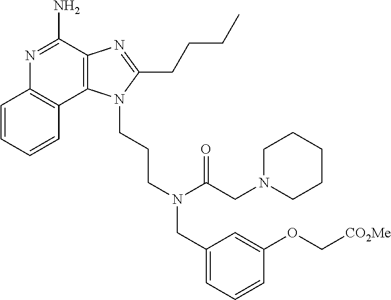

By the method of example 5 using the product from example 2 (261 mg) and piperidine, there was obtained the title compound as a colorless gum (254 mg). Yield 90%.

¹H NMR δ (DMSO-d₆) 8.01 (½H, d, J=8.0), 7.96 (½H, d, J=7.9), 7.63-7.60 (1H, m), 7.43 (1H, dd, J=8.0, 7.9), 7.27-7.17 (2H, m), 6.84-6.74 (3H, m), 6.47 (2H, brs), 4.77 (1H, s), 4.73 (1H, s), 4.54 (1H, brt, J=7.1), 4.47 (1H, s), 4.42 (1H, brt, J=7.8), 3.69 ( 3/2H, s), 3.68 ( 3/2H, s), 3.50 (1H, brt, J=7.6), 3.42 (1H, brt, J=6.9), 3.10 (1H, s), 2.95 (1H, s), 2.88-2.83 (2H, m), 2.39-2.32 (2H, m), 2.22-2.16 (2H, m), 2.16-2.07 (1H, m), 2.01-1.91 (1H, m), 1.82-1.73 (2H, m), 1.47-1.22 (8H, m), 0.95 ( 3/2H, t, J=7.3), 0.94 ( 3/2H, t, J=7.4).

### Example 11

### Methyl [3-({[3-(4-amino-2-butyl-1H-imidazo[4,5-c]quinolin-1-yl)propyl][(4-methylpiperazin-1-yl)acetyl]amino}methyl)phenoxy]acetate

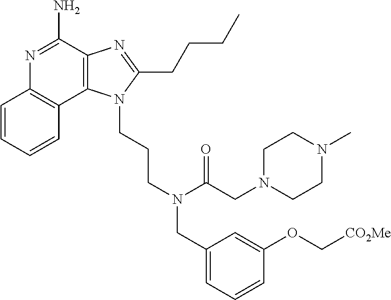

By the method of example 5 using the product from example 2 (396 mg) and 1-methylpiperazine, there was obtained the title compound as a colorless gum (303 mg). Yield 93%.

¹H NMR δ (CDCl₃) 8.01 (½H, d, J=8.1), 7.95 (½H, d, J=7.9), 7.63-7.59 (1H, m), 7.42 (1H, dd, J=8.1, 7.2), 7.28-7.17 (2H, m), 6.84-6.73 (3H, m), 6.46 (2H, brs), 4.77 (1H, s), 4.73 (1H, s), 4.68 (1H, s), 4.54 (1H, brt, J=6.9), 4.47 (1H, s), 4.39 (1H, brt, J=8.0), 3.69 ( 3/2H, s), 3.68 ( 3/2H, s), 3.50 (1H, brt, J=7.1), 3.45 (1H, brt, J=7.0), 3.18 (1H, s), 2.98 (1H, s), 2.86 (2H, dd, J=6.9, 14.9), 2.43-2.08 (5H, m), 2.08 ( 3/2H, s), 2.04 ( 3/2H, s), 2.01-1.90 (1H, m), 1.83-1.73 (2H, m), 1.48-1.38 (2H, m), 0.95 ( 3/2H, t, J=7.3), 0.94 ( 3/2H, t, J=7.4).

### Example 12

### Methyl {3-[([3-(4-amino-2-butyl-1H-imidazo[4,5-c]quinolin-1-yl)propyl]{[4-(2-methoxyethyl)piperazin-1-yl]acetyl}amino)methyl]phenoxy}acetate

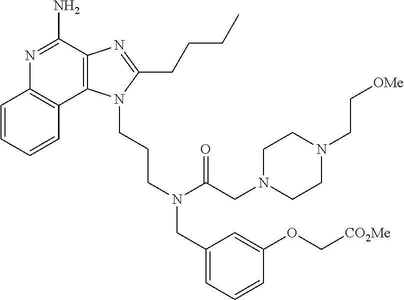

By the method of example 5 using the product from example 2 (300 mg) and 1-(2-methoxyethyl)piperazine, there was obtained the title compound as a pale yellow gum (300 mg). Yield 83%.

¹H NMR δ (DMSO-d₆) 7.99 (0.5H, d), 7.94 (0.5H, d), 7.61-7.58 (1H, m), 7.42-7.39 (1H, m), 7.25-7.16 (2H, m), 6.81-6.45 (3H, m), 6.44 (2H, brs), 4.75 (1H, s), 4.71 (1H, s), 4.67 (1H, s), 4.57-4.50 (1H, m), 4.45 (1H, s), 4.43-4.36 (1H, m), 3.67 (1.5H, s), 3.66 (1.5H, s), 3.52-3.39 (2H, m), 3.37-3.29 (2H, s), 3.18 (1.5H, s), 3.17 (1.5H, s), 3.11 (1H, s), 2.96 (1H, s), 2.88-2.80 (2H, m), 2.48-2.08 (11H, m), 2.00-1.92 (1H, m), 1.82-1.73 (2H, m), 1.49-1.37 (2H, m), 0.95-0.90 (3H, m).

MS:ESI 660(M+1)

### Example 13

Methyl (3-{[[3-(4-amino-2-butyl-1H-imidazo[4,5-c]quinolin-1-yl)propyl](pyrrolidin-1-ylacetyl)amino]methyl}phenoxy)acetate

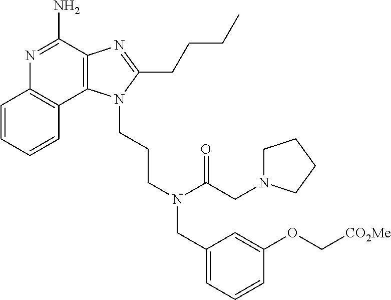

By the method of example 5 using the product from example 2 (441 mg) and pyrrolidine, there was obtained the title compound as a colorless gum (315 mg). Yield 67%.

¹H NMR δ (CDCl₃) 7.87 (1H, d, J=8.2 Hz), 7.83 (1H, dd, J=8.32, 0.84 Hz), 7.51 (1H, ddd, J=8.12, 8.12, 1.12 Hz), 7.33 (1H, m), 7.19 (1H, m), 6.79-6.77 (0.6H, m), 6.74-6.71 (2.4H, m), 5.61 (1.1H, brs), 5.51 (0.3H, brs), 4.68 (1.6H, s), 4.58 (2H, s), 4.55 (0.4H, s), 4.43 (2H, m), 3.79 (3H, s), 3.54-3.46 (2H, m), 3.67 (1.6H, s), 3.20 (0.4H, s), 2.85 (1.6H, t, J=7.68 Hz), 2.79 (0.4H, t, J=7.6 Hz), 2.60 (3H, brm), 2.45 (1H, brm), 2.19-2.16 (0.4H, m), 2.09-2.02 (1.6H, m), 1.89-1.78 (4H, m), 1.76 (3H, brm), 1.67 (1H, brm), 1.53-1.44 (2H, m), 0.99 (3H, t, J=7.32 Hz).

MS:ESI 587 (M+1)

### Example 14

### Methyl (3-{[[3-(4-amino-2-butyl-1H-imidazo[4,5-c]quinolin-1-yl)propyl](N,N-diethylglycyl)amino]methyl}phenoxy)acetate

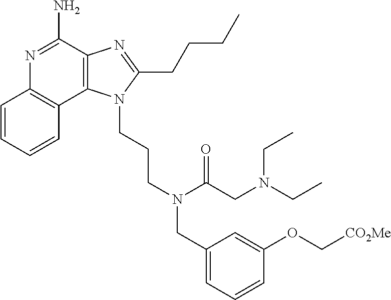

By the method of example 5 using the product from example 2 (441 mg) and Et₂NH, there was obtained the title compound as a colorless gum (348 mg). Yield 73%.

¹H NMR δ (CDCl₃) 7.88-7.83 (2H, m), 7.53 (1H, ddd, J=7.12, 7.12, 1.2 Hz), 7.34 (1H, m), 7.19 (1H, m), 6.79-6.71 (3H, m), 5.69 (1H, brs), 5.59 (0.3H, brs), 4.74 (1.6H, s), 4.59 (2H, s), 4.56 (0.4H, s), 4.45-4.39 (2H, m), 3.80 (3H, s), 3.58-3.50 (2H, m), 3.31 (1.6H, s), 3.25 (0.4H, s), 2.88-2.80 (2H, m), 2.61 (3.1H, q, J=7.16 Hz), 2.52 (0.9H, q, J=7.24 Hz), 2.22 (0.7H, m), 2.10-2.03 (1.3H, m), 1.94 (2H, brs), 1.88-1.81 (2H, m), 1.49 (2H, m), 0.99 (9H, m).

MS:ESI 589 (M+1)

### Example 15

### Methyl (3-{[[3-(4-amino-2-(2-methoxyethyl)-1H-imidazo[4,5-c]quinolin-1-yl)propyl]amino]methyl}phenoxy)acetate

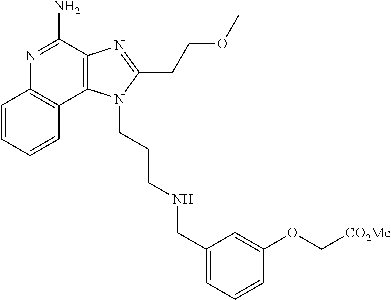

### i) tert-Butyl 3-[2-(2-methoxyethyl)-1H-imidazo[4,5-c]quinolin-1-yl]propylcarbamate

To the product of example 1 step (iii) (1.9 g) in NMP (25 mL), 3-methoxypropanoic acid (0.678 mL, 7.21 mmol) was added followed by HATU (2.74 g) and TEA (0.837 mL) under nitrogen. The resulting solution was stirred at 60° C. for 15 h. The reaction mixture was diluted with diethyl ether (300 mL) and EtOAc (300 mL), and washed with water (300 mL), sat. NaHCO₃ (200 mL), and saturated brine (200 mL). The organic layer was dried, filtered and evaporated to afford the subtitle product (3.5 g).

MS APCI+ve 385

### ii) 1-[3-(tert-Butoxycarbonylamino)propyl[-2-(2-methoxyethyl)-1H-imidazo[4,5-c]quinoline 5-oxide

The subtitle compound was prepared by the method of example 1 step (v) using the product from step (i).

MS APCI+ve: 401

### iii) tert-Butyl 3-[4-amino-2-(2-methoxyethyl)-1H-imidazo[4,5-c]quinolin-1-yl]propylcarbamate

The subtitle compound was prepared by the method of example 1 step (vi) using the product from step (ii).

MS APCI+ve: 400

### iv) 1-(3-Aminopropyl)-2-(2-methoxyethyl)-1H-imidazo[4,5-c]quinolin-4-amine

The subtitle compound was prepared by the method of example 1 step (vii) using the product of step (iii).

MS APCI+ve: 300

### v) Methyl (3-{[[3-(4-amino-2-(2-methoxyethyl)-1H-imidazo[4,5-c]quinolin-1-yl)propyl]amino]methyl}phenoxy)acetate

By the method of example 1 step (viii) using the product from step (iv) (197 mg) there was obtained the title compound, 234 mg (74%) as a white solid.

¹H NMR δ (CDCl₃) 8.06 (2H, dd, J=8.28, 1.00 Hz), 7.81 (1H, dd, J=8.36, 1.00 Hz), 7.49 (1H, m), 7.30-7.24 (2H, m), 6.97 (1H, d, J=7.6 Hz), 6.94 (1H, m), 6.79 (1H, dd, J=8.24, 2.12 Hz), 5.53 (1.6H, brs), 4.67-4.63 (4H, m), 3.88 (2H, t, J=6.6 Hz), 3.78 (3H, s), 3.36 (3H, s), 3.23 (2H, t, J=6.48 Hz), 2.73 (2H, t, J=6.28 Hz), 2.07 (2H, m).

MS:ESI 478 (M+1)

### Example 16

### Methyl (3-{[[3-(4-amino-2-(2-methoxyethyl)-1H-imidazo[4,5-c]quinolin-1-yl)propyl](chloroacetyl)amino]methyl}phenoxy)acetate hydrochloride

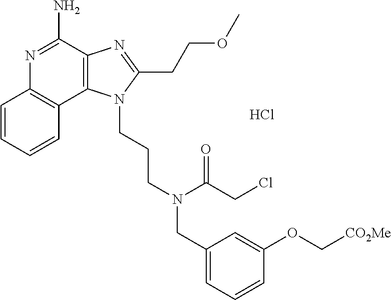

By the method of example 2 using the product of example 15 (100 mg), there was obtained the title compound, 166 mg (quant.) as a colorless gum.

¹H NMR δ (CDCl₃) 7.94 (2H, dd, J=8.0, 8.0 Hz), 7.61 (1H, dd, J=8.0, 8.0 Hz), 7.52 (1H, dd, J=8.0, 8.0 Hz), 7.24 (1H, m), 6.78-6.73 (3H, m), 4.62-4.59 (4H, m), 4.55 (2H, m), 4.12 (2H, s), 3.85 (2H, t, J=8.0 Hz), 3.79 (3H, s), 3.58 (2H, t, J=8.0 Hz), 3.35 (3H, s), 3.14 (2H, t, J=8.0 Hz), 2.13 (2H, m).

MS:ESI 554 (M+1)

### Example 17

### Methyl (3-{[[3-(4-amino-2-(2-methoxyethyl)-1H-imidazo[4,5-c]quinolin-1-yl)propyl](N,N-dimethylglycyl)amino]methyl}phenoxy)acetate

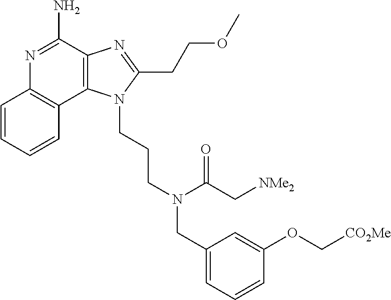

The title compound was prepared by the method of example 5 using the product from example 16 (166 mg), to give a colorless gum (72 mg). Yield 61%.

¹H NMR δ (CDCl₃) 7.89 (1H, d, J=8.4 Hz), 7.85 (1H, d, J=8.08 Hz), 7.53 (1H, t, J=7.36 Hz), 7.36 (1H, t, J=7.68 Hz), 7.21-7.17 (1H, m), 6.81-6.77 (1H, m), 6.74-6.71 (2H, m), 4.69 (1.5H, s), 4.58 (2H, s), 4.56 (0.5H, s), 4.49 (2H, m), 3.85 (2H, t, J=6.32 Hz), 3.79 (3H, s), 3.53-3.47 (2H, m), 3.35 (3H, s), 3.15-3.11 (3H, m), 3.08-3.05 (1H, m), 2.30 (4H, s), 2.12 (2H, s), 2.07 (2H, m).

MS:ESI 563 (M+1)

### Example 18

### Methyl (3-{[[3-(4-amino-2-(2-methoxyethyl)-1H-imidazo[4,5-c]quinolin-1-yl)propyl][(4-methylpiperazin-1-yl)acetyl]amino]methyl}phenoxy)acetate

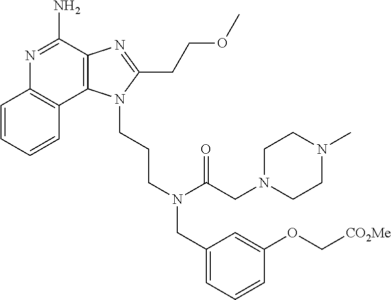

The title compound was prepared by the method of example 5 using the product from example 16 (137 mg) and 1-methylpiperazine, to give a colorless gum (99.6 mg). Yield 65%.

¹H NMR δ (CDCl₃) 7.87 (1H, d, J=8.24 Hz), 7.84 (1H, d, J=8.36 Hz), 7.52 (1H, dd, J=7.64, 7.64 Hz), 7.35 (1H, m), 7.19 (1H, m), 6.78-6.70 (3H, m), 5.63 (1H, brs), 4.65 (1.5H, s), 4.59 (2H, s), 4.55 (0.5H, s), 4.52-4.46 (2H, m), 3.86 (2H, s), 3.80 (3H, s), 3.52 (2H, m), 3.35 (3H, s), 3.22 (1.5H, s), 3.12 (2H, t, J=6.36 Hz), 3.06 (0.5H, s), 2.56-2.42 (6H, m), 2.25 (2H, s), 2.20 (1H, s), 2.11-2.04 (2H, m), 1.85 (2H, m).

MS:ESI 618 (M+1)

### Example 19

### Methyl (3-{[[3-(4-amino-2-(2-methoxyethyl)-1H-imidazo[4,5-c]quinolin-1-yl)propyl](piperidin-1-ylacetyl)amino]methyl}phenoxy)acetate

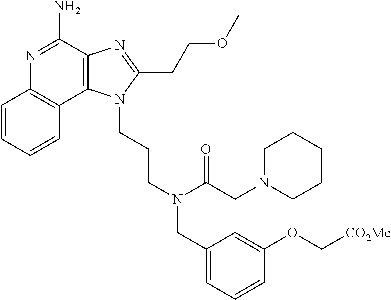

The title compound was prepared by the method of example 5 using the product from example 16 (137 mg) and piperidine, to give a colorless gum (110 mg). Yield 74%.

¹H NMR δ (CDCl₃) 7.88 (1H, d, J=8.2 Hz), 7.85 (1H, d, J=8.36 Hz), 7.53 (1H, dd, J=7.12, 7.12 Hz), 7.38-7.33 (1H, m), 7.21-7.17 (1H, m), 6.77-6.71 (3H, m), 4.71 (1.5H, s), 4.58-4.56 (2.5H, m), 4.53-4.45 (2H, m), 3.85 (2H, m), 3.80 (3H, s), 3.51 (2H, m), 3.34 (3H, s), 3.17 (1.5H, s), 3.14-3.06 (2.5H, m), 2.44 (3H, brm), 2.33 (1H, brm), 2.20 (0.6H, m), 2.06 (1.4H, m), 1.89 (2H, brs), 1.56-1.50 (4H, m), 1.39 (2H, m).

MS:ESI 603 (M+1)

### Example 20

### Methyl (3-{[[3-(4-amino-2-(2-methoxyethyl)-1H-imidazo[4,5-c]quinolin-1-yl)propyl](N,N-diethylglycyl)amino]methyl}phenoxy)acetate

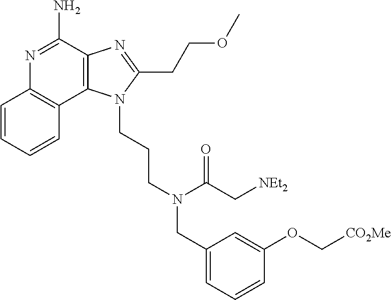

The title compound was prepared by the method of example 5 using the product from example 16 (331 mg) and Et₂NH, to give a white solid (231 mg). Yield 66%.

¹H NMR δ (CDCl₃) 7.88 (1H, d, J=7.48 Hz), 7.82 (1H, d, J=8.4 Hz), 7.51 (1H, m), 7.33 (1H, m), 7.19 (1H, m), 6.78-6.71 (3H, m), 5.46-5.43 (1.7H, brm), 4.75 (1.5H, s), 4.58 (2H, s), 4.56 (0.5H, s), 4.48 (2H, m), 3.86 (2H, t, J=6.36 Hz), 3.80 (3H, s), 3.59-3.49 (2H, m), 3.35 (2H, s), 3.33 (1H, s), 3.29 (1.5H, s), 3.26 (0.5H, s), 3.13 (1.5H, t, J=6.36 Hz), 3.08 (0.5H, t, J=6.28 Hz), 2.59 (3H, q, J=7.16 Hz), 2.52 (1H, q, J=7.08 Hz), 2.21 (0.5H, m), 2.08 (1.5H, m), 0.99 (6H, t, J=7.08 Hz).

MS:ESI 591 (M+1)

### Example 21

### Methyl (3-{[[3-(4-amino-2-butyl-1H-imidazo[4,5-c]quinolin-1-yl)propyl](3-morpholinopropyl)amino]methyl}phenoxy)acetate

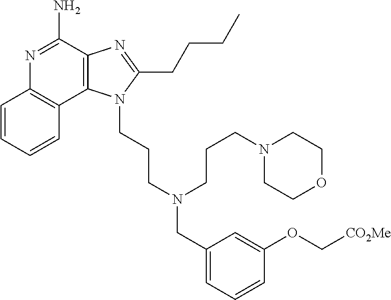

### (i)

### N-[3-(4-Amino-2-butyl-1H-imidazo[4,5-c]quinolin-1-yl)-propyl]-2-nitro-benzenesulfonamide

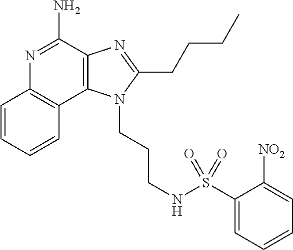

To a solution of the product from example 1 step (vii) (250 mg, 0.841 mmol) in CHCl₃ (8.4 mL), 2-nitrobenzenesulfonylchloride (186.4 mg, 0.841 mmol) was added at rt, and stirring rt for 3.5 h. Then sat. NaHCO₃ aq. was added and extracted with CHCl₃. The organic layer was dried over MgSO₄ then concentrated under reduced pressure. The crude product was purified by silica gel column chromatography to afford the subtitle compound (353 mg, yield 87%) as a pale yellow solid.

¹H NMR δ (CDCl₃) 8.07 (1H, dd, J=7.4, 1.64 Hz), 7.94 (1H, d, J=7.44 Hz), 7.85 (1H, dd, J=7.8, 1.68 Hz), 7.81 (1H, dd, J=8.36, 0.92 Hz), 7.70 (2H, m), 7.45 (1H, m), 7.36 (1H, m), 4.61 (2H, t, J=7.76 Hz), 3.27 (2H, t, J=6.32 Hz), 2.91 (2H, t, J=7.72 Hz), 2.18 (2H, m), 1.88 (2H, m), 1.52 (2H, m), 1.02 (3H, t, J=7.32 Hz).

MS:ESI 483 (M+1)

### (ii)

### N-[3-(4-Amino-2-butyl-1H-imidazo[4,5-c]quinolin-1-yl)-propyl]-N-(3-morpholin-4-yl-propyl)-2-nitro-benzenesulfonamide

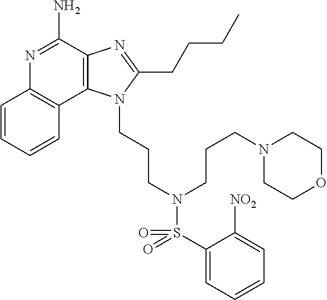

To a solution of the product from step (i) (164 mg, 0.340 mmol) in DMF (3.4 mL), 4-(3-bromopropyl)morpholine (141 mg, 0.680 mmol) was added at rt. After stirring at 60° C. for 3.5 h, sat. NaHCO₃ aq. was added and extracted with CHCl₃. The organic layer was dried over MgSO₄ then concentrated under reduced pressure. The crude residue was purified by silica gel column chromatography to afford the title compound (200 mg, yield 97%) as a pale yellow solid

¹H NMR δ (CDCl₃) 8.03 (1H, dd, J=7.56, 1.24 Hz), 7.98 (1H, d, J=8.16 Hz), 7.94 (1H, d, J=8.44 Hz), 7.72-7.58 (4H, m), 7.46 (1H, t, J=8.12 Hz), 4.57 (2H, m), 3.62-3.57 (6H, m), 3.39 (2H, t, J=7.44 Hz), 2.92 (2H, t, J=7.6 Hz), 2.28 (4H, m), 2.24-2.14 (4H, m), 1.89 (2H, m), 1.65 (2H, m), 1.53 (2H, m), 0.99 (3H, t, J=7.32 Hz).

MS:ESI 610 (M+1) 2-Butyl-1-[3-(3-morpholin-4-yl-propylamino)-propyl]-1H-imidazo[4,5-c]quinolin-4-ylamine

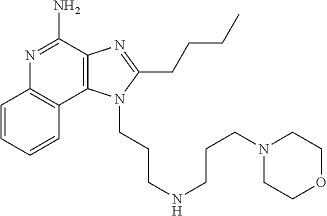

The sulfonamide from step (ii) (200 mg, 0.329 mmol) was dissolved into THF (5 mL), and to this solution, CsCO₃ (355 mg, 1.09 mmol) was added followed by

PS-thiophenol (485 mg of a resin with 1.49 mmol/g loading, 0.724 mmol). The reaction mixture was stirred at rt for 8 h. Additional PS-thiophenol was added (243 mg, 0.362 mmol) and the mixture was stirred for 16 h. Then the content of the flask was filtered, and the solid was washed several times with THF and CH₂Cl₂. The solvent was evaporated and the residue was isolated to give the subtitle compound, 130 mg (93% yield) as a white solid.

¹H NMR δ (CDCl₃) 8.06 (1H, d, J=8.16 Hz), 7.84 (1H, d, J=8.32 Hz), 7.52 (1H, m), 7.33 (1H, m), 5.63 (1.5H, brs), 4.59 (2H, dd, J=7.52, 7.12 Hz), 3.70 (4H, m), 2.96 (2H, dd, J=7.96, 7.76 Hz), 2.71 (4H, m), 2.44-2.41 (6H, m), 2.08 (2H, m), 1.87 (2H, m), 1.73 (4H, m), 1.51 (2H, m), 1.01 (3H, t, J=7.32 Hz).

MS:ESI 425 (M+1)

### (iv) Methyl (3-{[[3-(4-amino-2-butyl-1H-imidazo[4,5-c]quinolin-1-yl)propyl](3-morpholinopropyl)amino]methyl}phenoxy)acetate

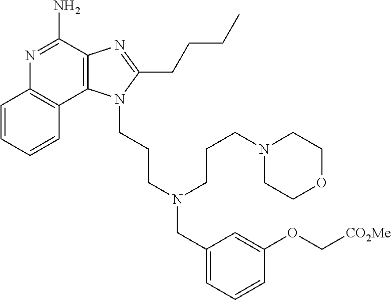

The product from step (iii) (134 mg, 0.316 mmol) was dissolved in MeOH, and to this solution, methyl 2-(4-formylphenoxy)acetate (56.3 mg, 0.316 mmol) was added followed by NaBH₃CN (39.7 mg, 0.632 mmol) and acetic acid (36.7 uL, 0.632 mmol) and stirred at 0° C. for 3 h. The reaction was quenched with sat. NaHCO₃ aq. and extracted with CHCl₃. The organic layer was dried over MgSO₄ then concentrated under reduced pressure. The resulting residue was purified by silica gel column chromatography to afford the title compound (86.4 mg, Yield 45%) as colorless oil.

¹H NMR δ (CDCl₃) 7.95 (1H, dd, J=8.24, 0.92 Hz), 7.84 (1H, dd, J=8.36, 0.92 Hz), 7.49 (1H, m), 7.25-7.20 (2H, m), 6.99 (1H, d, J=7.96 Hz), 6.96 (1H, m), 6.79 (1H, dd, J=7.72, 2.08 Hz), 5.75 (1.5H, brs), 4.62 (2H, s), 4.41 (2H, m), 3.76 (3H, s), 3.68 (4H, m), 3.61 (2H, s), 2.87 (2H, t, J=7.64 Hz), 2.61 (2H, t, J=6.32 Hz), 2.54 (2H, t, J=7.32 Hz), 2.40 (4H, brm), 2.34 (2H, t, J=7.28 Hz), 2.01 (2H, m), 1.88-1.72 (6H, m), 1.48 (2H, m), 0.99 (3H, t, J=7.32 Hz).

MS:ESI 603 (M+1)

### Example 22

### Methyl [4-({[({3-[4-amino-2-(2-methoxyethyl)-1H-imidazo[4,5-c]quinolin-1-yl]propyl}amino)carbonyl][3-(dimethylamino)propyl]amino}methyl)phenoxy]acetate

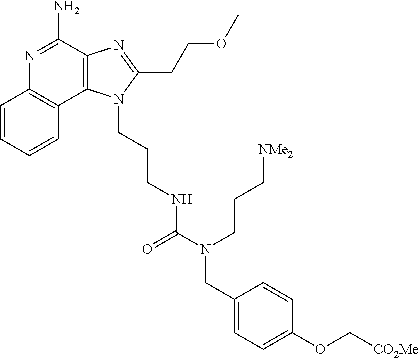

### (i) Methyl [4-({[3-(dimethylamino)propyl]amino}methyl)phenoxy]acetate

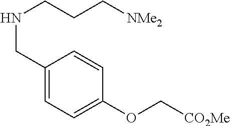

The suspension of copper sulfate (1.6 g 10 mmol), methyl (4-formylphenoxy)acetate (1.5 g 7.7 mmol) and 3-dimethylaminopropylamine (4.3 ml 35 mmol) in toluene (10 ml) was stirred at ambient temperature for 14 h. The reaction mixture was filtered and filtrate was concentrated in vacuo. Methanol (10 ml) was added to the residue, and then sodium borohydride (380 mg, 10 mmol) was added at 0° C. The resulting solution was stirred at 0-10° C. for 10 min, then sodium bicarbonate aq. (100 mL) was added and extracted with CHCl₃ (100 mL, twice). Organic layer was dried over sodium sulfate then concentrated in vacuo. The residue was purified by silica gel chromatography to give the 690 mg of subtitled compound as colourless oil. Yield 42%.

¹H NMR δ (DMSO-d₆) 7.19 (2H, d, J=6.8 Hz), 6.83 (2H, d, J=6.8 Hz), 4.75 (2H, s), 3.68 (3H, s), 3.57 (2H, s), 2.44 (1H, t, J=7.2 Hz), 2.18 (1H, t, J=7.2 Hz), 2.07 (6H, s), 1.97 (1H, brs), 1.54-1.46 (2H, m).

### (ii) Methyl [4-({[({3-[4-amino-2-(2-methoxyethyl)-1H-imidazo[4,5-c]quinolin-1-yl]propyl}amino)carbonyl][3-(dimethylamino)propyl]amino}methyl)phenoxy]acetate

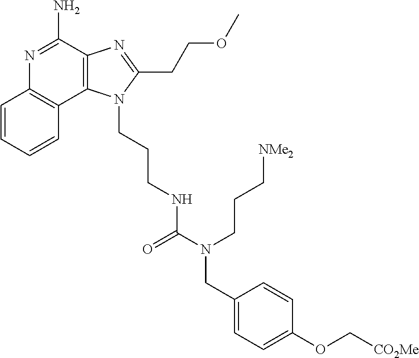

4-Nitrophenyl chloroformate (197 mg, 0.98 mmol) was add to the mixture of triethylamine (0.177 ml, 1.3 mmol) and the product from step (i) (276 mg, 0.98 mmol) in tetrahydrofuran (5 ml) at 0° C. and stirred for 0.5 h. Then the product from example 15 step (iv) (409 mg, 1.1 mmol), triethylamine (0.409 ml, 3 mmol) and DMSO (7 ml) were added to the reaction mixture and stirred at ambient temperature for 14 h. The reaction mixture was poured into ethyl acetate (50 ml) and the mixture was washed with potassium carbonate aq. Organic layer was dried over sodium sulfate then concentrated in vacuo. The residue was purified by silica gel chromatography to give the titled compound, 130 mg as a colourless gum. Yield 22%.

¹H NMR δ (DMSO-d₆) 8.04 (1H, d, J=7.6 Hz), 7.59 (1H, dd, J=0.8, 7.6 Hz), 7.43-7.35 (1H, m), 7.18-7.12 (3H, m), 7.03 (1H, brs), 6.84 (2H, d, J=7.6 Hz), 6.47 (2H, s), 4.74 (2H, s), 4.54 (1H, t, J=7.6 Hz), 4.36 (2H, s), 3.80 (1H, t, J=6.8 Hz), 3.67 (3H, s), 3.29-3.20 (5H, m), 3.19-3.15 (2H, m), 3.09 (2H, t, J=6.8 Hz), 2.11 (2H, t, J=6.8 Hz), 2.02-1.92 (8H, m), 1.57-1.50 (2H, m).

### Example 23

### Ethyl 2-[3-({3-[4-amino-2-(2-methoxyethyl)-1H-imidazo[4,5-c]quinolin-1-yl]propylamino}methyl)phenoxy]acetate

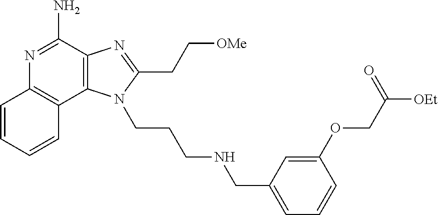

### (i) Ethyl 2-(3-formylphenoxy)acetate

To a solution of 3-hydroxybenzaldehyde (3.00 g, 24.6 mmol) in DMF (30 ml) ethyl bromoacetate (4.53 g, 27.1 mmol) and K₂CO₃ (3.75 g, 27.1 mmol) was added at rt. After stirring for 3 h at 80° C., diluted with AcOEt, and H₂O was added. The aq. layer was extracted with AcOEt, dried over Na₂SO₄, and concentrated. The residue was purified by flash column chromatography to give the title compound (5.22 g, 100%) as colorless oil.

¹H NMR δ (CDCl₃) 9.96 (1H, s), 7.50 (1H, ddd, J=7.48, 1.36, 1.32 Hz), 7.46 (1H, dd, J=7.56, 7.56 Hz), 7.35 (1H, m), 7.25 (1H, m), 4.68 (2H, s), 4.27 (2H, q, J=7.16 Hz), 1.29 (3H, t, J=7.12 Hz).

MS:ESI 209 (M+1)

### (ii) Ethyl 2-[3-({3-[4-amino-2-(2-methoxyethyl)-1H-imidazo[4,5-c]quinolin-1-yl]propylamino}methyl)phenoxy]acetate

By the method of example 1 step (viii) using the product from example 15 step (iv) (1.50 g) and ethyl 2-(3-formylphenoxy)acetate (1.04 g) to afford the title compound, 2.02 g (82%) as a white solid.

¹H NMR δ (CDCl₃) 8.10 (1H, dd, J=8.10, 0.84 Hz), 7.84 (1H, dd, J=8.36, 1.00 Hz), 7.51 (1H, m), 7.32-7.25 (2H, m), 6.98 (1H, d, J=7.48 Hz), 6.95 (1H, m), 6.81 (1H, dd, J=8.16, 2.04 Hz), 5.69 (2H, brs), 4.67 (2H, t, J=7.52 Hz), 4.64 (2H, s), 4.26 (2H, q, J=7.16 Hz), 3.90 (2H, t, J=6.56 Hz), 3.80 (2H, s), 3.38 (3H, s), 3.25 (2H, t, J=6.52 Hz), 2.75 (2H, t, J=6.2 Hz), 2.08 (2H, m), 1.29 (3H, m).

MS:ESI 492 (M+1)

### Example 24

### Ethyl 2-{3-[(N-{3-[4-amino-2-(2-methoxyethyl)-1H-imidazo[4,5-c]quinolin-1-yl]propyl}-2-chloroacetamido)methyl)phenoxy]acetate hydrochloride

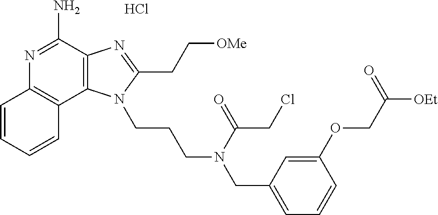

By the method of example 2 using the product of example 23 (2.01 g), there was obtained the title compound, 2.61 g (quant.) as a colorless gum.

¹H NMR δ (CDCl₃) 8.01 (1H, d, J=8.28 Hz), 7.96 (1H, d, J=7.96 Hz), 7.66 (1H, m), 7.54 (1H, m), 7.25 (1H, m), 6.80-6.73 (3H, m), 4.63 (2H, s), 4.61 (2H, s), 4.59-4.53 (2H, m), 4.27 (2H, q, J=7.16 Hz), 4.12 (2H, s), 3.87 (2H, t, J=6.00 Hz), 3.59 (2H, t, J=6.84 Hz), 3.36 (3H, s), 3.15 (2H, t, J=6.00 Hz), 2.13 (2H, m), 1.31 (3H, t, J=7.12 Hz).

MS:ESI 568 (M+1)

### Example 25

### Ethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate

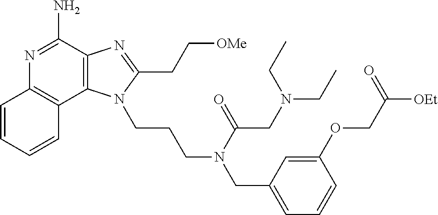

The title compound was prepared by the method of example 5 using the product from example 24 (2.60 g) and diethylamine, to give a colorless gum (2.27 g). Yield 92%.

¹H NMR δ (CDCl₃) 7.92-7.85 (2H, m), 7.58-7.52 (1H, m), 7.42-7.32 (1H, m), 7.22-7.18 (2H, m), 6.79-6.73 (3H, m), 4.76 (1.5H, s), 4.58 (2.5H, s), 4.49 (2H, m), 4.27 (2H, q, J=7.12 Hz), 3.86 (2H, t, J=6.28 Hz), 3.59-3.51 (2H, m), 3.36 (2.3H, s), 3.34 (0.7H, s), 3.32 (1.5H, s), 3.28 (0.5H, s), 3.15-3.07 (2H, m), 2.63-2.52 (4H, m), 2.22-2.04 (2H, m), 1.30 (3H, t, J=7.12 Hz), 1.00 (6H, t, J=7.08 Hz).

MS:ESI 605 (M+1)

### Example 26

### Propyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate

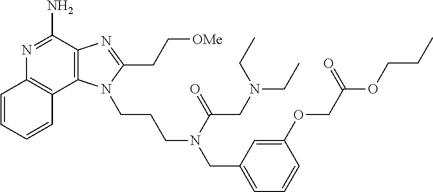

### (i) (3-{[[3-(4-Amino-2-(2-methoxyethyl)-1H-imidazo[4,5-c]quinolin-1-yl)propyl](N,N-diethylglycyl)amino]methyl}phenoxy)acetic acid

To a solution of example 25 (556.3 mg, 0.920 mmol) in THF/MeOH (1:1, 8 ml), 2N NaOH (1.6 mL) was added at rt. After stirring for 3 h at rt, the reaction mixture was neutralized with 1N HCl. The aq. layer was extracted with CHCl₃/EtOH (5/1), dried over Na₂SO₄, and concentrated to give the title compound (551.1 mg, quant.) as a white solid.

¹H NMR δ (CD₃OD) 7.92 (1H, d, J=8.24 Hz), 7.64 (1H, t, J=7.92 Hz), 7.49 (1H, m), 7.35 (1H, J=7.24 Hz), 7.15 (0.7H, t, J=8.04 Hz), 7.08 (0.3H, t, J=7.88 Hz), 6.80-6.62 (3H, m), 4.55-4.44 (4H, m), 4.36 (1.4H, s), 4.35 (0.6H, s), 3.88-3.82 (3.3H, m), 3.63 (0.7H, s), 3.52 (1.4H, t, J=7.4 Hz), 3.40-3.34 (3.6H, m), 3.15-3.10 (2H, m), 2.94 (2.7H, m), 2.81 (1.3H, m), 2.13-1.99 (2H, m), 1.15-1.05 (6H, m).

MS:ESI 577 (M+1)

### (ii) Propyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate

To a solution of the product from step (i) (161.2 mg) in nPrOH (3 mL), 4N HCl/dioxane solution (0.5 mL) was added. The reaction mixture was stirred at 50° C. for 3 h. After the removal of the solvent, the residue was diluted with CHCl₃ and then poured into 7% NH₃ solution, and extracted with CHCl₃. The combined extracts were dried over Na₂SO₄ and concentrated. The residue was purified by flash column chromatography to afford the title compound (99.6 mg, 65%) as a colorless gum.

¹H NMR δ (CDCl₃) 7.92-7.85 (2H, m), 7.58-7.52 (1H, m), 7.41-7.37 (1H, m), 7.2 (1H, dd, J=7.6, 7.6 Hz), 6.80-6.73 (3H, m), 4.76 (1.5H, s), 4.59 (2H, s), 4.58 (0.5H, s), 4.51-4.47 (2H, m), 4.16 (2H, t, J=6.72 Hz), 3.86 (2H, t, J=6.32 Hz), 3.59-3.51 (2H, m), 3.36 (2.3H, s), 3.34 (0.7H, s), 3.31 (1.5H, s), 3.28 (0.5H, s), 3.14 (1.5H, t, J=6.24 Hz), 3.08 (0.5H, t, J=6.24 Hz), 2.63-2.52 (4H, m), 2.22-2.01 (2H, m), 1.69 (2H, m), 1.00 (6H, t, J=7.08 Hz), 0.93 (3H, t, J=7.4 Hz).

MS:ESI 619 (M+1)

### Example 27

### Isopropyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate

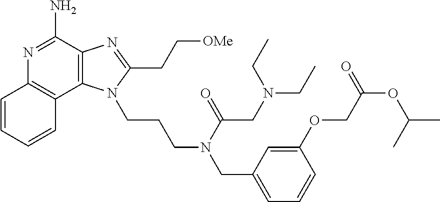

The title compound was prepared by the method of example 26 step (ii) using the product from example 26 step (i) (100.3 mg) and 2-propanol, to give a colorless gum (58.4 mg). Yield 54%.

¹H NMR δ (CDCl₃) 7.91-7.82 (2H, m), 7.52 (1H, m), 7.33 (1H, m), 7.20 (1H, m), 6.80-6.72 (3H, m), 5.43 (2H, m), 5.13 (1H, m), 4.76 (1.5H, s), 4.58 (0.5H, s), 4.54 (2H, s), 4.49 (2H, m), 3.87 (2H, m), 3.60-3.49 (2H, m), 3.36 (2.3H, s), 3.34 (0.7H, s), 3.30 (1.5H, s), 3.26 (0.5H, s), 3.14 (1.5H, t, J=6.36 Hz), 3.09 (0.5H, t, J=6.2 Hz), 2.63-2.50 (4H, m), 2.22-2.05 (2H, m), 1.28 (6H, d, J=6.28 Hz), 1.00 (6H, t, J=7.08 Hz).

MS:ESI 619 (M+1)

### Example 28

### Isobutyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate

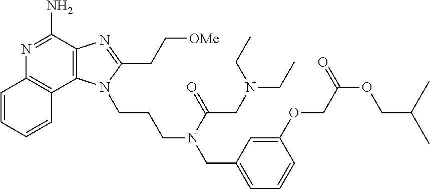

The title compound was prepared by the method of example 26 step (ii) using the product from example 26 step (i) (76.4 mg) and 2-methyl-1-propanol, to give a colorless gum (55.7 mg). Yield 67%.

¹H NMR δ (CDCl₃) 7.89-7.81 (2H, m), 7.51 (1H, m), 7.32 (1H, m), 7.18 (1H, m), 6.78-6.71 (3H, m), 5.44 (2H, brs), 4.74 (1.5H, s), 4.58 (2H, s), 4.56 (0.5H, s), 4.48 (2H, m), 3.97 (2H, d, J=6.68 Hz), 3.85 (2H, m), 3.51 (2H, m), 3.34 (2.3H, s), 3.33 (0.7H, s), 3.29 (1.5H, s), 3.25 (0.5H, s), 3.13 (1.5H, t, J=6.36 Hz), 3.07 (0.5H, t, J=6.24 Hz), 2.59 (3H, q, J=7.16 Hz), 2.52 (1H, q, J=7.08 Hz), 2.09-2.05 (2H, m), 1.94 (1H, m), 0.98 (6H, t, J=7.08 Hz), 0.90 (6H, d, J=6.72 Hz).

MS:ESI 633 (M+1)

### Example 29

### 2-Methoxyethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate

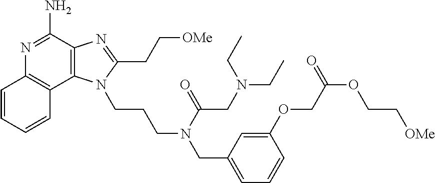

The title compound was prepared by the method of example 26 step (ii) using the product from example 26 step (i) (101.6 mg) and 2-methoxyethanol, to give a colorless gum (76.3 mg). Yield 68%.

¹H NMR δ (CDCl₃) 7.90-7.82 (2H, m), 7.52 (1H, m), 7.34 (1H, m), 7.20 (1H, dd, J=8.92, 7.16 Hz), 6.80-6.72 (3H, m), 5.45 (2H, brs), 4.76 (1.5H, s), 4.63 (2H, s), 4.57 (0.5H, s), 4.49 (2H, m), 4.36 (2H, m), 3.86 (2H, m), 3.75 (2H, m), 3.62 (2H, m), 3.41 (3H, s), 3.39 (3H, s), 3.30 (1.5H, s), 3.26 (0.5H, s), 3.14 (1.5H, t, J=6.36 Hz), 3.10 (0.5H, t, J=6.2 Hz), 2.63-2.52 (4H, m), 2.10-2.05 (2H, m), 1.00 (6H, t, J=7.12 Hz).

MS:ESI 635 (M+1)

### Example 30

### 2-Hydroxyethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate

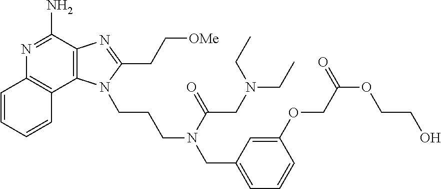

The title compound was prepared by the method of example 26 step (ii) using the product from example 26 step (i) (101.6 mg) and ethylenegrycol, to give a colorless gum (58.5 mg). Yield 54%.

¹H NMR δ (CDCl₃) 7.82-7.75 (2H, m), 7.50 (1H, m), 7.31-7.28 (1H, m), 7.11 (1H, m), 6.75 (1.25H, d, J=7.96 Hz), 6.60 (0.75H, dd, J=8.24, 2.08 Hz), 6.45 (0.25H, s), 6.38 (0.75H, s), 5.56 (2H, brs), 4.60 (1.5H, s), 4.50-4.41 (2.5H, m), 4.37-4.34 (2H, m), 4.31 (0.5H, s), 4.25 (1.5H, s), 3.93-3.88 (2H, m), 3.82 (2H, t, J=6.44 Hz), 3.50 (0.5H, m), 3.39-3.28 (6H, m), 3.26 (0.5H, s), 3.09 (1.5H, t, J=6.36 Hz), 3.04 (0.5H, t, J=6.16 Hz), 2.61-2.53 (4H, m), 2.00 (2H, m), 0.99 (6H, t, J=7.16 Hz).

MS:ESI 621 (M+1)

### Example 31

### Ethyl 2-{3-[(N-{3-[4-amino-2-(2-methoxyethyl)-1H-imidazo[4,5-c]quinolin-1-yl]propyl}-2-(pyrrolidin-1-yl)acetamido)methyl]phenoxy}acetate

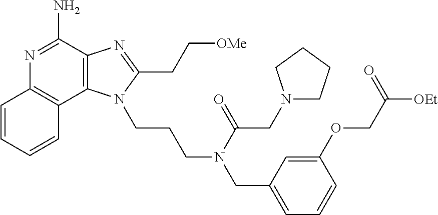

The title compound was prepared by the method of example 5 using the product from example 24 (195.7 mg) and pyrrolidine, to give a pale yellow gum (142.1 mg). Yield 68%.

¹H NMR δ (CDCl₃) 7.89-7.82 (2H, m), 7.52 (1H, m), 7.34 (1H, m), 7.21 (1H, m), 6.82-6.74 (3H, m), 5.41 (2H, brs), 4.70 (1.5H, s), 4.58 (2.5H, s), 4.50 (2H, m), 4.27 (2H, q, J=7.12 Hz), 3.86 (2H, m), 3.56-3.49 (2H, m), 3.37 (0.7H, s), 3.36 (2.3H, s), 3.35 (1.5H, s), 3.24 (0.5H, s), 3.15 (1.5H, t, J=6.44 Hz), 3.07 (0.5H, t, J=6.16 Hz), 2.61 (3H, brm), 2.48 (1H, brm), 2.19-2.05 (2H, m), 1.77-1.68 (4H, m), 1.30 (3H, t, J=7.12 Hz).

MS:ESI 603 (M+1)

### Example 32

### Ethyl 2-{3-[(N-{3-[4-amino-2-(2-methoxyethyl)-1H-imidazo[4,5-c]quinolin-1-yl]propyl}-2-(piperidin-1-yl)acetamido)methyl]phenoxy}acetate

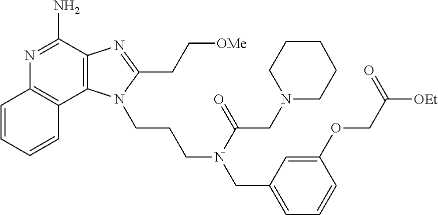

The title compound was prepared by the method of example 5 using the product from example 24 (186.9 mg) and piperidine, to give a colorless gum (179.4 mg). Yield 88%.

¹H NMR δ (CDCl₃) 7.90-7.81 (2H, m), 7.52 (1H, m), 7.34 (1H, m), 7.20 (1H, m), 6.80-6.73 (3H, m), 5.38 (2H, brs), 4.73 (1.5H, s), 4.58 (2H, s), 4.57 (0.5H, s), 4.54-4.47 (2H, m), 4.27 (2H, q, J=7.16 Hz), 3.87 (2H, t, J=6.4 Hz), 3.53 (2H, m), 3.36 (2.3H, s), 3.34 (0.7H, s), 3.18 (1.5H, s), 3.14 (1.5H, t, J=6.36 Hz), 3.09 (0.5H, t, J=6.16 Hz), 3.06 (0.5H, s), 2.45 (3H, brm), 2.34 (1H, brm), 2.10-2.06 (2H, m), 1.56-1.50 (4H, m), 1.40 (2H, brm), 1.31 (3H, t, J=7.12 Hz).

MS:ESI 617 (M+1)

### Example 33

### Ethyl 2-{3-[(N-{3-[4-amino-2-(2-methoxyethyl)-1H-imidazo[4,5-c]quinolin-1-yl]propyl}-2-(dimethylamino)acetamido)methyl]phenoxy}acetate

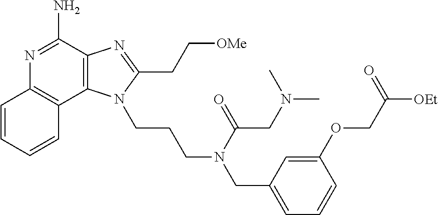

The title compound was prepared by the method of example 5 using the product from example 24 (201.0 mg) and dimethylamine, to give a colorless gum (188.0 mg). Yield 92%.

¹H NMR δ (CDCl₃) 7.90-7.80 (2H, m), 7.51 (1H, m), 7.32 (1H, m), 7.19 (1H, m), 6.80-6.72 (3H, m), 5.41 (2H, brs), 4.69 (1.5H, s), 4.56 (2.5H, s), 4.48 (2H, m), 4.25 (2H, q, J=7.16 Hz), 3.85 (2H, t, J=6.4 Hz), 3.54-3.46 (2H, m), 3.34 (2.3H, s), 3.33 (0.7H, s), 3.15-3.04 (4H, m), 2.30 (4.5H, s), 2.22-2.03 (2H, m), 2.11 (1.5H, s), 1.29 (3H, t, J=7.12 Hz).

MS:ESI 577 (M+1)

### Example 34

### Methyl 2-[4-({3-[4-amino-2-(2-methoxyethyl)-1H-imidazo[4,5-c]quinolin-1-yl]propylamino}methyl)phenoxy]acetate

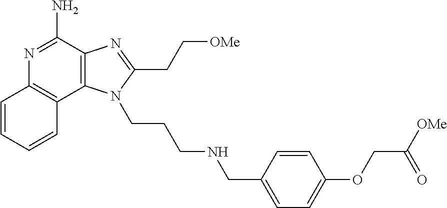

### (i) Methyl 2-(4-formylphenoxy)acetate

By the method of example 23 step (i) using the 4-hydroxybenzaldehyde (1.50 g) and the methyl bromoacetate (1.28 mL) to afford the title compound, 2.42 g (100%) as a white solid.

¹H NMR δ (CDCl₃) 9.91 (1H, s), 7.81 (2H, ddd, J=8.84, 2.68, 2.64 Hz), 7.02 (21H, ddd, J=8.76, 2.68, 2.64 Hz), 4.72 (2H, s), 3.83 (3H, s).

MS:ESI 195 (M+1)

### (ii) Methyl 2-[4-({3-[4-amino-2-(2-methoxyethyl)-1H-imidazo[4,5-c]quinolin-1-yl]propylamino}methyl)phenoxy]acetate

By the method of example 1 step (viii) using the product from example 15 step (iv) (500 mg) and methyl 2-(4-formylphenoxy)acetate (324.3 mg) to afford the title compound, 651.0 mg (81%) as a white solid.

¹H NMR δ (CDCl₃) 8.10 (1H, dd, J=8.24, 0.88 Hz), 7.84 (1H, dd, J=8.32, 0.92 Hz), 7.51 (1H, m), 7.31-7.25 (3H, m), 6.92-6.88 (2H, m), 5.68 (2H, brs), 4.67-4.62 (4H, m), 3.89 (2H, t, J=6.52 Hz), 3.82 (3H, s), 3.76 (2H, s), 3.38 (3H, s), 3.24 (2H, t, J=6.48 Hz), 2.74 (2H, t, J=6.28 Hz), 2.12-2.02 (2H, m).

MS:ESI 478 (M+1)

### Example 35

### Methyl 2-{4-[(N-{3-[4-amino-2-(2-methoxyethyl)-1H-imidazo[4,5-c]quinolin-1-yl]propyl}-2-chloroacetamido)methyl]phenoxy}acetate hydrochloride

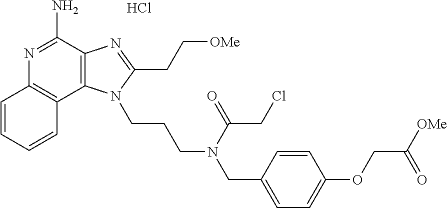

By the method of example 2 using the product of example 34 (328.1 mg), there was obtained the title compound, 340.0 mg (91%) as a colorless gum.

¹H NMR δ (CDCl₃) 8.00 (1H, d, J=8.2 Hz), 7.95 (1H, d, J=8.16 Hz), 7.64 (1H, m), 7.52-7.49 (1H, m), 7.10-7.04 (2H, m), 6.88-6.81 (2H, m), 4.64-4.52 (6H, m), 4.14 (2H, s), 3.87 (2H, t, J=6.04 Hz), 3.82 (3H, s), 3.57 (2H, t, J=7.24 Hz), 3.36 (3H, s), 3.14 (2H, t, J=6.04 Hz), 2.18-2.10 (2H, m).

MS:ESI 554 (M+1)

### Example 36

### Methyl 2-{4-[(N-{3-[4-amino-2-(2-methoxyethyl)-1H-imidazo[4,5-c]quinolin-1-yl]propyl}-2-(diethylamino)acetamido)methyl]phenoxy}acetate

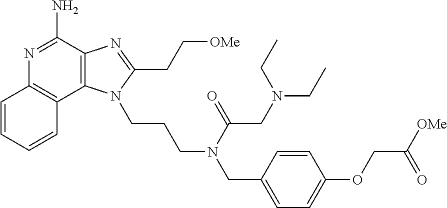

The title compound was prepared by the method of example 5 using the product from example 35 (348.0 mg) and diethylamine, to give a colorless gum (305.2 mg). Yield 82%.

¹H NMR 8 (CDCl₃) 7.90 (1H, d, J=8.36 Hz), 7.87 (1H, d, J=8.32 Hz), 7.55 (1H, m), 7.35 (1H, m), 7.09-7.04 (2H, m), 6.83-6.79 (2H, m), 5.95 (1.5H, brs), 5.72 (0.5H, brs), 4.69 (1.5H, s), 4.62 (0.5H, s), 4.61 (2H, s), 4.49 (2H, m), 3.86 (2H, t, J=6.32 Hz), 3.82 (3H, s), 3.57-3.47 (2H, m), 3.36 (2.3H, s), 3.35 (1.5H, s), 3.33 (0.7H, s), 3.27 (0.5H, s), 3.12 (1.5H, t, J=6.32 Hz), 3.08 (0.5H, t, J=6.12 Hz), 2.61 (3.0H, q, J=7.12 Hz), 2.54 (1H, q, J=7.12 Hz), 2.23-2.02 (2H, m), 0.99 (6H, t, J=7.12 Hz).

MS:ESI 591 (M+1)

### Example 37

### Ethyl 2-{4-[(N-{3-[4-amino-2-(2-methoxyethyl)-1H-imidazo[4,5-c]quinolin-1-yl]propyl}-2-(diethylamino)acetamido)methyl]phenoxy}acetate

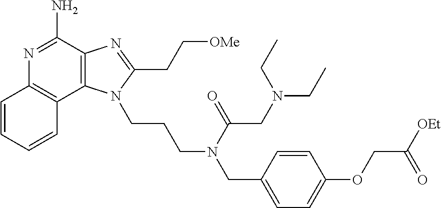

### (i) 2-{4-[(N-{3-[4-Amino-2-(2-methoxyethyl)-1H-imidazo[4,5-c]quinolin-1-yl]propyl}-2-(diethylamino)acetamido)methyl]phenoxy}acetic acid

The title compound was prepared by the method of example 26 step (i) using the product from example 36 (163.7 mg), to give a white solid (162.8 mg). Yield quant.

¹H NMR δ (DMSO-d₆) 7.99 (0.5H, d, J=7.96 Hz), 7.95 (0.5H, d, J=8.12 Hz), 7.59 (1H, dd, J=8.4, 1.48 Hz), 7.41 (1H, t, J=7.0 Hz), 7.24 (1H, q, J=7.08 Hz), 7.04 (1H, d, J=8.56 Hz), 7.00 (1H, d, J=8.6 Hz), 6.73 (1H, d, J=8.6 Hz), 6.68 (1H, d, J=8.64 Hz), 6.50 (2H, brs), 4.61 (1H, s), 4.52 (1H, m), 4.42 (1H, m), 4.36 (1H, s), 4.00 (1H, s), 3.99 (1H, s), 3.78 (2H, q, J=6.84 Hz), 3.43 (1H, m), 3.37 (1H, m), 3.26 (3H, s), 3.22 (1H, s), 3.16-3.09 (3H, m), 2.53-2.47 (2H, m), 2.36 (2H, m), 2.10 (1H, m), 1.94 (1H, m), 0.88 (3H, t, J=7.08 Hz), 0.81 (3H, t, J=7.04 Hz).

MS:ESI 577 (M+1)

### (ii) Ethyl 2-{4-[(N-{3-[4-amino-2-(2-methoxyethyl)-1H-imidazo[4,5-c]quinolin-1-yl]propyl}-2-(diethylamino)acetamido)methyl]phenoxy}acetate

The title compound was prepared by the method of example 26 step (ii) using the product from step (i) (65.6 mg) and EtOH, to give a colorless gum (44.8 mg). Yield 65%.

¹H NMR δ (CDCl₃) 7.90-7.82 (2H, m), 7.53 (1H, m), 7.32 (1H, m), 7.09-7.03 (2H, m), 6.83-6.80 (2H, m), 5.48 (2H, brs), 4.69 (1.5H, s), 4.60 (0.5H, s), 4.59 (2H, s), 4.49 (2H, m), 4.28 (2H, q, J=7.12 Hz), 3.86 (2H, t, J=6.4 Hz), 3.56-3.47 (2H, m), 3.36 (2.3H, s), 3.35 (0.7H, s), 3.22 (1.5H, s), 3.26 (0.5H, s), 3.13 (1.5H, t, J=6.4 Hz), 3.09 (0.5H, t, J=6.28 Hz), 2.61 (3H, q, J=7.16 Hz), 2.53 (1H, q, J=7.12 Hz), 2.22 (0.5H, m), 2.10-2.03 (1.5H, m), 1.31 (3H, t, J=7.16 Hz), 1.01 (6H, t, J=7.12 Hz).

MS:ESI 605 (M+1)

### Example 38

### Methyl 2-[2-({3-[4-amino-2-(2-methoxyethyl)-1H-imidazo[4,5-c]quinolin-1-yl]propylamino}methyl)phenoxy]acetate

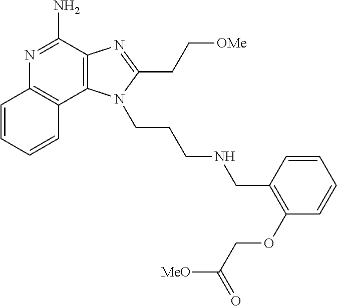

### (i) Methyl 2-(2-formylphenoxy)acetate

By the method of example 23 step (i) using the 2-hydroxybenzaldehyde (501.8 mg) and the methyl bromoacetate (408.9 uL) to afford the title compound, 734.0 mg (92%) as a white solid.

¹H NMR δ (CDCl₃) 10.58 (1H, s), 7.88 (1H, dd, J=7.68, 1.8 Hz), 7.55 (1H, m), 7.11 (1H, dd, J=7.52, 7.52 Hz), 6.87 (1H, d, J=8.36 Hz), 4.79 (2H, s), 3.83 (3H, s).

### (ii) Methyl 2-[2-({3-[4-amino-2-(2-methoxyethyl)-1H-imidazo[4,5-c]quinolin-1-yl]propylamino}methyl)phenoxy]acetate

By the method of example 1 step (viii) using the product from example 15 step (iv) (166.7 mg) and methyl 2-(2-formylphenoxy)acetate 108.2 mg) to afford the title compound, 222.8 mg (84%) as a colorless gum.

¹H NMR δ (CDCl₃) 8.08 (1H, dd, J=8.24, 0.96 Hz), 7.80 (1H, dd, J=8.36, 1.00 Hz), 7.48 (1H, m), 7.29-7.21 (3H, m), 6.97 (1H, m), 6.75 (1H, d, J=8.12 Hz), 5.39 (2H, brs), 4.68 (2H, s), 4.65 (2H, t, J=7.48 Hz), 3.90-3.87 (4H, m), 3.73 (3H, s), 3.37 (3H, s), 3.24 (2H, t, J=6.6 Hz), 2.70 (2H, t, J=6.36 Hz), 2.10 (2H, m).

MS:ESI 478 (M+1)

### Example 39

### Methyl 2-{2-[(N-{3-[4-amino-2-(2-methoxyethyl)-1H-imidazo[4,5-c]quinolin-1-yl]propyl}-2-chloroacetamido)methyl]phenoxy}acetate hydrochloride

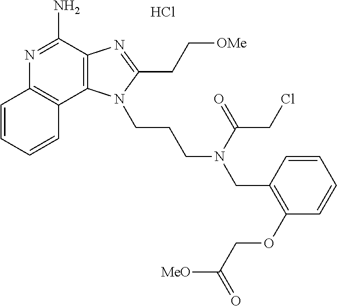

By the method of example 2 using the product of example 38 (213.9 mg), there was obtained the title compound, 286.6 mg (quant.) as a colorless gum.

¹H NMR δ (CDCl₃) 7.90-7.82 (2H, m), 7.53 (1H, m), 7.33 (1H, m), 7.16 (1H, m), 7.00 (1H, dd, J=7.8, 1.76 Hz), 6.86 (1H, m), 6.65 (1H, d, J=8.12 Hz), 5.49 (2H, brs), 4.75 (0.3H, s), 4.66 (1.7H, s), 4.60 (1.7H, s), 4.58 (0.3H, s), 4.50 (2H, m), 4.40 (1.7H, s), 4.06 (0.3H, s), 3.87 (2H, t, J=6.44 Hz), 3.78 (2.5H, s), 3.77 (0.5H, s), 3.54 (2H, t, J=6.96 Hz), 3.38 (3H, s), 3.13 (2H, t, J=6.44 Hz), 2.06 (2H, m).

MS:ESI 554 (M+1)

### Example 40

### Methyl 2-{2-[(N-{3-[4-amino-2-(2-methoxyethyl)-1H-imidazo[4,5-c]quinolin-1-yl]propyl}-2-(diethylamino)acetamido)methyl]phenoxy}acetate

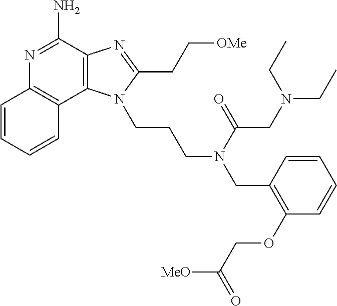

The title compound was prepared by the method of example 5 using the product from example 39 (249.2 mg) and diethylamine, to give a colorless gum (198.3 mg). Yield 75%.

¹H NMR δ (CDCl₃) 7.88 (1H, m), 7.80 (1H, d, J=8.36 Hz), 7.49 (1H, m), 7.34-7.24 (1H, m), 7.12 (1H, m), 7.05 (1H, dd, J=7.52, 1.36 Hz), 6.92-6.85 (1H, m), 6.66 (0.2H, d, J=7.88 Hz), 6.60 (0.8H, d, J=7.96 Hz), 5.56 (2H, brs), 4.79 (1.6H, s), 4.73 (0.4H, s), 4.55 (2H, s), 4.47 (2H, m), 3.84 (2H, m), 3.71 (3H, s), 3.66 (0.4H, m), 3.52 (1.6H, t, J=7.00 Hz), 3.35 (1.6H, s), 3.34 (2.4H, s), 3.33 (0.6H, s), 3.19 (0.4H, s), 3.11 (2H, t, J=6.48 Hz), 2.59 (3.0H, q, J=7.16 Hz), 2.46 (1H, q, J=7.16 Hz), 2.05 (2H, m), 0.99 (4.8H, t, J=7.12 Hz), 0.92 (1.2H, t, J=7.12 Hz).

MS:ESI 591 (M+1)

### Example 41

### Ethyl 2-{2-[(N-{3-[4-amino-2-(2-methoxyethyl)-1H-imidazo[4,5-c]quinolin-1-yl]propyl}-2-(diethylamino)acetamido)methyl]phenoxy}acetate

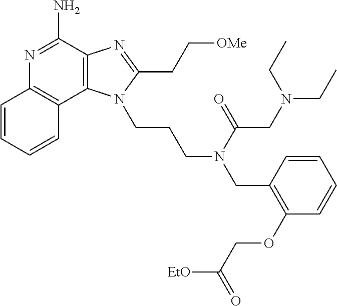

### (i) 2-{2[(N-{3-[4-Amino-2-(2-methoxyethyl)-1H-imidazo[4,5-c]quinolin-1-yl]propyl}-2-(diethylamino)acetamido)methyl]phenoxy}acetic acid

The title compound was prepared by the method of example 26 step (i) using the product from example 40 (145.2 mg), to give a white solid (140.5 mg). Yield 99%.

¹H NMR δ (DMSO-d₆) 8.02 (0.5H, d, J=8.52 Hz), 7.94 (0.5H, d, J=8.2 Hz), 7.55 (1H, m), 7.34 (1H, m), 7.25-7.07 (5H, m), 6.88-6.82 (2H, m), 4.75 (1H, s), 4.58-4.41 (5H, m), 3.78 (2H, m), 3.63 (2H, m), 3.47-3.42 (2H, m), 3.99 (1H, s), 3.27 (1.5H, s), 3.25 (1.5H, s), 3.21-3.09 (2H, m), 2.56 (2H, q, J=7.16 Hz), 2.39 (2H, q, J=7.12 Hz), 2.18 (1H, m), 1.96 (1H, m), 0.91 (3H, t, J=7.08 Hz), 0.83 (3H, t, J=7.16 Hz).

MS:ESI 577 (M+1)

### (ii) Ethyl 2-{2-[(N-{3-[4-amino-2-(2-methoxyethyl)-1H-imidazo[4,5-c]quinolin-1-yl]propyl}-2-(diethylamino)acetamido)methyl]phenoxy}acetate

The title compound was prepared by the method of example 26 step (ii) using the product from step (i) (62.9 mg) and EtOH, to give a colorless gum (58.1 mg). Yield 83%.

¹H NMR δ (CDCl₃) 7.94-7.90 (1H, m), 7.82 (1H, d, J=8.4 Hz), 7.50 (1H, m), 7.35-7.31 (1H, m), 7.21-7.12 (1H, m), 7.06 (1H, d, J=7.52 Hz), 6.95-6.87 (1H, m), 6.69 (0.3H, d, J=8.24 Hz), 6.63 (0.7H, d, J=7.88 Hz), 5.51 (2H, brs), 4.82 (1.5H, s), 4.75 (0.5H, s), 4.56 (2H, s), 4.49 (2H, m), 4.19 (2H, q, J=7.12 Hz), 3.86 (2H, t, J=6.52 Hz), 3.68 (0.5H, m), 3.55 (1.5H, t, J=7.00 Hz), 3.37 (1.5H, s), 3.36 (2.3H, s), 3.35 (0.7H, s), 3.20 (0.5H, s), 3.13 (2H, t, J=6.48 Hz), 2.61 (3H, q, J=7.12 Hz), 2.47 (1H, q, J=7.16 Hz), 2.28-2.05 (2H, m), 1.25 (3H, m), 1.01 (4.5H, t, J=7.12 Hz), 0.93 (1.5H, t, J=7.12 Hz).

MS:ESI 605 (M+1)

### Example 42

### Ethyl 2-[3-({4-[4-amino-2-(2-methoxyethyl)-1H-imidazo[4,5-c]quinolin-1-yl]butylamino}methyl)phenoxy]acetate

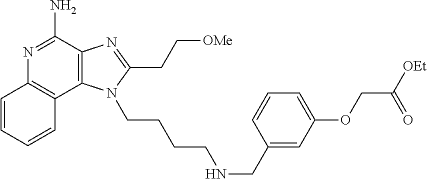

### (i) tert-Butyl 4-(3-nitroquinolin-4-ylamino)butylcarbamate

The subtitle compound (27.4 g) was prepared by the same procedure of example 1 step (ii) using 3-nitroquinolin-4-ol (15 g) and (4-aminobutyl)-carbamic acid tert-butyl ester (22.6 ml). Yield: 96%

¹H NMR δ (CDCl₃) 9.70 (1H, brs), 9.37 (1H, s), 8.30 (1H, dd, J=0.9, 8.6), 7.99 (1H, dd, J=1.1, 8.3), 7.77 (1H, ddd, J=1.3, 7.6, 7.7), 7.49 (1H, ddd, J=1.3, 7.7, 7.8), 4.63 (1H, brs), 4.00 (2H, m), 3.21 (2H, m), 1.88 (2H, m), 1.68 (2H, m), 1.44 (9H, s).

MS: ESI 361 (M+1)

### (ii) tert-Butyl 4-(3-aminoquinolin-4-ylamino)butylcarbamate

The subtitle compound (960 mg) was prepared by the same procedure of example 1 step (iii) using product from step (i) (1.06 g). Yield: 99%

¹H NMR δ (CDCl₃) 8.48 (1H, s), 7.98 (1H, dd, J=1.9, 8.5), 7.84 (1H, dd, J=1.8, 8.1), 7.50-7.43 (2H, m), 4.61 (1H, brs), 3.81 (2H, brs), 3.30 (2H, m), 3.17 (2H, m), 1.76-1.60 (4H, m), 1.44 (9H, s).

MS: ESI 331 (M+1)

### (iii) tert-Butyl 4-[2-(2-methoxyethyl)-1H-imidazo[4,5-c]quinolin-1-yl]butylcarbamate

The subtitle compound (1.69 g) was prepared by the same procedure of example 15 step (i) using product from step (ii) (1.49 g). Yield: 94%

¹H NMR δ (CDCl₃) 9.28 (1H, s), 8.35 (1H, d, J=8.2), 8.15 (1H, dd, J=1.5, 7.9), 7.68 (2H, m), 4.61 (3H, m), 3.97 (2H, t, J=6.5), 3.39 (3H, s), 3.27-3.21 (4H, m), 2.00 (2H, m), 1.71 (2H, m), 1.42 (9H, s).

MS: ESI 399 (M+1)

### (iv) 1-[4-(tert-Butoxycarbonylamino)butyl]-2-(2-methoxyethyl)-1H-imidazo[4,5-c]quinoline 5-oxide

The subtitle compound (1.32 g) was prepared by the same procedure of example 1 step (v) using product from step (iii) (1.29 g). Yield: 98%

¹H NMR δ (CDCl₃) 9.13 (1H, s), 9.03 (1H, m), 8.17 (1H, m), 7.80 (2H, m), 4.68 (1H, brs), 4.61 (2H, brs), 3.93 (2H, t, J=6.12), 3.38 (3H, s), 3.25-3.22 (4H, m), 2.00 (2H, m), 1.72 (2H, m), 1.42 (9H, s).

MS: ESI 415 (M+1)

### (v) tert-Butyl 4-[4-amino-2-(2-methoxyethyl)-1H-imidazo[4,5-c]quinolin-1-yl]butylcarbamate

The subtitle compound (1.18 g) was prepared by the same procedure of example 1 step (vi) using product from step (iv) (1.32 g). Yield: 91%

¹H NMR δ (CDCl₃) 7.86 (1H, d, J=8.2), 7.78 (1H, d, J=8.4), 7.46 (1H, t, J=7.2), 7.28 (1H, t, J=7.2), 5.45 (1H, brs), 4.65 (1H, brs), 4.48 (2H, m), 3.85 (2H, t, J=6.4), 3.34 (3H, s), 3.14 (2H, m), 1.91 (2H, m), 1.62 (2H, m), 1.38 (9H, s).

MS: ESI 414 (M+1)

### (vi) 1-(4-Aminobutyl)-2-(2-methoxyethyl)-1H-imidazo[4,5-c]quinolin-4-amine

The subtitle compound (766 mg) was prepared by the same procedure of example 1 step (vii) using product from step (v) (1.06 g). Yield: 95%

¹H NMR δ (CDCl₃) 7.96 (1H, d, J=8.1), 7.82 (1H, d, J=8.3), 7.51 (1H, dd, J=7.2, 8.1), 7.33 (1H, t, J=7.2, 8.1), 5.47 (2H, brs), 4.53 (2H, t, J=7.8), 3.90 (2H, t, J=6.5), 3.38 (3H, s), 3.19 (2H, t, J=6.5), 2.28 (2H, t, J=7.0), 2.03-1.93 (2H, m), 1.70-1.56 (4H, m).

MS: ESI 314 (M+1)

### (vii) Ethyl 2-[3-({4-[4-amino-2-(2-methoxyethyl)-1H-imidazo[4,5-c]quinolin-1-yl]butylamino}methyl)phenoxy]acetate

By the method of example 1 step (viii) using the product from step (vi) (780.0 mg) and ethyl 2-(3-formylphenoxy)acetate (511.6 mg) to afford the title compound, 740.9 mg (59%) as a white solid.

¹H NMR δ (CDCl₃) 7.97 (1H, dd, J=8.24, 0.96 Hz), 7.81 (1H, dd, J=8.36, 0.96 Hz), 7.50 (1H, m), 7.30 (1H, m), 7.22 (1H, dd, J=7.88, 7.88 Hz), 6.92 (1H, d, J=7.68 Hz), 6.89 (1H, d, J=2.24 Hz), 6.77 (1H, dd, J=8.16, 2.04 Hz), 5.39 (2H, brs), 4.59 (2H, s), 4.52 (2H, m), 4.25 (2H, q, J=7.16 Hz), 3.89 (2H, t, J=6.56 Hz), 3.75 (2H, s), 3.37 (3H, s), 3.18 (2H, t, J=6.52 Hz), 2.69 (2H, t, J=6.96 Hz), 1.99 (2H, m), 1.71-1.65 (2H, m), 1.28 (3H, t, J=7.16 Hz).

MS:ESI 506 (M+1)

### Example 43

### Ethyl 2-{3-[(N-{4-[4-amino-2-(2-methoxyethyl)-1H-imidazo[4,5-c]quinolin-1-yl]butyl}-2-chloroacetamido)methyl]phenoxy}acetate hydrochloride

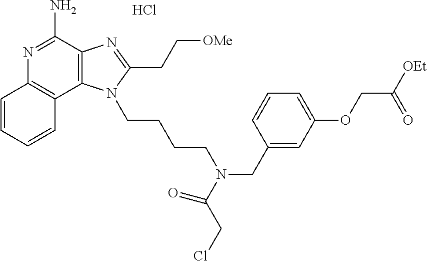

By the method of example 2 using the product of example 42 (381.5 mg), there was obtained the title compound, 481.7 mg (quant.) as a colorless gum.

¹H NMR δ (CDCl₃) 7.88-7.81 (2H, m), 7.53-7.48 (1H, m), 7.32 (1H, m), 7.24-7.18 (1H, m), 6.82-6.71 (3H, m), 5.54 (2H, brs), 4.59 (2H, s), 4.58 (0.5H, s), 4.56-4.48 (2H, m), 4.47 (1.5H, s), 4.25 (2H, m), 4.09 (0.5H, s), 3.99 (1.5H, s), 3.87 (2H, m), 3.42 (1.5H, t, J=7.24 Hz), 3.35 (3H, s), 3.28 (0.5H, t, J=7.16 Hz), 3.16 (1.5H, t, J=6.4 Hz), 3.11 (0.5H, t, J=6.16 Hz), 1.90 (2H, m), 1.70-1.63 (2H, m), 1.28 (3H, t, J=7.16 Hz).

MS:ESI 582 (M+1)

### Example 44

### Ethyl

### 2-{3-[(N-{4-[4-amino-2-(2-methoxyethyl)-1H-imidazo[4,5-c]quinolin-1-yl]butyl}-2-(diethylamino)acetamido)methyl]phenoxy}acetate

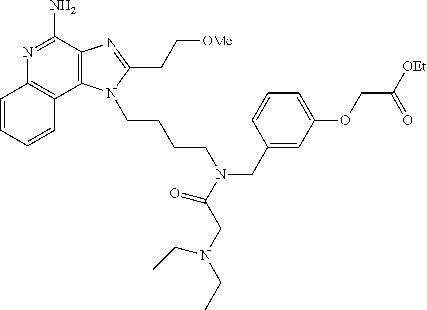

The title compound was prepared by the method of example 5 using the product from example 43 (480.2 mg) and diethylamine, to give a pale yellow gum (450.2 mg). Yield 96%.

¹H NMR δ (CDCl₃) 7.86-7.79 (2H, m), 7.49 (1H, m), 7.30 (1H, m), 7.23-7.19 (1H, m), 6.82-6.71 (3H, m), 5.39 (2H, brs), 4.67 (1.5H, s), 4.57 (2H, s), 4.54 (0.5H, s), 4.67-4.45 (2H, m), 4.24 (2H, q, J=7.12 Hz), 3.86 (2H, t, J=6.48 Hz), 3.39-3.34 (2H, m), 3.35 (3H, s), 3.23 (0.5H, s), 3.19 (1.5H, s), 3.15 (1.5H, t, J=6.44 Hz), 3.11 (0.5H, t, J=6.44 Hz), 2.50 (4H, m), 1.85 (2H, m), 1.71-1.63 (2H, m), 1.28 (3H, t, J=7.12 Hz), 0.95-0.89 (6H, m).

MS:ESI 619 (M+1)

### Example 45

### Isopropyl 2-{3-[(N-{4-[4-amino-2-(2-methoxyethyl)-1H-imidazo[4,5-c]quinolin-1-yl]butyl}-2-{diethylamino}acetamido)methyl]phenoxy}acetate

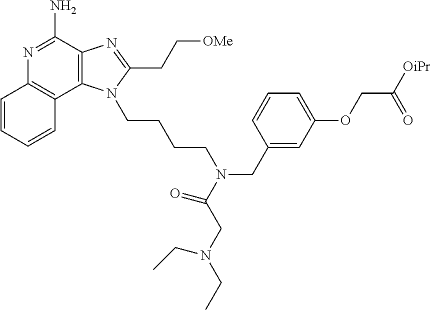

### (i) 2-{3-[(N-{4-[4-Amino-2-(2-methoxyethyl)-1H-imidazo[4,5-c]quinolin-1-yl]butyl}-2-{diethylamino}acetamido)methyl]phenoxy}acetic acid

The title compound was prepared by the method of example 26 step (i) using the product from example 44 (391.4 mg), to give a pale yellow solid (371.0 mg). Yield 99%

¹H NMR δ (DMSO-d₆) 7.87 (1H, m), 7.59 (0.5H, d, J=3.16 Hz), 7.57 (0.5H, d, J=3.8 Hz), 7.42 (1H, m), 7.28-7.20 (2H, m), 6.85-6.78 (3H, m), 4.65 (1H, s), 4.58 (1H, s), 4.56 (1H, s), 4.49 (1H, s), 4.44 (2H, m), 3.80 (2H, m), 3.40-3.32 (2H, m), 3.30 (1.5H, s), 3.29 (1.5H, s), 3.26 (2H, m), 3.14 (2H, m), 2.56-2.50 (4H, m), 1.70 (3H, m), 1.59 (1H, m), 0.92-0.87 (6H, m).

MS:ESI 591 (M+1)

### (ii) Isopropyl 2-{3-[(N-{4-[4-amino-2-(2-methoxyethyl)-1H-imidazo[4,5-c]quinolin-1-yl]butyl}-2-{diethylamino}acetamido)methyl]phenoxy}acetate

The title compound was prepared by the method of example 26 step (ii) using the product from step (i) (80.6 mg) and iPrOH, to give a colorless gum (58.9 mg). Yield 68%.

¹H NMR δ (CDCl₃) 7.91-7.82 (2H, m), 7.52 (1H, m), 7.33 (1H, m), 7.20 (1H, m), 6.80-6.72 (3H, m), 5.43 (2H, m), 5.13 (1H, m), 4.76 (1.5H, s), 4.58 (0.5H, s), 4.54 (2H, s), 4.49 (2H, m), 3.87 (2H, m), 3.60-3.49 (2H, m), 3.36 (2.3H, s), 3.34 (0.7H, s), 3.30 (1.5H, s), 3.26 (0.5H, s), 3.14 (1.5H, t, J=6.36 Hz), 3.09 (0.5H, t, J=6.2 Hz), 2.63-2.50 (4H, m), 2.22-2.05 (2H, m), 1.71-1.63 (2H, m), 1.28 (6H, d, J=6.28 Hz), 1.00 (6H, t, J=7.08 Hz).

MS:ESI 633 (M+1)

### Example 46

### tert-Butyl 2-[3-({4-[4-amino-2-(2-methoxyethyl)-1H-imidazo[4,5-c]quinolin-1-yl]butylamino}methyl)phenoxy]acetate

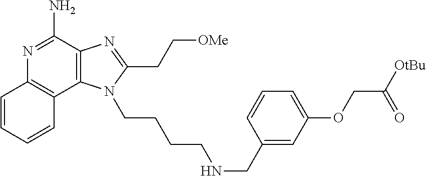

### (i) tert-Butyl 2-(3-formylphenoxy)acetate

By the method of example 23 step (i) using the 3-hydroxybenzaldehyde (500 mg) and the t-butyl bromoacetate (633.4 uL) to afford the title compound, 969.3 mg (100%) as colorless oil.

¹H NMR δ (CDCl₃) 9.96 (1H, s), 7.50 (1H, ddd, J=7.48, 1.4, 1.36 Hz), 7.46 (1H, dd, J=7.72, 7.48 Hz), 7.34 (1H, m), 7.22 (1H, ddd, J=7.8, 2.72, 1.48 Hz), 4.58 (2H, s), 1.49 (9H, s).

### (ii) tert-Butyl 2-[3-({4-[4-amino-2-(2-methoxyethyl)-1H-imidazo[4,5-c]quinolin-1-yl]butylamino}methyl)phenoxy]acetate

By the method of example 1 step (viii) using the product from example 42 step (vi) (183.0 mg) and tert-butyl 2-(3-formylphenoxy)acetate (138.9 mg) to afford the title compound, 217.0 mg (70%) as a pale yellow gum.

¹H NMR δ (CDCl₃) 7.99 (1H, dd, J=8.24, 1.00 Hz), 7.83 (1H, dd, J=8.32, 1.00 Hz), 7.52 (1H, m), 7.32 (1H, m), 7.23 (1H, dd, J=7.92, 7.84 Hz), 6.92 (1H, d, J=7.64 Hz), 6.88 (1H, d, J=2.24 Hz), 6.78 (1H, dd, J=8.12, 2.08 Hz), 5.42 (2H, brs), 4.54 (2H, m), 4.51 (2H, s), 3.90 (2H, t, J=6.56 Hz), 3.77 (2H, s), 3.39 (3H, s), 3.20 (2H, t, J=6.52 Hz), 2.70 (2H, t, J=6.96 Hz), 2.01 (2H, m), 1.72-1.63 (2H, m), 1.43 (9H, s).

MS:ESI 534 (M+1)

### Example 47

### tert-Butyl 2-{3-[(N-{4-[4-amino-2-(2-methoxyethyl)-1H-imidazo[4,5-c]quinolin-1-yl]butyl}-2-chloroacetamido)methyl]phenoxy}acetate hydrochloride

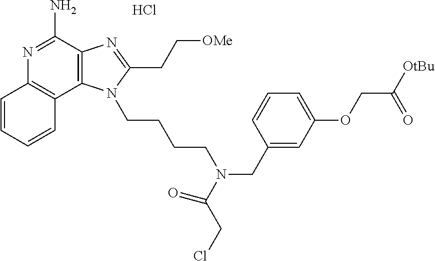

By the method of example 2 using the product of example 46 (209.0 mg), there was obtained the title compound, 301.2 mg (quant.) as a colorless gum.

¹H NMR δ (CDCl₃) 7.89-7.80 (2H, m), 7.53-7.48 (1H, m), 7.32 (1H, m), 7.24-7.19 (1H, m), 6.82-6.70 (3H, m), 5.46 (2H, brs), 4.57-4.47 (6H, m), 4.09 (0.5H, s), 4.00 (1.5H, s), 3.87 (2H, m), 3.43 (1.5H, t, J=7.2 Hz), 3.36 (2.3H, s), 3.35 (0.7H, s), 3.28 (0.5H, t, J=7.64 Hz), 3.17 (1.5H, t, J=6.4 Hz), 3.11 (0.5H, t, J=6.32 Hz), 1.94-1.86 (2H, m), 1.73-1.63 (2H, m), 1.43 (9H, s).

MS:ESI 610 (M+1)

### Example 48

### tert-Butyl 2-{3-[(N-{4-[4-amino-2-(2-methoxyethyl)-1H-imidazo[4,5-c]quinolin-1-yl]butyl}-2-{diethylamino}acetamido)methyl]phenoxy}acetate

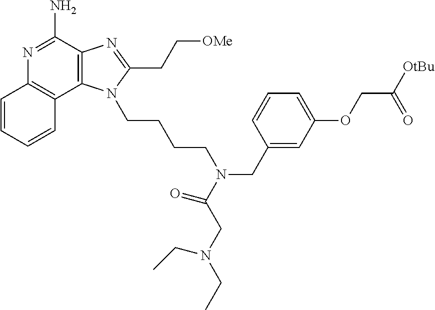

The title compound was prepared by the method of example 5 using the product from example 47 (226.0 mg) and diethylamine, to give a colorless gum (221.5 mg). Yield 92%.

¹H NMR δ (CDCl₃) 7.89-7.80 (2H, m), 7.49 (1H, m), 7.33-7.18 (2H, m), 6.82-6.71 (3H, m), 5.40 (2H, brs), 4.68 (1.4H, s), 4.55 (0.6H, s), 4.51-4.46 (4H, m), 3.87 (2H, t, J=6.48 Hz), 3.41-3.35 (5H, m), 3.24 (0.6H, s), 3.20 (1.4H, s), 3.16 (1.4H, t, J=6.48 Hz), 3.11 (0.6H, t, J=6.4 Hz), 2.51 (4H, m), 1.85 (2H, m), 1.70-1.62 (2H, m), 1.47 (9H, s), 0.95-0.90 (6H, m).

MS:ESI 647 (M+1)

### Example 49

### Methyl 2-[3-({3-[4-amino-2-(2-methoxyethyl)-1H-imidazo[4,5-c]quinolin-1-yl]propylamino}methyl)phenoxy]propanoate

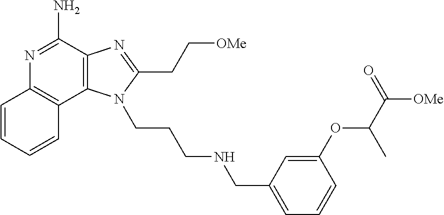

### (i) Methyl 2-(3-formylphenoxy)propanoate

By the method of example 23 step (i) using the 3-hydroxybenzaldehyde (500 mg) and the methyl 2-bromopropanoate (501 uL) to afford the title compound, 827.1 mg (97%) as colorless oil.

¹H NMR δ (CDCl₃) 9.96 (1H, s), 7.50 (2H, ddd, J=7.48, 1.32, 1.32 Hz), 7.46 (1H, dd, J=7.84, 7.52 Hz), 7.33 (1H, m), 7.18 (1H, m), 4.86 (1H, q, J=6.8 Hz), 3.78 (3H, s), 1.66 (3H, d, J=6.8 Hz).

MS:ESI 209 (M+1)

### (ii) Methyl 2-[3-({3-[4-amino-2-(2-methoxyethyl)-1H-imidazo[4,5-c]quinolin-1-yl]propylamino}methyl)phenoxy]propanoate

By the method of example 1 step (viii) using the product from example 15 step (iv) (200.0 mg) and methyl 2-(3-formylphenoxy)propanoate (139.1 mg) to afford the title compound, 289.4 mg (88%) as a white solid.

¹H NMR δ (CDCl₃) 8.08 (2H, dd, J=8.2, 0.88 Hz), 7.80 (1H, dd, J=8.36, 1.00 Hz), 7.48 (1H, m), 7.29-7.22 (2H, m), 6.95 (1H, d, J=7.6 Hz), 6.91 (1H, d, J=2.12 Hz), 6.75 (1H, dd, J=8.04, 2.16 Hz), 5.37 (2H, brs), 4.78 (2H, q, J=6.76 Hz), 4.65 (2H, t, J=7.4 Hz), 3.88 (2H., t, J=6.56 Hz), 3.77 (2H, s), 3.73 (3H, s), 3.37 (3H, s), 3.243 (2H, t, J=6.56 Hz), 2.73 (2H, t, J=6.28 Hz), 2.07 (2H, m), 1.61 (3H, d, J=6.8 Hz).

MS:ESI 492 (M+1)

### Example 50

### Methyl 2-{3-[(N-{3-[4-amino-2-(2-methoxyethyl)-1H-imidazo[4,5-c]quinolin-1-yl]propyl}-2-chloroacetamido)methyl]phenoxy}propanoate hydrochloride

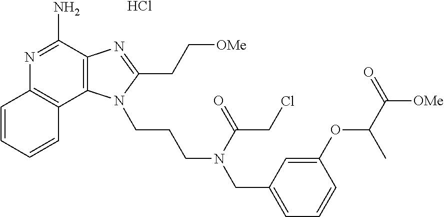

By the method of example 2 using the product of example 49 (285.4 mg), there was obtained the title compound, 341.8 mg (quant.) as a colorless gum.

¹H NMR δ (CDCl₃) 7.93-7.83 (2H, m), 7.53 (1H, m), 7.35 (1H, m), 7.23 (1H, m), 6.75-6.71 (3H, m), 5.33-5.47 (2H, m), 4.73 (1H, q, J=6.8 Hz), 4.60 (1.5H, s), 4.56-4.49 (2.5H, m), 4.10 (2H, s), 3.87 (2H, t, J=6.36 Hz), 3.75 (3H, s), 3.63-3.42 (2H, m), 3.37 (3H, s), 3.17-3.10 (2H, m), 2.28-2.13 (2H, m), 1.62 (3H, d, J=6.8 Hz).

MS:ESI 568 (M+1)

### Example 51

### Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}propanoate

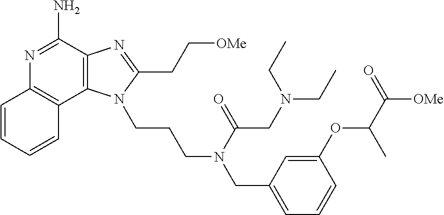

The title compound was prepared by the method of example 5 using the product from example 50 (185.0 mg) and diethylamine, to give a colorless gum (121.9 mg). Yield 62%.

¹H NMR δ (CDCl₃) 7.88-7.80 (2H, m), 7.50 (1H, m), 7.32 (1H, m), 7.18 (1H, m), 6.77-6.67 (3H, m), 5.40 (2H, brs), 4.74 (1.5H, s), 4.70 (1H, q, J=6.76 Hz), 4.56 (0.5H, s), 4.48 (2H, m), 3.85 (2H, m), 3.73 (3H, s), 3.56-3.51 (2H, m), 3.35 (2.3H, s), 3.33 (0.7H, s), 3.28 (1.5H, s), 3.25 (0.5H, s), 3.13 (1.5H, t, J=6.36 Hz), 3.08 (0.5H, t, J=6.24 Hz), 2.59 (3H, t, J=7.12 Hz), 2.51 (1H, t, J=7.12 Hz), 2.10-2.06 (2H, m), 1.60 (2H, d, J=6.8 Hz), 0.98 (6H, t, J=7.08 Hz).

MS:ESI 605 (M+1)

### Example 52

### Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}propanoate

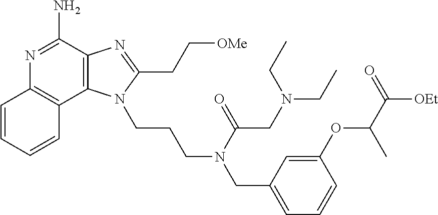

### (i) 2-{3-[(N-{3-[4-Amino-2-(2-methoxyethyl)-1H-imidazo[4,5-c]quinolin-1-yl]propyl}-2-{diethylamino}acetamido)methyl]phenoxy}propanoic acid

The title compound was prepared by the method of example 26 step (i) using the product from example 51 (79.9 mg), to give a white solid (78.0 mg). Yield quant.

¹H NMR δ (DMSO-d₆) 7.94 (0.5H, d, J=8.16 Hz), 7.90 (0.5H, d, J=8.48 Hz), 7.59 (1H, d, J=7.28 Hz), 7.44 (1H, m), 7.27-7.16 (2H, m), 7.12 (2H, brs), 6.78-6.72 (3H, m), 4.75-4.68 (2H, m), 4.51-4.48 (2H, m), 4.40 (1H, m), 3.79 (2H, m), 3.38 (2H, m), 3.28 (3H, s), 3.13 (2H, m), 2.68-2.58 (2H, m), 2.51-2.50 (2H, m), 2.09 (1H, m), 1.49 (1.5H, d, J=6.68 Hz), 1.48 (1.5H, d, J=6.72 Hz), 0.95-0.85 (6H, m).

MS:ESI 591 (M+1)

### (ii) Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}propanoate

The title compound was prepared by the method of example 26 step (ii) using the product from step (i) (57.8 mg) and EtOH, to give a colorless gum (42.5 mg). Yield 70%.

¹H NMR δ (CDCl₃) 7.92-7.84 (2H, m), 7.53 (1H, m), 7.38-7.31 (1H, m), 7.20 (1H, m), 6.79-6.71 (3H, m), 5.63 (2H, brs), 4.77-4.68 (2.5H, m), 4.58 (0.5H, m), 4.50 (2H, m), 4.20 (2H, m), 3.87 (2H, m), 3.59-3.53 (2H, m), 3.37 (2.3H, s), 3.35 (0.7H, s), 3.30 (1.5H, s), 3.27 (0.5H, s), 3.15 (1.5H, t, J=6.32 Hz), 3.10 (0.5H, t, J=6.24 Hz), 2.61 (3H, q, J=7.12 Hz), 2.54 (1H, q, J=7.08 Hz), 2.11-2.05 (2H, m), 1.61 (3H, d, J=6.80 Hz), 1.25 (3H, t, J=7.12 Hz), 1.00 (6H, t, J=7.12 Hz).

MS:ESI 619 (M+1)

### Example 53

### Ethyl 2-[3-({3-[4-amino-2-(2-methoxyethyl)-1H-imidazo[4,5-c]quinolin-1-yl]propylamino}methyl)phenoxy]-2-methylpropanoate

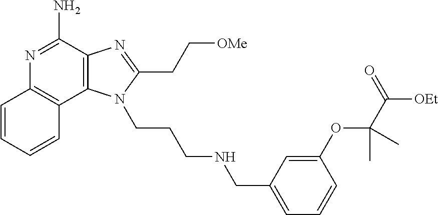

### (i) Ethyl 2-(3-formylphenoxy)-2-methylpropanoate

By the method of example 23 step (i) using the 3-hydroxybenzaldehyde (300 mg) and the ethyl 2-bromo-2-methylpropionate (582.6 mg) to afford the title compound, 244.2 mg (42%) as colorless oil.

¹H NMR δ (CDCl₃) 9.94 (1H, s), 7.51 (1H, ddd, J=7.52, 1.2, 1.2 Hz), 7.42 (1H, dd, J=7.96, 7.68 Hz), 7.32 (1H, m), 7.13 (1H, ddd, J=8.12, 2.64, 1.04 Hz), 4.25 (2H, q, J=7.12 Hz), 1.64 (6H, s), 1.26 (3H, t, J=7.12 Hz).

### (ii) Ethyl 2-[3-({3-[4-amino-2-(2-methoxyethyl)-1H-imidazo[4,5-c]quinolin-1-yl]propylamino}methyl)phenoxy]-2-methylpropanoate

By the method of example 1 step (viii) using the product from example 15 step (iv) (127.2 mg) and ethyl 2-(3-formylphenoxy)-2-methylpropanoate (100.4 mg) to afford the title compound, 189.1 mg (86%) as a white solid.

¹H NMR δ (CDCl₃) 8.09 (1H, dd, J=8.28, 1.00 Hz), 7.82 (1H, dd, J=8.36, 1.04 Hz), 7.50 (1H, m), 7.32-7.28 (1H, m), 7.21 (1H, dd, J=7.92, 7.8 Hz), 6.98 (1H, d, J=7.68 Hz), 6.89 (1H, m), 6.73 (1H, dd, J=8.2, 1.88 Hz), 5.40 (2H, brs), 4.66 (2H, t, J=7.4 Hz), 4.23 (2H, q, J=7.12 Hz), 3.90 (2H, t, J=6.56 Hz), 3.38 (3H, s), 3.25 (2H, t, J=6.48 Hz), 2.74 (2H, t, J=6.24 Hz), 2.08 (2H, m), 1.61 (6H, s), 1.24 (2H, t, J=7.12 Hz).

MS:ESI 520 (M+1)

### Example 54

### Ethyl

2-{3-[(N-{3-[4-amino-2-(2-methoxyethyl)-1H-imidazo[4,5-c]quinolin-1-yl]propyl}-2-chloroacetamido)methyl]phenoxy}-2-methylpropanoate hydrochloride

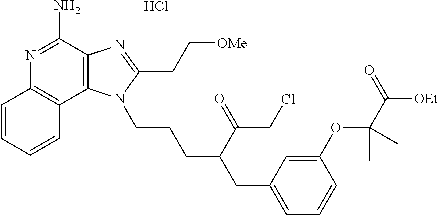

By the method of example 2 using the product of example 53 (186.3 mg), there was obtained the title compound, 222.4 mg (quant.) as a colorless gum.

¹H NMR δ (CDCl₃) 7.91 (1H, d, J=7.52 Hz), 7.82 (1H, d, J=8.36 Hz), 7.51 (1H, m), 7.34 (1H, m), 7.21-7.13 (1H, m), 6.74-6.67 (3H, m), 5.55 (1.5H, brs), 5.47 (0.5H, brs), 4.58 (1.5H, s), 4.54-4.46 (2.5H. m), 4.19 (2H, q, J=7.12 Hz), 4.07 (2H, s), 3.85 (2H, t, J=6.36 Hz), 3.58 (1.5H, t, J=6.72 Hz), 3.42-3.37 (0.5H, m), 3.34 (2.3H, s), 3.32 (0.7H, s), 3.14 (1.5H, t, J=6.32 Hz), 3.10 (0.5H, t, J=6.2 Hz), 2.23-2.09 (2H, m), 1.56 (4.5H, s), 1.55 (1.5H, s), 1.22 (3H, t, J=7.12 Hz).

MS:ESI 596 (M+1)

### Example 55

### Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}-2-methylpropanoate

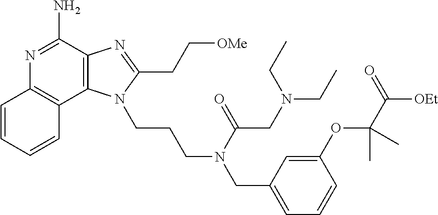

The title compound was prepared by the method of example 5 using the product from example 54 (217.8 mg) and diethylamine, to give a colorless gum (170.4 mg). Yield 74%.

¹H NMR δ (CDCl₃) 7.92-7.83 (2H, m), 7.53 (1H, m), 7.35 (1H, m), 7.19-7.16 (1H, m), 6.81-6.70 (3H, m), 5.43 (2H, brs), 4.77 (1.5H, s), 4.58 (0.5H, s), 4.51 (2H, m), 4.22 (2H, q, J=7.16 Hz), 3.88 (2H, m), 3.57 (2H, m), 3.37 (2.3H, s), 3.35 (0.7H, s), 3.30 (1.5H, s), 3.27 (0.5H, s), 3.17 (1.5H, t, J=6.36 Hz), 3.11 (0.5H, t, J=6.32 Hz), 2.61 (3H, q, J=7.2 Hz), 2.53 (1H, q, J=7.04 Hz), 2.19-2.09 (2H, m), 1.58 (6H, s), 1.24 (3H, q, J=7.16 Hz), 1.00 (6H, t, J=7.08 Hz).

MS:ESI 633 (M+1)

### Example 56

### Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}-2-methylpropanoate

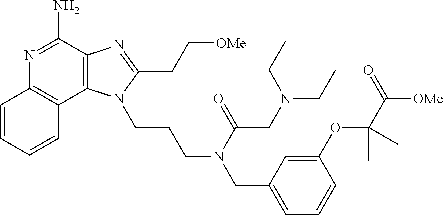

### (i) 2-{3-[(N-{3-[4-Amino-2-(2-methoxyethyl)-1H-imidazo[4,5-c]quinolin-1-yl]propyl}-2-{diethylamino}acetamido)methyl]phenoxy}-2-methylpropanoic acid

The title compound was prepared by the method of example 26 step (i) using the product from example 55 (147.2 mg), to give a white solid (135.9 mg). Yield 97%.

¹H NMR δ (DMSO-d₆) 7.88 (0.5H, d, J=8.24 Hz), 7.85 (0.5H, d, J=8.32 Hz), 7.59 (1H, d, J=8.24 Hz), 7.43 (1H, m), 7.23-7.12 (2H, m), 6.76-6.67 (3H, m), 4.68 (1H, s), 4.43 (2H, m), 4.33 (1H, m), 3.80-3.74 (4H, m), 3.50 (1H, m), 3.41 (1H, m), 3.22 (1H, s), 3.21 (1H, s), 3.10 (2H, m), 2.47-2.39 (2H, m), 2.04 (1H, m), 1.86 (1H, m), 1.48 (3H, s), 1.47 (3H, s), 0.88-0.82 (6H, m).

MS:ESI 605 (M+1)

### (ii) Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}-2-methylpropanoate

The title compound was prepared by the method of example 26 step (ii) using the product from step (i) (84.3 mg) and MeOH, to give a colorless gum (66.4 mg). Yield 74%.

¹H NMR δ (CDCl₃) 7.92-7.83 (2H, m), 7.53 (1H, m), 7.35 (1H, m), 7.22-7.17 (1H, m), 6.82-6.69 (3H, m), 5.41 (2H, brs), 4.77 (1.5H, s), 4.57 (0.5H, s), 4.51 (2H, m), 3.88 (2H, m), 3.76 (3H, s), 3.56 (2H, t, J=7.00 Hz), 3.37 (2.3H, s), 3.35 (0.7H, s), 3.30 (1.5H, s), 3.27 (0.5H, s), 3.17 (1.5H, t, J=6.36 Hz), 3.11 (0.5H, t, J=6.24 Hz), 2.61 (3H, q, J=7.16 Hz), 2.53 (1H, q, J=7.12 Hz), 2.15-2.07 (2H, m), 1.58 (6H, s), 1.00 (6H, t, J=7.08 Hz).

MS:ESI 619 (M+1)

### Example 57

### Ethyl 1-[3-({3-[4-amino-2-(2-methoxyethyl)-1H-imidazo[4,5-c]quinolin-1-yl]propylamino}methyl)phenoxy]cyclobutanecarboxylate

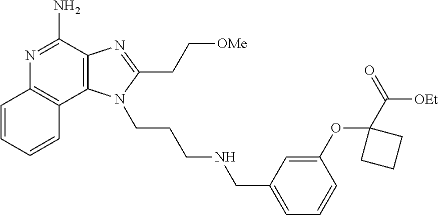

### (i) Ethyl 1-(3-formylphenoxy)cyclobutanecarboxylate

By the method of example 23 step (i) using the 3-hydroxybenzaldehyde (500 mg) and the ethyl 1-bromocyclobutanecarboxylate (694.6 uL) to afford the title compound, 76.2 mg (8%) as colorless oil.

¹H NMR δ (CDCl₃) 9.93 (1H, s), 7.46 (1H, m), 7.41 (1H, dd, J=7.88, 7.6 Hz), 7.13 (1H, m), 6.98 (1H, ddd, J=8.00, 2.64, 1.16 Hz), 4.21 (2H, q, J=7.08 Hz), 2.82-2.76 (2H, m), 2.51-2.43 (2H, m), 2.10-1.99 (2H, m), 1.18 (3H, t, J=7.12 Hz).

### (ii) Ethyl 1-[3-({3-[4-amino-2-(2-methoxyethyl)-1H-imidazo[4,5-c]quinolin-1-yl]propylamino}methyl)phenoxy]cyclobutanecarboxylate

By the method of example 1 step (viii) using the product from example 15 step (iv) (87.4 mg) and ethyl 2-(3-formylphenoxy)-2-methylpropanoate (100.4 mg) to afford the title compound, 109.4 mg (71%) as a white solid.

¹H NMR δ (CDCl₃) 8.09 (1H, m), 7.82 (1H, dd, J=8.32, 0.88 Hz), 7.51 (1H, m), 7.29 (1H, m), 7.21 (1H, dd, J=7.92, 7.84 Hz), 6.93 (1H, d, J=7.52 Hz), 6.93 (1H, m), 6.78 (1H, m), 6.54 (1H, dd, J=7.96, 2.24 Hz), 5.41 (2H, brs), 4.67 (2H, m), 4.19 (2H, q, J=7.12 Hz), 3.91 (2H, t, J=6.52 Hz), 3.78 (2H, s), 3.39 (3H, s), 3.26 (2H, t, J=6.52 Hz), 2.80-2.73 (4H, m), 2.51-2.43 (2H, m), 2.11-1.97 (4H, m), 1.17 (3H, t, J=7.08 Hz).

MS:ESI 532 (M+1)

### Example 58

### Ethyl 1-{3-[(N-{3-[4-amino-2-(2-methoxyethyl)-1H-imidazo[4,5-c]quinolin-1-yl]propyl}-2-chloroacetamido)methyl]phenoxy}cyclobutanecarboxylate hydrochloride

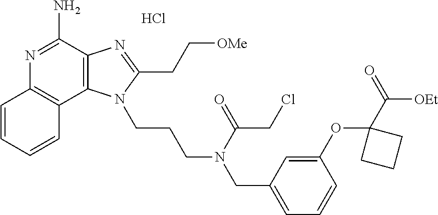

By the method of example 2 using the product of example 57 (107.1 mg), there was obtained the title compound, 116.1 mg (quant.) as a colorless gum.

¹H NMR δ (CDCl₃) 7.92 (1H, d, J=7.44 Hz), 7.84 (1H, d, J=8.28 Hz), 7.53 (1H, m), 7.36 (1H, m), 7.19 (1H, dd, J=7.88, 7.88 Hz), 6.71 (1H, d, J=7.52 Hz), 6.57 (1H, d, J=6.48 Hz), 6.53 (1H, dd, J=8.04, 2.24 Hz), 5.59 (2H, brs), 4.59 (2H, s), 4.56-4.51 (2H, m), 4.17 (2H, q, J=7.12 Hz), 4.07 (2H, s), 3.87 (2H, t, J=6.36 Hz), 3.60 (2H, t, J=6.68 Hz), 3.36 (2.3H, s), 3.34 (0.7H, s), 3.16 (1.5H, t, J=6.36 Hz), 3.12 (0.5H, m), 2.72 (2H, m), 2.42 (2H, m), 2.15 (2H, m), 2.03-1.97 (2H, m), 1.17 (3H, t, J=7.08 Hz).

MS:ESI 608 (M+1)

### Example 59

### Ethyl 1-{3-[(N-{3-[4-amino-2-(2-methoxyethyl)-1H-imidazo[4,5-c]quinolin-1-yl]propyl}-2-{diethylamino}acetamido)methyl]phenoxy}cyclobutanecarboxylate

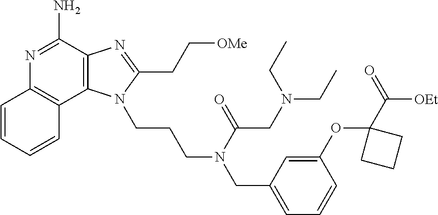

The title compound was prepared by the method of example 5 using the product from example 58 (116.1 mg) and diethylamine, to give a colorless gum (80.5 mg). Yield 77%.

¹H NMR δ (CDCl₃) 7.91-7.83 (2H, m), 7.53 (1H, m), 7.34 (1H, m), 7.17 (1H, dd, J=8.04, 7.92 Hz), 6.74-6.70 (1H, m), 6.63 (0.25H, s), 6.57 (0.75H, s), 6.49 (1H, dd, J=8.4, 2.4 Hz), 4.76 (1.5H, s), 4.56 (0.5H, s), 4.50 (2H, m), 4.17 (2H, q, J=7.12 Hz), 3.87 (2H, m), 3.55 (2H, m), 3.36 (2.3H, s), 3.34 (0.7H, s), 3.28 (2H, s), 3.15 (1.5H, t, J=6.36 Hz), 3.10 (0.5H, m), 2.72 (2H, m), 2.59 (3H, q, J=7.16 Hz), 2.52 (1H, q, J=7.08 Hz), 2.41 (2H, m), 2.09 (2H, m), 2.02-1.96 (2H, m), 1.16 (3H, q, J=7.08 Hz), 0.99 (6H, t, J=7.08 Hz).

MS:ESI 645 (M+1)

### Example 60

### Ethyl 2-[5-({3-[4-amino-2-(2-methoxyethyl)-1H-imidazo[4,5-c]quinolin-1-yl]propylamino}methyl)-2-methoxyphenoxy]acetate

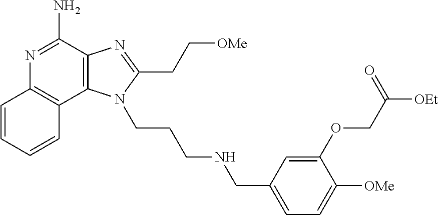

By the method of example 1 step (viii) using the product from example 15 step (iv) (200.0 mg) ethyl 2-(5-formyl-2-methoxyphenoxy)acetate (159.1 mg) there was obtained the title compound, 261.5 mg (75%) as a white solid.

¹H NMR δ (CDCl₃) 8.08 (1H, dd, J=8.20, 0.88 Hz), 7.81 (1H, dd, J=8.36, 1.00 Hz), 7.49 (1H, m), 7.28 (1H, m), 6.93 (1H, dd, J=8.12, 1.88 Hz), 6.93 (2H, m), 6.87-6.85 2H, m), 5.40 (2H, brs), 4.69 (2H, s), 4.64 (2H, t, J=7.36 Hz), 4.22 (2H, q, J=7.12 Hz), 3.89 (2H, t, J=6.64 Hz), 3.88 (3H, s), 3.72 (2H, s), 3.37 (3H, s), 3.24 (2H, t, J=6.60 Hz), 2.72 (2H, t, J=6.32 Hz), 2.07 (2H, m), 1.25 (3H, t, J=7.12 Hz).

MS:ESI 522 (M+1)

### Example 61

### Ethyl 2-{5-[(N-{3-[4-amino-2-(2-methoxyethyl)-1H-imidazo[4,5-c]quinolin-1-yl]propyl}2-chloroacetamido)methyl]-2-methoxyphenoxy}acetate hydrochloride

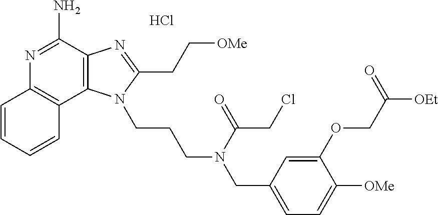

By the method of example 2 using the product of example 60 (261.5 mg), there was obtained the title compound, 310.9 mg (quant.) as a colorless gum.

¹H NMR δ (CDCl₃) 7.91-7.81 (2H, m), 7.53 (1H, d, J=7.96 Hz), 7.38-7.31 (1H, m), 6.80-6.65 (3H, m), 5.79 (1.5H, brs), 5.63 (0.5H, brs), 4.63 (1.5H, s), 4.62 (0.5H, s), 4.54-4.48 (4H, m), 4.22 (2H, q, J=7.16 Hz), 4.11 (1.5H, s), 4.05 (0.5H, s), 3.88-3.85 (5H, m), 3.54 (2H, t, J=7.00 Hz), 3.36 (3H, s), 3.15-3.09 (2H, m), 2.24-2.07 (2H, m), 1.28 (3H, t, J=7.12 Hz).

MS:ESI 598 (M+1)

### Example 62

### Ethyl 2-{5-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate

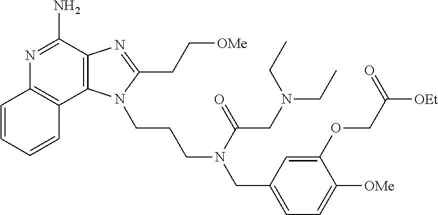

The title compound was prepared by the method of example 5 using the product from example 61 (306.0 mg) and diethylamine, to give a colorless gum (224.9 mg). Yield 71%.

¹H NMR δ (CDCl₃) 7.91-7.83 (2H, m), 7.53 (1H, m), 7.34 (1H, m), 7.35 (1H, m), 6.80-6.66 (3H, m), 5.48-5.453 (2H, brm), 4.65 (1.5H, s), 4.67 (1.5H, s), 4.63 (1.5H, s), 4.62 (0.5H, s), 4.51-4.47 (2.5H, m), 4.26-4.20 (2H, m), 3.89-3.85 (5H, m), 3.56-3.48 (2H, m), 3.37 (2.3H, s), 3.35 (0.7H, s), 3.32 (1.5H, s), 3.25 (0.5H, s), 3.14 (1.5H, t, J=6.4 Hz), 3.09 (0.5H, t, J=6.24 Hz), 2.62 (3H, q, J=7.16 Hz), 2.53 (1H, q, J=7.12 Hz), 2.21-2.06 (2H, m), 1.30-1.25 (3H, m), 1.03-0.97 (6H, m).

MS:ESI 635 (M+1)

### Example 63

### Methyl 2-{5-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate

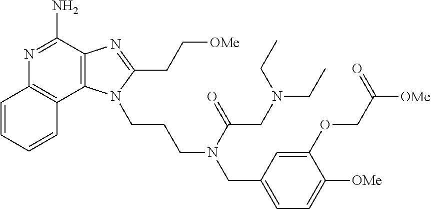

### (i) 2-{5-[(N-{3-[4-Amino-2-(2-methoxyethyl)-1H-imidazo[4,5-c]quinolin-1-yl]propyl}-2-{diethylamino}acetamido)methyl]-2-methoxyphenoxy}acetic acid

The title compound was prepared by the method of example 26 step (i) using the product from example 62 (124.7 mg), to give a white solid (121.0 mg). Yield quant.

¹H NMR δ (DMSO-d₆) 7.95 (0.5H, d, J=8.68 Hz), 7.92 (0.5H, d, J=8.56 Hz), 7.57 (1H, d, J=8.12 Hz), 7.42 (1H, m), 7.27 (0.5H, d, J=7.24 Hz), 7.24 (0.5H, d, J=6.56 Hz), 6.89 (1H, d, J=8.24 Hz), 6.83 (1H, d, J=8.16 Hz), 6.75-6.64 (2H, m), 4.61 (1H, s), 4.50 (1H, m), 4.40-4.39 (4H, m), 3.79 (2H, m), 3.74 (2H, m), 3.28 (3H, s), 3.26 (1H, s), 3.18 (1H, s), 3.11 (2H, m), 2.58-2.50 (2H, m), 2.42 (2H, m), 2.08 (1H, m), 1.92 (1H, m), 0.91 (3H, t, J=7.10 Hz), 0.85 (3H, d, J=7.14 Hz).

MS:ESI 607 (M+1)

### (ii) Methyl 2-{5-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate

The title compound was prepared by the method of example 26 step (ii) using the product from step (i) (59.8 mg) and MeOH, to give a colorless gum (37.2 mg). Yield 61%.

¹H NMR δ (CDCl₃) 7.91-7.83 (2H, m), 7.53 (1H, m), 7.34 (1H, m), 7.35 (1H, m), 6.80-6.66 (3H, m), 5.48-5.453 (2H, brm), 4.65 (1.5H, s), 4.67 (1.5H, s), 4.63 (1.5H, s), 4.62 (0.5H, s), 4.51-4.47 (2.5H, m), 3.89-3.85 (5H, m), 3.75 (3H, s), 3.56-3.48 (2H, m), 3.37 (2.3H, s), 3.35 (0.7H, s), 3.32 (1.5H, s), 3.25 (0.5H, s), 3.14 (1.5H, t, J=6.4 Hz), 3.09 (0.5H, t, J=6.24 Hz), 2.62 (3H, q, J=7.16 Hz), 2.53 (1H, q, J=7.12 Hz), 2.21-2.06 (2H, m), 1.03-0.97 (6H, m).

MS:ESI 621 (M+1)

### Example 64

### Ethyl 2-[5-({3-[4-amino-2-(2-methoxyethyl)-1H-imidazo[4,5-c]quinolin-1-yl]propylamino}methyl)-2-methylphenoxy]acetate

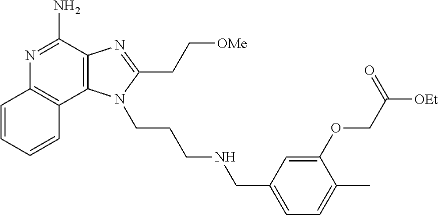

### (i) 5-(Hydroxymethyl)-2-methylphenol

To a solution of BH₃.THF (1.06M in THF solution, 4.66 mL, 4.94 mmol) 3-hydroxy-4-methylbenzoic acid (500 mg, 3.29 mmol) and B(OMe)₃ (683.7 mg, 6.58 mmol) in THF (3.3 ml) was added at rt. After stirring for 6 h at rt, cooled to 0° C., and H₂O was added. The aq. layer was extracted with AcOEt, dried over Na₂SO₄, and concentrated. The mixture was stirred at rt for 30 min, and concentrated. The residue was diluted with AcOEt, H₂O was added. The aq. layer was extracted with AcOEt, dried over Na₂SO₄, and concentrated. The residue was purified by flash column chromatography to give the title compound (446.2 mg, 98%) as a white solid.

¹H NMR δ (CDCl₃) 7.10 (1H, d, J=7.4 Hz), 6.83 (1H, d, J=8.16 Hz), 6.81 (1H, s), 4.90 (1H, m), 4.62 (2H, d, J=5.08 Hz), 2.24 (3H, s), 1.64 (1H, t, J=7.12 Hzbrs).

### (ii) 3-Hydroxy-4-methylbenzaldehyde

To a solution of the product step (i) (440.0 mg, 3.18 mmol) in THF (4.4 mL) MnO₂ (552.9 mg, 6.36 mmol) was added at rt. After stirring for 6 h at 50° C., the mixture was filtered through the celite pad. The filtrate was concentrated, and the residue was purified by flash column chromatography to give the title compound (46.1 mg, 11%) as a pale yellow solid.

¹H NMR δ (CDCl₃) 9.90 (1H, s), 7.36 (1H, dd, J=7.76, 1.44 Hz), 7.30 (1H, s), 7.29 (1H, d, J=5.52 Hz), 5.33 (1H, s), 2.33 (3H, s).

### (iii) Ethyl 2-(5-formyl-2-methylphenoxy)acetate

To a solution of the product step (ii) (43.6 mg, 0.32 mmol) in DMF (0.5 ml) ethylbromoacetate (37.3 ul, 0.336 mmol) and K₂CO₃ (46.4 mg, 0.336 mmol) was added at rt. After stirring for 3 h at 60° C., diluted with AcOEt and H₂O was added. The aq. layer was extracted with AcOEt, dried over Na₂SO₄, and concentrated. The residue was purified by flash column chromatography to give the title compound (68.9 mg, 97%) as colorless oil.

¹H NMR δ (CDCl₃) 9.91 (1H, s), 7.41 (1H, dd, J=7.52, 1.32 Hz), 7.34 (1H, d, J=7.56 Hz), 7.22 (1H, m), 4.72 (2H, s), 4.28 (2H, q, J=7.16 Hz), 2.38 (3H, s), 1.31 (3H, t, J=7.12 Hz).

### (iv) Ethyl 2-[5-({3-[4-amino-2-(2-methoxyethyl)-1H-imidazo[4,5-c]quinolin-1-yl]propylamino}methyl)-2-methylphenoxy]acetate

By the method of example 1 step (viii) using the product from example 15 step (iv) (91.0 mg) and ethyl 2-(5-formyl-2-methylphenoxy)acetate (67.5 mg) there was obtained the title compound, 123.7 mg (81%) as a white solid.

¹H NMR δ (CDCl₃) 8.08 (1H, dd, J=8.24, 0.92 Hz), 7.82 (1H, dd, J=8.36, 1.04 Hz), 7.50 (1H, m), 7.30-7.26 (1H, m), 7.12 (1H, d, J=7.56 Hz), 6.88 (1H, dd, J=7.44, 1.00 Hz), 6.73 (1H, s), 5.42 (2H, brs), 4.65 (4H, m), 4.23 (2H, q, J=7.126 Hz), 3.89 (2H, t, J=6.64 Hz), 3.75 (2H, s), 3.38 (3H, s), 3.25 (2H, t, J=6.52 Hz), 2.73 (2H, t, J=6.32 Hz), 2.29 (3H, s), 2.08 (2H, m), 1.26 (3H, t, J=7.16 Hz).

MS:ESI 506 (M+1)

### Example 65

### Ethyl 2-{5-[(N-{3-[4-amino-2-(2-methoxyethyl)-1H-imidazo[4,5-c]quinolin-1-yl]propyl}-2-chloroacetamido)methyl]-2-methylphenoxy}acetate hydrochloride

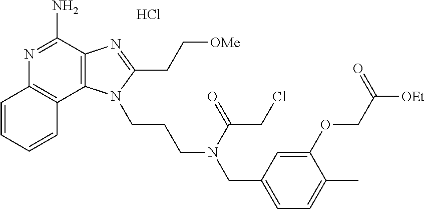

By the method of example 2 using the product of example 64 (122.5 mg), there was obtained the title compound, 146.9 mg (quant.) as a colorless gum.

¹H NMR δ (CDCl₃) 7.91-7.81 (2H, m), 7.53 (1H, m), 7.34 (1H, m), 7.10 (0.75H, d, J=7.48 Hz), 7.04 (0.25H, d, J=7.16 Hz), 6.66-6.61 (1H, m), 6.54 (0.25H, s), 6.46 (0.75H, s), 5.61-5.52 (2H, brm), 4.57-4.48 (6H, m), 4.27-4.21 (2H, m), 4.11 (1.5H, s), 4.06 (0.5H, s), 3.87 (2H, t, J=6.40 Hz), 3.57 (2H, t, J=6.88 Hz), 3.36 (2.3H, s), 3.34 (0.7H, s), 3.14 (1.5H, t, J=6.32 Hz), 3.10 (0.5H, t, J=6.04 Hz), 2.31-2.23 (3.5H, m), 2.13 (1.5H, m), 1.25 (3H, t, J=7.08 Hz).

MS:ESI 582 (M+1)

### Example 66

### Ethyl 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetate

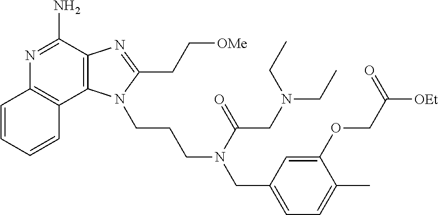

The title compound was prepared by the method of example 5 using the product from example 65 (128.2 mg) and diethylamine, to give a colorless gum (132.4 mg). Yield 97%.

¹H NMR δ (CDCl₃) 7.91-7.82 (2H, m), 7.52 (1H, m), 7.33 (1H, m), 7.09 (0.75H, d, J=7.76 Hz), 7.05 (0.25H, d, J=7.44 Hz), 6.66 (1H, d, J=7.52 Hz), 5.46-5.45 (2H, brm), 4.72 (1.5H, s), 4.56 (2H, s), 4.53 (0.5H, s), 4.49 (2H, m), 4.24 (2H, q, J=7.12 Hz), 3.87 (2H, m), 3.56-3.50 (2H, m), 3.36 (2.3H, s), 3.35 (0.7H, s), 3.31 (1.5H, s), 3.26 (0.5H, s), 3.14 (1.5H, t, J=6.36 Hz), 3.14 (0.5H, t, J=6.16 Hz), 2.61 (3H, q, J=7.12 Hz), 2.54 (1H, q, J=7.16 Hz), 2.26 (3H, s), 2.20-2.06 (2H, m), 1.29 (3H, t, J=7.16 Hz), 1.02-0.98 (6H, m).

MS:ESI 619 (M+1)

### Example 67

### Isopropyl 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetate

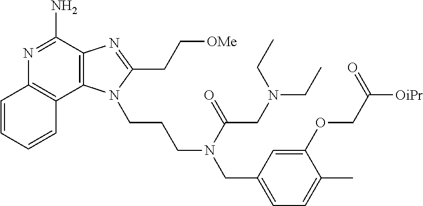

### (i) 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetic acid

The title compound was prepared by the method of example 26 step (i) using the product from example 66 (93.2 mg), to give a white solid (91.7 mg). Yield quant.

¹H NMR δ (DMSO-d₆) 7.97 (0.5H, d, J=8.72 Hz), 7.95 (0.5H, d, J=8.44 Hz), 7.59 (1H, d, J=8.32 Hz), 7.42 (0.5H, d, J=7.24 Hz), 7.40 (0.5H, d, J=7.16 Hz), 7.26 (0.5H, d, J=8.12 Hz), 7.21 (0.5H, d, J=7.84 Hz), 7.03 (0.5H, d, J=7.64 Hz), 6.98 (0.5H, d, J=7.68 Hz), 6.63 (2H, brs), 6.58 (1H, d, J=7.76 Hz), 6.55 (1H, d, J=7.32 Hz), 4.65 (1H, s), 4.51 (2H, m), 4.44-4.40 (2H, m), 4.24 (2H, m), 3.78 (2H, m), 3.26 (3H, s), 3.21 (2H, s), 3.15 (2H, s), 3.12 (2H, t, J=6.68 Hz), 2.53-2.49 (2H, m), 2.37 (2H, m), 2.12 (3H, s), 2.08 (1H, m), 1.95 (1H, m). 0.90-0.80 (6H, m).

MS:ESI 591 (M+1)

### (ii) Isopropyl 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetate

The title compound was prepared by the method of example 26 step (ii) using the product from step (i) (48.8 mg) and iPrOH, to give a colorless gum (45.5 mg). Yield 87%.

¹H NMR δ (CDCl₃) 7.91-7.82 (2H, m), 7.52 (1H, m), 7.33 (1H, m), 7.08 (0.75H, d, J=7.68 Hz), 7.05 (0.25H, d, J=7.36 Hz), 6.66 (1H, d, J=7.32 Hz), 6.60 (0.25H, s), 6.47 (0.75H, s), 5.49 (2H, brm), 5.10 (1H, m), 4.72 (1.5H, s), 4.53-4.46 (4.5H, m), 3.86 (2H, m), 3.57-3.50 (2H, m), 3.36 (2.3H, s), 3.34 (0.7H, s), 3.31 (1.5H, s), 3.25 (0.5H, s), 3.13 (1.5H, t, J=6.4 Hz), 3.06 (0.5H, t, J=6.2 Hz), 2.61 (3H, q, J=7.12 Hz), 2.53 (1H, q, J=7.2 Hz), 2.27 (3H, s), 2.20-2.06 (2H, m), 1.26 (6H, d, J=6.28 Hz), 1.00 (6H, m).

MS:ESI 633 (M+1)

### Example 68

### Methyl 2-[3-({3-[4-amino-2-(2-methoxyethyl)-1H-imidazo[4,5-c]quinolin-1-yl]propylamino}methyl)phenoxy]butanoate

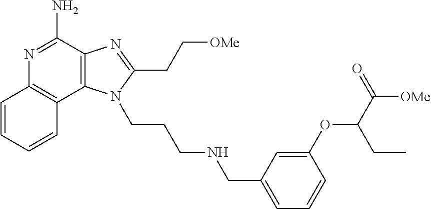

### (i) Methyl 2-(3-formylphenoxy)butanoate

By the method of example 23 step (i) using the 3-hydroxybenzaldehyde (500 mg) and the methyl 2-bromobutanoate (535.6 uL) to afford the title compound, 743.9 mg (82%) as colorless oil.

¹H NMR δ (CDCl₃) 9.96 (1H, s), 7.50 (2H, ddd, J=7.48, 1.32, 1.32 Hz), 7.46 (1H, dd, J=7.84, 7.52 Hz), 7.33 (1H, m), 7.18 (1H, m), 4.86 (1H, q, J=6.8 Hz), 3.78 (3H, s), 1.66 (3H, d, J=6.8 Hz).

MS:ESI 223 (M+1)

### (ii) Methyl 2-[3-({3-[4-amino-2-(2-methoxyethyl)-1H-imidazo[4,5-c]quinolin-1-yl]propylamino}methyl)phenoxy]butanoate

By the method of example 1 step (viii) using the product from example 15 step (iv) (201.2 mg) and methyl 2-(3-formylphenoxy)butanoate (149.3 mg) to afford the title compound, 252.7 mg (74%) as a white solid.

¹H NMR δ (CDCl₃) 8.10 (1H, d, J=7.36 Hz), 7.82 (1H, dd, J=8.36, 0.92 Hz), 7.50 (1H, m), 7.31-7.23 (2H, m), 6.96 (1H, d, J=7.56 Hz), 6.94 (1H, m), 6.77 (1H, dd, J=7.96, 2.2 Hz), 5.45 (2H, brs), 4.67 (2H, m), 4.61 (2H, t, J=6.12 Hz), 3.90 (2H., t, J=6.56 Hz), 3.79 (2H, s), 3.74 (3H, s), 3.38 (3H, s), 3.26 (2H, t, J=6.52 Hz), 2.75 (2H, t, J=6.28 Hz), 2.12-2.05 (2H, m), 2.03-1.96 (2H, m), 1.08 (3H, d, J=7.48 Hz).

MS:ESI 506 (M+1)

### Example 69

### Methyl 2-{3-[(N-{3-[4-amino-2-(2-methoxyethyl)-1H-imidazo[4,5-c]quinolin-1-yl]propyl}-2-chloroacetamido)methyl]phenoxy}butanoate hydrochloride

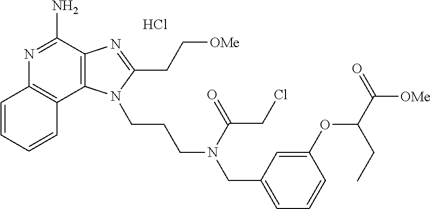

By the method of example 2 using the product of example 68 (239.6 mg), there was obtained the title compound, 280.9 mg (quant.) as a colorless gum.

¹H NMR δ (CDCl₃) 7.943-7.83 (2H, m), 7.55 (1H, m), 7.38 (1H, m), 7.24 (1H, m), 6.77-6.73 (3H, m), 5.75-5.69 (2H, m), 4.62 (1.5H, s), 4.57-4.53 (3.5H, m), 4.10 (2H, s), 3.88 (2H, t, J=6.28 Hz), 3.75 (3H, s), 3.61 (2H, m), 3.37 (3H, s), 3.18-3.11 (2H, m), 2.27-2.14 (2H, m), 2.05-1.96 (2H, m), 1.08 (3H, t, J=7.44 Hz).

MS:ESI 582 (M+1)

### Example 70

### Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}butanoate

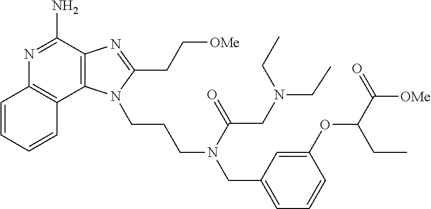

The title compound was prepared by the method of example 5 using the product from example 69 (277.9 mg) and diethylamine, to give a colorless gum (264.5 mg). Yield 90%.

¹H NMR δ (CDCl₃) 7.92-7.82 (2H, m), 7.52 (1H, m), 7.33 (1H, m), 7.20 (1H, m), 6.80-6.71 (3H, m), 5.49 (2H, brs), 4.76 (1.5H, s), 4.58-4.48 (3.5H, m), 3.85 (2H, m), 3.87 (2H, t, J=6.36 Hz), 3.74 (3H, s), 3.59-3.53 (2H, m), 3.36 (2.3H, s), 3.34 (0.7H, s), 3.30 (1.5H, s), 3.27 (0.5H, s), 3.15 (1.5H, t, J=6.36 Hz), 3.10 (0.5H, t, J=6.24 Hz), 2.60 (3H, t, J=7.12 Hz), 2.53 (1H, t, J=7.08 Hz), 2.23 (0.5H, m), 2.10 (1.5H, m), 1.98 (2H, m), 1.07 (3H, t, J=7.44 Hz), 1.00 (6H, t, J=7.12 Hz).

MS:ESI 619 (M+1)

### Example 71

### Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}butanoate

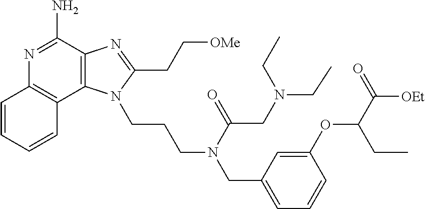

### (i) 2-{3-[(N-{3-[4-Amino-2-(2-methoxyethyl)-1H-imidazo[4,5-c]quinolin-1-yl]propyl}-2-{diethylamino}acetamido)methyl]phenoxy}butanoic acid

The title compound was prepared by the method of example 26 step (i) using the product from example 70 (182.4 mg), to give a white solid (144.1 mg). Yield 81%.

¹H NMR δ (DMSO-d₆) 7.94 (0.5H, d, J=8.12 Hz), 7.90 (0.5H, d, J=7.96 Hz), 7.58 (1H, d, J=8.32 Hz), 7.42 (1H, m), 7.25-7.16 (2H, m), 7.08 (1H, brs), 7.00 (1H, brs), 6.77-6.72 (3H, m), 4.69 (1H, s), 4.55-4.46 (3H, m), 4.40 (1H, m), 3.78 (2H, m), 3.56-3.42 (4H, m), 3.27 (3H, s), 3.11 (2H, m), 2.56-2.45 (4H, m), 2.09 (1H, m), 1.94 (3H, m), 0.98 (3H, t, J=7.36 Hz), 0.92-0.84 (6H, m).

MS:ESI 605 (M+1)

### (ii) Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}butanoate

The title compound was prepared by the method of example 26 step (ii) using the product from step (i) (71.7 mg) and EtOH, to give a colorless gum (61.6 mg). Yield 82%.

¹H NMR δ (CDCl₃) 7.93-7.84 (2H, m), 7.53 (1H, m), 7.38-7.32 (1H, m), 7.21 (1H, m), 6.81-6.72 (3H, m), 5.61 (2H, brs), 4.77 (1.5H, d, J=4.16 Hz), 4.59-4.48 (3.5H, m), 4.20 (2H, m), 3.87 (2H, t, J=6.4 Hz), 3.55 (2H, m), 3.37 (2.3H, s), 3.35 (0.7H, s), 3.31 (1.5H, s), 3.27 (0.5H, s), 3.16 (1.5H, t, J=6.32 Hz), 3.11 (0.5H, t, J=6.16 Hz), 2.60 (3H, q, J=7.12 Hz), 2.54 (1H, q, J=7.00 Hz), 2.24 (0.5H, m), 2.10 (1.5H, m), 1.99 (2H, m), 1.25 (3H, t, J=7.12 Hz), 1.08 (3H, t, J=7.4 Hz), 1.00 (6H, t, J=7.08 Hz).

MS:ESI 633 (M+1)

### Example 72

### Isopropyl 2-{5-[(N-{3-[4-amino-2-(2-methoxyethyl)-1H-imidazo[4,5-c]quinolin-1-yl]propyl}-2-{diethylamino}acetamido)methyl]-2-methoylphenoxy}acetate

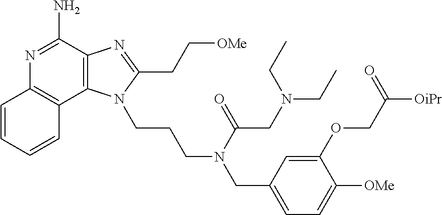

The title compound was prepared by the method of example 26 step (ii) using the product from example 63 step (i) (37.2 mg) and iPrOH, to give a colorless gum (31.4 mg). Yield 79%.

¹H NMR δ (CDCl₃) 7.91-7.83 (2H, m), 7.53 (1H, m), 7.34 (1H, m), 7.35 (1H, m), 6.80-6.66 (3H, m), 5.48-5.45 (2H, brm), 5.08 (1H, m), 4.65 (1.5H, s), 4.67 (1.5H, s), 4.63 (1.5H, s), 4.62 (0.5H, s), 4.51-4.47 (2.5H, m), 3.89-3.85 (5H, m), 3.56-3.48 (2H, m), 3.37 (2.3H, s), 3.35 (0.7H, s), 3.32 (1.5H, s), 3.25 (0.5H, s), 3.14 (1.5H, t, J=6.4 Hz), 3.09 (0.5H, t, J=6.24 Hz), 2.62 (3H, q, J=7.16 Hz), 2.53 (1H, q, J=7.12 Hz), 2.21-2.06 (2H, m), 1.26-1.23 (6H, m), 1.03-0.97 (6H, m).

MS:ESI 649 (M+1)

### Example 73

### Isopropyl 2-[5-({3-[4-amino-2-(2-methoxyethyl)-1H-imidazo[4,5-c]quinolin-1-yl]propylamino}methyl)-2-methoxyphenoxy]acetate

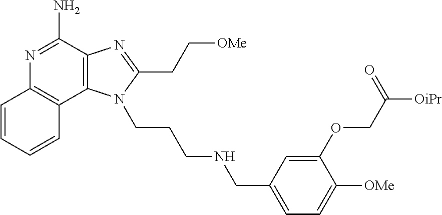

### (i) Isopropyl 2-(5-formyl-2-methoxyphenoxy)acetate

By the method of example 23 step (i) using the 4-methoxy-3-hydroxybenzaldehyde (1.00 g) and the isopropyl 2-bromoacetate (898.6 uL) to afford the title compound, 1.62 g (98%) as a white solid.

¹H NMR δ (CDCl₃) 9.82 (1H, s), 7.50 (1H, dd, J=8.24, 1.84 Hz), 7.31 (1H, d, J=1.84 Hz), 7.00 (1H, d, J=8.24 Hz), 5.12 (1H, m), 4.70 (2H, s), 3.97 (3H, s), 1.26 (6H, d, J=6.28 Hz).

MS:ESI 253 (M+1)

### (ii) Isopropyl 2-[5-({3-[4-amino-2-(2-methoxyethyl)-1H-imidazo[4,5-c]quinolin-1-yl]propylamino}methyl)-2-methoxyphenoxy]acetate

By the method of example 1 step (viii) using the product from example 15 step (iv) (604.7 mg) and isopropyl 2-(5-formyl-2-methoxyphenoxy)acetate (509.2 mg) to afford the title compound, 729.6 mg (67%) as a white solid.

¹H NMR δ (CDCl₃) 8.08 (1H, dd, J=7.4, 0.84 Hz), 7.82 (1H, dd, J=0.96, 8.36 Hz), 7.50 (1H, m), 7.29 (1H, m), 6.93 (1H, dd, J=1.84, 8.2 Hz), 6.87-6.85 (2H, m), 5.52 (2H, brs), 5.10 (1H, m), 4.66-4.61 (4H, m), 3.92-3.88 (5H, m), 3.72 (2H, s), 3.38 (3H, s), 3.25 (2H, t, J=6.52 Hz), 2.73 (2H, t, J=6.36 Hz), 2.08 (2H, m), 1.23 (6H, d, J=6.28 Hz).

MS:ESI 536 (M+1)

### Example 74

### Isopropyl 2-{5-[(N-{3-[4-amino-2-(2-methoxyethyl)-1H-imidazo[4,5-c]quinolin-1-yl]propyl}-2-chloroacetamido)methyl]-2-methoxyphenoxy}acetate hydrochloride

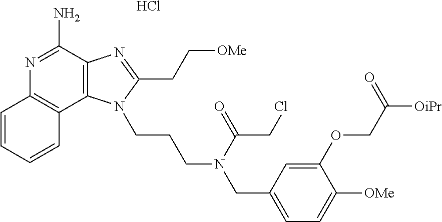

By the method of example 2 using the product of example 73 (714.6 mg), there was obtained the title compound, 821.1 mg (quant.) as a colorless gum.

¹H NMR δ (CDCl₃) 7.93-7.82 (2H, m), 7.55 (1H, m), 7.38 (1H, m), 6.80 (0.8H, d, J=8.24 Hz), 6.75-6.69 (2.2H, m), 6.18 (2H, brs), 5.08 (1H, m), 4.61 (2H, s), 4.54-4.49 (4H, m), 4.12 (1.6H, s), 4.06 (0.4H, s), 3.88-3.85 (3H, s), 3.55 (2H, t, J=6.96 Hz), 3.36 (3H, s), 3.18-3.09 (2H, m), 2.22-2.08 (2H, m), 1.25 (6H, d, J=6.24 Hz).

MS:ESI 612 (M+1)

### Example 75

### Isopropyl 2-{5-[(N-{3-[4-amino-2-(2-methoxyethyl)-1H-imidazo[4,5-c]quinolin-1-yl]propyl}-2-{dimethylamino}acetamido)methyl]-2-methoxyphenoxy}acetate

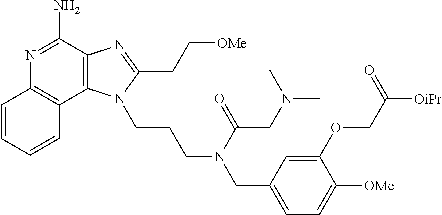

The title compound was prepared by the method of example 5 using the product from example 74 (374.7 mg) and dimethylamine (2.0M THF solution, 1.53 mL), to give a colorless gum (242.7 mg). Yield 64%.

¹H NMR δ (CDCl₃) 7.92-7.85 (2H, m), 7.54 (1H, m), 7.38-7.32 (1H, m), 6.80-6.73 (1.5H, m), 6.69-6.68 (1.5H, m), 5.78 (1.5H, brs), 5.65 (0.5H, brs), 5.08 (1H, m), 4.61-4.60 (3.5H, s), 4.51-4.47 (2.5H, m), 3.88-3.85 (5H, m), 3.53-3.44 (2H, m), 3.36 (3H, s), 3.17 (1.5H, s), 3.13 (1.5H, t, J=6.36 Hz), 3.09 (0.5H, t, J=6.2 Hz), 2.32 (4.5H, s), 2.22-2.18 (0.5H, m), 2.12 (1.5H, s), 2.10-2.03 (1.5H, m), 1.26-1.23 (6H, m).

MS:ESI 621 (M+1)

### Example 76

### Isopropyl 2-{5-[(N-{3-[4-amino-2-(2-methoxyethyl)-1H-imidazo[4,5-c]quinolin-1-yl]propyl}-2-{ethyl(methyl)amino}acetamido)methyl]-2-methoxyphenoxy}acetate

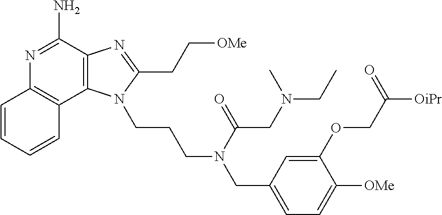

The title compound was prepared by the method of example 5 using the product from example 74 (285.6 mg) and N-ethylmethylamine, to give a colorless gum (240.1 mg). Yield 81%.

¹H NMR δ (CDCl₃) 7.91-7.83 (2H, m), 7.52 (1H, m), 7.37-7.31 (1H, m), 6.79-6.67 (3H, m), 5.58 (1.5H, brs), 5.50 (0.5H, brs), 5.07 (1H, m), 4.64 (1.5H, s), 4.60 (1.5H, s), 4.59 (0.5H, s), 4.51-4.47 (2.5H, m), 3.88-3.85 (5H, m), 3.50 (2H, t, J=7.16 Hz), 3.36 (2.3H, s), 3.35 (0.7H, s), 3.23 (1.5H, s), 3.15-3.07 (2.5H, m), 2.50 (1.5H, q, J=7.16 Hz), 2.36 (0.5H, q, J=7.2 Hz), 2.30 (2.3H, s), 2.21-2.17 (0.5H, m), 2.30 (0.7H, s), 2.10-2.03 (1.5H, s), 1.26-1.23 (6H, m), 1.05 (2.3H, t, J=7.08 Hz), 0.99 (0.7H, t, J=7.12 Hz).

MS:ESI 635 (M+1)

### Example 77

### Methyl 1-[3-({3-[4-amino-2-(2-methoxyethyl)-1H-imidazo[4,5-c]quinolin-1-yl]propylamino}methyl)phenoxy]cyclopropanecarboxylate

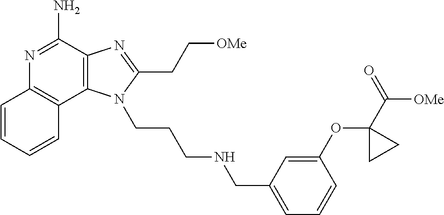

### (i) 3-(2-Oxotetrahydrofuran-3-yloxy)benzaldehyde

To a solution of 3-hydroxybenzaldehyde (500 mg, 4.09 mmol) in acetone (10 ml) 2-bromo-gamma-butyrolactone (755.7 uL, 8.18 mmol) and K₂CO₃ (1.70 g, 12.3 mmol) was added at rt. After refluxed for 12 h, cooled to rt, then the mixture was concentrated. The residue was purified by flash column chromatography to give the title compound (206.7 mg, 24%) as colorless oil.

¹H NMR δ (CDCl₃) 9.98 (1H, s), 7.56-7.47 (3H, m), 7.33 (1H, m), 5.04 (1H, t, J=7.96 Hz), 4.55 (1H, m), 4.38 (1H, m), 2.78 (1H, m), 2.48 (1H, m).

### (ii) Methyl 2-(3-formylphenoxy)-4-hydroxybutanoate

To a solution of the product of step (i) (201.1 mg, 0.975 mmol) in MeOH (5 ml) HCl (0.5 mL) was added at rt. After refluxed for 6 h, diluted with AcOEt, and H₂O was added. The aq. layer was extracted with AcOEt, dried over Na₂SO₄, and concentrated. The residue was purified by flash column chromatography to give the title compound (71.3 mg, 31%) as colorless oil.

¹H NMR δ (CDCl₃) 9.95 (1H, s), 7.50 (1H, m), 7.45 (1H, t, J=7.84 Hz), 7.36 (1H, m), 7.19 (1H, m), 4.97 (1H, t, J=6.08 Hz), 3.89 (2H, brm), 3.77 (3H, s), 2.22 (2H, m).

### (iii) Methyl 1-(3-formylphenoxy)cyclopropanecarboxylate

To a solution of the product of step (ii) (68.8 mg, 0.289 mmol) and Et₃N (50.3 uL, 0.361 mmol) in CH₂Cl₂ (2 ml), p-toluenesulfonylchloride (55.7 mg, 0.292 mmol) was added at 0° C. and the mixture was stirred overnight at room temperature. Water was added and the mixture extracted with AcOEt, dried over Na₂SO₄, and concentrated. This crude material was dissolved in THF (2 mL), Cs₂CO₃ (282.5 mg, 0.867 mmol) was added to the solution, and stirred overnight at room temperature. Water was added and the mixture extracted with AcOEt, dried over Na₂SO₄, and concentrated. The residue was purified by flash column chromatography to give the title compound (45.1 mg, 71%) as colorless oil.

¹H NMR δ (CDCl₃) 9.95 (1H, s), 7.51-7.43 (2H, m), 7.35 (1H, m), 7.18 (1H, m), 3.74

(3H, s), 2.54-2.43 (4H, m).

### (iv) Methyl 1-[3-({3-[4-amino-2-(2-methoxyethyl)-1H-imidazo[4,5-c]quinolin-1-yl]propylamino}methyl)phenoxy]cyclopropanecarboxylate

By the method of example 1 step (viii) using the product from example 15 step (iv) (664.6 mg) and methyl 1-(3-formylphenoxy)cyclopropanecarboxylate (488.4 mg) to afford the title compound, 1.03 g (92%) as a pale yellow solid.

¹H NMR δ (CDCl₃) 8.09 (1H, m), 7.82 (1H, dd, J=8.32, 0.88 Hz), 7.51 (1H, m), 7.32-7.28 (1H, m), 7.21 (1H, dd, J=7.92, 7.84 Hz), 6.93 (1H, d, J=7.52 Hz), 6.93 (1H, m), 6.78 (1H, m), 6.54 (1H, dd, J=7.96, 2.24 Hz), 5.41 (2H, brs), 4.67 (2H, m), 3.91 (2H, t, J=6.52 Hz), 3.78 (2H, s), 3.74 (3H, s), 3.39 (3H, s), 3.26 (2H, t, J=6.52 Hz), 2.80-2.73 (4H, m), 2.51-2.43 (2H, m), 2.11-2.02 (2H, m).

MS:ESI 504 (M+1)

### Example 78

### Methyl 1-{3-[(N-{3-[4-amino-2-(2-methoxyethyl)-1H-imidazo[4,5-c]quinolin-1-yl]propyl}-2-chloroacetamido)methyl]phenoxy}cyclopropanecarboxylate hydrochloride

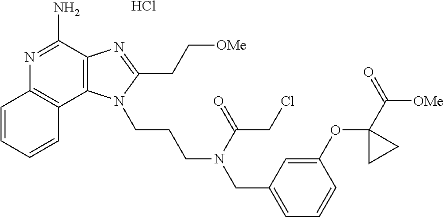

By the method of example 2 using the product of example 77 (252.6 mg), there was obtained the title compound, 243.2 mg (83%) as a colorless gum.

¹H NMR δ (CDCl₃) 7.92 (1H, d, J=7.44 Hz), 7.82 (1H, d, J=8.28 Hz), 7.53 (1H, m), 7.36 (1H, m), 7.19 (1H, dd, J=7.88, 7.88 Hz), 6.71 (1H, d, J=7.52 Hz), 6.57 (1H, d, J=6.48 Hz), 6.53 (1H, dd, J=8.04, 2.24 Hz), 5.59 (2H, brs), 4.59 (2H, s), 4.56-4.51 (2H, m), 4.07 (2H, s), 3.87 (2H, t, J=6.36 Hz), 3.73 (3H, s), 3.60 (2H, t, J=6.68 Hz), 3.36 (2.3H, s), 3.34 (0.7H, s), 3.16 (1.5H, t, J=6.36 Hz), 3.12 (0.5H, m), 2.72 (2H, m), 2.42 (2H, m), 2.15 (2H, m).

MS:ESI 581 (M+1)

### Example 79

### Methyl 1-{3-[(N-{3-[4-amino-2-(2-methoxyethyl)-1H-imidazo[4,5-c]quinolin-1-yl]propyl}-2-{diethylamino}acetamido)methyl]phenoxy}cyclopropanecarboxylate

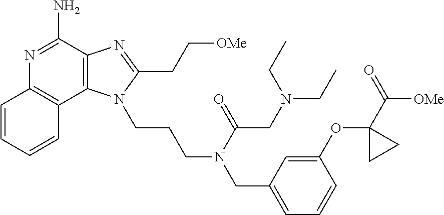

The title compound was prepared by the method of example 5 using the product from example 78 (88.7 mg) and diethylamine, to give a colorless gum (63.2 mg). Yield 67%.

¹H NMR δ (CDCl₃) 7.91-7.83 (2H, m), 7.53 (1H, m), 7.34 (1H, m), 7.17 (1H, dd, J=8.04, 7.92 Hz), 6.74-6.70 (1H, m), 6.63 (0.25H, s), 6.57 (0.75H, s), 6.49 (1H, dd, J=8.4, 2.4 Hz), 4.76 (1.5H, s), 4.56 (0.5H, s), 4.50 (2H, m), 3.87 (2H, m), 3.74 (3H, s), 3.55 (2H, m), 3.36 (2.3H, s), 3.34 (0.7H, s), 3.28 (2H, s), 3.15 (1.5H, t, J=6.36 Hz), 3.10 (0.5H, m), 2.72 (2H, m), 2.59 (3H, q, J=7.16 Hz), 2.52 (1H, q, J=7.08 Hz), 2.41 (2H, m), 2.09 (2H, m), 0.99 (6H, t, J=7.08 Hz).

MS:ESI 617 (M+1)

### Example 80

### Cyclopentyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate

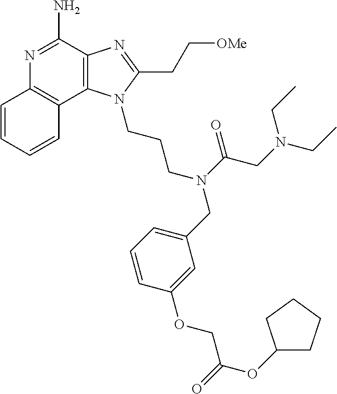

By the method of example 26 step (ii) using the product from example 26 step (i) (0.21 g, 0.36 mmol) and cyclopentanol (6.0 mL) and CH₃CN (1.0 ml) to afford the title compound (0.19 g, 83%) as a colorless gum.

¹H NMR δ (CDCl₃) 7.90-7.83 (2H, m), 7.53 (1H, t, J=7.3 Hz), 7.35 (1H, t, J=7.0 Hz), 7.21 (1H, t, J=8.4 Hz), 6.80-6.73 (3H, m), 5.52-5.16 (2H, m), 5.31-5.28 (1H, m), 4.77 (1.5H, s), 4.58-4.48 (4.5H, m), 3.87 (2H, t, J=6.4 Hz), 3.59-3.52 (2H, m), 3.37-3.27 (5H, m), 3.17-3.08 (2H, m), 2.64-2.52 (4H, m), 2.28-2.18 (0.5H, m), 2.13-2.00 (1.5H, m), 1.93-1.84 (2H, m), 1.72-1.27 (6H, m), 1.01 (6H, t, J=7.1 Hz)

ESI-MS [M+2H]²⁺: 323

### Example 81

### Cyclobutyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate

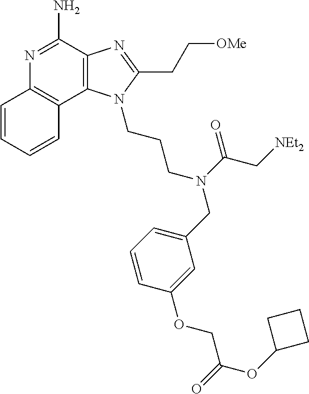

By the method of example 26 step (ii) using the product from example 26 step (i) (0.11 g, 0.18 mmol) and cyclobutanol (1.0 mL) to afford the title compound (0.096 g, 83%) as a colorless gum.

¹H NMR δ (CDCl₃) 7.90-7.85 (2H, m), 7.54 (1H, t, J=8.0 Hz), 7.40-7.31 (1H, m), 7.21 (1H, t, J=8.4 Hz), 6.80-6.73 (3H, m), 5.75-5.62 (2H, m), 5.15-5.07 (0.75H, m), 4.76 (1.5H, s), 4.58-4.50 (4.5H, m), 4.31-4.21 (0.25H, m), 3.87 (2H, t, J=6.4 Hz), 3.63-3.52 (2H, m), 3.37-3.28 (5H, m), 3.16-3.09 (2H, m), 2.64-2.53 (4H, m), 2.40-2.29 (4H, m), 2.14-2.07 (4H, m), 1.01 (6H, t, J=7.1 Hz)

ESI-MS [M+2H]²⁺: 331

### Example 82

### Tetrahydro-2H-pyran-4-yl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate

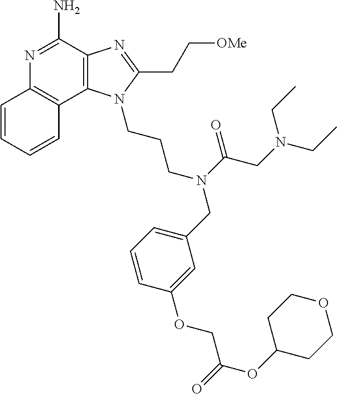

By the method of example 26 step (ii) using the product from example 26 step (i) (0.10 g, 0.17 mmol) and toluene (2.0 mL), CH₃CN (1.0 ml) and Tetrahydro-4H-pyrane-4-ol (0.20 mL) to afford the title compound (7.4 mg, 6%) as a pale yellow gum.

¹H NMR δ (CDCl₃) 7.83-7.76 (2H, m), 7.47 (1H, t, J=8.0 Hz), 7.29-7.27 (1H, m), 7.13 (1H, t, J=8.0 Hz), 6.73-6.64 (3H, m), 5.53-5.48 (2H, m), 5.05-4.99 (1H, m), 4.69 (1.5H, s), 4.51-4.50 (2.5H, m), 4.42 (2H, t, J=6.7 Hz), 3.82-3.70 (4H, m), 3.60-3.43 (4H, m), 3.29-3.19 (5H, m), 3.19-3.01 (2H, m), 2.56-2.45 (4H, m), 2.18-2.12 (0.5H, m), 2.03-2.00 (1.5H, m), 1.89-1.84 (2H, m), 1.67-1.61 (2H, m), 0.93 (6H, t, J=7.1 Hz)

ESI-MS [M+2H]²⁺: 318

### Example 83

### Butyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate

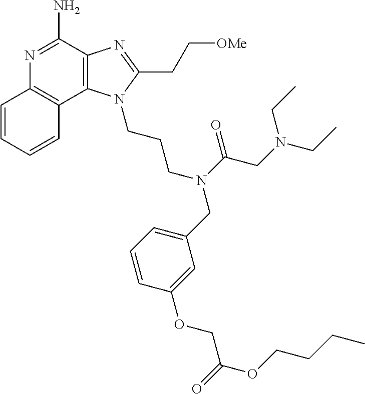

By the method of example 26 step (ii) using the product from example 26 step (i) (0.11 g, 0.19 mmol) in n-butanol (2.0 mL) and CH₃CN (1.0 ml) to afford the title compound (0.095 g, 81%) as a colorless gum.

¹H NMR δ (CDCl₃) 7.91-7.85 (2H, m), 7.85-7.83 (1H, m), 7.55-7.51 (1H, m), 7.36-7.32 (1H, m), 7.21 (1H, t, J=7.8 Hz), 6.80-6.73 (3H, m), 5.49-5.46 (2H, m), 4.76 (2H, s), 4.59 (2H, s), 4.50 (2H, t, J=7.8 Hz), 4.21 (2H, t, J=6.7 Hz), 3.89-3.86 (2H, t, J=6.4 Hz), 3.54 (2H, t, J=7.1 Hz), 3.37-3.27 (5H, m), 3.15 (1.5H, t, J=6.4 Hz), 3.10 (0.5H, t, J=6.4 Hz), 2.61 (3H, q, J=7.1 Hz), 2.61 (1H, q, J=7.1 Hz), 2.28-2.22 (0.5H, m), 2.11-2.07 (1.5H, m), 1.65 (2H, quint, J=7.4 Hz), 1.36 (2H, sext, J=7.4 Hz), 1.01 (6H, t, J=7.1 Hz), 0.93 (3H, t, J=7.4 Hz)

ESI-MS [M+2H]²⁺: 317

### Example 84

### tert-Butyl 2-[3-({3-[4-amino-2-(2-methoxyethyl)-1H-imidazo[4,5-c]quinolin-1-yl]propylamino}methyl)phenoxy]acetate

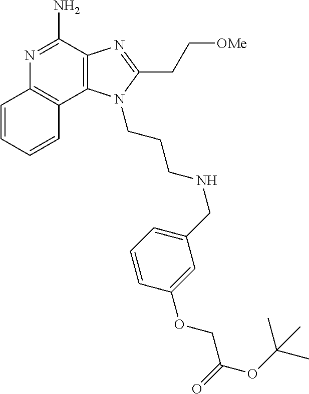

By the method of example 1 step (viii) using the product of example 15 step (iv) (0.20 g, 0.67 mmol) and the product of example 46 step (i) (0.60 g, 0.67 mmol) to afford the title compound (0.27 g, 78%) as a pale yellow solid.

¹H NMR δ (CDCl₃) 8.10 (1H, dd, J=8.4, 1.0 Hz), 7.84 (1H, dd, J=8.4 Hz, 1.0 Hz), 7.53-7.49 (1H, m), 7.32-7.25 (2H, m), 6.99-6.94 (2H, m), 6.81 (1H, dd, J=7.7 Hz, 2.1 Hz), 5.55 (2H, brs), 4.67 (2H, t, J=7.3 Hz), 4.54 (2H, s), 3.91 (2H, t, J=6.5 Hz), 3.80 (2H, s), 3.39 (3H, m), 3.26 (2H, t, J=6.5 Hz), 2.75 (2H, t, J=6.3 Hz), 2.09 (2H, quint, J=6.5 Hz), 1.73 (1H, brs), 1.49 (9H, s)

ESI-MS [M+H]⁺: 520

### Example 85

### tert-Butyl 2-{3-[(N-{3-[4-amino-2-(2-methoxyethyl)-1H-imidazo[4,5-c]quinolin-1-yl]propyl}-2-chloroacetamido)methyl]phenoxy}acetate Hydrochloride

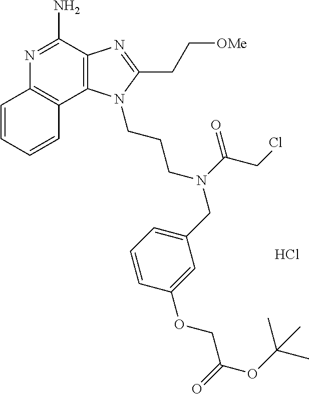

By the method of example 2 using the product of example 84 (0.27 g, 0.51 mmol), there was obtained the title compound (0.26 g, 82%) as a colorless gum.

¹H NMR δ (CDCl₃) 7.93 (1H, d, J=8.4 Hz), 7.88 (1H, d, J=7.6 Hz), 7.58-7.54 (1H, m), 7.40 (1H, t, J=7.1 Hz), 7.26-7.22 (1H, m), 6.78-6.74 (3H, m), 4.62 (2H, s), 4.57-4.54 (2H, m), 4.52-4.50 (2H, m), 4.11-4.09 (2H, m), 3.88 (2H, t, J=6.3 Hz), 3.60 (2H, t, J=6.8 Hz), 3.37-3.35 (3H, m), 3.16 (2H, t, J=6.3 Hz), 2.17-2.13 (2H, m), 1.50 (9H, 1.50)

ESI-MS [M+H]⁺: 596

### Example 86

### tert-Butyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate

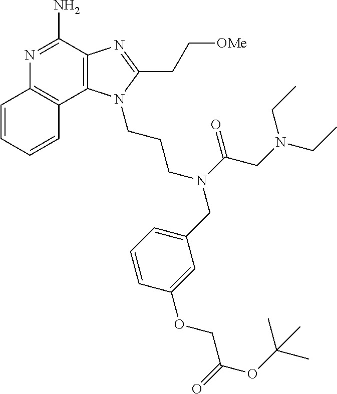

The title compound was prepared by the method of example 5 using the product from example 85 (0.26 g, 0.44 mmol) and diethylamine, to give the title compound as a pale yellow gum (0.22 g, 84%).

¹H NMR δ (CDCl₃) 7.92-7.84 (2H, m), 7.53 (1H, t, J=8.1 Hz), 7.35 (1H, t, J=7.0 Hz), 7.23-7.21 (1H, m), 6.80-6.73 (3H, m), 5.52-5.48 (2H, m), 4.77 (1.5H, s), 4.59 (0.5H, s), 4.51-4.48 (4H, m), 3.89-3.86 (2H, m), 3.60-3.53 (2H, m), 3.37-3.27 (5H, m), 3.15 (1.5H, t, J=6.4 Hz), 3.09 (0.5H, t, J=6.4 Hz), 2.61 (3H, q, J=7.1 Hz), 2.54 (1H, t, J=7.1 Hz), 2.26-2.23 (0.5H, m), 2.24-2.21 (1.5H, m), 1.55 (9H, s), 1.01 (6H, t, J=7.1 Hz)

ESI-MS [M+H]⁺: 633

### Example 87

### Ethyl 2-[3-({3-[4-amino-2-(2-methoxyethyl)-1H-imidazo[4,5-c]quinolin-1-yl]propylamino}methyl)-2-methoxyphenoxy]acetate

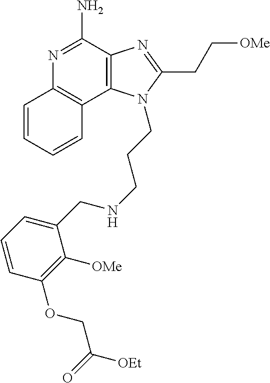

### (i) 3-Hydroxy-2-methoxybenzaldehyde

A solution of 2,3-dihydroxybenzaldehyde (1.0 g, 7.24 mmol) in DMF (10 mL) was treated with K₂CO₃ (1.0 g, 7.24 mmol) and the mixture was stirred at rt for 30 min. Iodomethane (0.50 ml, 7.96 mmol) was added and the reaction was further stirred for 20 h. The reaction was quenched with H₂O and extracted with Et₂O. The organic layer was dried over Na₂SO₄ and concentrated. The residue was purified flash column chromatography to afford the title compound (0.63 g, 57%) as a colorless needle.

¹H NMR δ (CDCl₃) 10.28 (1H, s), 7.39 (1H, dd, J=7.8 Hz, 1.8 Hz), 7.25 (1H, dd, J=7.8 Hz, 1.8 Hz), 7.17 (1H, t, J=7.8 Hz), 5.81 (1H, s), 3.99 (1H, s)

ESI-MS [M+H]⁺: 153

### (ii) Ethyl 2-(3-formyl-2-methoxyphenoxy)acetate

The title compound was prepared by the method of example 23 step (i) using the product from step (i) (0.63 g, 0.41 mmol) and Ethyl bromoacetate (0.48 mL, 4.35 mmol), to give the title compound as colorless oil (0.90 g, 91%).

¹H NMR δ (CDCl₃) 10.43 (1H, d, J=1.0 Hz), 7.48 (1H, dd, J=7.4 Hz, 2.0 Hz), 7.13-7.06 (2H, m), 4.72 (2H, s), 4.27 (2H, q, J=7.1 Hz), 4.07 (3H, s), 1.30 (3H, t, J=7.1 Hz)

ESI-MS [M+H]⁺: 239

### (iii) Ethyl 2-[3-({3-[4-amino-2-(2-methoxyethyl)-1H-imidazo[4,5-c]quinolin-1-yl]propylamino}methyl)-2-methoxyphenoxy]acetate

By the method of example 1 step (viii) using the product of example 15 step (iv) (0.64 g, 2.12 mmol) and the product from step (ii) (0.51 g, 2.12 mmol) to afford the title compound (0.80 g, 72%) as a pale yellow solid.

¹H NMR δ (CDCl₃) 8.07 (1H, dd, J=8.2 Hz, 1.0 Hz), 7.81 (1H, dd, J=8.4 Hz, 1.0 Hz), 7.49 (1H, td, J=7.7 Hz, 1.2 Hz), 7.30-7.27 (1H, m), 7.01-6.92 (2H, m), 6.76 (1H, dd, J=8.0 Hz, 1.6 Hz), 5.41 (2H, brs), 4.68 (2H, s), 4.64 (2H, t, J=6.4 Hz), 4.27 (2H, q, J=7.1 Hz), 3.93 (3H, s), 3.89 (2H, t, J=6.6 Hz), 3.83 (2H, s), 3.36 (3H, s), 3.24 (2H, t, J=6.6 Hz), 2.70 (2H, t, J=6.4 Hz), 2.07 (2H, quint, J=6.4 Hz), 1.74 (1H, brs), 1.30 (3H, t, J=7.1 Hz)

ESI-MS [M+H]⁺: 522

### Example 88

### Ethyl 2-{3-[(N-{3-[4-amino-2-(2-methoxyethyl)-1H-imidazo[4,5-c]quinolin-1-yl]propyl}-2-chloroacetamido)methyl]-2-methoxyphenoxy}acetate hydrochloride

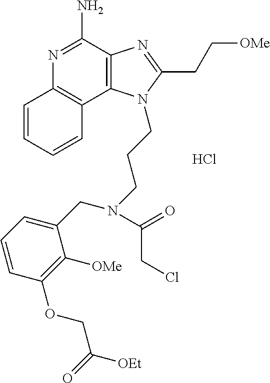

By the method of example 2 using the product of example 87 (0.39 g, 0.74 mmol), there was obtained the title compound (0.46 g, 98%) as pale yellow amorphousness.

¹H NMR δ (CDCl₃) 7.92-7.88 (2H, m), 7.57 (1H, t, J=7.1 Hz), 7.41 (1H, t, J=7.1 Hz), 6.86 (1H, t, J=8.0 Hz), 6.68 (1H, dd, J=8.3 Hz, 1.2 Hz), 6.62 (1H, d, J=7.6 Hz), 6.42 (1H, brs), 4.68-4.59 (4H, m), 4.52 (2H, t, J=7.9 Hz), 4.28-4.23 (3.5H, m), 4.06 (0.5H, s), 3.88-3.83 (5H, m), 3.55-3.43 (2H, m), 3.36-3.34 (3H, m), 3.14 (2H, t, J=6.2 Hz), 2.14-2.05 (2H, m), 1.30 (3H, t, J=7.2 Hz)

ESI-MS [M+H]⁺: 598

### Example 89

### Ethyl 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate

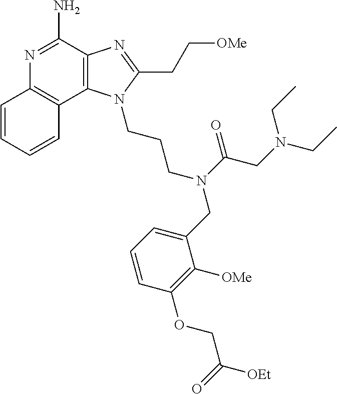

The title compound was prepared by the method of example 5 using the product from example 88 (0.45 g, 0.75 mmol) and diethylamine (0.78 mL, 7.50 mmol) to give the title compound as a pale yellow gum (0.33 g, 72%).

¹H NMR δ (CDCl₃) 7.92-7.89 (1H, m), 7.82 (1H, J=8.4 Hz), 7.51 (1H, t, J=7.1 Hz), 7.33 (1H, t, J=7.6 Hz), 6.94-6.84 (1H, m), 6.73-6.64 (2H, m), 5.49 (2H, brs), 4.76 (1.5H, s), 4.70 (0.5H, s), 4.66 (0.5H, s), 4.61 (1.5H, s), 4.51-4.46 (2H, m), 4.30-4.23 (2H, m), 3.88-3.84 (5H, m), 3.59-3.48 (2H, m), 3.37-3.34 (4.5H, m), 3.23 (0.5H, s), 3.14 (2H, t, J=6.4 Hz), 2.61 (3H, q, J=7.1 Hz), 2.51 (1H, q, J=7.1 Hz), 2.28-2.22 (0.5H, m), 2.09-2.04 (1.5H, m), 1.32-1.25 (3H, m), 1.02-0.94 (6H, m)

ESI-MS [M+2H]²⁺: 318

### Example 90

### Isopropyl 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate

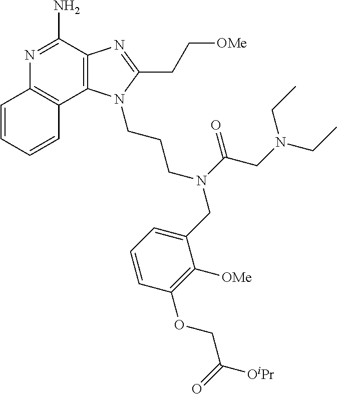

### (i) 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetic acid

The title compound was prepared by the method of example 26 step (i) using the product from example 89 (0.24 g, 0.37 mmol) to give the title compound as a white solid (0.22 g, 97%).

¹H NMR δ (CDCl₃) 8.04 (0.25H, d, J=8.2 Hz), 7.95 (0.75H, d, J=8.2 Hz), 7.71-7.68 (1H, m), 7.55 (1H, t, J=8.2 Hz), 7.42-7.37 (1H, m), 6.79-6.74 (2H, m), 6.60-6.56 (1H, m), 4.63 (0.5H, t, J=6.5 Hz), 4.52-4.47 (2H, m), 4.45 (1.5H, s), 4.41 (0.5H, s), 4.38 (1.5H, s), 4.09 (1.5H, s), 3.93-3.88 (2.5H, m), 3.85 (2.25H, s), 3.77 (0.5H, s), 3.68 (0.75H, s), 3.45 (1.5H, t, J=7.5 Hz), 3.37-3.36 (3H, m), 3.20 (0.5H, t, J=6.1 Hz), 3.15 (1.5H, t, J=6.1 Hz), 3.09 (3H, q, J=7.2 Hz), 2.92 (1H, q, J=7.2 Hz), 2.20-2.14 (0.5H, m), 2.15-1.96 (1.5H, m), 1.24 (4.5H, t, J=7.2 Hz), 1.45 (1.5H, t, J=7.2 Hz)

ESI-MS [M+2H]²⁺: 304

### (ii) Isopropyl 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate

The title compound was prepared by the method of example 26 step (ii) using the product from step (i) (0.22 g, 0.36 mmol) and isopropylalcohol to give the title compound as a pale yellow gum (0.18 g, 77%).

¹H NMR δ (CDCl₃) 7.93-7.90 (1H, m), 7.83 (1H, J=8.4 Hz), 7.51 (1H, t, J=7.5 Hz), 7.34 (1H, t, J=7.3 Hz), 6.94-6.84 (1H, m), 6.72-6.63 (2H, m), 5.48 (2H, brs), 5.15-5.09 (1H, m), 4.76 (1.5H, s), 4.71 (0.5H, s), 4.63 (0.5H, s), 4.58 (1.5H, s), 4.49 (2H, t, J=7.6 Hz), 3.88-3.85 (4H, m), 3.71 (1H, s), 3.55 (0.5H, t, J=7.0 Hz), 3.50 (1.5H, t, J=7.0 Hz), 3.37-3.34 (4.5H, m), 3.23 (0.5H, s), 3.15 (2H, t, J=6.4 Hz), 2.61 (3H, q, J=7.1 Hz), 2.51 (1H, q, J=7.1 Hz), 2.30-2.26 (0.5H, m), 2.10-2.05 (1.5H, m), 1.28-1.26 (6H, m), 1.02-0.94 (6H, m)

ESI-MS [M+2H]²⁺: 325

### Example 91

### Ethyl 2-[3-({3-[4-amino-2-(2-methoxyethyl)-1H-imidazo[4,5-c]quinolin-1-yl]propylamino}methyl)-2-fluorophenoxy]acetate

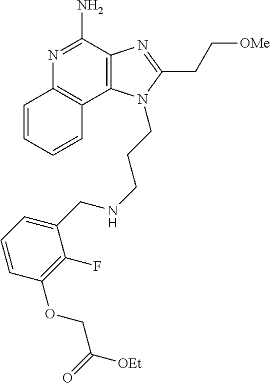

### (i) Ethyl 2-(2-fluoro-3-formylphenoxy)acetate

The title compound was prepared by the method of example 23 step (i) using 2-fluoro-3-hydroxybenzaldehyde (obtained from J. Med. Chem. 1996, 29, 1982) (0.79 g, 5.65 mmol) and Ethyl bromoacetate (0.69 mL, 6.21 mmol), to give the title compound as a colorless needle (1.0 g, 81%).

¹H NMR δ (CDCl₃) 10.39 (1H, s), 7.52-7.48 (1H, m), 7.19-7.16 (2H, m), 4.74 (2H, s), 4.28 (2H, q, J=7.1 Hz), 1.30 (3H, t, J=7.1 Hz)

ESI-MS [M+H]⁺: 227

### (ii) Ethyl 2-[3-({3-[4-amino-2-(2-methoxyethyl)-1H-imidazo[4,5-c]quinolin-1-yl]propylamino}methyl)-2-fluorophenoxy]acetate

By the method of example 1 step (viii) using the product of example 15 step (iv) (0.75 g, 2.51 mmol) and the product from step (i) (0.57 g, 2.51 mmol) to afford the title compound (0.84 g, 65%) as a colorless solid.

¹H NMR δ (CDCl₃) 8.07 (1H, d, J=8.4 Hz), 7.81 (1H, d, J=8.4 Hz,), 7.51-7.47 (1H, m), 7.31-7.28 (1H, m), 7.04-6.94 (2H, m), 6.84 (1H, td, J=7.9 Hz, 1.8 Hz), 5.45 (2H, brs), 4.69 (2H, s), 4.65 (2H, t, J=7.3 Hz), 4.27 (2H, q, J=7.2 Hz), 3.91-3.88 (4H, m), 3.37 (3H, s), 3.24 (2H, t, J=6.4 Hz), 2.72 (2H, t, J=6.4 Hz), 2.08 (2H, quint, J=7.3 Hz), 1.66 (1H, brs), 1.30 (3H, t, J=7.2 Hz)

ESI-MS [M+H]⁺: 511

### Example 92

### Ethyl 2-{3-[(N-{3-[4-amino-2-(2-methoxyethyl)-1H-imidazo[4,5-c]quinolin-1-yl]propyl}-2-chloroacetamido)methyl]-2-fluorophenoxy}acetate hydrochloride

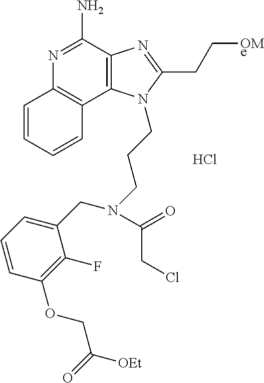

By the method of example 2 using the product of example 91 (0.54 g, 1.06 mmol), there was obtained the title compound (0.60 g, 90%) as colorless amorphousness.

¹H NMR δ (CDCl₃) 7.89 (2H, t, J=8.7 Hz), 7.55 (1H, t, J=7.7 Hz), 7.40-7.36 (1H, m), 6.98-6.88 (1H, m), 6.82-6.73 (1H, m), 6.62 (1H, d, J=7.1 Hz), 4.68 (1H, s), 4.64-4.63 (3H, m), 4.52 (2H, t, J=7.7 Hz), 4.26 (2H, q, J=7.1 Hz), 4.19 (1.5H, s), 4.04 (0.5H, s), 3.87 (2H, t, J=6.2 Hz), 3.55-3.47 (2H, m), 3.37-3.35 (3H, m) 3.15 (2H, t, J=6.2 Hz), 2.33-2.30 (0.5H, m), 2.14-2.07 (1.5H, m), 1.32-1.28 (3H, m)

ESI-MS [M+H]⁺: 586

### Example 93

### Ethyl 2-{3-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate

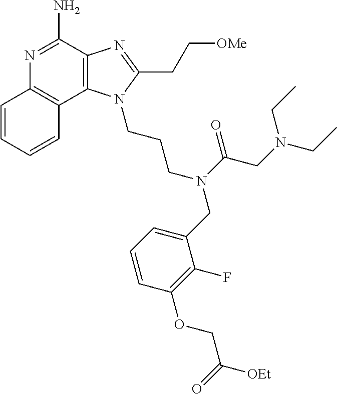

The title compound was prepared by the method of example 5 using the product from example 92 (0.59 g, 1.01 mmol) and diethylamine (1.1 mL, 10.1 mmol) to give the title compound as a pale yellow gum (0.33 g, 55%).

¹H NMR δ (CDCl₃) 7.93-7.88 (1H, m), 7.83 (1H, d, J=8.4 Hz), 7.52 (1H, t, J=7.8 Hz), 7.35 (1H, t, J=7.8 Hz), 7.00-6.66 (3H, m), 5.53 (2H, brs), 4.85 (1.5H, s), 4.69-4.68 (1H, m), 4.63 (1.5H, s), 4.56-4.47 (2H, m), 4.30-4.23 (2H, m), 3.87 (2H, t, J=6.4 Hz), 3.61 (0.5H, t, J=7.0), 3.51 (1.5H, t, J=7.0 Hz), 3.36-3.34 (4.5H, m), 3.22 (0.5H, s), 3.15 (2H, t, J=6.4 Hz), 2.59 (3H, q, J=7.1 Hz), 2.48 (1H, q, J=7.1 Hz), 2.31-2.28 (0.5H, m), 2.10-2.05 (1.5H, m), 1.32-1.28 (3H, m), 1.00 (4.5H, t, J=7.1 Hz), 0.95 (1.5H, t, J=7.1 Hz)

ESI-MS [M+2H]²⁺: 312

### Example 94

### Isopropyl 2-{3-[(N-{3-[4-amino-2-(2-methoxyethyl)-1H-imidazo[4,5-c]quinolin-1-yl]propyl}-2-{diethylamino}acetamido)methy]-2-fluorophenoxy}acetate

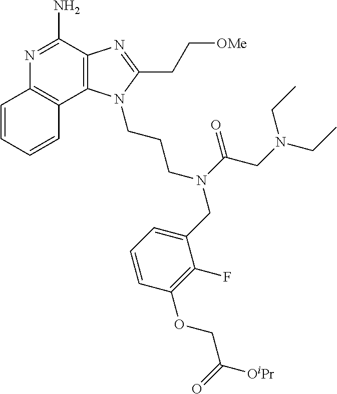

### (i)

### 2-{3-[(N-{3-[4-Amino-2-(2-methoxyethyl)-1H-imidazo[4,5-c]quinolin-1-yl]propyl}-2-{diethylamino}acetamido)methyl]-2-fluorophenoxy}acetic acid

The title compound was prepared by the method of example 26 step (i) using the product from example 93 (0.32 g, 0.51 mmol) to give the title compound as a pale yellow solid 0.32 g (quant).

¹H NMR δ (CDCl₃) 8.11 (0.25H, d, J=8.2 Hz), 8.03 (0.75H, d, J=8.2 Hz), 7.74-7.71 (1H, m), 7.58 (1H, t, J=7.2 Hz), 7.47-7.41 (1H, m), 6.84-6.80 (2H, m), 6.62-6.58 (1H, m), 4.65 (0.5H, t, J=6.5 Hz), 4.56-4.51 (3.5H, m), 4.44 (0.5Hz, s), 4.40 (1.5H, s), 4.02 (1.5H, s), 3.93-3.87 (2H, m), 3.49 (0.5H, s), 3.47 (1.5H, t, J=6.6 Hz), 3.37-3.31 (3.5H, m), 3.23-3.15 (2H, m), 3.06 (3H, q, J=7.1 Hz), 2.85 (1H, q, J=7.1 Hz), 2.22-2.19 (0.5H, m), 2.07-1.99 (1.5H, m), 1.21 (4.5H, t, J=7.1 Hz), 1.11 (1.5H, t, J=7.1 Hz)

ESI-MS [M+2H]²⁺: 298

### (ii) Isopropyl 2-{3-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate

The title compound was prepared by the method of example 5 using the product from step (i) (0.18 g, 0.30 mmol) and isopropylalcohol to give the title compound as a pale yellow gum (0.14 g, 74%).

¹H NMR δ (CDCl₃) 7.92-7.88 (1H, m), 7.82 (1H, d, J=8.3 Hz), 7.51 (1H, t, J=7.2 Hz), 7.33 (1H, t, J=7.1 Hz), 6.97-6.65 (3H, m), 5.46 (2H, brs), 5.12 (1H, sept, J=6.3 Hz) 4.85 (1.5H, s), 4.69 (0.5H, s), 4.64 (0.5H, s), 4.60 (1.5H, s), 4.55-4.46 (2H, m), 3.86 (2H, t, J=6.4 Hz), 3.61 (0.5H, t, J=7.1 Hz), 3.51 (1.5H, t, J=7.1 Hz), 3.36-3.33 (4.5H, m), 3.21 (0.5H, s), 3.16-3.12 (2H, m), 2.59 (3H, q, J=7.1 Hz), 2.48 (1H, q, J=7.1 Hz), 2.31-2.28 (0.5H, m), 2.10-2.04 (1.5H, m), 1.27-1.26 (6H, m), 0.99 (4.5H, t, J=7.1 Hz), 0.93 (1.5H, t, J=7.1 Hz)

ESI-MS [M+2H]²⁺: 319

### Example 95

### Ethyl 2-{3-[(N-{3-[4-amino-2-(2-methoxyethyl)-1H-imidazo[4,5-c]quinolin-1-yl]propyl}-2-{dimethylamino}acetamido)methyl]-2-fluorophenoxy}acetate

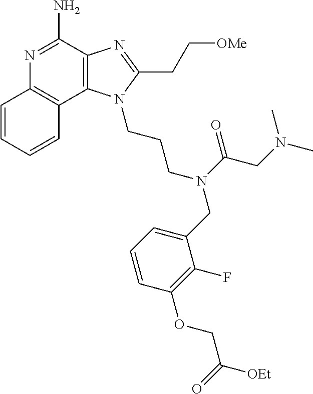

The title compound was prepared by the method of example 5 using the product from example 92 (0.46 g, 0.741 mmol) and dimethylamine in THF (2M, 3.7 mL) to give the title compound as a pale yellow gum (0.41 g, 93%).

¹H NMR δ (CDCl₃) 7.94-7.89 (1H, m), 7.84 (1H, d, J=8.4 Hz), 7.55-7.51 (1H, m), 7.38-7.34 (1H, m), 7.00-6.88 (1.3H, m), 6.83-6.79 (0.3H, m), 6.76-6.72 (0.7H, m), 6.69-6.65 (0.7H, m), 5.64-5.55 (2H, m), 4.78 (1.4H, s), 4.69-4.68 (1.2H, m), 4.64 (1.4H, s), 4.56-4.49 (2H, m), 4.30-4.23 (2H, m), 3.89-3.85 (2H, m), 3.52 (2H, t, J=7.2 Hz), 3.36-3.35 (3H, m), 3.20 (1.4H, s), 3.18-3.12 (2H, m), 3.01 (0.6H, s), 2.28 (4.2H, s), 2.28-2.22 (0.6H, m), 2.08 (1.8H, s), 2.10-2.05 (1.4H, m), 1.32-1.28 (3H, m)

ESI-MS [M+2H]²⁺: 298

### Example 96

### Isopropyl 2-{3-[(N-{3-[4-amino-2-(2-methoxyethyl)-1H-imidazo[4,5-c]quinolin-1-yl]propyl}-2-{dimethylamino}acetamido)methyl]-2-fluorophenoxy}acetate

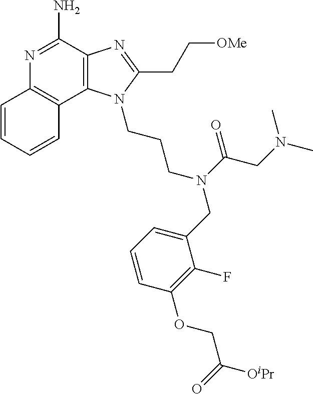

### (i) 2-{3-[(N-{3-[4-Amino-2-(2-methoxyethyl)-1H-imidazo[4,5-c]quinolin-1-yl]propyl}-2-{dimethylamino}acetamido)methyl]-2-fluorophenoxy}acetic acid

The title compound was prepared by the method of example 26 step (i) using the product from example 95 (0.22 g, 0.372 mmol) to give the title compound as colorless solid (0.19, g 88%).

¹H NMR δ (MeOD-d4) 8.16 (0.4H, d, J=8.3 Hz), 8.08 (0.6H, d, J=8.3 Hz), 7.76 (1H, d, J=7.1 Hz), 7.66 (1H, t, J=7.1 Hz), 7.53-7.49 (1H, m), 6.86-6.78 (2H, m), 6.65 (0.6H, t, J=6.5 Hz), 6.65 (0.4H, t, J=6.5 Hz), 4.72 (1.6H, t, J=6.8 Hz), 4.59-4.49 (2.4H, m), 4.41-4.39 (2H, m), 4.04 (1.2H, s), 3.95-3.90 (2.8H, m), 3.48-3.40 (2H, m), 3.37 (3H, s), 3.21 (1.6H, t, J=6.2 Hz), 3.13 (0.4H, t, J=6.2 Hz), 2.75 (3.6H, s), 2.68 (2.4H, s), 2.24-2.18 (0.8H, m), 2.11-2.01 (1.2H, m)

ESI-MS [M+2H]²⁺: 284

### (ii) Isopropyl 2-{3-[(N-{3-[4-amino-2-(2-methoxyethyl)-1H-imidazo[4,5-c]quinolin-1-yl]propyl}-2-{dimethylamino}acetamido)methyl]-2-fluorophenoxy}acetate

The title compound was prepared by the method of example 26 step (ii) using the product from step (i) (0.13 g, 0.246 mmol) and isopropylalcohol (3.5 mL) to give the title compound as pale yellow gum (0.13 g, 88%).

¹H NMR δ (CDCl₃) 7.94-7.87 (1H, m), 7.86 (1H, d, J=8.2 Hz), 7.52 (1H, t, J=7.9 Hz), 7.38 (1H, t, J=7.5 Hz), 6.98-6.89 (1.4H, m), 6.79-6.63 (1.6H, m), 5.82-5.71 (2H, m), 5.12 (1H, quint, J=6.3 Hz), 4.69 (1.4H, s), 4.65 (0.6H, s), 4.61 (0.6H, s), 4.55 (1.4H, s), 4.54-4.48 (2H, m), 3.89-3.85 (2H, m), 3.53 (2H, t, J=7.2 Hz), 3.36-3.35 (3H, m), 3.20 (1.4H, s), 3.16-3.12 (2H, m), 3.02 (0.6H, s), 2.31-2.22 (4.8H, m), 2.13-2.05 (3.2H, m), 1.29-1.26 (6H, m)

ESI-MS [M+2H]²⁺: 305

### Example 97

### Ethyl 2-{3-[(N-{3-[4-amino-2-(2-methoxyethyl)-1H-imidazo[4,5-c]quinolin-1-yl]propyl}-2-{ethyl(methyl)amino}acetamido)methyl]-2-fluorophenoxy}acetate

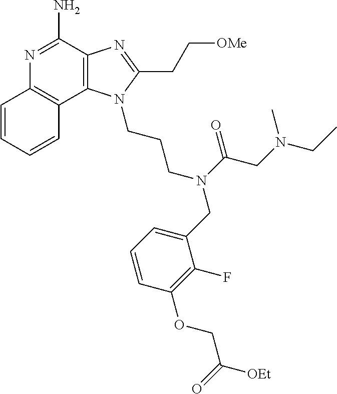

The title compound was prepared by the method of example 5 using the product from example 92 (0.50 g, 0.797 mmol) and N-Ethylmethylamine (0.34 mL, 3.99 mmol) to give the title compound as a pale yellow gum (0.39 g, 81%).

¹H NMR δ (CDCl₃) 7.93-7.89 (1H, m), 7.85-7.83 (1H, m), 7.53 (1H, t, J=8.2 Hz), 7.38-7.31 (1H, m), 7.00-6.88 (1.3H, m), 6.83-6.79 (0.3H, m), 6.76-6.66 (1.4H, m), 5.64-5.55 (2H, m), 4.82 (1.4H, s), 4.69-4.68 (1.2H, m), 4.64 (1.4H, s), 4.56-4.48 (2H, m), 4.30-4.24 (2H, m), 3.89-3.85 (2H, m), 3.59-3.50 (2H, m), 3.36-3.35 (3H, m), 3.25 (1.4H, s), 3.16-3.12 (2H, m), 3.09 (0.6H, s), 2.49 (1.4H, q, J=7.2 Hz), 2.36-2.29 (3.1H, m), 2.14-2.05 (2.5H, m), 1.32-1.28 (3H, m), 1.04 (2.1H, t, J=7.2 Hz), 0.95 (0.9H, t, J=7.2 Hz)

ESI-MS [M+2H]²⁺: 305

### Example 98

### Isopropyl 2-{3-[(N-{3-[4-amino-2-(2-methoxyethyl)-1H-imidazo[4,5-c]quinolin-1-yl]propyl}-2-{ethyl(methyl)amino}acetamido)methyl]-2-fluorophenoxy}acetate

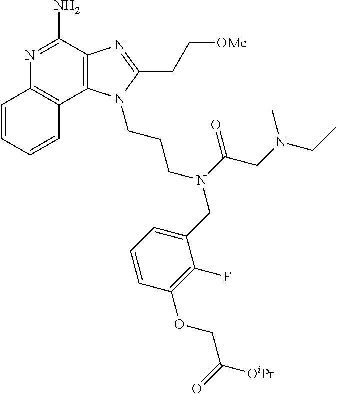

### (i) 2-{3-[(N-{3-[4-Amino-2-(2-methoxyethyl)-1H-imidazo[4,5-c]quinolin-1-yl]propyl}-2-{ethyl(methyl)amino}acetamido)methyl]-2-fluorophenoxy}acetic acid

The title compound was prepared by the method of example 26 step (i) using the product from example 97 (0.30 g, 0.493 mmol) to give the title compound as a colorless solid (0.19 g, 66%).

¹H NMR δ (MeOD-d4) 8.01 (0.3H, d, J=8.1 Hz), 7.91 (0.7H, d, J=8.1 Hz), 7.68-7.63 (1H, m), 7.55 (1H, t, J=7.6 Hz), 7.46-7.39 (1H, m), 6.90-6.78 (2H, m), 6.69-6.66 (0.7H, m), 6.60-6.58 (0.3H, m), 4.61 (0.7H, t, J=6.8 Hz), 4.52-4.44 (5.3H, m), 4.08 (1.4H, s), 3.94-3.88 (2.6H, m), 3.46 (1.4H, t, J=7.6 Hz), 3.37-3.35 (3H, m), 3.20 (0.6H, t, J=6.0 Hz), 3.13 (1.4H, t, J=6.0 Hz), 3.06 (1.4H, q, J=7.2 Hz), 2.93 (0.6H, q, J=7.2 Hz), 2.75 (2.1H, s), 2.63 (0.9H, s), 2.17-2.14 (0.6H, m), 2.02-1.96 (1.4H, m), 1.28-1.16 (3H, m)

ESI-MS [M+2H]²⁺: 291

### (ii) 2-{3-[(N-{3-[4-Amino-2-(2-methoxyethyl)-1H-imidazo[4,5-c]quinolin-1-yl]propyl}-2-{ethyl(methyl)amino}acetamido)methyl]-2-fluorophenoxy}acetate

The title compound was prepared by the method of example 26 step (ii) using the product from step (i) (0.13 g, 0.231 mmol) and isopropylalcohol (3.5 mL) to give the title compound as a pale yellow gum (0.14 g, 100%).

¹H NMR δ (CDCl₃) 7.94-7.90 (1H, m), 7.84 (1H, d, J=8.4 Hz), 7.53 (1H, t, J=7.2 Hz), 7.37 (1H, t, J=7.7 Hz), 6.96-6.89 (1.3H, m), 6.84-6.78 (0.3H, m), 6.75-6.68 (1.4H, m), 5.65-5.56 (2H, m), 5.12 (1H, quint, J=6.3 Hz), 4.82 (1.4H, s), 4.70 (0.6H, s), 4.65 (0.6H, s), 4.61 (1.4H, s), 4.54-4.48 (2H, m), 3.88-3.85 (2H, m), 3.57-3.51 (2H, m), 3.36-3.35 (3H, m), 3.25 (1.4H, s), 3.16-3.13 (2H, m), 3.09 (0.6H, s), 2.49 (1.4H, q, J=7.2 Hz), 2.33-2.29 (3.3H, m), 2.12-2.07 (2.3H, m), 1.29-1.26 (6H, m), 1.04 (2.1H, t, J=7.2 Hz), 0.95 (0.9H, t, J=7.2 Hz)

ESI-MS [M+2H]²⁺: 312

### Example 99

### Ethyl 2-[3-({2-[4-amino-2-(2-methoxyethyl)-1H-imidazo[4,5-c]quinolin-1-yl]ethylamino}methyl)phenoxy]acetate

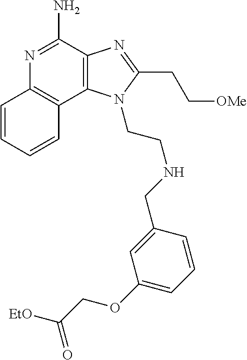

### (i) tert-Butyl 2-(3-nitroquinolin-4-ylamino)ethylcarbamate

By the method of example 1 step (ii) using the product of example step (i) (2.0 g, 10.5 mmol) and, tert-butyl 2-aminoethylcarbamate (1.8 g, 0.11 mmol) there was obtained the title compound (2.7 g, 77%) as a yellow solid.

¹H NMR δ (DMSO-d₆) 9.05 (1H, s), 8.89 (1H, brs), 8.43 (1H, d, J=8.3 Hz), 7.90 (1H, dd, J=1.2 Hz, 8.3 Hz), 7.85-7.81 (1H, m), 7.61-7.56 (1H, m), 7.04 (1H, t, J=5.5 Hz), 3.60-3.52 (2H, m), 3.34 (2H, m), 1.29 (9H, s)

ESI-MS [M+H]⁺: 333

### (ii) tert-Butyl 2-(3-aminoquinolin-4-ylamino)ethylcarbamate

By the method of example 1 step (iii) using the product of step (i) (2.7 g, 8.12 mmol) there was obtained the title compound (1.6 g, 67%) as dark amorphousness.

¹H NMR δ (DMSO-d₆) 8.36 (1H, s), 8.06-7.98 (1H, m), 7.78-7.70 (1H, m), 7.42-7.34 (2H, m), 6.96 (1H, t, J=5.5 Hz), 5.05 (2H, brs), 3.32-3.20 (2H, m), 3.16-3.06 (2H, m), 1.35 (9H, s)

ESI-MS [M+H]⁺: 303

### (iii) tert-Butyl 2-[2-(2-methoxyethyl)-1H-imidazo[4,5-c]quinolin-1-yl]ethylcarbamate

By the method of example 1 step (iv) using the product of step (ii) (1.6 g, 8.12 mmol) there was obtained the title compound (1.7 g, 85%) as a pale yellow solid.

¹H NMR δ (DMSO-d₆) 9.10 (1H, s), 8.49-8.43 (1H, m), 8.18-8.12 (1H, m), 7.73-7.65 (2H, m), 7.10 (1H, t, J=5.9 Hz), 4.67 (2H, t, J=6.0 Hz), 3.87 (2H, t, J=6.9 Hz), 3.52-3.45 (2H, m), 3.32 (3H, s), 3.21 (2H, t, J=6.9 Hz), 1.30 (9H, s)

ESI-MS [M+H]⁺: 371

### (iv) 1-[2-(tert-Butoxycarbonylamino) ethyl]-2-(2-methoxyethyl)-1H-imidazo[4,5-c]quinoline 5-oxide

By the method of example 1 step (v) using the product of step (iii) (1.7 g, 4.62 mmol), there was obtained the title compound (1.7 g, 96%) as pale yellow solid.

¹H NMR δ (CDCl₃) 9.02-8.98 (2H, m), 8.38 (1H, d, J=8.0 Hz), 7.81-7.74 (2H, m), 5.13 (1H, brs), 4.76 (2H, t, J=6.3 Hz), 3.92 (2H, t, J=6.2 Hz), 3.69-3.65 (2H, m), 3.37 (3H, s), 3.23 (2H, t, J=6.1 Hz), 1.43 (9H, s)

ESI-MS [M+H]⁺: 387

### (v) tert-Butyl 2-[4-amino-2-(2-methoxyethyl)-1H-imidazo[4,5-c]quinolin-1-yl]ethylcarbamate

By the method of example 1 step (vi) using the product of step (iv) (1.7 g, 4.43 mmol) to afford the title compound as a pale yellow solid (1.7 g).

ESI-MS [M+H]⁺: 386

### (vi) 1-(2-Aminoethyl)-2-(2-methoxyethyl)-1H-imidazo[4,5-c]quinolin-4-amine

By the method of example 1 step (vii) using the product of step (v) (1.7 g), there was obtained the title compound (1.1 g, 96% from step (iv)) as a yellow solid.

¹H NMR δ (MeOD-d₄) 8.13 (1H, d, J=7.4 Hz), 7.69 (1H, dd, J=8.4 Hz, 1.0 Hz), 7.49 (1H, td, J=7.0 Hz, 1.2 Hz), 7.37-7.33 (1H, m), 4.64 (2H, t, J=7.4 Hz), 3.90 (2H, t, J=6.2 Hz), 3.38 (3H, s), 3.30 (2H, t, J=6.2 Hz), 3.12 (2H, t, J=7.4 Hz)

ESI-MS [M+H]⁺: 286

### (vii) Ethyl 2-[3-({2-[4-amino-2-(2-methoxyethyl)-1H-imidazo[4,5-c]quinolin-1-yl]ethylamino}methyl)phenoxy]acetate

By the method of example 1 step (viii) using the product of step (vi) (0.79 g, 2.75 mmol) and the product of example 23 step (i) (0.57 g, 2.75 mmol) to afford the title compound (0.73 g, 56%) as pale yellow amorphousness.

¹H NMR δ (CDCl₃) 7.95 (1H, dd, J=8.2 Hz, 0.80 Hz), 7.82 (1H, dd, J=8.4 Hz, 1.2 Hz), 7.53-7.49 (1H, m), 7.31 (1H, td, J=7.2 Hz, 1.2 Hz), 7.19 (1H, t, J=7.8 Hz), 6.88-6.84 (2H, m), 6.76 (1H, dd, J=8.0 Hz, 2.3 Hz), 5.63 (2H, brs), 4.62 (2H, t, J=6.6 Hz), 4.58 (2H, s), 4.26 (2H, q, J=7.2 Hz), 3.88 (2H, t, J=6.4 Hz), 3.76 (2H, s), 3.34 (3H, s), 3.24 (2H, t, J=6.4 Hz), 3.16 (2H, t, J=6.6 Hz), 1.84 (1H, brs), 1.29 (3H, t, J=7.2 Hz)

ESI-MS [M+H]⁺: 478

### Example 100

### Ethyl 2-{3-[(N-{2-[4-amino-2-(2-methoxyethyl)-1H-imidazo[4,5-c]quinolin-1-yl]ethyl}-2-chloroacetamido)methyl]phenoxy}acetate hydrochloride

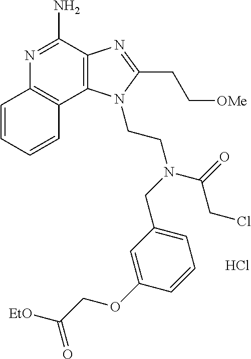

By the method of example 2 using the product of example 99 (0.73 g, 1.53 mmol), there was obtained the title compound (0.83 g, 91%) as pale yellow amorphousness.

¹H NMR δ (CDCl₃) 8.20 (1H, d, J=8.0 Hz), 7.93 (1H, d, J=8.3 Hz), 7.60 (1H, t, J=7.4 Hz), 7.49 (1H, t, J=7.4 Hz), 7.26-7.22 (1H, m), 6.83 (1H, td, J=8.2 Hz, 2.1 Hz), 6.77-6.76 (2H, m), 4.70 (2H, t, J=7.2 Hz), 4.60 (2H, s), 4.49 (2H, s), 4.25 (2H, q, J=7.1 Hz), 4.18 (2H, s), 3.81-3.76 (4H, m), 3.26 (3H, s), 2.96 (2H, t, J=5.8 Hz), 1.29 (3H, t, J=7.1 Hz)

ESI-MS [M+H]⁺: 554

### Example 101

### Ethyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate

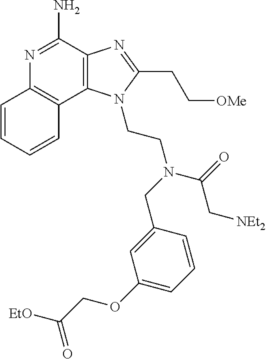

By the method of example 5 using the product of example 100 (0.83 g, 1.40 mmol) and diethylamine (1.5 mL, 14.0 mmol) to give the title compound as a pale yellow solid (0.66 g, 80%).

¹H NMR δ (CDCl₃) 8.05 (0.9H, d, J=8.0 Hz), 7.82 (0.1H, d, J=8.0 Hz), 7.79 (1H, d, J=8.3 Hz), 7.50-7.47 (1H, m), 7.33-7.21 (2H, m), 6.81-6.72 (3H, m), 5.53-5.46 (2H, m), 4.78 (0.2H, t, J=7.6 Hz), 4.66 (1.8H, t, J=7.6 Hz), 4.58-4.20 (4H, m), 4.23 (2H, q, J=7.1 Hz), 4.03-3.99 (0.2, m), 3.84-3.81 (1.8H, m), 3.71 (2H, t, J=7.6 Hz), 3.33 (2H, s), 3.29 (3H, s), 3.07 (2H, t, J=6.1 Hz), 2.65-2.60 (3.6H, m), 2.00-1.97 (0.4H, m), 1.25 (3H, t, J=7.1 Hz), 1.03 (5.4H, t, J=7.1 Hz), 0.79 (0.6H, t, J=7.1 Hz)

ESI-MS [M+2H]²⁺: 296

### Example 102

### Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate

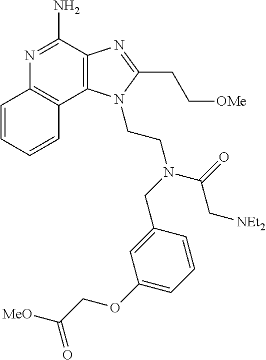

### (i) 2-{3-[(N-{2-[4-Amino-2-(2-methoxyethyl)-1H-imidazo[4,5-c]quinolin-1-yl]ethyl}-2-{diethylamino}acetamido)methyl]phenoxy}acetic acid

The title compound was prepared by the method of example 26 step (i) using the product from example 101 (0.56 g, 0.945 mmol) to give the title compound as a pale yellow solid (0.53 g, quant).

¹H NMR δ (MeOD-d₄) 8.29 (1H, brs), 7.46-7.42 (2H, m), 7.37 (1H, t, J=7.3 Hz), 7.28-7.24 (1H, m), 7.17-7.14 (1H, m), 7.01 (1H, d, J=7.3 Hz), 6.95 (1H, s), 4.89-4.77 (4H, m), 4.28 (2H, brs), 4.28 (2H, brs), 3.67-3.63 (2H, m), 3.31 (3H, s), 3.25 (4H, brs), 2.53 (2H, brs), 1.39 (6H, t, J=7.1 Hz)

ESI-MS [M+2H]²⁺: 282

### (ii) Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate

The title compound was prepared by the method of example 26 step (ii) using the product from step (i) (0.10 g, 0.181 mol) and MeOH (3.5 mL) to give the title compound as a pale yellow solid (0.99 g, 94%).

¹H NMR δ (CDCl₃) 8.05 (1H, d, J=7.7 Hz), 7.80 (1H, d, J=8.4 Hz), 7.51-7.47 (1H, m), 7.33-7.22 (2H, m), 6.81-6.71 (3H, m), 5.57-5.46 (2H, m), 4.66 (2H, t, J=7.2 Hz), 4.58-4.53 (2H, m), 3.83 (2H, t, J=6.1 Hz), 3.77 (2H, s), 3.72-3.69 (5H, m), 3.33 (2H, s), 3.29 (3H, s), 3.07 (2H, t, J=7.2 Hz), 2.63 (3.6H, q, J=7.1 Hz). 2.36 (0.4H, t, J=7.1 Hz), 1.03 (5.4H, t, J=7.1 Hz), 0.80 (0.6H, t, J=7.1 Hz)

ESI-MS [M+2H]²⁺: 289

### Example 103

### Isopropyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate

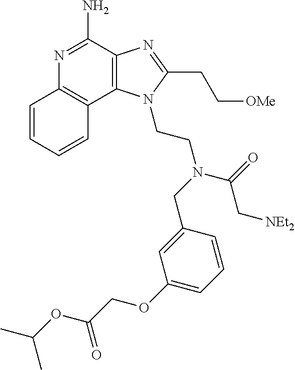

The title compound was prepared by the method of example 26 step (ii) using the product from example 102 step (i) (0.11 g, 0.194 mmol) and isopropylalcohol (3.0 mL) to give the title compound as a yellow solid (0.11 g, 94%).

¹H NMR δ (CDCl₃) 8.07 (1H, d, J=8.2 Hz), 7.86-7.79 (1H, m), 7.52-7.41 (1H, m), 7.34-7.30 (1H, m), 7.26-7.22 (1H, m), 6.80-6.74 (3H, m), 5.62-5.48 (2H, m), 5.10 (1H, quint, J=6.3 Hz), 4.66 (2H, t, J=7.6 Hz), 4.59-4.57 (2H, m), 4.53 (2, s), 3.83 (2H, t, J=6.1 Hz), 3.77 (2H, s), 3.72-3.69 (2H, m), 3.34 (2H, s), 3.29 (3H, s), 3.06 (2H, t, J=6.1 Hz), 2.64 (3.6H, q, J=7.1 Hz). 2.35 (0.4H, t, J=7.1 Hz), 1.27-1.19 (6H, m), 1.03 (5.4H, t, J=7.1 Hz), 0.79 (0.6H, t, J=7.1 Hz)

ESI-MS [M+2H]²⁺: 303

### Example 104

### Ethyl 2-[3-({2-[4-amino-2-(2-methoxyethyl)-1H-imidazo[4,5-c]quinolin-1-yl]ethylamino}methyl)phenoxy]-2-methylpropanoate

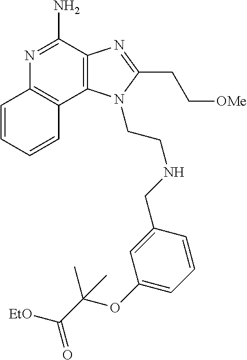

By the method of example 1 step (viii) using the example 99 step (vi) (0.32 g, 1.11 mmol) and example 53 step (i) (0.26 g, 1.11 mmol) to afford the title compound (0.41 g, 72%) as pale yellow amorphousness

¹H NMR δ (CDCl₃) 7.95 (1H, dd, J=8.4 Hz, 1.0 Hz), 7.83 (1H, dd, J=8.4 Hz, 1.0 Hz), 7.56-7.50 (1H, m), 7.34-7.30 (1H, m), 7.16 (1H, t, J=7.8 Hz), 6.88 (1H, d, J=7.7 Hz), 6.82 (1H, s), 6.69 (1H, dd, J=7.2 Hz, 2.0 Hz), 5.66 (2H, brs), 4.63 (2H, t, J=6.8 Hz), 4.20 (2H, q, J=7.1 Hz), 3.88 (2H, t, J=6.4 Hz), 3.74 (2H, s), 3.34 (3H, s), 3.25 (2H, t, J=6.4 Hz), 3.15 (2H, t, J=6.8 Hz), 2.17 (1H, brs), 1.57 (6H, s), 1.20 (3H, t, J=7.1 Hz)

ESI-MS [M+H]⁺: 506

### Example 105

### Ethyl 2-{3-[(N-{2-[4-amino-2-(2-methoxyethyl)-1H-imidazo[4,5-c]quinolin-1-yl]ethyl}-2-chloroacetamido)methyl]phenoxy}-2-methylpropanoate hydrochloride

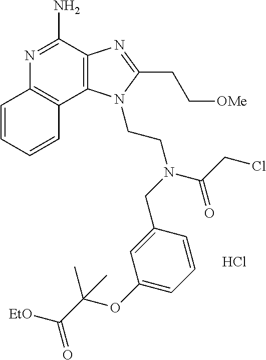

By the method of example 2 using the product of example 104 (0.41 g, 0.80 1 mmol), there was obtained the title compound (0.42 g, 86%) as colorless amorphousness.

¹H NMR δ (CDCl₃) 8.11 (1H, d, J=8.1 Hz), 7.87 (1H, d, J=8.4 Hz), 7.57-7.52 (1H, m), 7.40 (1H, t, J=8.2 Hz), 7.20 (1H, t, J=7.9 Hz), 6.74-6.66 (3H, m), 6.43 (1H, brs), 4.72 (2H, t, J=7.1 Hz), 4.33 (2H, s), 4.17 (2H, q, J=7.2 Hz), 4.10 (2H, s) 3.87-3.78 (4H, m), 3.26 (3H, s), 3.06 (2H, t, J=5.9 Hz), 1.57 (6H, s), 1.19 (3H, t, J=7.2 Hz)

ESI-MS [M+H]⁺: 582

### Example 106

### Ethyl 2-{3-[(N-{2-[4-amino-2-(2-methoxyethyl)-1H-imidazo[4,5-c]quinolin-1-yl]ethyl}-2-{diethylamino}acetamido)methyl]phenoxy}-2-methylpropanoate

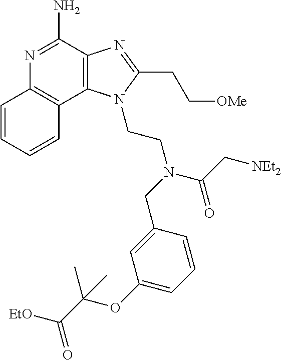

By the method of example 5 using the product of example 105 (0.42 g, 0.685 mmol) and diethylamine (0.72 mL, 6.85 mmol), to give the title compound as a pale yellow gum (0.38 g, 91%).

¹H NMR δ (CDCl₃) 8.08 (1H, d, J=8.2 Hz), 7.80 (1H, d, J=8.3 Hz), 7.50 (1H, t, J=8.2 Hz), 7.34 (1H, t, J=7.1 Hz), 7.21-7.17 (1H, m), 6.75-6.69 (3H, m), 5.60-5.47 (2H, m), 4.77 (0.2H, t, J=7.6 Hz), 4.67 (1.8H, t, J=7.6 Hz), 4.59 (2H, s), 4.16 (2H, q, J=7.1 Hz), 4.01 (0.2H, t, J=6.1 Hz), 3.83 (2H, t, J=6.1 Hz), 3.72 (1.8H, t, J=7.2 Hz), 3.35 (0.3H, s), 3.31 (2H, s), 3.28 (2.7H, s), 3.11 (2H, t, J=6.1 Hz), 2.61 (3.6H, q, J=7.1 Hz), 2.36 (0.4H, q, J=7.1 Hz), 1.56-1.54 (6H, m), 1.17 (3H, t, J=7.1 Hz), 1.02 (5.4H, t, J=7.1 Hz), 0.79 (0.6H, t, J=7.1 Hz)

ESI-MS [M+2H]²⁺: 310

### Example 107

### Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}-2-methylpropanoate

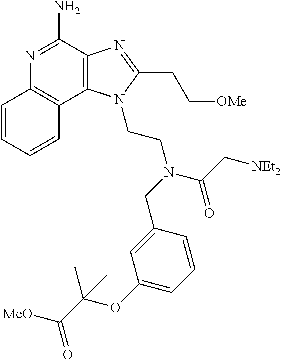

### (i) 2-{3-[(N-{2-[4-Amino-2-(2-methoxyethyl)-1H-imidazo[4,5-c]quinolin-1-yl]ethyl}-2-{diethylamino}acetamido)methyl]phenoxy}-2-methylpropanoic acid

The title compound was prepared by the method of example 26 step (i) using the product from example 106 (0.29 g, 0.464 mmol) to give the title compound as a colorless solid (0.26 g, 96%).

¹H NMR 6 (MeOD-d₄) 8.19 (0.8H, d, J=7.9 Hz), 7.97 (0.2H, d, J=7.9 Hz), 7.64-7.43 (1.2H, m), 7.36-7.31 (1.8H, m), 7.27-7.18 (1H, m), 6.99-6.95 (1.8H, m), 6.86-6.77 (0.2H, m), 4.88 (2H, brs), 4.56 (2H, brs), 3.90-3.88 (0.4H, m), 3.77-3.74 (3.6H, m), 3.69-3.57 (2H, m), 3.38 (0.3H, s), 3.32 (2.7H, s), 2.84-2.78 (5.6H, m), 2.49-2.43 (0.4H, m), 1.63 (5.4H, s), 1.52 (0.6H, s), 1.13 (5.4H, t, J=7.1 Hz), 0.78 (0.6H, t, J=7.1 Hz)

ESI-MS [M+2H]²⁺: 296

### (ii) Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}-2-methylpropanoate

The title compound was prepared by the method of example 26 step (ii) using the product from step (i) (0.12 g, 0.204 mmol) and MeOH (5 m L), to give the title compound as a pale yellow solid (0.89 g, 72%).

¹H NMR δ (CDCl₃) 8.07 (1H, d, J=7.6 Hz), 7.86-7.78 (1H, m), 7.49-7.47 (1H, m), 7.35 (1H, t, J=7.6 Hz), 7.12-7.17 (1H, m), 6.74 (1H, d, J=7.7 Hz), 6.69-6.68 (2H, m), 5.59-5.47 (2H, m), 4.78 (0.2H, t, J=7.4 Hz), 4.67 (1.8H, t, J=7.4 Hz), 4.58 (2H, s), 3.98 (0.2H, t, J=6.1 Hz), 3.84 (1.8H, t, J=6.1 Hz), 3.73-3.69 (5H, m), 3.35 (0.3H, s), 3.31 (1.8H, s), 3.28 (2.7H, s), 2.91 (0.2H, s), 2.61 (3.6H, q, J=7.1 Hz), 2.36 (0.4H, q, J=7.1 Hz), 1.56-1.54 (6H, m), 1.02 (5.4H, t, J=7.1 Hz), 0.80 (0.6H, t, J=7.1 Hz)

ESI-MS [M+2H]²⁺: 303

### Example 108

### Cyclopentyl 2-[3-({3-[4-amino-2-(2-methoxyethyl)-1H-imidazo[4,5-c]quinolin-1-yl]propylamino}methyl)phenoxy]acetate

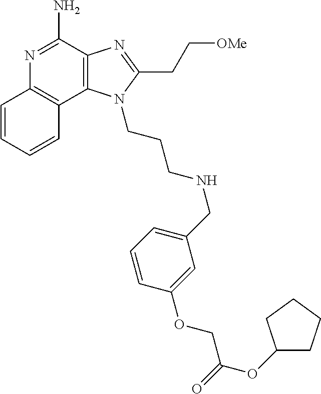

To a solution of the product of example 15 (0.53 g, 1.12 mmol) in MeOH (17 mL), 2N NaOH aq (7.0 mL) were added. After being stirred at 50° C. for 2 h and neutralized with 6N HCl aq, the resulting mixture was extracted with CHCl₃/EtOH (3/1), dried over Na₂SO₄ and concentrated in vacuo. After the obtained solid was suspended with cyclopentanol (10 mL) and CH₃CN (7.0 mL), 4N HCl/dioxane (1.5 mL) was added to the suspension. After being stirred at 50° C. for 3 h and at rt for 60 h, the resulting mixture was quenched with 7% NH₃ aq. The solution was extracted with CHCl₃ and the organic layer was washed with brine, dried over Na₂SO₄ and concentrated in vacuo. The residue was purified by flash column chromatography to afford the title compound (0.49 g, 83%) as a yellow solid.

¹H NMR δ (CDCl₃) 8.09 (1H, dd, J=8.2 Hz, 0.8 Hz), 7.82 (1H, dd, J=8.4 Hz, 1.0 Hz), 7.50 (1H, td, J=7.2 Hz, 1.2 Hz), 7.30-7.24 (2H, m), 6.98-6.93 (2H, m), 6.80 (1H, dd, J=8.0 Hz, 2.2 Hz), 5.50 (2H, brs), 5.28-5.27 (1H, m), 4.66 (2H, t, J=7.4 Hz), 4.59 (2H, s), 3.89 (2H, t, J=6.4 Hz), 3.79 (2H, s), 3.38 (3H, s), 3.25 (2H, t, J=6.4 Hz), 2.74 (2H, t, J=6.3 Hz), 2.10-2.06 (2H, m), 1.87-1.84 (3H, m), 1.71-1.57 (6H, m)

ESI-MS [M+H]⁺: 532

### Example 109

### Cyclopentyl 2-{3-[(N-{3-[4-amino-2-(2-methoxyethyl)-1H-imidazo[4,5-c]quinolin-1-yl]propyl}-2-chloroacetamido)methyl]phenoxy}acetate hydrochloride

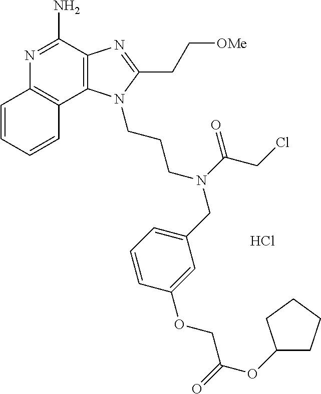

By the method of example 2 using the product of example 108 (0.49 g, 0.927 mmol), there was obtained the title compound (0.54 g, 90%) as colorless amorphousness.

¹H NMR δ (CDCl₃) 7.91 (1H, d, J=7.5 Hz), 7.85-7.80 (1H, m), 7.53 (1H, td, J=7.2 Hz, 1.2 Hz), 7.24-7.16 (2H, m), 6.78-6.73 (3H, m), 5.58-5.50 (2H, m), 5.30-5.26 (1H, m), 4.60 (1.5H, s), 4.55-4.48 (4.5H, m), 4.10-4.07 (2H, m), 3.87 (2H, t, J=6.4 Hz), 3.58 (1.5H, t, J=6.8 Hz), 3.42 (0.5H, t, J=6.8 Hz), 3.34-3.32 (3H, m), 3.17-3.09 (2H, m), 2.24-2.20 (0.5H, m), 2.17-2.10 (1.5H, m), 1.92-1.85 (2H, m), 1.72-1.61 (6H, m)

ESI-MS [M+H]⁺: 608

### Example 110

### Cyclopentyl 2-{3-[(N-{3-[4-amino-2-(2-methoxyethyl)-1H-imidazo[4,5-c]quinolin-1-yl]propyl}-2-{dimethylamino}acetamido)methyl]phenoxy}acetate

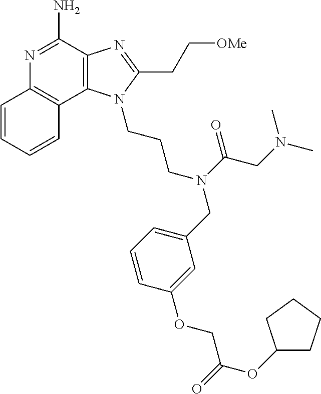

By the method of example 5 using the product of example 109 (0.29 g, 0.450 mmol) and dimethylamine in THF (2M, 2.3 mL), to give the title compound as a pale yellow gum (0.24 g, 87%).

¹H NMR δ (CDCl₃) 7.91 (1H, d, J=7.7 Hz), 7.85 (1H, d, J=8.3 Hz), 7.54 (1H, td, J=7.1 Hz, 1.2 Hz), 7.36 (1H, t, J=7.1 Hz), 7.20 (1H, t, J=8.0 Hz), 6.79-6.73 (3H, m), 5.80-5.62 (2H, m), 5.28-5.27 (1H, m), 4.70 (1.5H, s), 4.58 (0.5H, s), 4.54 (2H, s), 4.52-4.48 (2H, m), 3.86 (2H, t, J=6.4 Hz), 3.55-3.47 (2H, m), 3.36-3.35 (3H, m), 3.16-3.06 (4H, m), 2.31 (4.5H, s), 2.25-2.18 (0.5H, m), 2.13 (1.5H, s), 2.13-2.08 (1.5H, m), 1.88-1.85 (5H, m), 1.71-1.57 (3H, m)

ESI-MS [M+2H]²⁺: 309

### Example 111

### Cyclopentyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}acetate

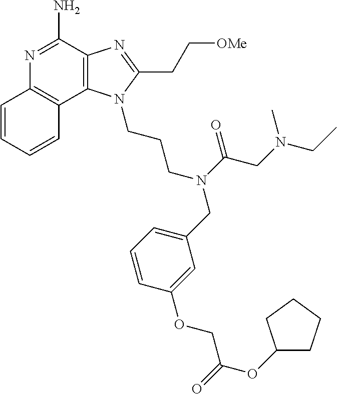

By the method of example 5 using the product of example 109 (0.25 g, 0.388 mmol) and ethylmethylamine (0.33 mL, 3.88 mmol), to give the title compound as a pale yellow gum (0.18 g, 73%).

¹H NMR δ (CDCl₃) 7.91 (1H, d, J=7.8 Hz), 7.86 (1H, d, J=8.3 Hz), 7.54 (1H, t, J=8.0 Hz), 7.39-7.24 (1H, m), 7.20 (1H, t, J=7.8 Hz), 6.80-6.72 (3H, m), 5.83-5.65 (2H, m), 5.29-5.26 (1H, m), 4.73 (1.5H, s), 4.58 (0.5H, s), 4.54 (2H, s), 4.58-4.48 (2H, m), 3.86 (2H, t, J=6.4 Hz), 3.53 (2H, t, J=7.0 Hz), 3.36-3.34 (3H, m), 3.22 (1.5H, s), 3.16-3.14 (2H, m), 3.12 (0.5H, t, J=6.4 Hz), 2.49 (1.5H, q, J=7.2 Hz), 2.35 (0.5H, q, J=7.2 Hz), 2.30 (2.25H, s), 2.25-2.20 (0.5H, m), 2.17 (0.75H, s), 2.18-2.10 (1.5H, m), 1.92-1.86 (5H, m), 1.73-1.57 (3H, m), 1.06-0.99 (3H, m)

ESI-MS [M+2H]²⁺: 316

### Example 112

### Isopropyl 2-{3-[(N-{3-[4-amino-2-(2-methoxyethyl)-1H-imidazo[4,5-c]quinolin-1-yl]propyl}-2-morpholinoacetamido)methyl]phenoxy}acetate

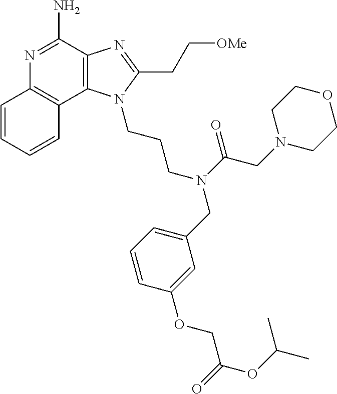

### (i) Isopropyl 2-[3-({3-[4-amino-2-(2-methoxyethyl)-1H-imidazo[4,5-c]quinolin-1-yl]propylamino}methyl)phenoxy]acetate

By the method of example 1 step (viii) using the product from example 15 step (iv) (0.40 g, 1.34 mmol) and isopropyl 2-(3-formylphenoxy)acetate (0.30 g, 1.34 mmol) there was obtained the title compound, 0.57 g (1.13 mmol, 85%) as a pale yellow gum.

### (ii) Isopropyl 2-{3-[(N-{3-[4-amino-2-(2-methoxyethyl)-1H-imidazo[4,5-c]quinolin-1-yl]propyl}-2-chloroacetamido)methyl]phenoxy}acetate hydrochloride

By the method of example 2 using the product of step (i) (0.57 g, 1.13 mmol), there was obtained the title compound, 0.59 g (0.95 mmol, 84%) as a colorless gum.

### (iii) Isopropyl 2-{3-[(N-{3-[4-amino-2-(2-methoxyethyl)-1H-imidazo[4,5-c]quinolin-1-yl]propyl}-2-morpholinoacetamido)methyl]phenoxy}acetate

The title compound was prepared by the method of example 5 using the product from step iii) (0.26 g, 0.45 mmol) and morpholine (0.50 mL, 4.8 mmol), to give the title compound as a colorless gum (0.27 g, 84%).

¹H NMR δ (CDCl₃) 7.91-7.83 (2H, m), 7.53 (1H, td, J=8.1 Hz, 1.2 Hz), 7.35 (1H, td, J=7.6 Hz, 1.2 Hz), 7.22 (1H, t, J=8.0 Hz), 6.79-6.73 (3H, m), 5.50-5.45 (2H, m), 5.14 (1H, sept, J=6.6 Hz), 4.66 (1.5H, s), 4.57-4.48 (4.5H, m), 3.87 (2H, t, J=6.4 Hz), 3.70 (3H, t, J=4.5 Hz), 3.63-3.61 (1H, m), 3.55 (1.5H, t, J=7.1 Hz), 3.47 (0.5H, t, J=7.1 Hz), 3.37-3.35 (3H, m), 3.24 (1.5H, s), 3.14 (1.5H, t, J=6.4 Hz), 3.11 (0.5H, t, J=6.4 Hz), 3.00 (0.5H, s), 2.55-2.53 (3H, m), 2.36-2.34 (1H, m), 2.26-2.18 (0.5H, m), 2.14-2.08 (1.5H, m), 1.29 (6H, d, J=6.6 Hz)

ESI-MS [M+2H]²⁺: 317

### Example 113

### Isopropyl 2-{3-[(N-{3-[4-amino-2-(2-methoxyethyl)-1H-imidazo[4,5-c]quinolin-1-yl]propyl}-2-{dimethylamino}acetamido)methyl]phenoxy}acetate

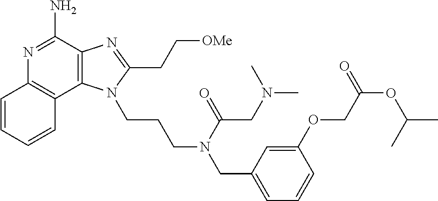

The title compound was prepared by the method of example 5 using the product from example 112 step (ii) (0.15 g) and dimethylamine, to give a colorless gum (0.14 g). Yield 97%.

¹H NMR δ (DMSO-d₆) 8.02-7.95 (1H, m), 7.61 (1H, d, J=8.3 Hz), 7.44 (1H, dd, J=7.7 Hz, 7.4 Hz), 7.27-7.17 (2H, m), 6.82-6.73 (3H, m), 6.48 (2H, brs), 5.08-4.91 (1H, m), 4.71-4.65 (3H, m), 4.57-4.40 (3H, m), 3.80 (2H, q, J=6.7 Hz), 3.50-3.39 (2H, m), 3.27 (3H, s), 3.16-3.12 (2H, m), 3.08 (1H, s), 2.99 (1H, s), 2.19 (3H, s), 2.18-2.06 (1H, m), 2.00 (3H, s), 2.00-1.95 (1H, m), 1.19 (3H, d, J=6.0 Hz), 1.17 (3H, d, J=5.2 Hz).

MS:ESI 591 (M+1)

### Example 114

### Isopropyl 2-{3-[(N-{3-[4-amino-2-(2-methoxyethyl)-1H-imidazo[4,5-c]quinolin-1-yl]propyl}-2-{ethyl(methyl)amino}acetamido]methyl)phenoxy}acetate

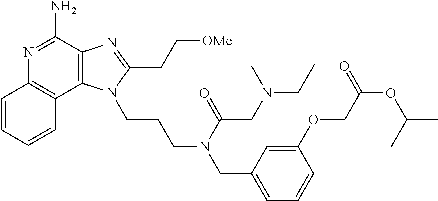

The title compound was prepared by the method of example 5 using the product from example 112 step (ii) (0.22 g) and ethylmethylamine, to give a pale yellow gum (0.21 g). Yield 99%.

¹H NMR δ (DMSO-d₆) 8.02-7.95 (1H, m), 7.61 (1H, d, J=8.2 Hz), 7.43 (1H, dd, J=7.7 Hz, 7.3 Hz), 7.27-7.17 (2H, m), 6.82-6.74 (3H, m), 6.49 (2H, brs), 5.04-4.93 (1H, m), 4.72-4.65 (3H, m), 4.55-4.40 (3H, m), 3.80 (2H, q, J=6.4 Hz), 3.52-3.38 (2H, m), 3.27 (3H, s), 3.16-3.12 (3H, m), 3.06 (1H, s), 2.40 (1H, q, J=7.0 Hz), 2.24 (1H, q, J=7.0 Hz), 2.17 (1.5H, s), 2.17-2.06 (1H, m), 2.01 (1.5H, s), 2.01-1.90 (1H, m), 1.18 (3H, d, J=5.6 Hz), 1.17 (3H, d, J=5.5 Hz), 0.92 (1.5H, t, J=7.0 Hz), 0.83 (1.5 H, t, J=7.0 Hz).

MS:ESI 605 (M+1)

### Example 115

### Isopropyl 2-{3-[(N-{3-[4-amino-2-(2-methoxyethyl)-1H-imidazo[4,5-c]quinolin-1-yl]propyl}-2-{(2-methoxyethyl)(methyl)amino}acetamido)methyl]phenoxy}acetate

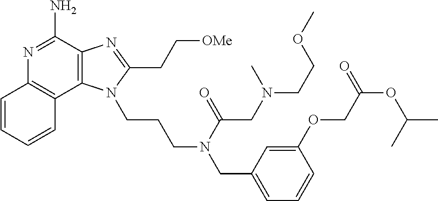

The title compound was prepared by the method of example 5 using the product from example 112 step (ii) (0.22 g) and methoxyethylmethylamine, to give a colorless gum (0.17 g). Yield 77%.

¹H NMR δ (DMSO-d₆) 8.02-7.95 (1H, m), 7.61 (1H, d, J=8.3 Hz), 7.43 (1H, dd, J=8.1 Hz, 7.2 Hz), 7.28-7.17 (2H, m), 6.82-6.74 (3H, m), 6.48 (2H, brs), 5.03-4.93 (1H, m), 4.72 (1H, s), 4.70 (1H, s), 4.66 (1H, s), 4.55-4.50 (1H, m), 4.47-4.42 (2H, m), 3.80 (2H, q, J=6.9 Hz), 3.55-3.38 (2H, m), 3.34-3.25 (2H, m), 3.28 (3H, s), 3.24-3.18 (2H, m), 3.14-3.10 (2H, m), 3.12 (1.5H, s), 3.10 (1.5H, s), 2.55 (1H, t, J=5.5 Hz), 2.50 (1H, t, J=6.5 Hz), 2.23 (1.5H, s), 2.13 (1.5H, s), 2.13-2.06 (1H, m), 2.03-1.95 (1H, m), 1.18 (3H, d, J=6.0 Hz), 1.17 (3H, d, J=6.0 Hz).

MS:ESI 635 (M+1)

### Example 116

### Isopropyl 2-[5-({3-[4-amino-2-(2-methoxyethyl)-1H-imidazo[4,5-c]quinolin-1-yl]propylamino}methyl)-2-fluorophenoxy]acetate

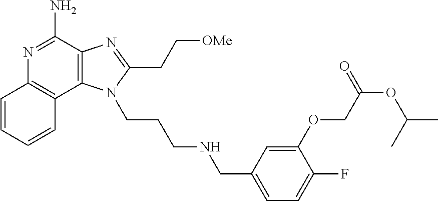

### (i) 2-Fluoro-5-(hydroxymethyl)phenol

To a suspension of LiBH₄ (2.37 g, 109 mmol) in THF (50 ml) was added methyl 4-fluoro-3-hydroxybenzoate (5.0 g, 27.2 mmol) at room temperature. After stirring for 24 h under reflux, the reaction mixture was concentrated. The residue was partitioned between EtOAc (100 ml) and 1N HCl (100 ml). The aqueous layer was extracted with EtOAc (50 ml, twice), the combined organic layers were washed with brine, dried over MgSO₄, and concentrated. The residue was purified by flash column chromatography to give the title compound (2.76 g, 19.4 mmol, 66%) as a white solid.

¹H NMR δ (CDCl₃) 7.09-7.00 (2H, m), 6.88-6.82 (1H, m), 5.24 (1H, d, J=4.0 Hz), 4.60 (2H, s), 1.68 (1H, brs).

### (ii) 4-Fluoro-3-hydroxybenzaldehyde

The title compound was prepared by the method of example 64 step (ii) using the product from step (i) (2.74 g, 19.3 mmol), to give a white solid (0.22 g, 1.57 mmol). Yield 8%.

¹H NMR δ (CDCl₃) 9.91 (1H, s), 7.55 (1H, dd, J=8.4 Hz, 2.0 Hz), 7.47-7.42 (1H, m), 7.27-7.22 (1H, m), 5.44 (1H, d, J=4.2 Hz).

### (iii) Isopropyl 2-(2-fluoro-5-formylphenoxy)acetate

The title compound was prepared by the method of example 64 step (iii) using the product from step (ii) (0.22 g, 1.57 mmol), to give the title compound (0.34 g, 1.40 mmol. 89%) as colorless oil.

¹H NMR δ (CDCl₃) 9.90 (1H, s), 7.53-7.48 (1H, m), 7.44 (1H, dd, J=8.0 Hz, 1.7 Hz), 7.30-7.24 (1H, m), 5.15 (1H, hept, J=6.3 Hz), 4.74 (2H, s), 1.28 (6H, d, J=6.3 Hz).

### (iv) Isopropyl 2-[5-({3-[4-amino-2-(2-methoxyethyl)-1H-imidazo[4,5-c]quinolin-1-yl]propylamino}methyl)-2-fluorophenoxy]acetate

By the method of example 1 step (viii) using the product from example 15 step (iv) (0.41 g, 1.37 mmol) and isopropyl 2-(2-fluoro-5-formylphenoxy)acetate (0.33 g, 1.37 mmol) there was obtained the title compound, 0.51 g (0.97 mmol, 71%) as a white solid

¹H NMR δ (CDCl₃) 8.07 (1H, d, J=7.3 Hz), 7.82 (1H, dd, J=8.4 Hz, 1.0 Hz), 7.53-7.49 (1H, m), 7.30-7.25 (1H, m), 7.09-7.03 (1H, m), 6.98-6.92 (2H, m), 5.47 (2H, brs), 5.11 (1H, hept, J=6.3 Hz), 4.67 (2H, s), 4.67-4.62 (2H, m), 3.90 (2H, t, J=6.6 Hz), 3.74 (2H, s), 3.38 (3H, s), 3.25 (2H, t, J=6.6 Hz), 2.73 (2H, t, J=6.3 Hz), 2.09 (2H, tt, J=7.1, 6.5 Hz), 1.25 (6H, d, J=6.3 Hz).

MS:ESI 524 (M+1)

### Example 117

### Isopropyl 2-{5-[(N-{3-[4-amino-2-(2-methoxyethyl)-1H-imidazo[4,5-c]quinolin-1-yl]propyl}-2-chloroacetamido)methyl]-2-fluorophenoxy}acetate hydrochloride

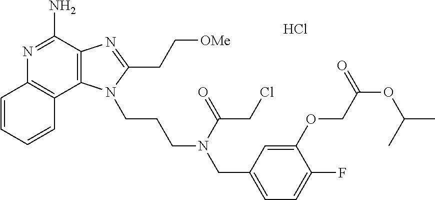

By the method of example 2 using the product of example 116 (0.51 g, 0.97 mmol), there was obtained the title compound, 0.61 g (0.96 mmol, 99%) as a colorless gum.

¹H NMR δ (CDCl₃) 7.91-7.80 (2H, m), 7.56-7.51 (1H, m), 7.37-7.33 (1H, m), 7.07-6.95 (1H, m), 6.75-6.65 (2H, m), 5.75-5.51 (2H, brm), 5.08 (1H, hept, d=6.4 Hz), 4.63-4.47 (6H, m), 4.09 (1.5H, s), 4.05 (0.5H, s), 3.87 (2H, t, J=6.3 Hz), 3.54 (1.5H, t, J=6.9 Hz), 3.40-3.32 (0.5H, m), 3.36 (2.3H, s), 3.34 (0.7H, s), 3.16-3.09 (2H, m), 2.26-2.09 (3H, m), 1.25 (6H, d, J=6.4 Hz).

MS:ESI 601 (M+1)

### Example 118

### Isopropyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate

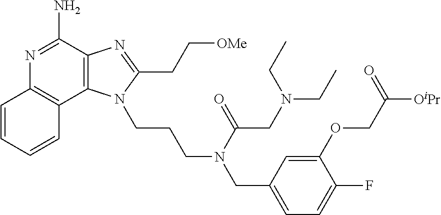

The title compound was prepared by the method of example 5 using the product from example 117 (0.61 g, 0.96 mmol) and diethylamine, to give a pale yellow gum (0.58 g). Yield 95%.

¹H NMR δ (DMSO-d₆) 8.00-7.93 (1H, m), 7.61 (1H, d, J=8.3 Hz), 7.43 (1H, dd, J=8.0 Hz, 7.2 Hz), 7.25-7.12 (2H, m), 6.95-6.90 (1H, m), 6.85-6.74 (1H, m), 6.49 (2H, brs), 4.96-4.90 (1H, m), 4.80 (1H, s), 4.73 (1H, s), 4.69 (1H, s), 4.55-4.48 (1H, m), 4.45-4.40 (1H, m), 4.43 (1H, s), 3.80 (2H, q, J=6.7 Hz), 3.52-3.38 (2H, m), 3.27 (3H, s), 3.22 (1H, s), 3.17 (1H, s), 3.15-3.09 (2H, m), 2.50 (2H, q, J=7.1 Hz), 2.38 (2H, q, J=7.1 Hz), 2.17-2.06 (1H, m), 2.01-1.88 (1H, m), 1.18 (3H, d, J=6.3 Hz), 1.15 (3H, d, J=6.3 Hz), 0.88 (3H, t, J=7.1 Hz), 0.83 (3H, t, J=7.1 Hz).

MS:ESI 637 (M+1)

### Example 119

### Ethyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate

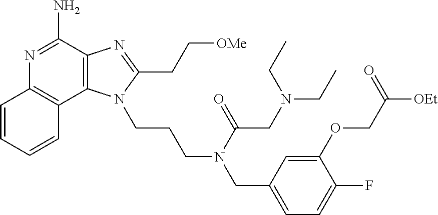

### (i) 2-{5-[(N-{3-[4-Amino-2-(2-methoxyethyl)-1H-imidazo[4,5-c]quinolin-1-yl]propyl}-2-{diethylamino}acetamido)methyl]-2-fluorophenoxy}acetic acid

The title compound was prepared by the method of example 26 step (i) using the product from example 118 (0.44 g), to give a white solid (0.35 g). Yield 86%.

¹H NMR δ (DMSO-d₆) 7.89-7.82 (1H, m), 7.70 (2H, brs), 7.83-7.82 (1H, m), 7.43-7.37 (1H, m), 7.29-7.24 (1H, m), 7.19-7.11 (1H, m), 7.13-6.94 (1H, m), 6.83-6.65 (1H, m), 4.64 (1H, s), 4.59-4.50 (2H, m), 4.44-4.38 (2H, m), 4.34-4.30 (1H, m), 3.80-3.72 (2H, m), 3.50-3.42 (1H, m), 3.36-3.40 (1H, m), 3.28 (3H, s), 3.23 (1H, s), 3.13-3.06 (3H, m), 2.55-2.48 (2H, m), 2.45-2.40 (2H, m), 2.09-1.95 (1H, m), 1.93-1.85 (1H, m), 0.90 (3H, t, J=7.0 Hz), 0.85 (3H, t, J=7.1 Hz).

MS:ESI 595 (M+1)

### (ii) Ethyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate

The title compound was prepared by the method of example 26 step (ii) using the product from step (i) (0.17 g) and ethanol, to give a pale yellow gum (0.16 g). Yield 93%.

¹H NMR δ (DMSO-d₆) 8.00-7.93 (1H, m), 7.60 (1H, d, J=8.2 Hz), 7.42 (1H, dd, J=7.7 Hz, 7.4 Hz), 7.26-7.10 (2H, m), 6.93-6.87 (1H, m), 6.80-6.74 (1H, m), 6.50 (2H, brs), 4.83 (1H, s), 4.77 (1H, s), 4.68 (1H, s), 4.55-4.48 (1H, m), 4.45-4.40 (2H, m), 4.16-4.06 (2H, m), 3.80 (2H, q, J=6.1 Hz), 3.52-3.35 (2H, m), 3.27 (3H, s), 3.23 (1H, s), 3.18 (1H, s), 3.15-3.09 (2H, m), 2.55-2.45 (2H, m), 2.42-2.36 (2H, m), 2.12-2.06 (1H, m), 1.96-1.88 (1H, m), 1.17 (3H, d, J=6.9 Hz), 0.89 (3H, t, J=6.7 Hz), 0.83 (3H, t, J=6.8 Hz).

MS:ESI 623 (M+1)

### Example 120

### Methyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate

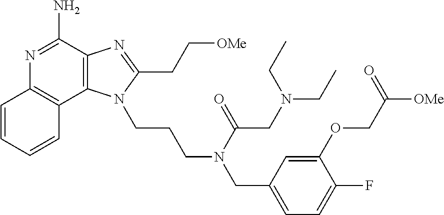

The title compound was prepared by the method of example 26 step (ii) using the product from example 119 step (i) (0.15 g) and methanol, to give a pale yellow gum (0.15 g). Yield 96%.

¹H NMR δ (DMSO-d₆) 8.01-7.93 (1H, m), 7.61 (1H, d, J=8.3 Hz), 7.43 (1H, dd, J=7.5 Hz, 7.4 Hz), 7.24-7.10 (2H, m), 6.96-6.88 (1H, m), 6.80-6.74 (1H, m), 6.51 (2H, brs), 4.85 (1H, s), 4.80 (1H, s), 4.68 (1H, s), 4.53 (1H, t, J=7.2 Hz), 4.47-4.44 (2H, m), 3.80 (2H, q, J=6.6 Hz), 3.68 (1.5H, s), 3.66 (1.5H, s), 3.52-3.35 (2H, m), 3.24 (3H, s), 3.24 (1H, s), 3.19 (1H, s), 3.16-3.09 (2H, m), 2.54-2.45 (2H, m), 2.42-2.36 (2H, m), 2.12-2.06 (1H, m), 1.96-1.88 (1H, m), 0.89 (3H, t, J=7.1 Hz), 0.83 (3H, t, J=7.1 Hz).

MS:ESI 609 (M+1)

### Example 121

### Isopropyl 2-[3-({3-[4-amino-2-(2-methoxyethyl)-1H-imidazo[4,5-c]quinolin-1-yl]propylamino}methyl)-5-fluorophenoxy]acetate

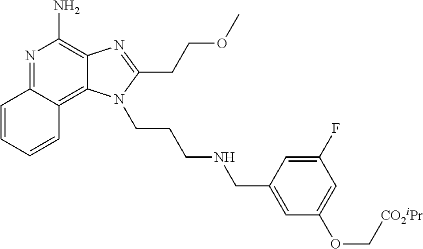

### (i) (3-Bromo-5-fluorophenoxy) (tert-butyl) dimethylsilane

To a solution of 3-bromo-5-fluorophenol (1.50 g, 7.88 mmol) in THF (15 ml) was added tert-butyldimethylsilyl chloride (1.54 g, 10.2 mmol) and imidazole (1.07 g, 15.8 mmol) at 0° C. After stirring for 3 h at room temperature, the reaction mixture was quenched with aqueous citric acid. The mixture was extracted with EtOAc (50 ml, twice), the combined organic layers were washed with brine, dried over MgSO₄, and concentrated. The residue was purified by flash column chromatography to give the title compound (2.21 g, 7.23 mmol, 92%) as a white solid.

¹H NMR δ (CDCl₃) 6.89-6.84 (1H, m), 6.81-6.79 (1H, m), 6.52-6.46 (1H, m), 0.98 (9H, s), 0.22 (6H, s).

### (ii) 3-Fluoro-5-hydroxybenzaldehyde

To a solution of the product from step (i) (2.20 g, 7.23 mmol) in THF (20 ml) was added n-butyllithium (1.6 M hexane solution, 4.97 ml, 7.95 mmol) at −78° C. After stirring for 30 min at −78° C., DMF (1.57 ml, 10.8 mmol) was added to the reaction mixture, and then stirred for 1.5 h at 0° C. Water was added to the mixture, and then the mixture was extracted with EtOAc (50 ml, twice), the combined organic layers were washed with brine, dried over MgSO₄, and concentrated. The residue was purified by flash column chromatography to give 3-(tert-butyldimethylsilyloxy)-5-fluorobenzaldehyde. The compound was dissolved in THF 1.6 ml, thereto tetrabutylammonium fluoride (1.0 M THF solution, 3.38 ml, 3.38 mmol) was added and stirred for 4 h. The reaction was quenched with aqueous citric acid. The mixture was extracted with EtOAc (50 ml, twice), the combined organic layers were washed with brine, dried over MgSO₄, and concentrated. The residue was purified by flash column chromatography to give the title compound (0.13 g, 0.92 mmol, 13%) as a white solid.

¹H NMR δ (CDCl₃) 9.90 (1H, s), 7.19-7.14 (2H, m), 6.89-6.84 (1H, m), 5.56 (1H, brs).

### (iii) Isopropyl 2-(3-fluoro-5-formylphenoxy)acetate

The title compound was prepared by the method of example 64 step (iii) using the product from step (ii) (0.13 g, 0.89 mmol), to give the title compound (0.18 g, 0.76 mmol. 85%) as colorless oil.

¹H NMR δ (CDCl₃) 9.91 (1H, s), 7.24-7.18 (2H, m), 6.95-6.90 (1H, m), 5.15 (1H, hept, J=6.3 Hz), 4.65 (2H, s), 1.28 (6H, d, J=6.3 Hz).

### (iv) Isopropyl 2-[3-({3-[4-amino-2-(2-methoxyethyl)-1H-imidazo[4,5-c]quinolin-1-yl]propylamino}methyl)-5-fluorophenoxy]acetate

By the method of example 1 step (viii) using the product from example 15 step (iv) (0.22 g, 0.73 mmol) and isopropyl 2-(3-fluoro-5-formylphenoxy)acetate (0.18 g, 0.73 mmol) there was obtained the title compound, 0.29 g (0.56 mmol, 77%) as a white solid.

¹H NMR δ (CDCl₃) 8.08 (1H, d, J=8.1 Hz), 7.83 (1H, d, J=8.3 Hz), 7.51 (1H, dd, J=8.1 Hz, 7.2 Hz), 7.32-7.25 (1H, m), 6.76-6.72 (2H, m), 6.55-6.50 (1H, m), 5.57 (2H, brs), 5.14 (1H, hept, J=6.3 Hz), 4.67 (2H, t, J=7.4 Hz), 4.59 (2H, s), 3.90 (2H, t, J=6.5 Hz), 3.77 (2H, s), 3.38 (3H, s), 3.25 (2H, t, J=6.5 Hz), 2.74 (2H, t, J=6.2 Hz), 2.13-2.04 (2H, m), 1.26 (6H, d, J=6.3 Hz).

MS:ESI 524 (M+1)

### Example 122

### Isopropyl 2-{3-[(N-{3-[4-amino-2-(2-methoxyethyl)-1H-imidazo[4,5-c]quinolin-1-yl]propyl}-2-chloroacetamido)methyl]-5-fluorophenoxy}acetate hydrochloride

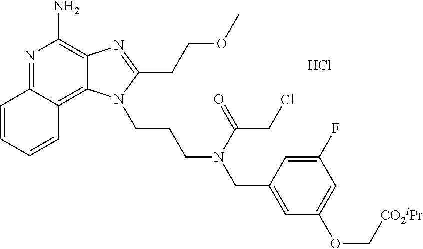

By the method of example 2 using the product of example 121 (0.29 g, 0.55 mmol), there was obtained the title compound, 0.33 g (0.52 mmol, 94%) as a colorless gum.

¹H NMR δ (CDCl₃) 7.93-7.80 (2H, m), 7.57-7.51 (1H, m), 7.40-7.34 (1H, m), 6.53-6.48 (3H, m), 5.90-5.51 (2H, brm), 5.13 (1H, hept, d=6.3 Hz), 4.60-4.50 (6H, m), 4.08 (2H, s), 3.87 (2H, t, J=6.2 Hz), 3.59 (1.5H, t, J=6.9 Hz), 3.50-3.40 (0.3H, m), 3.37 (2H, s), 3.34 (0.7H, s), 3.18-3.09 (2H, m), 2.26-2.04 (3H, m), 1.28 (6H, d, J=6.4 Hz).

MS:ESI 601 (M+1)

### Example 123

### Isopropyl 2-{3-[(N-{3-[4-amino-2-(2-methoxyethyl)-1H-imidazo[4,5-c]quinolin-1-yl]propyl}-2-{diethylamino}acetamido)methyl]-5-fluorophenoxy}acetate

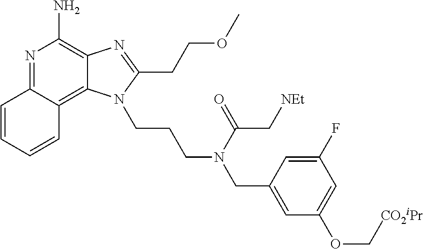

The title compound was prepared by the method of example 5 using the product from example 122 (0.32 g, 0.51 mmol) and diethylamine, to give a colorless gum (0.26 g). Yield 82%.

¹H NMR δ (CDCl₃) 7.95-7.88 (2H, m), 7.59 (1H, dd, J=7.7 Hz, 7.4 Hz), 7.47-7.42 (1H, m), 6.57-6.50 (2H, m), 6.49-6.44 (1H, m), 5.85-5.55 (2H, brm), 5.13 (1H, hept, J=6.3 Hz), 4.76 (1.5H, s), 4.55-4.47 (4.5H, m), 3.87 (2H, t, J=6.2 Hz), 3.67-3.50 (2H, m), 3.36-3.28 (5H, m), 3.16-3.08 (2H, m), 2.62-2.53 (4H, m), 2.30-2.05 (2H, m), 1.28 (6H, d, J=6.3 Hz), 1.01-0.98 (6H, m).

MS:ESI 637 (M+1)

### Example 124

### Ethyl 2-{3-[(N-{3-[4-amino-2-(2-methoxyethyl)-1H-imidazo[4,5-c]quinolin-1-yl]propyl}-2-{diethylamino}acetamido)methyl]-5-fluorophenoxy}acetate

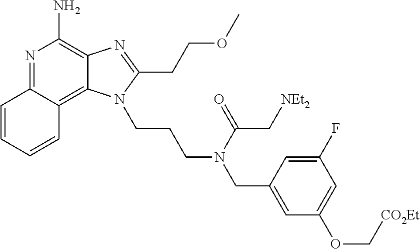

### (i) 2-{3-[(N-{3-[4-Amino-2-(2-methoxyethyl)-1H-imidazo[4,5-c]quinolin-1-yl]propyl}-2-{diethylamino}acetamido)methyl]-5-fluorophenoxy}acetic acid

The title compound was prepared by the method of example 26 step (i) using the product from example 123 (0.16 g), to give a white solid (0.16 g). Yield 100%.

¹H NMR δ (DMSO-d₆) 7.99-7.91 (1H, m), 7.59 (1H, d, J=8.3 Hz), 7.44 (1H, dd, J=7.7 Hz, 7.5 Hz), 7.29-7.22 (1H, m), 7.13 (2H, brs), 6.66-6.52 (3H, m), 4.71 (1H, s), 4.55-4.40 (5H, m), 3.79 (2H, q, J=6.8 Hz), 3.56-3.51 (1H, m), 3.44-3.28 (1H, m), 3.27 (3H, s), 3.25 (1H, s), 3.23 (1H, s), 3.16-3.10 (2H, m), 2.55-2.48 (2H, m), 2.45-2.40 (2H, m), 2.15-2.05 (1H, m), 2.00-1.92 (1H, m), 0.89 (3H, t, J=7.0 Hz), 0.84 (3H, t, J=7.1 Hz).

MS:ESI 595 (M+1)

### (ii) Ethyl 2-{3-[(N-{3-[4-amino-2-(2-methoxyethyl)-1H-imidazo[4,5-c]quinolin-1-yl]propyl}-2-{diethylamino}acetamido)methyl]-5-fluorophenoxy}acetate

The title compound was prepared by the method of example 26 step (ii) using the product from step (i) (0.093 g) and ethanol, to give a pale yellow gum (0.088 g). Yield 91%.

¹H NMR δ (CDCl₃) 7.91-7.83 (2H, m), 7.54 (1H, dd, J=7.4 Hz, 7.4 Hz), 7.38-7.28 (1H, m), 6.66-6.44 (3H, m), 5.85-5.55 (2H, brm), 4.75 (1.5H, s), 4.56-4.46 (4.5H, m), 4.28 (2H, q, J=7.1 Hz), 3.87 (2H, t, J=6.3 Hz), 3.65-3.49 (2H, m), 3.36-3.34 (3H, m), 3.28-3.26 (2H, m), 3.17-3.09 (2H, m), 2.62-2.50 (4H, m), 2.30-1.90 (2H, m), 1.30 (3H, t, J=7.1 Hz), 0.97 (6H, t, J=7.1 Hz).

MS:ESI 623 (M+1)

### Example 125

### Ethyl 2-{4-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate

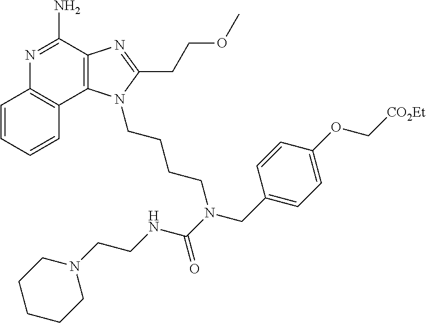

### (i) Ethyl 2-(4-formylphenoxy)acetate

To a solution of 2-(4-formylphenoxy)acetic acid (4.00 g, 22.2 mmol) in EtOH (100 ml) was added conc. H₂SO₄ (1 ml). After stirring at reflux temperature for 4 h, the reaction mixture was concentrated, neutralized with satd. NaHCO₃ aq. (200 ml), and extracted with AcOEt (100 ml×2). The combined extracts were dried over MgSO₄ and concentrated to afford the subtitle compound (4.45 g, 96%) as a white solid.

### (ii) Ethyl 2-{4-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate

To a solution of the product of example 42 step (vi) (277 mg, 0.883 mmol) and product from step (i) (184 mg, 0.882 mmol) in MeOH (10 ml) were added AcOH (101 ul, 1.77 mmol) and NaBH₃CN (56.1 mg, 0.893 mmol) at 0° C. After stirring at between 0° C. and room temperature over night, the reaction mixture was concentrated, and poured with satd. NaHCO₃ aq. (50 ml). The aq. Layer was extracted with CHCl₃-MeOH (20:1, 50 ml×2), dried over Na₂SO₄, and concentrated. The residue was purified by flash column chromatography to give subtitle compound (223 mg, 50%) as a colorless gum.

### (iii) Ethyl 2-{4-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate

To a solution of the product from step (ii) (221 mg, 0.437 mmol) and i-Pr₂NEt (188 ul, 1.09 mmol) in THF (5 ml). The mixture was added 4-nitrophenyl carbonochloridate (116 mg, 0.576 mmol) at 0° C. After stirring at the same temperature for 30 min, the mixture was added 2-(piperidin-1-yl)ethanamine (73.8 mg, 0.576 mmol) and DMSO (5 ml). After further stirring at room temperature over night, the reaction mixture was diluted with satd. NaHCO₃ aq. (30 ml), and extracted with AcOEt (50 ml×2). The extracts were washed with H₂O (50 ml×2) and brine (50 ml×1), dried over Na₂SO₄, and concentrated. The residue was purified by flash column chromatography to afford the title compound (207 mg, 72%) as a colorless gum.

¹H NMR δ (DMSO-d₆) 7.97 (1H, d, J=8.2), 7.61 (1H, dd, J=1.0, 8.2), 7.42 (1H, dt, J=1.0, 7.1), 7.25 (1H, dt, J=1.0, 7.1), 7.15-7.10 (2H, m), 6.87 (2H, m), 6.47 (2H, brs), 6.19-6.12 (1H, m), 4.73 (2H, s), 4.48 (2H, t, J=7.1), 4.33 (2H, s), 4.15 (2H, q, J=7.1), 3.81 (2H, t, J=6.8), 3.29 (3H, s), 3.19-3.08 (6H, m), 2.31-2.18 (6H, m), 1.79-1.68 (2H, m), 1.65-1.54 (2H, m), 1.44-1.29 (6H, m), 1.20 (3H, t, J=7.1).

### MS: ESI 660 (M+1)

### Example 126

### Ethyl 2-{3-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate

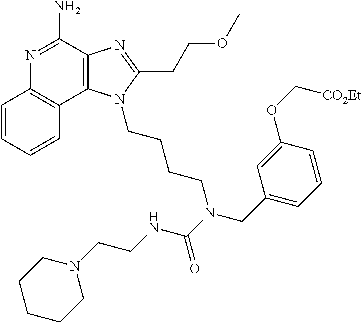

The title compound (149 mg) was obtained by the same procedure of example 125 step (iii) using the product of example 42 (151 mg, 0.299 mmol). Yield 76%

¹H NMR δ (DMSO-d₆) 7.98 (1H, d, J=8.0), 7.61 (1H, d, J=8.0), 7.42 (1H, t, J=7.5), 7.25 (1H, t, J=7.5), 7.19 (1H, d, J=8.0), 6.83-6.72 (3H, m), 6.48 (2H, brs), 6.22-6.17 (1H, m), 4.72 (2H, s), 4.49 (2H, t, J=7.2), 4.38 (2H, s), 4.15 (2H, q, J=7.1), 3.81 (2H, t, J=6.7), 3.29 (3H, s), 3.21-3.08 (6H, m), 2.32-2.19 (6H, m), 1.80-1.79 (2H, m), 1.65-1.55 (2H, m), 1.43-1.28 (6H, m), 1.20 (3H, t, J=7.1).

### MS: ESI 660 (M+1)

### Example 127

### Ethyl 2-{3-[(1-{4-[4-amino-2-(2-methoxyethyl)-1H-imidazo[4,5-c]quinolin-1-yl]butyl}-3-{2-(dimethylamino)ethyl}ureido)methyl]phenoxy}acetate

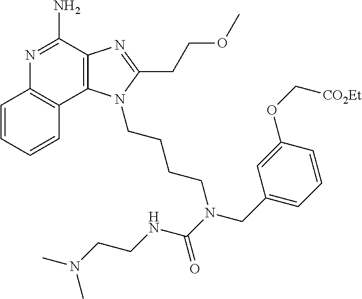

The title compound (50.4 mg) was obtained by the same procedure of example 125 step (iii) using the product of example 42 (124 mg, 0.246 mmol). Yield 33%

¹H NMR δ (DMSO-d₆) 7.98 (1H, d, J=8.2), 7.61 (1H, d, J=8.2), 7.42 (1H, t, J=7.6), 7.27-7.18 (2H, m), 6.81-6.74 (3H, m), 6.47 (2H, brs), 6.24 (1H, t, J=5.4), 4.72 (2H, s), 4.49 (2H, t, J=7.4), 4.38 (2H, s), 4.15 (2H, q, J=7.1), 3.81 (2H, t, J=6.7), 3.29 (3H, s), 3.20-3.06 (6H, m), 2.21 (2H, t, J=6.9), 2.07 (6H, s), 1.79-1.70 (2H, m), 1.64-1.54 (2H, m), 1.20 (3H, t, J=7.1).

MS: ESI 620 (M+1)

### Example 128

### Ethyl

### 2-{3-[(1-{4-[4-amino-2-(2-methoxyethyl)-1H-imidazo[4,5-c]quinolin-1-yl]butyl}-3-{3-(piperidin-1-yl)propyl}ureido)methyl]phenoxy}acetate

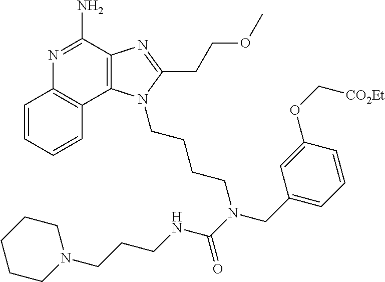

The title compound (38.4 mg) was obtained by the same procedure of example 125 step (iii) using the product of example 42 (141 mg, 0.278 mmol). Yield 21%

¹H NMR δ (DMSO-d₆) 7.82 (1H, dd, J=0.8, 8.2), 7.73 (1H, dd, J=1.0, 8.4), 7.44-7.39 (1H, m), 7.26-7.21 (1H, m), 7.16-7.12 (1H, m), 6.76-6.67 (3H, m), 5.93 (1H, t, J=4.6), 5.42 (2H, brs), 4.51 (2H, s), 4.43 (2H, t, J=7.5), 4.28 (2H, s), 4.19 (2H, q, J=7.1), 3.80 (2H, t, J=6.5), 3.30 (3H, s), 3.30-3.19 (2H, m), 3.09 (2H, t, J=6.5), 2.25-2.11 (6H, m), 1.88-1.77 (2H, m), 1.65-1.51 (4H, m), 1.27-1.18 (9H, m).

### MS: ESI 674 (M+1)

### Example 129

### Ethyl 2-{3-[(1-{4-[4-amino-2-(2-methoxyethyl)-1H-imidazo[4,5-c]quinolin-1-yl]butyl}-3-{3-(dimethylamino)propyl}ureido)methyl]phenoxy}acetate

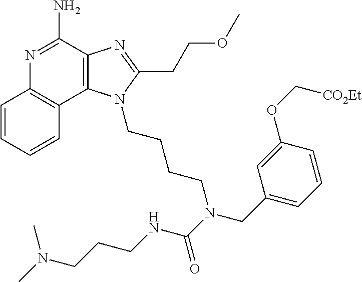

The title compound (30.5 mg) was obtained by the same procedure of example 125 step (iii) using the product of example 42 (133 mg, 0.262 mmol). Yield 18%

¹H NMR δ (DMSO-d₆) 7.82 (1H, dd, J=0.8, 8.2), 7.74 (1H, dd, J=1.0, 8.4), 7.44-7.40 (1H, m), 7.26-7.21 (1H, m), 7.15 (1H, t, J=8.0), 6.75-6.65 (3H, m), 6.50 (1H, t, J=4.4), 5.48 (2H, brs), 4.51 (2H, s), 4.44 (2H, t, J=7.6), 4.26 (2H, s), 4.18 (2H, q, J=7.1), 3.81 (2H, t, J=6.5), 3.30 (3H, s), 3.32-3.26 (2H, m), 3.26-3.20 (2H, m), 3.10 (2H, t, J=6.5), 2.19 (2H, t, 5.8), 2.13-1.92 (4H, m), 1.88-1.78 (2H, m), 1.87 (6H, s), 1.69-1.58 (2H, m), 1.54-1.47 (2H, m), 1.23 (3H, t, J=7.1).

### MS: ESI 634 (M+1)

### Example 130

### Ethyl 2-{3-[(3-{4-[4-amino-2-(2-methoxyethyl)-1H-imidazo[4,5-c]quinolin-1-yl]butyl}-1-{2-(piperidin-1-yl)ethyl}ureido)methyl]phenoxy}acetate

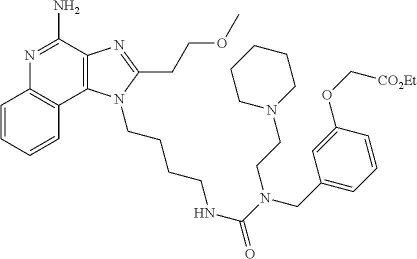

To a solution of ethyl 2-(3-formylphenoxy)acetate (500 mg, 2.40 mmol) and 2-(piperidin-1-yl)ethanamine (308 mg, 2.40 mmol) in MeOH (20 ml) were added AcOH (275 ul, 4.81 mmol) and NaBH₃CN (151 mg, 2.41 mmol) at 0° C. After stirring at between 0° C. and room temperature over night, the reaction mixture was concentrated, and poured with satd. NaHCO₃ aq. (50 ml). The aq. layer was extracted with CHCl₃-MeOH (20:1, 50 ml×2), dried over Na₂SO₄, and concentrated. The residue was purified by flash column chromatography to give ethyl 2-(3((2-(piperidin-1-yl)ethylamino)methyl)phenoxy)acetate. (555 mg, 72%) as a colorless gum.

The title compound (178 mg) was obtained by the same procedure of example 22 step (i) using the product of example 42 step (vi) and ethyl 2-(3-((2-(piperidin-1-yl)ethylamino)methyl)phenoxy)acetate. Yield 60%

¹H NMR δ (DMSO-d₆) 8.00 (1H, d, J=7.8), 7.61 (1H, dd, J=1.1, 8.3), 7.41 (1H, dt, J=1.1, 7.1), 7.24-7.14 (2H, m), 7.03 (1H, brs), 6.77-6.72 (3H, m), 6.48 (2H, brs), 4.71 (2H, s), 4.54 (2H, t, J=7.3), 4.37 (2H, s), 4.14 (2H, q, J=7.1), 3.82 (2H, t, J=6.8), 3.29 (3H, s), 3.19 (2H, t, J=6.8), 3.15-3.05 (4H, m), 2.28-2.12 (6H, m), 1.87-1.77 (2H, m), 1.63-1.52 (2H, m), 1.40-1.25 (6H, m), 1.19 (3H, t, J=7.1).

### MS: ESI 660 (M+1)

### Example 131

### Ethyl 2-{4-[(3-{4-[4-amino-2-(2-methoxyethyl)-1H-imidazo[4,5-c]quinolin-1-yl]butyl}-1-{2-(piperidin-1-yl)ethyl}ureido)methyl]phenoxy}acetate

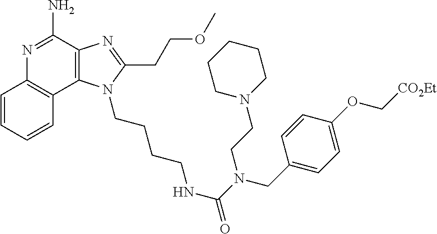

The title compound (190 mg) was obtained by the same procedure of example 130 using the product of example 42 step (vi) (174 mg, 0.555 mmol). Yield 52%

¹H NMR δ (DMSO-d₆) 8.00 (1H, d, J=8.2), 7.61 (1H, d, J=8.2), 7.41 (1H, t, J=7.2), 7.22 (1H, t, J=7.2), 7.11-7.06 (2H, m), 7.03-6.97 (1H, m), 6.83-6.79 (2H, m), 6.48 (2H, brs), 4.73 (2H, s), 4.54 (2H, t, J=7.2), 4.32 (2H, s), 4.16 (2H, q, J=7.1), 3.82 (2H, t, J=6.7), 3.29 (3H, s), 3.19 (2H, t, J=6.7), 3.14-3.02 (4H, m), 2.28-2.10 (6H, m), 1.87-1.76 (2H, m), 1.63-1.52 (2H, m), 1.39-1.24 (6H, m), 1.21 (3H, t, J=7.1).

### MS: ESI 660 (M+1)

### Example 132

### Isopropyl 2-{3-[({4-[4-amino-2-(2-methoxyethyl)-1H-imidazo[4,5-c]quinolin-1-yl]butyl}{2-[dimethylamino]ethyl}amino)methyl]phenoxy}acetate

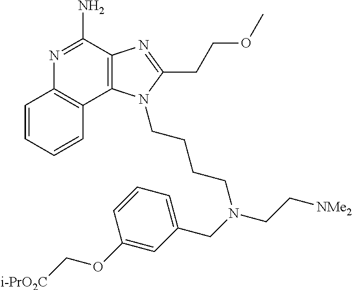

### (i) N-{4-[4-Amino-2-(2-methoxyethyl)-1H-imidazo[4,5-c]quinolin-1-yl]butyl}-2-nitrobenzenesulfonamide

To a solution of the product of example 42 step (vi) (1.51 g, 4.80 mmol) in CHCl₃ (150 ml) and THF (30 ml) was added o-nitrobenzenesulfonyl chloride (1.18 g, 5.31 mmol) at 0° C. After stirring at room temperature for 2 h, the reaction mixture was quenched by satd. NaHCO₃ aq. (100 ml), and extracted with CHCl₃ (200 ml×2). The combined extracts were dried over Na₂SO₄ and concentrated. The residue was purified by flash column chromatography to afford the subtitle compound (2.23 g, 93%) as a white solid.

### (ii) N-{4-[4-Amino-2-(2-methoxyethyl)-1H-imidazo[4,5-c]quinolin-1-yl]butyl}-N-[2-(dimethylamino)ethyl]-2-nitrobenzenesulfonamide

To a solution of the product of step (i) (703 mg, 1.41 mmol) PPh₃ (1.11 g, 4.22 mmol) and 2-(dimethylamino)ethanol (262 ul, 2.20 mmol) in THF (30 ml) was added DIAD (2.21 ml, 4.20 mmol) at 50° C. After stirring at the same temperature for 30 min, the reaction mixture was concentrated and purified by flash column chromatography to give the subtitle compound (589 mg, 73%) as colorless amorphous.

### (iii) N1-{4-[4-Amino-2-(2-methoxyethyl)-1H-imidazo[4,5-c]quinolin-1-yl]butyl}N2,N2-dimethylethane-1,2-diamine

To a solution of the product of step (ii) (589 mg, 1.03 mmol) in DMF (15 ml) were added 2-mercaptoacetic acid (485 ul, 7.00 mmol) and lithium hydroxide (334 mg, 13.9 mmol) at room temperature. After stirring at the same temperature over night, the reaction mixture was quenched by satd. NaHCO₃ aq. (50 ml) and extracted with CHCl₃ (50 ml×3). The combined organic layers were dried over Na₂SO₄ and concentrated. The residue was purified by flash column chromatography to afford the subtitle compound (286 mg, 53%) as a white solid.

¹H NMR δ (DMSO-d₆) 8.07 (1H, dd, J=1.1, 8.3), 7.60 (1H, dd, J=1.2, 8.3), 7.42 (1H, ddd, J=1.1, 7.0, 8.2), 7.25 (1H, ddd, J=1.2, 7.0, 8.2), 6.47 (2H, s), 4.54 (2H, t, J=7.4), 3.83 (2H, t, J=6.9), 3.30 (3H, s), 3.19 (2H, t, J=6.7), 2.58-2.51 (4H, m), 2.25 (2H, t, J=6.4), 2.09 (6H, s), 1.88-1.79 (2H, m), 1.60-1.51 (2H, m).

MS: ESI 385 (M+1)

### (iv) Isopropyl 2-{3-[({4-[4-amino-2-(2-methoxyethyl)-1H-imidazo[4,5-c]quinolin-1-yl]butyl}{2-[dimethylamino]ethyl}amino)methyl]phenoxy}acetate

To a solution of the product of step (iii) (187 mg, 0.424 mmol) in THF (5 ml) were added isopropyl 2-(3-formylphenoxy)acetate (292.4 mg, 1.32 mmol), acetic acid (48 ul, 0.839 mmol) and NaBH(OAc)₃ (273 mg, 1.29 mmol) at room temperature. After stirring for 5 days at the same temperature, the reaction mixture was quenched by satd. NaHCO₃ aq. (50 ml) and extracted with CHCl₃ (50 ml×2). The combined organic layers were dried over Na₂SO₄ and concentrated. The residue was purified by flash column chromatography to afford the title compound (57.6 mg, 28%) as a colorless gum.

¹H NMR δ (DMSO-d₆) 8.03 (1H, d, J=7.8), 7.60 (1H, d, J=7.8), 7.41 (1H, dd, J=7.2, 7.6), 7.25 (1H, dd, J=7.2, 8.0), 7.15 (1H, dd, J=7.8, 7.9), 6.87-6.79 (2H, m), 6.74 (1H, dd, J=2.2, 7.9), 6.47 (2H, s), 4.93 (1H, sep, J=6.1), 4.62 (2H, s), 4.51 (2H, t, J=7.9), 3.82 (2H, t, J=6.7), 3.50 (2H, s), 3.28 (3H, s), 3.17 (2H, t, J=6.7), 2.48-2.39 (4H, m), 2.27-2.21 (2H, m), 2.03 (6H, s), 1.88-1.77 (2H, m), 1.65-1.54 (2H, m), 1.15 (6H, d, J=6.1).

### MS: ESI 591 (M+1)

### Example 133

### Isopropyl 2-{3-[({4-[4-amino-2-(2-methoxyethyl)-1H-imidazo[4,5-c]quinolin-1-yl]butyl}{3-morpholinopropyl}amino)methyl]phenoxy}acetate

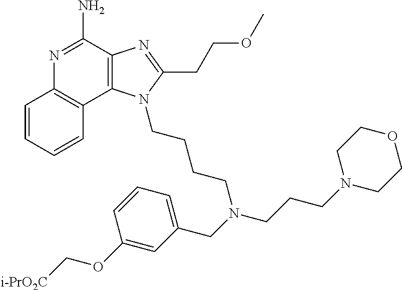

### (i) N-{4-[4-Amino-2-(2-methoxyethyl)-1H-imidazo[4,5-c]quinolin-1-yl]butyl}-N-(3-morpholinopropyl)-2-nitrobenzenesulfonamide

The subtitle compound (421 mg) was prepared by the same procedure of example 131 step (ii) using the product of example 131 step (i) (1.17 g). Yield: ˜100%

### (ii) 2-(2-Methoxyethyl)-1-[4-(3-morpholinopropylamino)butyl]-1H-imidazo[4, 5-c]quinolin-4-amine

The subtitle compound (421 mg) was prepared by the same procedure of example 131 step (iii) using the product of step (i) (1.17 g). Yield: 68%

¹H NMR δ (DMSO-d₆) 8.07 (1H, d, J=8.0), 7.60 (1H, d, J=7.9), 7.45-7.39 (1H, m), 7.28-7.22 (1H, m), 6.48 (2H, brs), 4.54 (2H, t, J=7.3), 3.83 (2H, t, J=6.8), 3.55-3.48 (4H, m), 3.30 (3H, s), 3.19 (2H, t, J=6.8), 2.32-2.20 (6H, m), 1.90-1.80 (2H, m), 1.60-1.58 (4H, m).

MS: ESI 441 (M+1).

### (iii) Isopropyl 2-{3-[({4-[4-amino-2-(2-methoxyethyl)-1H-imidazo[4,5-c]quinolin-1-yl]butyl}{3-morpholinopropyl}amino)methyl]phenoxy}acetate

The title compound (99.0 mg) was synthesized by the same procedure of example 131 step (iv) using the product from step (ii) (187 mg). Yield: 36%

¹H NMR δ (DMSO-d₆) 8.03 (1H, d, J=8.0), 7.60 (1H, d, J=8.0), 7.41 (1H, dd, J=7.4, 8.0), 7.23 (1H, dd, J=7.3, 7.4), 7.15 (1H, dd, 7.8, 8.0), 6.85-6.79 (2H, m), 6.75-6.71 (1H, m), 6.47 (2H, brs), 4.98-4.88 (1H, m), 4.62 (2H, s), 4.51 (2H, t, J=7.3), 3.82 (2H, t, J=6.7), 3.50-3.42 (6H, m), 3.35-3.22 (2H, m), 3.28 (3H, s), 3.17 (2H, t, J=6.7), 2.44-2.37 (2H, m), 2.37-2.30 (2H, m), 2.23-2.10 (6H, m), 1.87-1.76 (2H, m), 1.63-1.55 (2H, m), 1.55-1.44 (2H, m), 1.15 (6H, d, J=6.2).

MS: ESI 647 (M+1).

### Example 134

### Ethyl 2-{3-[({4-[4-amino-2-(2-methoxyethyl)-1H-imidazo[4,5-c]quinolin-1-yl]butyl}{2-(dimethylamino)ethyl}amino)methyl]phenoxy}-2-methylpropanoate

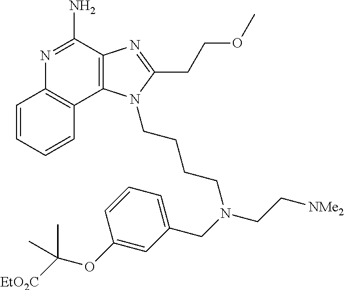

The title compound (132 mg) was synthesized by the same procedure of example 132 step (iv) using the product of example 132 step (iii) (149 mg) and example 53 step (i). Yield: 56%

¹H NMR δ (DMSO-d₆) 8.02 (1H, d, J=7.9), 7.60 (1H, dd, J=1.0, 8.3), 7.41 (1H, dd, 7.1, 7.2), 7.23 (1H, ddd, J=1.0, 7.1, 8.0), 7.13 (1H, dd, J=7.8, 7.9), 6.84 (1H, d, J=7.6), 6.72 (1H, m), 6.62 (1H, dd, J=2.0, 8.0), 6.47 (2H, s), 4.51 (2H, t, J=7.4), 4.08 (2H, q, J=7.1), 3.82 (2H, t, J=6.8), 3.48 (2H, s), 3.28 (3H, s), 3.17 (2H, t, 6.8), 2.47-2.38 (4H, m), 2.26-2.21 (2H, m), 2.03 (6H, s), 1.86-1.76 (2H, m), 1.65-1.56 (2H, m), 1.43 (6H, s), 1.10 (3H, t, J=7.1).

MS: ESI 605 (M+1).

### Example 135

### Methyl

### 2-{3-[({4-[4-amino-2-(2-methoxyethyl)-1H-imidazo[4,5-c]quinolin-1-yl]butyl}{2-(dimethylamino)ethyl}amino)methyl]phenoxy}-2-methylpropanoate

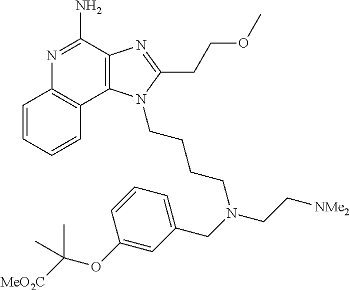

### (i) 2-{3-[({4-[4-Amino-2-(2-methoxyethyl)-1H-imidazo[4,5-c]quinolin-1-yl]butyl}{2-(dimethylamino)ethyl}amino)methyl]phenoxy}-2-methylpropanoic acid

To a solution of the product of example 134 (118 mg, 0.195 mmol) in THF (3 ml) was added 1N NaOH (1 ml) at 0° C. After stirring at 60° C. for 8 h, the reaction mixture was neutralized by 1N HCl (1 ml), diluted with brine (10 ml) and extracted with CHCl₃-EtOH (3:1, 15 ml×3). The combined extracts were dried over Na₂SO₄ and concentrated to give the subtitle compound (116 mg, ˜100%) as a white solid.

### (ii) Methyl 2-{3-[({4-[4-amino-2-(2-methoxyethyl)-1H-imidazo[4,5-c]quinolin-1-yl]butyl}{2-(dimethylamino)ethyl}amino)methyl]phenoxy}-2-methylpropanoate

To a solution of the product of step (i) (115 mg, 0.200 mmol) in MeOH (5 ml) was added conc. sulfuric acid (5 drops) at 0° C. After stirring at 60° C. for 8 h, the reaction mixture was quenched by satd. NaHCO₃ aq. (20 ml) and extracted with CHCl₃ (30 ml×2). The combined extracts were dried over Na₂SO₄ and concentrated. The residue was purified by flash column chromatography to afford the title compound (34.9 mg, 30%) as a colorless gum.

¹H NMR δ (DMSO-d₆) 8.03 (1H, d, J=7.7), 7.61 (1H, dd, J=1.1, 8.3), 7.44-7.39 (1H, m), 7.25-7.21 (1H, m), 7.13 (1H, t, J=7.8), 6.85 (1H, d, J=7.6), 6.71 (1H, m), 6.60 (1H, dd, 2.0, 8.1), 6.48 (2H, brs), 4.51 (2H, t, J=7.5), 3.82 (2H, t, J=6.8), 3.63 (3H, s), 3.48 (2H, s), 3.48 (2H, s), 3.28 (3H, s), 3.17 (2H, t, J=6.7), 2.47-2.38 (4H, m), 2.27-2.21 (2H, m), 2.03 (6H, s), 1.86-1.76 (2H, m), 1.64-1.54 (2H, m), 1.43 (6H, s).

MS: ESI 591 (M+1).

### Example 136

### Ethyl 2-{3-[({4-[4-amino-2-(2-methoxyethyl)-1H-imidazo[4,5-c]quinolin-1-yl]butyl}{3-morpholinopropyl}amino)methyl]phenoxy}-2-methylpropanoate

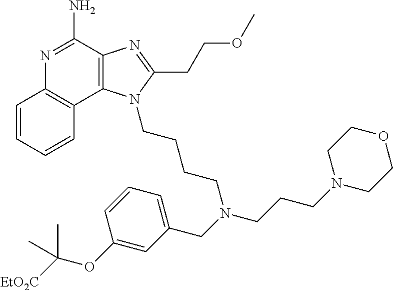

The title compound (175 mg) was synthesized by the same procedure of example 131 step (iv) using the product of example 133 step (ii). Yield: 52%

¹H NMR δ (DMSO-d₆) 8.02 (1H, d, J=7.9), 7.61 (1H, dd, J=0.1, 8.3), 7.43-7.39 (1H, m), 7.27-7.21 (1H, m), 7.12 (1H, dd, J=7.8, 7.9), 6.82 (1H, d, J=7.5), 6.73-6.69 (1H, m), 6.61 (1H, dd, 2.1, 7.9), 6.50 (2H, brs), 4.51 (2H, t, J=7.5), 4.08 (2H, q, J=7.1), 3.82 (2H, t, J=6.8), 3.49-3.41 (6H, m), 3.28 (3H, s), 3.17 (2H, t, J=6.8), 2.39 (2H, t, J=6.5), 2.34 (2H, t, J=6.8), 2.23-2.11 (6H, m), 1.86-1.75 (2H, m), 1.64-1.53 (2H, m), 1.53-1.45 (2H, m), 1.44 (6H, s), 1.10 (3H, t, J=7.1).

MS: ESI 661 (M+1).

### Example 137

### Methyl 2-{3-[({4-[4-amino-2-(2-methoxyethyl)-1H-imidazo[4,5-c]quinolin-1-yl]butyl}{3-morpholinopropyl}amino)methyl]phenoxy}-2-methylpropanoate

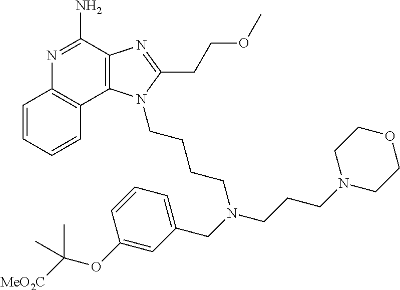

### (i) 2-{3-[({4-[4-Amino-2-(2-methoxyethyl)-1H-imidazo[4,5-c]quinolin-1-yl]butyl}{3-morpholinopropyl}amino)methyl]phenoxy}-2-methylpropanoic acid

The subtitle compound (149 mg) was prepared by the same procedure of example 26 step (i) using product of example 136 (159 mg). Yield: 98%

### (ii) Methyl 2-{3-[({4-[4-amino-2-(2-methoxyethyl)-1H-imidazo[4,5-c]quinolin-1-yl]butyl}{3-morpholinopropyl}amino)methyl]phenoxy}-2-methylpropanoate

The title compound (102 mg) was synthesized by the same procedure of example 26 step (ii) using the product from step (i) (138 mg). Yield: 72%

¹H NMR δ (DMSO-d₆) 8.02 (1H, d, J=8.0), 7.60 (1H, dd, J=1.0 8.3), 7.43-7.39 (1H, m), 7.25-7.21 (1H, m), 7.12 (1H, dd, J=7.8, 7.9), 6.83 (1H, d, J=7.8), 6.70 (1H, m), 6.60 (1H, dd, 2.0, 7.8), 6.48 (2H, brs), 4.51 (2H, t, J=7.2), 3.82 (2H, t, J=6.8), 3.62 (3H, s), 3.50-3.42 (6H, m), 3.28 (3H, s), 3.17 (2H, t, J=6.7), 2.42-2.30 (4H, m), 2.23-2.12 (6H, m), 1.87-1.66 (2H, m), 1.65-1.55 (2H, m), 1.55-1.45 (2H, m), 1.44 (6H, s). MS: ESI 647 (M+1).

### Example 138

### Isopropyl 2-[5-({4-[4-amino-2-(2-methoxyethyl)-1H-imidazo[4,5-c]quinolin-1-yl]butylamino}methyl)-2-fluorophenoxy]acetate

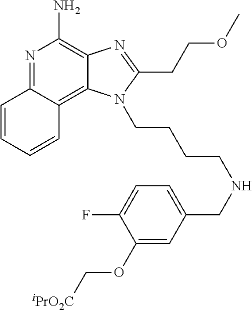

By the method of example 1 step (viii) using the product from example 42 step (vi) (0.66 g, 2.10 mmol) and example 116 step (iii) (0.50 g, 2.10 mmol) there was obtained the title compound, 0.76 g (1.41 mmol, 67%) as yellow oil.

¹H NMR δ (CDCl₃) 7.97 (1H, d, J=8.1 Hz), 7.82 (1H, d, J=8.3 Hz), 7.53-7.49 (1H, m), 7.33-7.28 (1H, m), 7.06-6.98 (1H, m), 6.93-6.85 (2H, m), 5.45 (2H, brs), 5.13-5.05 (1H, m), 4.63 (2H, s), 4.53 (2H, t, J=7.8 Hz), 3.90 (2H, d, J=6.6 Hz), 3.70 (2H, s), 3.38 (3H, s), 3.17 (2H, t, J=8.3 Hz), 2.67 (2H, t, J=7.0 Hz), 2.04-1.95 (2H, m), 1.71-1.63 (2H, m), 1.24 (6H, d, J=6.3 Hz).

MS:ESI 538 (M+1)

### Example 139

### Isopropyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate

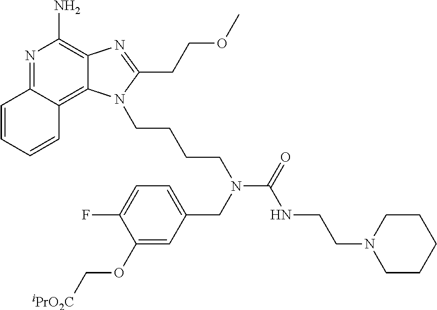

The title compound was prepared by the method of example 125 step (iii) using the product from example 138 (0.39 g) to give a colorless gum (0.19 g). Yield 39%.

¹H NMR δ (CDCl₃) 7.91 (1H, d, J=7.8 Hz), 7.82 (1H, d, J=8.4 Hz), 7.53-7.49 (1H, m), 7.36-7.30 (1H, m), 7.06-6.99 (1H, m), 6.83-6.77 (2H, m), 5.52 (2H, brs), 5.09 (1H, hept, J=6.3 Hz), 4.62 (2H, s), 4.54 (2H, t, J=7.6 Hz), 4.36 (2H, s), 3.89 (2H, t, J=6.4 Hz), 3.38-3.30 (7H, m), 3.17 (2H, t, J=6.4 Hz), 2.55-2.42 (5H, m), 2.00-1.60 (11H, m), 1.60-1.30 (7H, m), 1.25 (6H, d, J=6.3 Hz).

MS:ESI 692 (M+1)

### Example 140

### Ethyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate

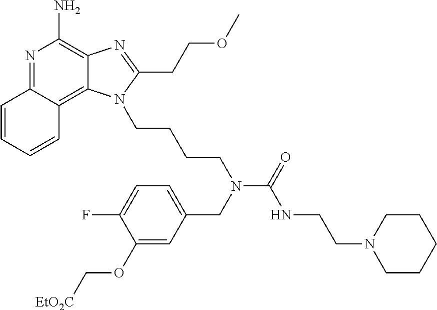

### (i) 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetic acid

The title compound was prepared by the method of example 26 step (i) using the product from example 139 (0.13 g), to give a white solid (0.10 g). Yield 86%.

¹H NMR δ (DMSO-d₆) 7.92 (1H, d, J=8.0 Hz), 7.59 (1H, d, J=7.6 Hz), 7.42 (1H, dd, J=7.4 Hz, 7.3 Hz), 7.27-7.22 (1H, m), 7.13-7.06 (1H, m), 7.07 (2H, brs), 6.85-6.82 (1H, m), 6.74-6.70 (1H, m), 6.54-6.48 (1H, brt), 4.47 (2H, t, J=7.1 Hz), 4.37 (2H, s), 4.35 (2H, s), 3.80 (2H, t, J=6.7 Hz), 3.30-3.25 (4H, m), 3.21 (2H, t, J=7.0 Hz), 3.14 (2H, t, J=6.7 Hz), 3.02-2.80 (7H, m), 1.75-1.63 (8H, m), 1.51-1.40 (2H, m).

MS:ESI 650 (M+1)

### (ii) Ethyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate

The title compound was prepared by the method of example 26 step (ii) using the product from step (i) (0.042 g) and ethanol, to give a pale yellow gum (0.036 g). Yield 85%.

¹H NMR δ (CDCl₃) 7.91 (1H, d, J=8.2 Hz), 7.82 (1H, dd, J=8.4 Hz, 1.0 Hz), 7.53-7.48 (1H, m), 7.35-7.31 (1H, m), 7.05-6.99 (1H, m), 6.83-6.78 (2H, m), 5.70-5.42 (3H, m), 4.65 (2H, s), 4.53 (2H, t, J=7.6 Hz), 4.35 (2H, s), 4.24 (2H, q, J=7.1 Hz), 3.88 (2H, t, J=6.5 Hz), 3.38-3.30 (7H, m), 3.17 (2H, t, J=6.5 Hz), 2.50-2.30 (6H, m), 2.00-1.88 (2H, m), 1.75-1.60 (2H, m), 1.70-1.35 (6H, m), 1.28 (3H, t, J=7.1 Hz).

MS:ESI 678 (M+1)

### Example 141

### Methyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate

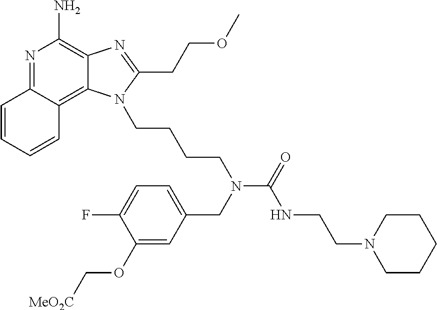

The title compound was prepared by the method of example 26 step (ii) using the product from example 140 step (i) (0.040 g) and methanol, to give a pale yellow gum (0.037 g). Yield 90%.

¹H NMR δ (CDCl₃) 7.91 (1H, dd, J=8.2 Hz, 0.9 Hz), 7.82 (1H, dd, J=8.4 Hz, 1.0 Hz), 7.51-7.47 (1H, m), 7.34-7.30 (1H, m), 7.03-6.99 (1H, m), 6.83-6.78 (2H, m), 5.44 (2H, brs), 4.67 (2H, s), 4.53 (2H, t, J=7.6 Hz), 4.34 (2H, s), 3.88 (2H, t, J=6.5 Hz), 3.78 (3H, s), 3.38-3.31 (5H, m), 3.30-3.27 (2H, m), 3.17 (2H, t, J=6.5 Hz), 2.48-2.30 (6H, m), 1.98-1.87 (2H, m), 1.76-1.67 (2H, m), 1.72-1.25 (6H, m).

MS:ESI 664 (M+1)

### Example 142

### Isopropyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate

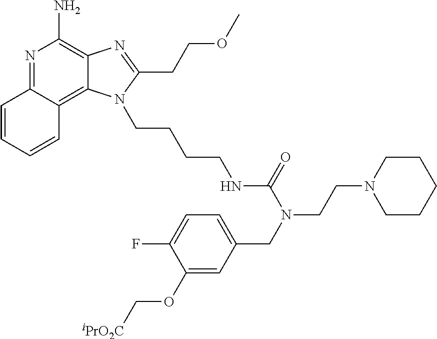

### (i) Isopropyl 2-(2-fluoro-5-{[2-(piperidin-1-yl)ethylamino]methyl}phenoxy)acetate

By the method of example 22 step (i) using 2-(piperidin-1-yl)ethanamine (0.31 ml, 2.19 mmol) and the product from example 116 step (iii) (0.46 g, 1.91 mmol) there was obtained the title compound, 0.57 g (1.61 mmol, 84%) as yellow oil.

¹H NMR δ (CDCl₃) 7.06-7.00 (1H, m), 6.95-6.86 (2H, m), 7.30-7.24 (1H, m), 5.14 (1H, hept, J=6.3 Hz), 4.67 (2H, s), 3.73 (2H, s), 2.67 (2H, t, J=6.2 Hz), 2.45 (2H, t, J=6.2 Hz), 2.40-2.31 (4H, m), 1.60-1.54 (4H, m), 1.50-1.38 (2H, m), 1.28 (6H, d, J=6.3 Hz).

### (ii) Isopropyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate

The title compound was prepared by the method of example 22 step (ii) using the product from step (i) (0.57 g) to give a pale yellow gum (0.69 g). Yield 78%.

¹H NMR δ (CDCl₃) 8.12 (1H, brs), 7.95 (1H, d, J=7.5 Hz), 7.83 (1H, d, J=8.2 Hz), 7.49 (1H, dd, J=7.2 Hz, 7.1 Hz), 7.31-7.25 (1H, m), 7.03-6.96 (1H, m), 6.89-6.85 (1H, m), 6.85-6.77 (1H, m), 5.47 (2H, brs), 5.11 (1H, hept, J=6.3 Hz), 4.63 (2H, s), 4.56 (2H, t, J=7.7 Hz), 4.39 (2H, s), 3.89 (2H, t, J=6.5 Hz), 3.38 (3H, s), 3.29-3.22 (2H, m), 3.21 (2H, t, J=6.5 Hz), 3.05-3.00 (2H, m), 2.30-2.20 (6H, m), 2.05-1.94 (2H, m), 1.75-1.65 (2H, m), 1.40-1.30 (6H, m), 1.26 (6H, d, J=6.3 Hz).

MS:ESI 692 (M+1)

### Example 143

### Ethyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate

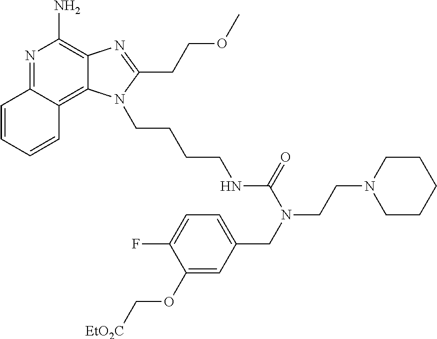

### (i) 2-{5-[(3-{4-[4-Amino-2-(2-methoxyethyl)-1H-imidazo[4,5-c]quinolin-1-yl]butyl}-1-{2-(piperidin-1-yl)ethyl}ureido)methyl]-2-fluorophenoxy}acetic acid

The title compound was prepared by the method of example 26 step (i) using the product from example 142 (0.52 g), to give a white solid (0.42 g). Yield 87%.

¹H NMR δ (DMSO-d₆) 7.94 (1H, d, J=8.2 Hz), 7.60 (1H, d, J=7.7 Hz), 7.50 (2H, brs), 7.41 (1H, dd, J=7.5 Hz, 7.5 Hz), 7.22 (1H, dd, J=7.3 Hz, 7.3 Hz), 7.09-7.02 (1H, m), 7.00 (1H, brt), 6.87 (1H, d, J=8.3 Hz), 6.87-6.64 (1H, m), 4.50 (2H, t, J=7.3 Hz), 4.48 (2H, s), 4.28 (2H, s), 3.81 (2H, t, J=6.7 Hz), 3.28 (3H, s), 3.17 (2H, t, J=6.7 Hz), 3.12-3.04 (4H, m), 2.35-2.25 (6H, m), 1.79-1.70 (2H, m), 1.62-1.51 (2H, m), 1.43-1.35 (4H, m), 1.33-1.26 (2H, m).

MS:ESI 650 (M+1)

### (ii) Ethyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate

The title compound was prepared by the method of example 26 step (ii) using the product from step (i) (0.19 g) and ethanol, to give a pale yellow gum (0.19 g). Yield 97%.

¹H NMR δ (CDCl₃) 8.10 (1H, brs), 7.95 (1H, d, J=7.5 Hz), 7.83 (1H, d, J=8.2 Hz), 7.49 (1H, dd, J=7.1 Hz, 7.1 Hz), 7.32-7.25 (1H, m), 7.04-6.96 (1H, m), 6.90-6.85 (1H, m), 6.85-6.77 (1H, m), 5.56 (2H, brs), 4.66 (2H, s), 4.56 (2H, t, J=7.7 Hz), 4.39 (2H, s), 4.25 (2H, q, J=7.2 Hz), 3.89 (2H, t, J=6.5 Hz), 3.38 (3H, s), 3.29-3.22 (2H, m), 3.22 (2H, t, J=6.9 Hz), 3.08-3.00 (2H, m), 2.30-2.20 (6H, m), 2.05-1.94 (2H, m), 1.75-1.65 (2H, m), 1.40-1.30 (6H, m), 1.29 (3H, t, J=7.2 Hz).

MS:ESI 678 (M+1)

### Example 144

### Methyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate

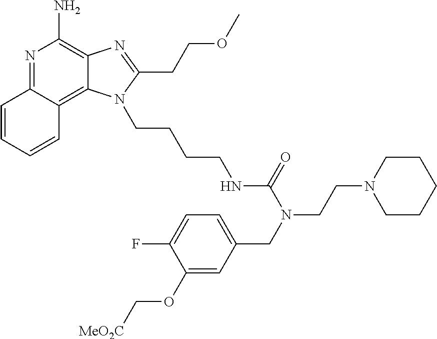

The title compound was prepared by the method of example 26 step (ii) using the product from example 143 step (i) (0.16 g) and methanol, to give a pale yellow gum (0.17 g). Yield 100%.

¹H NMR δ (CDCl₃) 8.10 (1H, brs), 7.96 (1H, d, J=7.7 Hz), 7.82 (1H, d, J=7.6 Hz), 7.50 (1H, dd, J=7.2 Hz, 7.2 Hz), 7.32-7.25 (1H, m), 7.04-6.96 (1H, m), 6.90-6.85 (1H, m), 6.85-6.80 (1H, m), 5.57 (2H, brs), 4.66 (2H, s), 4.57 (2H, t, J=7.8 Hz), 4.39 (2H, s), 3.89 (2H, t, J=6.5 Hz), 3.79 (3H, s), 3.38 (3H, s), 3.29-3.22 (2H, m), 3.21 (2H, t, J=6.5 Hz), 3.08-3.00 (2H, m), 2.30-2.20 (6H, m), 2.05-1.94 (2H, m), 1.75-1.65 (2H, m), 1.40-1.30 (6H, m).

MS:ESI 664 (M+1)

### Example 145

### Isopropyl 2-[3-({3-[4-amino-2-(2-methoxyethyl)-1H-imidazo[4,5-c]quinolin-1-yl]propylamino}methyl)-2-methylphenoxy]acetate

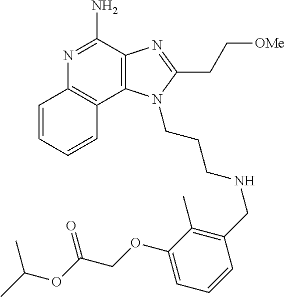

### (i) Isopropyl 2-[3-(hydroxymethyl)-2-methylphenoxy]acetate

To a solution of 3-hydroxy-2-methylbenzyl alcohol (0.77 g, 5.59 mmol) in DMF (25 mL), K₂CO₃ (1.2 g, 3.39 mmol) and Isopropyl bromoacetate (0.80 mL, 6.15 mmol) were added. After being stirred at rt overnight, the reaction mixture was diluted with EtOAc and H₂O was added to the mixture. The resulting mixture was extracted with EtOAc (×3). The combined extracts were washed with brine, dried over Na₂SO₄ and concentrated in vacuo. The residue was purified by flash column chromatography to afford the title compound (1.3 g, 95%) as colorless oil.

¹H NMR δ (CDCl₃) 7.14 (1H, t, J=7.9 Hz), 7.03 (1H, d, J=7.9 Hz), 6.69 (1H, d, J=7.9 Hz), 5.13 (1H, quint, J=6.3 Hz), 4.70 (2H, s), 4.60 (2H, s), 2.29 (3H, s), 1.27 (6H, d, J=6.3 Hz)

ESI-MS [M−H₂O+H]⁺: 221.12

### (ii) N-{3-[4-amino-2-(2-methoxyethyl)-1H-imidazo[4,5-c]quinolin-1-yl]propyl}-2-nitrobenzenesulfonamide

The title compound was prepared by the same method of example 21 step (i) using the material from example 15 step (iv) (1.07 g, 3.57 mmol) to afford titled compound as a pale yellow solid. 1.32 g, 2.74 mmol, 77%.

¹H NMR δ (CDCl₃) ¹H NMR δ (CDCl₃) 8.07 (1H, d, J=7.5 Hz), 7.93 (1H, d, J=7.5 Hz), 7.85-7.79 (2H, m), 7.70-7.64 (2H, m), 7.55-7.49 (1H, m), 7.38-7.30 (1H, m), 5.62 (2H, brs), 4.67 (2H, t, J=7.3 Hz), 3.95 (2H, t, J=5.9 Hz), 3.38 (3H, s), 3.21 (2H, t, J=5.9 Hz), 2.28-2.18 (2H, m), 1.72-1.65 (2H, m).

### (iii) Isopropyl 2-{3-[(N-{3-[4-amino-2-(2-methoxyethyl)-1H-imidazo[4,5-c]quinolin-1-yl]propyl}-2-nitrophenylsulfonamido)methyl]-2-methylphenoxy}acetate

By the method of example 21 step (ii) using the product from step (i) (0.44 g, 1.86 mmol) and the product from step (ii) (0.60 g, 1.24 mmol), to give the title compound as pale yellow amorphousness (0.69 g, 79%)

¹H NMR δ (CDCl₃) 7.85-7.81 (2H, m), 7.73-7.71 (1H, m), 7.61-7.56 (4H, m), 7.32-7.29 (1H, m), 6.87 (1H, t, J=8.0 Hz), 6.71 (1H, d, J=7.5 Hz), 6.51 (1H, d, J=8.0 Hz), 5.62 (2H, brs), 5.08 (1H, quint, J=6.3 Hz), 4.50 (2H, s), 4.49 (2H, s), 4.36 (2H, t, J=7.4 Hz), 3.84 (2H, t, J=6.3 Hz), 3.39-3.34 (5H, m), 3.04 (2H, t, J=6.3 Hz), 2.17 (3H, s), 1.96 (2H, q, J=7.4 Hz), 1.28-1.25 (6H, m)

ESI-MS [M+H]⁺: 705

### (iv) Isopropyl 2-[3-({3-[4-amino-2-(2-methoxyethyl)-1H-imidazo[4,5-c]quinolin-1-yl]propylamino}methyl)-2-methylphenoxy]acetate

By the method of example 22 step (iii) using the product from step (iv) (0.69 g, 0.972 mmol), to give the title compound as a yellow gum (0.40 g, 80%).

ESI-MS [M+H]⁺: 520

### Example 146

### Isopropyl 2-{3-[(N-{3-[4-amino-2-(2-methoxyethyl)-1H-imidazo[4,5-c]quinolin-1-yl]propyl}-2-chloroacetamido)methyl]-2-methylphenoxy}acetate hydrochloride

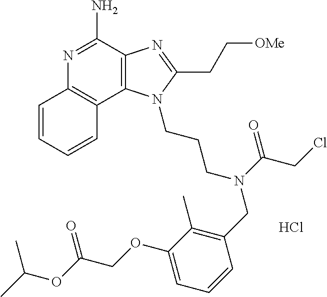

By the method of example 2 using the product of example 145 (0.40 g, 0.770 mmol), there was obtained the title compound (0.23 g, 48%) as a pale yellow gum.

¹H NMR δ (CDCl₃) 7.95 (1H, d, J=7.9 Hz), 7.87 (1H, d, J=8.2 Hz), 7.57-7.53 (1H, m), 7.40-7.37 (1H, m), 7.10 (1H, t, J=8.1 Hz), 6.66-6.61 (2H, m), 5.13 (1H, quint, J=6.3 Hz), 5.17-5.10 (5.7H, m), 4.48-4.44 (0.3H, m), 4.08 (0.5H, s), 4.05 (1.5H, s), 3.89-3.84 (2H, m), 3.62 (2H, t, J=6.7 Hz), 3.40 (3H, s), 3.17 (1.7H, t, J=6.3 Hz), 3.08 (0.3H, t, J=6.3 Hz), 2.20-2.14 (5H, m), 1.29-1.28 (6H, m)

ESI-MS [M+H]⁺: 596

### Example 147

### Isopropyl 2-{3-[(N-{3-[4-amino-2-(2-methoxyethyl)-1H-imidazo[4,5-c]quinolin-1-yl]propyl}-2-{diethylamino}acetamido)methyl]-2-methylphenoxy}acetate

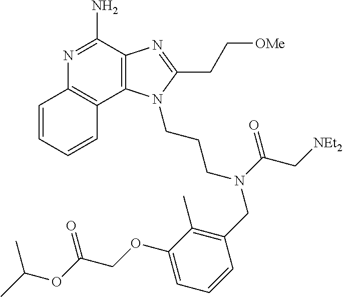

By the method of example 5 using the product of example 146 (0.23 g, 0.367 mmol) and diethylamine (0.38 mL, 3.67 mmol), to give the title compound as a pale yellow gum (0.17 g, 75%).

¹H NMR δ (CDCl₃) 7.94 (0.8H, d, J=7.6 Hz), 7.87-7.81 (1.2H, m), 7.55-7.52 (1H, m), 7.39-7.36 (1H, m), 7.08 (0.8H, t, J=8.0 Hz), 6.99 (0.2H, t, J=8.0 Hz), 6.67-6.62 (2H, m), 5.81-5.65 (2H, m), 5.14 (1H, quint, J=6.3 Hz), 4.78 (1.6H, s), 4.64 (0.4H, s), 4.59-4.57 (2H, m), 4.54-4.50 (1.6H, m), 4.46-4.42 (0.4H, m), 3.89-3.83 (2H, m), 3.58 (2H, t, J=6.9 Hz), 3.36 (2.4H, s), 3.34 (0.6H, s), 3.28 (0.4H, s), 3.16 (1.6H, t, J=6.3 Hz), 3.09 (0.4H, t, J=6.3 Hz), 2.61-2.53 (4H, m), 2.18-2.09 (5H, m), 1.03-0.95 (6H, m)

ESI-MS [M+2H]²⁺: 317

### Example 148

### Ethyl 2-{3-[(N-{3-[4-amino-2-(2-methoxyethyl)-1H-imidazo[4,5-c]quinolin-1-yl]propyl}-2-{dimethylamino}acetamido)methyl]phenoxy}-2-methylpropanoate

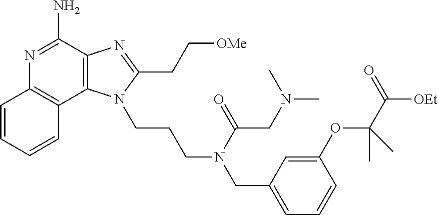

The title compound was prepared by the method of example 5 using the product from example 54 (0.39 g) and dimethylamine, to give a colorless gum (0.31 g). Yield 84%.

¹H NMR δ (CDCl₃) 7.91 (0.75H, d, J=7.7 Hz), 7.83 (0.25H, d, J=8.0 Hz), 7.81 (1H, d, J=7.5 Hz), 7.54-7.49 (1H, m), 7.36-7.31 (1H, m), 7.21-7.16 (1H, m), 6.80-6.69 (3H, m), 5.53 (2H, brs), 4.70 (1.5H, s), 4.56 (0.5H, s), 4.53-4.46 (2H, m), 4.21 (2H, q, J=7.1 Hz), 3.86 (2H, t, J=6.4 Hz), 3.55 (1.5H, t, J=6.8 Hz), 3.50-3.46 (0.5H, m), 3.35 (2.25H, s), 3.34 (0.75H, s), 3.20-3.13 (3H, m), 3.09 (0.5H, t, J=6.3 Hz), 3.05 (0.5H, s), 2.50-2.35 (2H, m), 2.31 (4.5H, s), 2.30-2.22 (0.5H, m), 2.13-2.05 (3H, m), 1.57 (6H, s), 1.25-1.21 (3H, m)

MS:ESI 605 (M+1)

### Example 149

### Methyl 2-{3-[(N-{3-[4-amino-2-(2-methoxyethyl)-1H-imidazo[4,5-c]quinolin-1-yl]propyl}-2-{dimethylamino}acetamido)methyl]phenoxy}-2-methylpropanoate

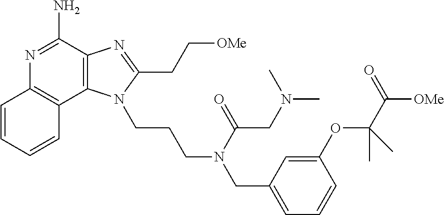

### (i) 2-{3-[(N-{3-[4-Amino-2-(2-methoxyethyl)-1H-imidazo[4,5-c]quinolin-1-yl]propyl}-2-{dimethylamino}acetamido)methyl]phenoxy}-2-methylpropanoic acid

The title compound was prepared by the method of example 26 step (i) using the product from example 148 (0.18 g), to give a white solid (0.17 g). Yield 98%.

¹H NMR δ (DMSO-d₆) 7.95-7.86 (1H, m), 7.62-7.59 (1H, m), 7.44 (1H, dd, J=8.0 Hz, 7.2 Hz), 7.24 (2H, brs), 7.24-7.21 (1H, m), 7.20-7.15 (1H, m), 6.79-6.66 (3H, m), 4.62 (1H, s), 4.47 (1H, t, J=7.4 Hz), 4.44 (1H, s), 4.37 (1H, t, J=7.5 Hz), 3.81-3.75 (2H, m), 3.47-3.41 (2H, m), 3.27 (3H, s), 3.17-3.09 (4H, m), 2.23 (3H, s), 2.08 (3H, s), 2.10-2.02 (1H, m), 1.95-1.86 (1H, m), 1.49 (3H, s), 1.47 (3H, s).

MS:ESI 577 (M+1)

### (ii) Methyl 2-{3-[(N-{3-[4-amino-2-(2-methoxyethyl)-1H-imidazo[4,5-c]quinolin-1-yl]propyl}-2-{dimethylamino}acetamido)methyl]phenoxy}-2-methylpropanoate

The title compound was prepared by the method of example 26 step (ii) using the product from step (i) (0.14 g) and MeOH, to give a colorless gum (0.12 g). Yield 85%.

¹H NMR δ (CDCl₃) 7.92 (0.75H, d, J=7.8 Hz), 7.88 (0.25H, d, J=8.3 Hz), 7.83 (1H, d, J=8.3 Hz), 7.55-7.49 (1H, m), 7.38-7.32 (1H, m), 7.22-7.16 (1H, m), 6.83-6.67 (3H, m), 5.56 (1.5H, brs), 5.50 (0.5H, brs), 4.71 (1.5H, s), 4.57 (0.5H, s), 4.54-4.48 (2H, m), 3.87 (2H, t, J=6.3 Hz), 3.75 (3H, s), 3.56 (1.5H, t, J=6.8 Hz), 3.50-3.46 (0.5H, m), 3.36 (2.25H, s), 3.35 (0.75H, s), 3.18-3.15 (3H, m), 3.08 (0.5H, t, J=7.4 Hz), 3.06 (0.5H, s), 2.31 (4.5H, s), 2.30-2.22 (0.5H, m), 2.13-2.03 (3H, m), 1.57 (6H, s).

MS:ESI 591 (M+1)

### Example 150

### Ethyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}-2-methylpropanoate

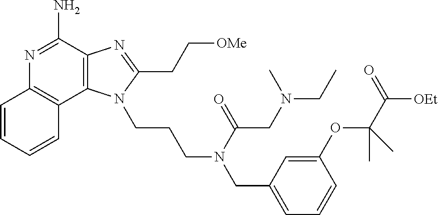

The title compound was prepared by the method of example 5 using the product from example 54 (0.40 g) and ethylmethylamine, to give a colorless gum (0.34 g). Yield 88%.

¹H NMR δ (CDCl₃) 7.92 (0.75H, d, J=7.8 Hz), 7.86 (0.25H, d, J=8.0 Hz), 7.83 (1H, d, J=8.4 Hz), 7.55-7.50 (1H, m), 7.38-7.33 (1H, m), 7.21-7.16 (1H, m), 6.80-6.69 (3H, m), 5.54 (1.5H, brs), 5.50 (0.5H, brs), 4.74 (1.5H, s), 4.57 (0.5H, s), 4.53-4.47 (2H, m), 4.21 (2H, q, J=7.1 Hz), 3.87 (2H, t, J=6.4 Hz), 3.58-3.50 (2H, m), 3.36 (2.25H, s), 3.34 (0.75H, s), 3.20 (1.5H, s), 3.18-3.07 (2.5H, m), 2.47 (1.5H, q, J=7.1 Hz), 2.40-2.32 (0.5H, m), 2.29 (2.25H, s), 2.28-2.06 (2.75H, m), 1.58 (6H, s), 1.26-1.22 (3H, m), 1.06-0.96 (3H, m).

MS:ESI 619 (M+1)

### Example 151

### Methyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}-2-methylpropanoate

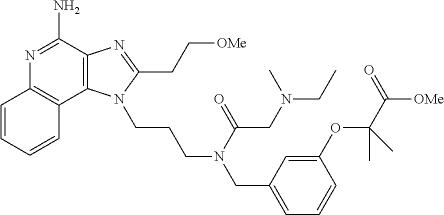

### (i) 2-{3-[(N-{3-[4-Amino-2-(2-methoxyethyl)-1H-imidazo[4,5-c]quinolin-1-yl]propyl}-2-{dimethylamino}acetamido)methyl]phenoxy}-2-methylpropanoic acid

The title compound was prepared by the method of example 26 step (i) using the product from example 150 (0.24 g), to give a white solid (0.21 g). Yield 97%.

¹H NMR δ (DMSO-d₆) 7.96-7.88 (1H, m), 7.62-7.58 (1H, m), 7.43 (1H, dd, J=7.5 Hz, 7.4 Hz), 7.26-7.11 (2H, m), 6.98 (2H, brs), 6.77-6.67 (3H, m), 4.67 (1H, s), 4.47 (1H, t, J=7.0 Hz), 4.43 (1H, s), 4.38 (1H, t, J=8.0 Hz), 3.82-3.75 (2H, m), 3.47-3.40 (2H, m), 3.27 (3H, s), 3.15-3.08 (4H, m), 2.40 (1H, q, J=7.1 Hz), 2.27 (1H, q, J=7.1 Hz), 2.17 (1.5H, s), 2.10-2.05 (1H, m), 2.07 (1.5H, s), 1.98-1.90 (1H, m), 1.46 (3H, s), 1.45 (3H, s), 0.91 (1.5H, t, J=7.1 Hz), 0.83 (1.5H, t, J=7.2 Hz).

MS:ESI 577 (M+1)

### (ii) Methyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}-2-methylpropanoate

The title compound was prepared by the method of example 26 step (ii) using the product from step (i) (0.16 g) and MeOH, to give a colorless gum (0.15 g). Yield 74%.

¹H NMR δ (CDCl₃) 7.92 (0.75H, d, J=8.1 Hz), 7.86 (0.25H, d, J=8.0 Hz), 7.83 (1H, d, J=8.3 Hz), 7.55-7.50 (1H, m), 7.38-7.33 (1H, m), 7.22-7.16 (1H, m), 6.82-6.66 (3H, m), 5.55 (1.5H, brs), 5.49 (0.5H, brs), 4.74 (1.5H, s), 4.57 (0.5H, s), 4.53-4.47 (2H, m), 3.87 (2H, t, J=6.4 Hz), 3.75 (3H, s), 3.58-3.54 (2H, m), 3.36 (2.25H, s), 3.34 (0.75H, s), 3.20 (1.5H, s), 3.18-3.08 (2.5H, m), 2.47 (1.5H, q, J=7.1 Hz), 2.37 (0.5H, q, J=7.1 Hz), 2.29 (2.25H, s), 2.26-2.03 (2.75H, m), 1.58 (6H, s), 1.03 (2.25H, t, J=7.1 Hz), 0.97 (0.75H, t, J=7.1 Hz).

### Example 152

### Isopropyl 2-(3-{[1-(2-{2-[4-amino-2-(2-methoxyethyl)-1H-imidazo[4,5-c]quinolin-1-yl]ethoxy}ethyl)-3-{2-(piperidin-1-yl)ethyl}ureido]methyl}phenoxy)acetate

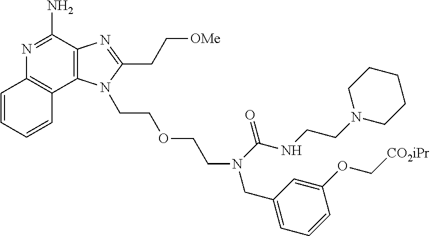

### (i) 2-[2-(3-Nitroquinolin-4-ylamino)ethoxy[ethanol

The title compound was prepared by the method of example 1 step (ii) using the product from example 1 step (i) (11.5 g) and 2-(2-aminoethoxy)ethanol, to give the title compound (6.14 g). Yield 84%. MS:ESI 278 (M+1)

### (ii) 2-{2-[2-(3-Nitroquinolin-4-ylamino)ethoxy]ethyl}isoindoline-1,3-dione

To a solution of the product of steo (i) (5.0 g, 18.1 mmol), triphenylphosphine (6.63 g, 25.3 mmol), and phthalimide (3.74 g, 25.4 mmol) in THF (100 ml) was added DIAD (13.3 ml, 1.9 M in THF, 25.3 mmol) at room temperature. After stirring for 45 min at the same temperature, the reaction mixture was concentrated. The residue was suspended in MeOH (150 ml) and filtered. The obtained solids were washed with MeOH to give the subtitle compound (6.30 g, 86%) as white solids. MS: ESI 407 (M+1)

### (iii) 2-{2-[2-(3-Aminoquinolin-4-ylamino)ethoxy]ethyl}isoindoline-1,3-dione

The title compound was prepared by the method of example 1 step (iii) using the product from step (ii) (6.30 g), to give the title compound (4.91 g). Yield 84%.

MS:ESI 377 (M+1)

### (iv) 2-(2-{2-[2-(2-Methoxyethyl)-1H-imidazo[4,5-c]quinolin-1-yl]ethoxy}ethyl)isoindoline-1,3-dione

The title compound was prepared by the method of example 15 step (i) using the product from step (iii) (4.78 g), to give the title compound (3.89 g). Yield 69%.

MS:ESI 445 (M+1)

### (v) 2-(2-{2-[4-Amino-2-(2-methoxyethyl)-1H-imidazo[4,5-c]quinolin-1-yl]ethoxylethyl)isoindoline-1,3-dione

The title compound was prepared by the method of example 1 step (v) and (vi) using the product from step (iv) (3.89 g), to give the title compound (3.19 g). Yield 79%. MS:ESI 460 (M+1)

### (vi) 1-[2-(2-aminoethoxy)ethyl]-2-(2-methoxyethyl)-1H-imidazo[4,5-c]quinolin-4-amine

To a solution of the product from step (v) (3.03 g, 6.59 mmol) in EtOH (70 ml) was added hydrazine monohydrate (5 ml, 10.3 mmol) at room temperature. After stirring for 6 h at reflux temperature, the reaction mixture was concentrated. The residue was diluted with 20% K₂CO₃ aq. (200 ml) and extracted with CHCl₃-EtOH (3:1) (200 ml×3). The combined organic layer was dried over Na₂SO₄ and concentrated to afford the crude of the title compound.

MS:ESI 330 (M+1)

### (vii) Isopropyl 2-{3-[(2-{2-[4-amino-2-(2-methoxyethyl)-1H-imidazo[4,5-c]quinolin-1-yl]ethoxy}ethylamino)methyl]phenoxy}acetate

The title compound was prepared by the method of example 1 step (viii) using the product from step (vi) (0.615 g), to give the title compound (0.212 g). Yield 32%. (by 2 steps) MS:ESI 536 (M+1)

### (viii) Isopropyl 2-(3-{[1-(2-{2-[4-amino-2-(2-methoxyethyl)-1H-imidazo[4,5-c]quinolin-1-yl]ethoxy}ethyl)-3-(2-{piperidin-1-yl}ethyl)ureido]methyl}phenoxy)acetate

The title compound was prepared by the method of example 125 step (iii) using the product from step (vii) (0.211 g), to give the title compound (0.159 g). Yield 59%.

¹H NMR δ (DMSO-d₆) 8.03 (1H, d, J=8.1), 7.61 (1H, dd, J=0.9, 8.3), 7.43-7.39 (1H, m), 7.25-7.19 (1H, m), 7.25-7.19 (1H, m), 7.16 (1H, t, J=7.9), 6.74 (1H, dd, J=2.2, 8.3), 6.68-6.61 (2H, m), 6.48 (2H, brs), 6.12-6.08 (1H, m), 5.20-4.90 (1H, m), 4.77-4.70 (2H, m), 4.67 (2H, s), 4.25 (2H, s), 3.85-3.79 (4H, m), 3.41-3.37 (2H, m), 3.27 (3H, s), 3.24-3.16 (4H, m), 3.11-3.02 (2H, m), 2.28-2.20 (6H, m), 1.43-1.35 (4H, m), 1.35-1.25 (2H, m), 1.19 (6H, d, J=6.2).

MS: ESI 690 (M+1)

### Example 153

### Ethyl 2-[3-({N-[3-(4-amino-2-butyl-1H-imidazo[4,5-c]quinolin-1-yl)propyl]-2-(diethylamino)acetamido}methyl)phenoxy]-2-methylpropanoate

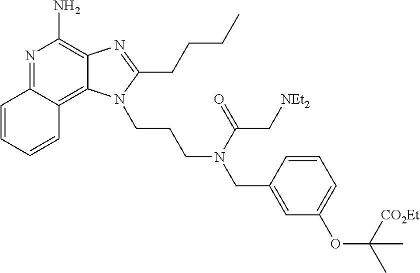

### (i) Ethyl 2-(3-{[3-(4-amino-2-butyl-1H-imidazo[4,5-c]quinolin-1-yl)propylamino]methyl}phenoxy)-2-methylpropanoate

By the method of example 1 step (viii) using the product from example 1 step (vii) (0.30 g) and ethyl 2-(3-formylphenoxy)-2-methylpropanoate (0.24 g) there was obtained the title compound, 0.43 g (83%) as a white solid

¹H NMR δ (CDCl₃) 8.07 (1H, d, J=8.2 Hz), 7.82 (1H, dd, J=8.3 Hz, 0.9 Hz), 7.58-7.46 (1H, m), 7.32-7.26 (1H, m), 7.21 (1H, dd, J=7.9 Hz, 7.8 Hz), 6.97 (1H, d, J=7.5 Hz), 6.91-6.87 (1H, m), 6.72 (1H, dd, J=8.1 Hz, 1.9 Hz), 5.55 (2H, brs), 4.61 (2H, t, J=7.5 Hz), 4.23 (2H, q, J=7.1 Hz), 3.77 (2H, s), 2.96 (2H, t, J=7.9 Hz), 2.75 (2H, t, J=6.2 Hz), 2.11-2.05 (2H, m), 1.90-1.82 (2H, m), 1.61 (6H, s), 1.55-1.45 (2H, m), 1.24 (3H, t, J=7.1 Hz), 1.01 (3H, t, J=7.4 Hz). MS:ESI 518 (M+1)

### (ii) Ethyl 2-[3-({N-[3-(4-amino-2-butyl-1H-imidazo[4,5-c]quinolin-1-yl)propyl]-2-chloroacetamido}methyl)phenoxy]-2-methylpropanoate hydrochloride

By the method of example 2 using the product of step (i) (0.43 g), there was obtained the title compound, 0.50 g (96%) as colorless gum.

¹H NMR δ (CDCl₃) 7.92 (1H, d, J=8.5 Hz), 7.60-7.55 (1H, m), 7.44 (1H, dd, J=7.3 Hz, 7.1 Hz), 7.21 (1H, dd, J=7.9 Hz, 7.8 Hz), 6.77-6.68 (4H, m), 4.61 (1.5H, s), 4.55 (0.5H, s), 4.52-4.47 (2H, m), 4.22 (2H, q, J=7.1 Hz), 4.11 (1.5H, s), 4.09 (0.5H, s), 3.61 (1.5H, t, J=6.6 Hz), 3.60-3.50 (0.5H, m), 2.88 (2H, t, J=7.8 Hz), 2.30-2.20 (0.5H, m), 2.15-2.07 (1.5H, m), 1.89-1.80 (2H, m), 1.59 (4.5H, s), 1.54 (1.5H, s), 1.53-1.47 (2H, m), 1.24 (3H, t, J=7.1 Hz), 1.01 (3H, t, J=7.4 Hz).

MS:ESI 595 (M+1)

### (iii) Ethyl 2-[3-({N-[3-(4-amino-2-butyl-1H-imidazo[4,5-c]quinolin-1-yl)propyl]-2-(diethylamino)acetamido}methyl)phenoxy]-2-methylpropanoate

The title compound was prepared by the method of example 5 using the product from step (ii) (0.26 g) to give a colorless gum (0.22 g). Yield 87%.

¹H NMR δ (CDCl₃) 7.89 (0.75H, d, J=7.7 Hz), 7.82 (1.25H, d, J=7.9 Hz), 7.54-7.49 (1H, m), 7.36-7.31 (1H, m), 7.20-7.13 (1H, m), 6.80-6.69 (3H, m), 5.51 (2H, brs), 4.75 (1.5H, s), 4.56 (0.5H, s), 4.46-4.42 (2H, m), 4.21 (2H, q, J=7.1 Hz), 3.55 (2H, t, J=6.8 Hz), 3.29 (1.5H, s), 3.24 (0.5H, s), 2.89-2.80 (2H, m), 2.60 (3H, q, J=7.1 Hz), 2.57-2.50 (1H, m), 2.30-2.22 (0.5H, m), 2.19-2.00 (1.5H, m), 1.95-1.80 (2H, m), 1.57 (6H, s), 1.56-1.45 (2H, m), 1.26-1.21 (3H, m), 1.02-0.98 (9H, m). MS:ESI 631 (M+1)

### Example 154

### Methyl 2-[3-({N-[3-(4-amino-2-butyl-1H-imidazo[4,5-c]quinolin-1-yl)propyl]-2-(diethylamino)acetamido}methyl)phenoxy]-2-methylpropanoate

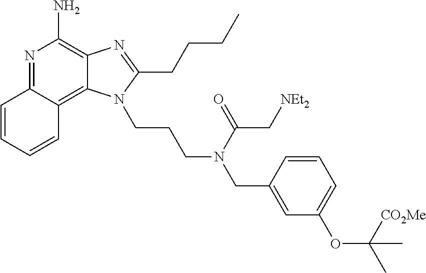

### (i) 2-[3-({N-[3-(4-Amino-2-butyl-1H-imidazo[4,5-c]quinolin-1-yl)propyl]-2-(diethylamino)acetamido}methyl)phenoxy]2-methylpropanoic acid

The title compound was prepared by the method of example 26 step (i) using the product from example 153 (0.20 g), to give a white solid (0.17 g). Yield 91%.

¹H NMR δ (DMSO-d₆) 7.92-7.87 (1H, m), 7.59 (1H, d, J=8.4 Hz), 7.42 (1H, dd, J=7.9 Hz, 7.4 Hz), 7.24-7.18 (2H, m), 7.18-7.00 (2H, m), 6.75-6.68 (3H, m), 4.68 (1H, s), 4.42-4.38 (2H, m), 4.35-4.30 (1H, m), 3.50 (1H, t, J=7.3 Hz), 3.42 (1H, t, J=7.1 Hz), 3.20 (2H, s), 2.85-2.79 (2H, m), 2.55-2.45 (1.5H, m), 2.41 (2.5H, q, J=7.1 Hz), 2.15-2.03 (1H, m), 1.95-1.80 (1H, m), 1.79-1.70 (2H, m), 1.45-1.38 (8H, m), 0.94 (3H, t, J=7.4 Hz), 0.89-0.82 (6H, m). MS:ESI 603 (M+1)

### (ii) Methyl 2-[3-({N-[3-(4-amino-2-butyl-1H-imidazo[4,5-c]quinolin-1-yl)propyl]-2-(diethylamino)acetamido}methyl)phenoxy]-2-methylpropanoate

The title compound was prepared by the method of example 26 step (ii) using the product from step (i) (0.13 g) and methanol, to give a colorless gum (0.11 g). Yield 80%.

¹H NMR δ (CDCl₃) 7.89 (0.75H, d, J=7.7 Hz), 7.83 (1.25H, d, J=7.9 Hz), 7.54-7.49 (1H, m), 7.37-7.31 (1H, m), 7.20-7.14 (1H, m), 6.76-6.65 (3H, m), 5.50 (2H, brs), 4.76 (1.5H, s), 4.55 (0.5H, s), 4.47-4.40 (2H, m), 3.75 (3H, s), 3.55 (2H, t, J=7.0 Hz), 3.29 (1.5H, s), 3.25 (0.5H, s), 2.89-2.80 (2H, m), 2.60 (3H, q, J=7.1 Hz), 2.57-2.50 (1H, m), 2.30-2.22 (0.5H, m), 2.19-2.00 (1.5H, m), 1.95-1.80 (2H, m), 1.57 (6H, s), 1.56-1.45 (2H, m), 1.02-0.96 (9H, m). MS:ESI 617 (M+1)

### Example 155

### Ethyl 2-[3-({N-[3-(4-amino-2-butyl-1H-imidazo[4,5-c]quinolin-1-yl)propyl]-2-(dimethylamino)acetamido}methyl)phenoxy]-2-methylpropanoate

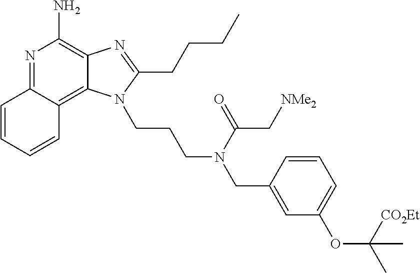

The title compound was prepared by the method of example 5 using the product from example 153 step (ii) (0.25 g) to give a colorless gum (0.20 g). Yield 85%.

¹H NMR δ (CDCl₃) 7.92-7.82 (2H, m), 7.52 (1H, dd, J=8.0 Hz, 7.3 Hz), 7.37-7.30 (1H, m), 7.20-7.14 (1H, m), 6.80-6.68 (3H, m), 5.51 (2H, brs), 4.70 (1.5H, s), 4.56 (0.5H, s), 4.48-4.42 (2H, m), 4.21 (2H, q, J=7.1 Hz), 3.56 (1.5H, t, J=6.7 Hz), 3.50-3.40 (0.5H, m), 3.16 (1.5H, s), 3.03 (0.5H, s), 2.90-2.84 (2H, m), 2.32 (4.5H, s), 2.28-2.20 (0.5H, m), 2.10-2.00 (3H, m), 1.95-1.80 (2H, m), 1.58 (6H, s), 1.56-1.45 (2H, m), 1.26-1.20 (3H, m), 1.00 (3H, t, J=7.3 Hz). MS:ESI 603 (M+1)

### Example 156

### Methyl 2-[3-({N-[3-(4-amino-2-butyl-1H-imidazo[4,5-c]quinolin-1-yl)propyl]-2-(dimethylamino)acetamido}methyl)phenoxy]-2-methylpropanoate

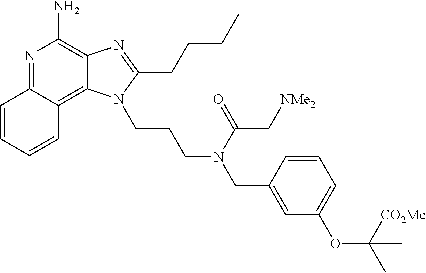

### (i) 2-[3-({N-[3-(4-Amino-2-butyl-1H-imidazo[4,5-c]quinolin-1-yl)propyl]-2-(dimethylamino)acetamido}methyl)phenoxy]-2-methylpropanoic acid

The title compound was prepared by the method of example 26 step (i) using the product from example 155 (0.18 g), to give a white solid (0.15 g). Yield 89%.

¹H NMR δ (DMSO-d₆) 7.90-7.82 (1H, m), 7.59 (1H, d, J=8.4 Hz), 7.43 (1H, dd, J=7.8 Hz, 7.4 Hz), 7.24-7.12 (4H, m), 6.76-6.68 (3H, m), 4.61 (1H, s), 4.43-4.38 (2H, m), 4.30-4.26 (1H, m), 3.46-3.36 (2H, m), 3.09 (1H, s), 3.05 (1H, s), 2.83-2.77 (2H, m), 2.20 (3H, s), 2.04 (3H, s), 2.04-2.00 (1H, m), 1.90-1.80 (1H, m), 1.75-1.68 (2H, m), 1.49 (3H, s), 1.47 (3H, s), 1.45-1.37 (2H, m), 0.94 (3H, t, J=7.3 Hz). MS:ESI 575 (M+1)

### (ii) Methyl 2-[3-({N-[3-(4-amino-2-butyl-1H-imidazo[4,5-c]quinolin-1-yl)propyl]-2-(dimethylamino)acetamido}methyl)phenoxy]-2-methylpropanoate

The title compound was prepared by the method of example 26 step (ii) using the product from step (i) (0.12 g) and methanol, to give a colorless gum (0.11 g). Yield 92%.

¹H NMR δ (CDCl₃) 7.92-7.82 (2H, m), 7.52 (1H, dd, J=8.0 Hz, 7.2 Hz), 7.37-7.30 (1H, m), 7.20-7.14 (1H, m), 6.77-6.66 (3H, m), 5.57 (2H, brs), 4.71 (1.5H, s), 4.56 (0.5H, s), 4.48-4.42 (2H, m), 3.75 (3H, s), 3.56 (1.5H, t, J=6.8 Hz), 3.50-3.40 (0.5H, m), 3.16 (1.5H, s), 3.04 (0.5H, s), 2.90-2.82 (2H, m), 2.32 (4.5H, s), 2.28-2.20 (0.5H, m), 2.10-2.00 (3H, m), 1.90-1.80 (2H, m), 1.58 (6H, s), 1.56-1.45 (2H, m), 1.00 (3H, t, J=7.3 Hz). MS:ESI 589 (M+1)

### Example 157

### Isopropyl 2-[3-({N-[3-(4-amino-2-butyl-1H-imidazo[4,5-c]quinolin-1-yl)propyl]-2-(diethylamino)acetamido}methyl)phenoxy]acetate

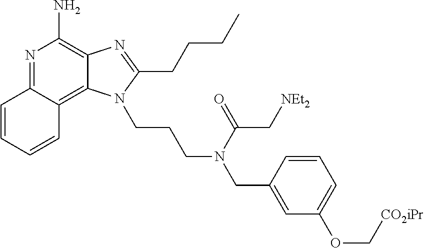

### (i) Isopropyl 2-(3-{[3-(4-amino-2-butyl-1H-imidazo[4,5-c]quinolin-1-yl)propylamino]methyl}phenoxy)acetate

By the method of example 1 step (viii) using the product from example 1 step (vii) (0.50 g) and isopropyl 2-(3-formylphenoxy)acetate (0.37 g) there was obtained the title compound, 0.70 g (83%) as a white solid

¹H NMR δ (CDCl₃) 8.06 (1H, d, J=7.7 Hz), 7.82 (1H, dd, J=8.3 Hz, 0.8 Hz), 7.52-7.46 (1H, m), 7.32-7.24 (2H, m), 6.97 (1H, d, J=7.6 Hz), 6.94 (1H, s), 6.80 (1H, dd, J=8.2 Hz, 2.2 Hz), 5.48 (2H, brs), 5.17-5.08 (1H, m), 4.60 (2H, t, J=7.5 Hz), 4.60 (2H, s), 3.80 (2H, s), 2.95 (2H, t, J=7.9 Hz), 2.75 (2H, t, J=6.3 Hz), 2.12-2.03 (2H, m), 1.90-1.82 (2H, m), 1.55-1.45 (2H, m), 1.26 (6H, d, J=6.3 Hz), 1.00 (3H, t, J=7.4 Hz). MS:ESI 504 (M+1)

### (ii) Isopropyl 2-[3-({N-[3-(4-amino-2-butyl-1H-imidazo[4,5-c]quinolin-1-yl)propyl]-2-chloroacetamido}methyl)phenoxy]acetate

By the method of example 2 using the product of step (i) (0.61 g), there was obtained the title compound, 0.75 g (quant.) as colorless gum.

¹H NMR δ (CDCl₃) 8.00 (1H, d, J=8.3 Hz), 7.94 (1H, d, J=8.1 Hz), 7.67-7.61 (1H, m), 7.54-7.49 (1H, m), 7.26-7.23 (1H, m), 6.80-6.77 (3H, m), 5.18-5.10 (1H, m), 4.64 (1.5H, s), 4.61 (2H, s), 4.58 (0.5H, s), 4.53-4.47 (2H, m), 4.14 (1.5H, s), 4.12 (0.5H, s), 3.61 (2H, t, J=6.7 Hz), 2.88 (2H, t, J=7.7 Hz), 2.30-2.20 (0.5H, m), 2.15-2.07 (2H, m), 1.93-1.83 (2.5H, m), 1.57-1.46 (2H, m), 1.29 (6H, d, J=6.3 Hz), 1.03 (3H, t, J=7.4 Hz). MS:ESI 581 (M+1)

### (iii) Isopropyl 2-[3-({N-[3-(4-amino-2-butyl-1H-imidazo[4,5-c]quinolin-1-yl)propyl]-2-(diethylamino)acetamido}methyl)phenoxy]acetate

The title compound was prepared by the method of example 5 using the product from step (ii) (0.25 g) to give a colorless gum (0.18 g). Yield 71%.

¹H NMR δ (CDCl₃) 7.87 (0.75H, d, J=7.6 Hz), 7.83 (1.25H, d, J=8.4 Hz), 7.54-7.49 (1H, m), 7.36-7.30 (1H, m), 7.21-7.15 (1H, m), 6.79-6.70 (3H, m), 5.55 (2H, brs), 5.17-5.08 (1H, m), 4.75 (1.5H, s), 4.56 (0.5H, s), 4.54 (2H, s), 4.46-4.40 (2H, m), 3.52 (2H, t, J=7.0 Hz), 3.31 (1.5H, s), 3.25 (0.5H, s), 2.88-2.80 (2H, m), 2.60 (3H, q, J=7.1 Hz), 2.52 (1H, q, J=7.2 Hz), 2.30-2.22 (0.5H, m), 2.13-2.00 (1.5H, m), 1.90-1.80 (2H, m), 1.53-1.44 (2H, m), 1.27 (6H, d, J=6.3 Hz), 1.02-0.96 (9H, m). MS:ESI 617 (M+1)

### Example 158

### Isopropyl 2-[3-({N-[3-(4-amino-2-butyl-1H-imidazo[4,5-c]quinolin-1-yl)propyl]-2-[ethyl(methyl)amino]acetamido}methyl)phenoxy]acetate

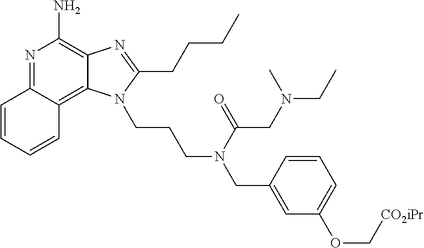

The title compound was prepared by the method of example 5 using the product from example 157 step (ii) (0.29 g) to give a colorless gum (0.21 g). Yield 74%.

¹H NMR δ (CDCl₃) 7.88 (0.75H, d, J=7.9 Hz), 7.85 (1.25H, d, J=8.4 Hz), 7.56-7.50 (1H, m), 7.37-7.30 (1H, m), 7.22-7.16 (1H, m), 6.80-6.70 (3H, m), 5.69 (2H, brs), 5.17-5.08 (1H, m), 4.73 (1.5H, s), 4.57 (0.5H, s), 4.54 (2H, s), 4.47-4.40 (2H, m), 3.54 (2H, t, J=6.9 Hz), 3.22 (1.5H, s), 3.12 (0.5H, s), 2.89-2.80 (2H, m), 2.50 (1.5H, q, J=7.1 Hz), 2.42-2.32 (0.5H, m), 2.31 (2.25H, s), 2.30-2.22 (0.5H, m), 2.15 (0.75H, s), 2.13-2.00 (1.5H, m), 1.90-1.80 (2H, m), 1.53-1.44 (2H, m), 1.27 (6H, d, J=6.3 Hz), 1.05 (3H, t, J=7.1 Hz), 1.00 (3H, t, J=7.3 Hz). MS:ESI 603 (M+1)

### Example 159

### Isopropyl 2-[3-({N-[3-(4-amino-2-butyl-1H-imidazo[4,5-c]quinolin-1-yl)propyl]-2-(dimethylamino)acetamido}methyl)phenoxy]acetate

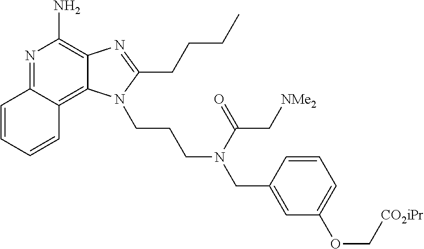

### (i) 2-[3-({N-[3-(4-Amino-2-butyl-1H-imidazo[4,5-c]quinolin-1-yl)propyl]-2-[ethyl(methyl)amino]acetamido}methyl)phenoxy]acetic acid

The title compound was prepared by the method of example 26 step (i) using the product from example 158 (0.12 g), to give a white solid (0.11 g). Yield 98%.

¹H NMR δ (DMSO-d₆) 7.96-7.88 (1H, m), 7.57 (1H, d, J=8.1 Hz), 7.43 (1H, dd, J=7.5 Hz, 6.6 Hz), 7.40-7.15 (4H, m), 6.78-6.70 (3H, m), 4.67 (1H, s), 4.57-4.40 (4H, m), 4.38-4.30 (1H, m), 3.50-3.45 (1H, m), 3.43-3.37 (1H, m), 3.19 (1H, s), 3.11 (1H, s), 2.83 (2H, t, J=7.4 Hz), 2.50-2.40 (1H, m), 2.32-2.25 (1H, m), 2.19 (1.5H, s), 2.10-2.00 (2.5H, m), 1.95-1.90 (1H, m), 1.79-1.70 (2H, m), 1.45-1.38 (2H, m), 0.96-0.83 (6H, m). MS:ESI 561 (M+1)

### (ii) Isopropyl 2-[3-({N-[3-(4-amino-2-butyl-1H-imidazo[4,5-c]quinolin-1-yl)propyl]-2-(dimethylamino)acetamido}methyl)phenoxy]acetate

The title compound was prepared by the method of example 26 step (ii) using the product from step (i) (0.26 g) to give a colorless gum (0.18 g). Yield 72%.

¹H NMR δ (CDCl₃) 7.87 (0.75H, d, J=8.9 Hz), 7.84 (1.25H, d, J=8.3 Hz), 7.55-7.49 (1H, m), 7.37-7.33 (1H, m), 7.22-7.15 (1H, m), 6.80-6.70 (3H, m), 5.61 (2H, brs), 5.17-5.08 (1H, m), 4.70 (1.5H, s), 4.57 (0.5H, s), 4.54 (2H, s), 4.47-4.40 (2H, m), 3.54 (1.5H, t, J=6.8 Hz), 3.50-3.40 (0.5H, m), 3.17 (1.5H, s), 3.03 (0.5H, s), 2.89-2.80 (2H, m), 2.32 (4.5H, s), 2.30-2.04 (3.5H, m), 1.90-1.80 (2H, m), 1.53-1.44 (2H, m), 1.27 (6H, d, J=6.3 Hz), 1.00 (3H, t, J=7.3 Hz). MS:ESI 589 (M+1)

### Example 160

### Isopropyl 2-[3-({N-[3-(4-amino-2-propyl-1H-imidazo[4,5-c]quinolin-1-yl)propyl]-2-(diethylamino)acetamido}methyl)phenoxy]acetate

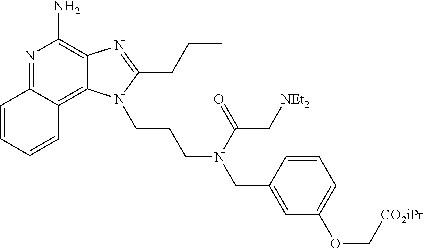

### (i) tert-Butyl 3-(2-propyl-1H-imidazo[4,5-c]quinolin-1-yl)propylcarbamate

The title compound was prepared by the method of example 1 step (iv) using the product from example 1 step (iii) (5.0 g), to give the title compound (5.0 g). Yield 85%.

¹H NMR δ (DMSO-d₆) 9.13 (1H, s), 8.37-8.34 (1H, m), 8.15-8.12 (1H, m), 7.70-7.67 (2H, m), 7.14 (1H, t, J=5.2 Hz), 4.61-4.56 (2H, m), 3.12-3.08 (2H, m), 2.95-2.91 (2H, m), 1.96-1.84 (4H, m), 1.35 (9H, s), 1.03 (3H, t, J=7.6 Hz).

MS: ESI 369 (M+1)

### (ii) tert-Butyl 3-(4-amino-2-propyl-1H-imidazo[4,5-c]quinolin-1-yl)propylcarbamate

The title compound was prepared by the method of example 1 step (v-vi) using the product from previous step (5.0 g), to give the title compound (3.8 g). Yield 73%.

¹H NMR δ (DMSO-d₆) 8.03 (1H, d, J=8.0 Hz), 7.59 (1H, dd, J=1.2, 8.0 Hz), 7.42-7.38 (1H, m), 7.23-7.19 (1H, m), 7.13 (1H, t, J=5.2 Hz), 6.45 (2H, s), 4.50-4.46 (2H, m), 3.10-3.06 (2H, m), 2.89-2.84 (2H, m), 1.93-1.81 (4H, m), 1.39 (9H, s), 1.05-0.99 (3H, m).

MS: ESI 384 (M+1)

### (iii) 1-(3-Aminopropyl)-2-propyl-1H-imidazo[4,5-c]quinolin-4-amine

The title compound was prepared by the method of example 1 step (vii) using the product from step (ii) (3.52 g) to give a pale yellow solid (2.40 g). Yield 92%.

¹H NMR δ (DMSO-d₆) 8.13 (1H, d, J=7.6 Hz), 7.60 (1H, dd, J=8.3 Hz, 1.0 Hz), 7.43-7.38 (1H, m), 7.25-7.21 (1H, m), 6.44 (2H, brs), 4.57 (2H, t, J=7.6 Hz), 2.92 (2H, t, J=7.6 Hz), 2.67 (2H, t, J=6.4 Hz), 1.90-1.78 (4H, m), 1.60 (2H, brs), 1.03 (3H, t, J=7.4 Hz). MS: ESI 284 (M+1)

### (iv) Isopropyl 2-(3-{[3-(4-amino-2-propyl-1H-imidazo[4,5-c]quinolin-1-yl)propylamino]methyl}phenoxy)acetate

By the method of example 1 step (viii) using the product from example 1 step (vii) (0.25 g) and isopropyl 2-(3-formylphenoxy)acetate (0.20 g) there was obtained the title compound, 0.32 g (75%) as a white solid

¹H NMR δ (CDCl₃) 8.10 (1H, d, J=8.2 Hz), 7.84 (1H, dd, J=8.4 Hz, 1.0 Hz), 7.55-7.46 (1H, m), 7.34-7.24 (2H, m), 7.00-6.93 (2H, m), 6.85-6.80 (1H, m), 5.78 (2H, brs), 5.17-5.08 (1H, m), 4.66-4.56 (4H, m), 3.80 (2H, s), 2.96-2.90 (2H, m), 2.76 (2H, t, J=6.2 Hz), 2.12-2.03 (2H, m), 1.96-1.86 (2H, m), 1.26 (6H, d, J=6.3 Hz), 1.08 (3H, t, J=7.4 Hz). MS:ESI 490 (M+1)

### (v) Isopropyl 2-[3-({N-[3-(4-amino-2-propyl-1H-imidazo[4,5-c]quinolin-1-yl)propyl]-2-chloroacetamido}methyl)phenoxy]acetate

By the method of example 2 using the product of step (iv) (0.32 g), there was obtained the title compound, 0.38 g (96%) as colorless gum.

¹H NMR δ (CDCl₃) 7.96 (1H, d, J=8.1 Hz), 7.92 (1H, d, J=7.7 Hz), 7.64-7.58 (1H, m), 7.53-7.46 (1H, m), 7.26-7.17 (1H, m), 6.78-6.75 (3H, m), 5.18-5.07 (1H, m), 4.62 (2H, s), 4.56 (2H, s), 4.50-4.42 (2H, m), 4.10 (2H, s), 3.59 (2H, t, J=6.7 Hz), 2.84 (2H, t, J=7.6 Hz), 2.15-2.05 (2H, m), 1.97-1.86 (2H, m), 1.28 (6H, d, J=6.3 Hz), 1.08 (3H, t, J=7.4 Hz). MS:ESI 567 (M+1)

### (vi) Isopropyl 2-[3-({N-[3-(4-amino-2-propyl-1H-imidazo[4,5-c]quinolin-1-yl)propyl]-2-(diethylamino)acetamido}methyl)phenoxy]acetate

The title compound was prepared by the method of example 5 using the product from step (v) (0.37 g) to give a colorless gum (0.32 g). Yield 85%.

¹H NMR δ (CDCl₃) 7.87 (0.75H, d, J=8.1 Hz), 7.83 (1.25H, d, J=8.4 Hz), 7.54-7.49 (1H, m), 7.36-7.30 (1H, m), 7.22-7.16 (1H, m), 6.80-6.70 (3H, m), 5.57 (2H, brs), 5.17-5.08 (1H, m), 4.75 (1.5H, s), 4.56 (0.5H, s), 4.54 (2H, s), 4.46-4.40 (2H, m), 3.53 (2H, t, J=6.9 Hz), 3.31 (1.5H, s), 3.25 (0.5H, s), 2.87-2.77 (2H, m), 2.60 (3H, q, J=7.1 Hz), 2.52 (1H, q, J=7.1 Hz), 2.10-2.03 (2H, m), 1.93-1.83 (2H, m), 1.27 (6H, d, J=6.3 Hz), 1.08 (3H, t, J=7.4 Hz), 1.01-0.97 (6H, m). MS:ESI 603 (M+1)

### Example 161

### Isopropyl 2-{3-[(N-{3-[4-amino-2-(ethoxymethyl)-1H-imidazo[4,5-c]quinolin-1-yl]propyl}-2-{diethylamino}acetamido)methyl]phenoxy}acetate

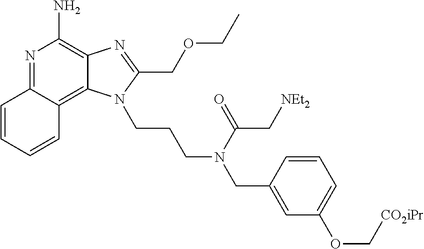

### (i) tert-Butyl 3-[2-(ethoxymethyl)-1H-imidazo[4,5-c]quinolin-1-yl]propylcarbamate

To the product of example 1 step (iii) (5.0 g) in NMP (25 mL), 2-ethoxyaceic acid (1.9 mL, 20 mmol) was added followed by WSC HCl (3.8 g) and HOBt (2.7 mL) under nitrogen. The resulting solution was stirred at 120° C. for 4 h. The reaction mixture was diluted with EtOAc (100 mL), and washed with sat. NaHCO₃ (100 mL×2), and saturated brine (100 mL). The organic layer was dried, filtered and evaporated. The organic residue was purified by silica gel chromatography to afford the subtitle product (4.2 g). Yield 70%.

¹H NMR δ (DMSO-d₆) 9.17 (1H, s), 8.38-8.30 (1H, m), 8.18-8.15 (1H, m), 7.70-7.67 (2H, m), 7.13 (1H, t, J=5.6 Hz), 4.81 (2H, s), 4.68-4.63 (2H, m), 3.56 (2H, q, J=7.2 Hz), 3.16-3.11 (2H, m), 2.03-1.99 (2H, m), 1.39 (9H, s), 1.16 (3H, t, J=7.2 Hz).

MS: ESI 385 (M+1)

### (ii) tert-Butyl 3-[4-amino-2-(ethoxymethyl)-1H-imidazo[4,5-c]quinolin-1-yl]propylcarbamate

The product from step (i) (4.2 g) was dissolved in DCM (100 mL) and cooled to 5° C. 3-Chloroperoxybenzoic acid (3.4 g) was added and the reaction was allowed to warm to room temperature. The reaction mixture was stirred for 12 h. The reaction mixture was washed with saturated sodium thiosulfate solution and sodium bicarbonate solution, dried, filtered and evaporated to give a product.

p-Toluenesulphonyl chloride (2.5 g) was added portionwise to a vigorously stirred mixture of the product in DCM (40 mL) and ammonium hydroxide solution (35%, 12 mL) at 0° C. The mixture was allowed to warm to rt over night then partitioned between water/DCM, washed with saturated sodium bicarbonate solution, dried, filtered and the solvent evaporated. The organic residue was purified by silica gel chromatography to give the subtitle compound (3.6 g). Yield 83%.

¹H NMR 8 (DMSO-d₆) 8.04 (1H, d, J=7.6 Hz), 7.60 (1H, dd, J=1.2, 8.0 Hz), 7.47-7.42 (1H, m), 7.26-7.22 (1H, m), 7.11 (1H, t, J=5.4 Hz), 6.60 (2H, s), 4.75 (2H, s), 4.57-4.53 (2H, m), 3.54 (2H, q, J=6.8 Hz), 3.16-3.08 (2H, m), 2.00-1.95 (2H, m), 1.39 (9H, s), 1.18-1.14 (3H, m).

MS: ESI 400 (M+1)

### (iii) 1-(3-Aminopropyl)-2-(ethoxymethyl)-1H-imidazo[4,5-c]quinolin-4-amine

The product from step (ii) (3.49 g) was suspended in MeOH (14 mL) and 6N HCl (14 mL) was added. The reaction mixture was stirred at 50° C. for 1 h. After the removal of the solvent, water was added to the residue, washed with chloroform twice and then poured into 28% NH₃ solution, extracted with EtOH/CHCl₃ (1/3), dried and evaporated to give the subtitle compound as a pale yellow solid (2.11 g). Yield 81%.

¹H NMR δ (DMSO-d₆) 8.16 (1H, d, J=7.6 Hz), 7.60 (1H, dd, J=8.3 Hz, 1.0 Hz), 7.46-7.42 (1H, m), 7.29-7.24 (1H, m), 6.60 (2H, brs), 4.75 (2H, s), 4.63 (2H, t, J=7.7 Hz), 3.54 (2H, q, J=7.0 Hz), 2.69 (2H, t, J=6.4 Hz), 1.96-1.87 (2H, m), 1.64 (2H, brs), 1.16 (3H, t, J=7.0 Hz). MS: ESI 300 (M+1)

### (iv) Isopropyl 2-[3-({3-[4-amino-2-(ethoxymethyl)-1H-imidazo[4,5-c]quinolin-1-yl]propylamino}methyl)phenoxy]acetate

To a solution of the product from step (iii) (0.25 g, 0.84 mmol) in MeOH (5 ml) were added isopropyl 2-(3-formylphenoxy)acetate (0.19 g, 0.84 mmol), AcOH (0.096 ml, 1.67 mmol) and NaBH₃CN (0.11 g, 1.67 mmol) at room temperature. After stirring for 4 h at the same temperature, 4% NH₃ aq. was added to the reaction mixture, and extracted with CHCl₃ (30 ml×2). The combined extracts were dried over MgSO₄ and concentrated. The residue was purified by flash column chromatography to afford the subtitle compound 0.34 g 80%) as a white solid

¹H NMR δ (CDCl₃) 8.12 (1H, d, J=7.4 Hz), 7.85 (1H, dd, J=8.3 Hz, 0.8 Hz), 7.55-7.52 (1H, m), 7.34-7.26 (2H, m), 7.00-6.94 (2H, m), 6.83-6.78 (1H, m), 5.76 (2H, brs), 5.17-5.08 (1H, m), 4.84 (2H, s), 4.74 (2H, t, J=7.7 Hz), 4.61 (2H, s), 3.81 (2H, s), 3.61 (2H, q, J=7.0 Hz), 2.79 (2H, t, J=6.3 Hz), 2.18-2.10 (2H, m), 1.30-1.20 (9H, m). MS:ESI 506 (M+1)

### (v) Isopropyl 2-{3-[(N-{3-[4-amino-2-(ethoxymethyl)-1H-imidazo[4,5-c]quinolin-1-yl]propyl}-2-chloroacetamido)methyl]phenoxy}acetate

To a solution of the product of step (iv) (0.33 g, 0.66 mmol) in CHCl₃ (5 ml) was added chloroacetyl chloride (0.052 ml, 0.66 mmol) at 0° C. After stirring for 1.5 h at the same temperature, the reaction mixture was concentrated. The residue was purified by flash column chromatography to give the title compound, 0.41 g (quant.) as colorless gum.

¹H NMR δ (CDCl₃) 7.95 (1H, d, J=8.1 Hz), 7.90 (1H, d, J=8.1 Hz), 7.63-7.57 (1H, m), 7.48-7.42 (1H, m), 7.26-7.20 (1H, m), 6.79-6.72 (3H, m), 6.65 (2H, brs), 5.17-5.07 (1H, m), 4.77 (2H, s), 4.63 (2H, s), 4.62-4.53 (4H, m), 4.10 (2H, s), 3.66-3.58 (4H, m), 2.26-2.16 (2H, m), 1.29-1.18 (9H, m). MS:ESI 583 (M+1)

### (vi) Isopropyl 2-{3-[(N-{3-[4-amino-2-(ethoxymethyl)-1H-imidazo[4,5-c]quinolin-1-yl]propyl}-2-{diethylamino}acetamido)methyl]phenoxy}acetate

The product from step (v) (0.40 g, 0.65 mmol) was dissolved in MeCN (5 ml) and Et₂NH (0.68 ml, 6.5 mmol) was added. After stirring for 17 h, 4% NH₃ aq. was added to the reaction mixture, and extracted with CHCl₃ (30 ml×2). The combined extracts were dried over MgSO₄ and concentrated. The residue was purified by silica gel chromatography which afforded 0.34 g mg of the desired product as a colorless gum. The colorless gum was triturated with ether to give the desired compound as a white solid. Yield 84%.

¹H NMR δ (CDCl₃) 7.92 (0.75H, d, J=7.8 Hz), 7.89-7.82 (1.25H, m), 7.57-7.52 (1H, m), 7.38-7.33 (1H, m), 7.23-7.17 (1H, m), 6.80-6.72 (3H, m), 5.55 (2H, brs), 5.17-5.08 (1H, m), 4.78-4.74 (3.5H, m), 4.59-4.52 (4.5H, m), 3.64-3.52 (4H, m), 3.30 (1.5H, s), 3.26 (0.5H, s), 2.60 (3H, q, J=7.1 Hz), 2.52 (1H, q, J=7.1 Hz), 2.35-2.20 (1H, m), 2.20-2.12 (1H, m), 1.27 (6H, d, J=6.3 Hz), 1.26-1.21 (3H, m), 0.99 (6H, t, J=7.1 Hz). MS:ESI 619 (M+1)

### Example 162

### Ethyl 2-{3-[(N-{3-[4-amino-2-(ethoxymethyl)-1H-imidazo[4,5-c]quinolin-1-yl]propyl}-2-{diethylamino}acetamido)methyl]phenoxy}-2-methylpropanoate

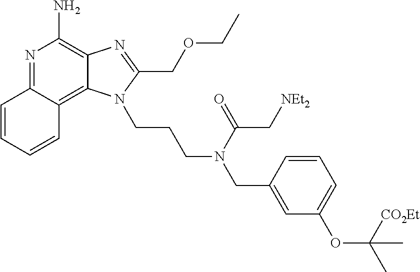

### (i) Ethyl 2-[3-({3-[4-amino-2-(ethoxymethyl)-1H-imidazo[4,5-c]quinolin-1-yl]propylamino}methyl)phenoxy]-2-methylpropanoate

To a solution of the product from example 161 step (iii) (0.25 g, 0.83 mmol) in MeOH (5 ml) were added ethyl 2-(3-formylphenoxy)-2-methylpropanoate (0.20 g, 0.83 mmol), AcOH (0.095 ml, 1.67 mmol) and NaBH₃CN (0.11 g, 1.67 mmol) at room temperature. After stirring for 26 h at the same temperature, 4% NH₃ aq. was added to the reaction mixture, and extracted with CHCl₃ (30 ml×2). The combined extracts were dried over MgSO₄ and concentrated. The residue was purified by flash column chromatography to afford the subtitle compound, 0.36 g (82%) as a white solid.

¹H NMR δ (CDCl₃) 8.12 (1H, d, J=7.4 Hz), 7.83 (1H, dd, J=8.3 Hz, 0.8 Hz), 7.56-7.50 (1H, m), 7.33-7.26 (1H, m), 7.21 (1H, dd, J=7.9 Hz, 7.8 Hz), 6.97 (1H, d, J=7.6 Hz), 6.91-6.88 (1H, m), 6.72 (1H, dd, J=8.0 Hz, 2.1 Hz), 5.62 (2H, brs), 4.84 (2H, s), 4.73 (2H, t, J=7.7 Hz), 4.22 (2H, q, J=7.1 Hz), 3.78 (2H, s), 3.60 (2H, q, J=7.0 Hz), 2.78 (2H, t, J=6.3 Hz), 2.18-2.09 (2H, m), 1.61 (6H, s), 1.24 (6H, t, J=7.1 Hz). MS:ESI 520 (M+1)

### (ii) Ethyl 2-{3-[(N-{3-[4-amino-2-(ethoxymethyl)-1H-imidazo[4,5-c]quinolin-1-yl]propyl}-2-chloroacetamido)methyl]phenoxy}-2-methylpropanoate

To a solution of the product of step (i) (0.35 g, 0.68 mmol) in CHCl₃ (5 ml) was added chloroacetyl chloride (0.054 ml, 0.68 mmol) at 0° C. After stirring for 20 min at the same temperature, the reaction mixture was concentrated. The residue was purified by flash column chromatography to give the title compound, 0.43 g (quant.) as colorless gum.

¹H NMR δ (CDCl₃) 7.94 (1H, d, J=8.2 Hz), 7.88-7.79 (1H, m), 7.57 (1H, dd, J=7.8 Hz, 7.5 Hz), 7.44-7.37 (1H, m), 7.27-7.10 (1H, m), 6.78-6.68 (3H, m), 6.42 (2H, brs), 4.77 (2H, s), 4.62 (2H, s), 4.62-4.51 (2H, m), 4.21 (2H, q, J=7.1 Hz), 4.15 (2H, s), 3.61 (4H, t, J=7.0 Hz), 2.32-2.16 (2H, m), 1.58 (4.5H, s), 1.56 (1.5H, s), 1.30-1.18 (6H, m). MS:ESI 597 (M+1)

### (iii) Ethyl 2-{3-[(N-{3-[4-amino-2-(ethoxymethyl)-1H-imidazo[4,5-c]quinolin-1-yl]propyl}-2-{diethylamino}acetamido)methyl]phenoxy}-2-methylpropanoate

The product from step (ii) (0.43 g, 0.68 mmol) was dissolved in MeCN (5 ml) and Et₂NH (0.71 ml, 6.8 mmol) was added. After stirring for 20 h, 4% NH₃ aq. was added to the reaction mixture, and extracted with CHCl₃ (30 ml×2). The combined extracts were dried over MgSO₄ and concentrated. The residue was purified by silica gel chromatography which afforded 0.36 g of the desired product as a colorless gum. Yield 83%.

¹H NMR δ (CDCl₃) 7.95 (0.75H, d, J=7.6 Hz), 7.85-7.82 (1.25H, m), 7.58-7.52 (1H, m), 7.40-7.31 (1H, m), 7.21-7.13 (1H, m), 6.78-6.67 (3H, m), 5.57 (2H, brs), 4.77 (1.5H, s), 4.77-4.75 (2H, m), 4.59-4.56 (2H, m) 4.55 (0.5H, s), 4.21 (2H, q, J=7.1 Hz), 3.64-3.55 (4H, m), 3.29 (1.5H, s), 3.26 (0.5H, s), 2.60 (3H, q, J=7.1 Hz), 2.52 (1H, q, J=7.1 Hz), 2.30-2.22 (0.5H, m), 2.19-2.10 (1.5H, m), 1.57 (6H, s), 1.28-1.19 (6H, m), 0.99 (6H, t, J=7.1 Hz). MS:ESI 633 (M+1)

### Example 163

### Methyl 2-{3-[(N-{3-[4-amino-2-(ethoxymethyl)-1H-imidazo[4,5-c]quinolin-1-yl]propyl}-2-{diethylamino}acetamido)methyl]phenoxy}-2-methylpropanoate

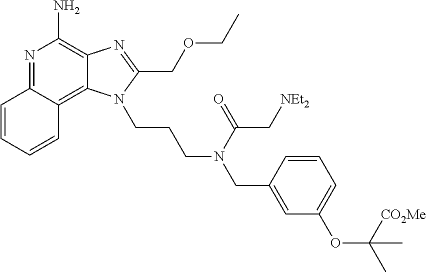

### (i) 2-{3-[(N-{3-[4-Amino-2-(ethoxymethyl)-1H-imidazo[4,5-c]quinolin-1-yl]propyl}-2-{diethylamino}acetamido)methyl]phenoxy}-2-methylpropanoic acid

To a solution of example 162 (0.27 g, 0.42 mmol) in EtOH (5 ml), 1N NaOH (5 mL) was added at rt. After stirring for 30 min at 50° C., the reaction mixture was neutralized with 1N HCl at 0° C. The aq. layer was extracted with CHCl₃/EtOH (3/1, twice), dried over MgSO₄, and concentrated to give the title compound (0.25 g, 96%) as a white solid.

¹H NMR δ (DMSO-d₆) 8.01-7.93 (1H, m), 7.61 (1H, d, J=8.4 Hz), 7.46 (1H, dd, J=7.3 Hz, 7.2 Hz), 7.30-7.17 (2H, m), 6.87 (2H, brs), 6.74-6.67 (3H, m), 4.75-4.73 (2H, m), 4.70 (1H, s), 4.60-4.55 (1H, m), 4.50-4.45 (1H, m), 4.44 (2H, s), 3.55-3.48 (4H, m), 3.45 (1H, t, J=6.8 Hz), 3.20 (1H, s), 3.19 (1H, s), 2.42 (2H, q, J=7.2 Hz), 2.20-2.10 (1H, m), 2.08-1.99 (1H, m), 1.42 (6H, s), 1.15-1.09 (3H, m), 0.89-0.82

(6H, m). MS:ESI 605 (M+1)

### (ii) Methyl 2-{3-[(N-{3-[4-amino-2-(ethoxymethyl)-1H-imidazo[4,5-c]quinolin-1-yl)propyl)-2-{diethylamino}acetamido)methyl]phenoxy}-2-methylpropanoate

To a solution of the product from step (i) (0.18 g, 0.29 mmol) in methanol (5 mL), 4N HCl/dioxane solution (1 mL) was added. The reaction mixture was stirred at room temperature for 6 h, and then 4% NH₃ aq. was added to the reaction mixture, and extracted with CHCl₃ (30 ml×3). The combined extracts were dried over MgSO₄ and concentrated to afford the title compound (0.17 g, 80%) as a white solid.

¹H NMR δ (CDCl₃) 7.95 (0.75H, d, J=7.7 Hz), 7.90-7.82 (1.25H, m), 7.58-7.53 (1H, m), 7.40-7.32 (1H, m), 7.22-7.14 (1H, m), 6.80-6.66 (3H, m), 5.66 (2H, brs), 4.77 (1.5H, s), 4.76-4.74 (2H, m), 4.60-4.54 (2.5H, m), 3.76 (3H, s), 3.65-3.55 (4H, m), 3.29 (1.5H, s), 3.27 (0.5H, s), 2.60 (3H, q, J=7.1 Hz), 2.51 (1H, q, J=7.1 Hz), 2.32-2.22 (0.5H, m), 2.19-2.00 (1.5H, m), 1.58 (6H, s), 1.22 (3H, t, J=7.0 Hz), 0.99 (6H, t, J=7.1 Hz). MS:ESI 619 (M+1)

### Example 164

### Isopropyl

### 2-[3-({N-[3-(4-amino-2-butyl-1H-imidazo[4,5-c]quinolin-1-yl)propyl]-2-(diethylamino)acetamido}methyl)-2-fluorophenoxy]acetate

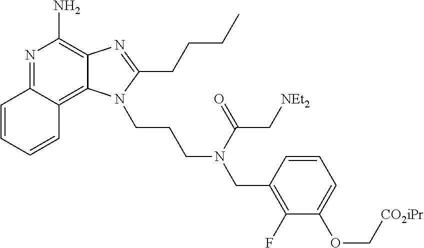

### (i) Isopropyl 2-(3-{[3-(4-amino-2-butyl-1H-imidazo[4,5-c]quinolin-1-yl)propylamino]methyl}-2-fluorophenoxy)acetate

By the method of example 1 step (viii) using the product from example lstep (vii) (0.30 g) and isopropyl 2-(2-fluoro-3-formylphenoxy)acetate (0.24 g) there was obtained the title compound, 0.53 g (quant.) as a white solid.

¹H NMR δ (CDCl₃) 8.06 (1H, d, J=8.3 Hz), 7.83 (1H, dd, J=8.4 Hz, 0.9 Hz), 7.53-7.47 (1H, m), 7.33-7.24 (2H, m), 7.03-7.00 (1H, m), 7.00-6.95 (1H, m), 6.85-6.80 (1H, m), 5.59 (2H, brs), 5.18-5.10 (1H, m), 4.68 (2H, s), 4.60 (2H, t, J=7.4 Hz), 3.88 (2H, s), 2.95 (2H, t, J=7.9 Hz), 2.73 (2H, t, J=6.3 Hz), 2.12-2.03 (2H, m), 1.90-1.82 (2H, m), 1.55-1.45 (2H, m), 1.27 (6H, d, J=6.3 Hz), 1.00 (3H, t, J=7.4 Hz). MS:ESI 522 (M+1)

### (ii) Isopropyl 2-[3-({N-[3-(4-amino-2-butyl-1H-imidazo[4,5-c]quinolin-1-yl)propyl]-2-chloroacetamido}methyl)-2-fluorophenoxy]acetate

By the method of example 2 using the product of step (i) (0.53 g), there was obtained the title compound, 0.60 g (94%) as colorless gum.

¹H NMR δ (CDCl₃) 8.00 (1H, d, J=8.1 Hz), 7.93 (1H, d, J=8.0 Hz), 7.65-7.62 (1H, m), 7.58-7.52 (1H, m), 7.00-6.95 (1H, m), 6.83-6.77 (1H, m), 6.73-6.55 (3H, m), 5.15-5.08 (1H, m), 4.68 (1.5H, s), 4.66 (0.5H, s), 4.64 (2H, s), 4.51-4.47 (2H, m), 4.22 (1.5H, s), 4.12 (0.5H, s), 3.56 (2H, t, J=6.8 Hz), 2.86 (2H, t, J=7.7 Hz), 2.40-2.30 (0.5H, m), 2.15-2.07 (1.5H, m), 1.93-1.83 (2H, m), 1.57-1.46 (2H, m), 1.27 (6H, d, J=6.3 Hz), 1.01 (3H, t, J=7.3 Hz). MS:ESI 599 (M+1)

### (iii) Isopropyl 2-[3-({N-[3-(4-amino-2-butyl-1H-imidazo[4,5-c]quinolin-1-yl)propyl]-2-(diethylamino)acetamido}methyl)-2-fluorophenoxy]acetate

The title compound was prepared by the method of example 5 using the product from step (ii) (0.30 g) to give a colorless gum (0.25 g). Yield 85%.

¹H NMR δ (CDCl₃) 7.95-7.85 (1H, m), 7.82 (1H, d, J=8.3 Hz), 7.54-7.49 (1H, m), 7.36-7.30 (1H, m), 7.05-6.80 (1.5H, m), 6.73-6.64 (1.5H, m), 5.44 (2H, brs), 5.17-5.08 (1H, m), 4.84 (1.5H, s), 4.69 (0.5H, s), 4.65 (0.5H, s), 4.59 (1.5H, s), 4.46-4.40 (2H, m), 3.62-3.57 (0.5H, m), 3.48 (1.5H, t, J=7.1 Hz), 3.35 (1.5H, s), 3.19 (0.5H, s), 2.89-2.83 (2H, m), 2.60 (3H, q, J=7.1 Hz), 2.46 (1H, q, J=7.2 Hz), 2.35-2.28 (0.5H, m), 2.13-2.00 (1.5H, m), 1.90-1.80 (2H, m), 1.53-1.44 (2H, m), 1.27 (6H, d, J=6.2 Hz), 1.02-0.90 (9H, m). MS:ESI 635 (M+1)

### Example 165

### Isopropyl 2-[3-({N-[3-(4-amino-2-butyl-1H-imidazo[4,5-c]quinolin-1-yl)propyl]-2-(dimethylamino)acetamido}methyl)-2-fluorophenoxy]acetate

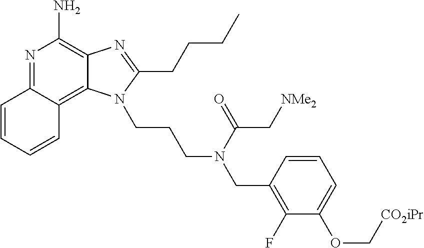

The title compound was prepared by the method of example 5 using the product from example 164 step (ii) (0.30 g) to give a colorless gum (0.25 g). Yield 86%.

¹H NMR δ (CDCl₃) 7.91-7.87 (1H, m), 7.82 (1H, d, J=8.3 Hz), 7.54-7.49 (1H, m), 7.36-7.30 (1H, m), 6.98-6.80 (1.5H, m), 6.74-6.60 (1.5H, m), 5.41 (2H, brs), 5.16-5.08 (1H, m), 4.78 (1.5H, s), 4.69 (0.5H, s), 4.65 (0.5H, s), 4.60 (1.5H, s), 4.47-4.40 (2H, m), 3.54-3.47 (2H, m), 3.21 (1.5H, s), 2.97 (0.5H, s), 2.87 (2H, t, J=7.8 Hz), 2.32 (4.5H, s), 2.32-2.28 (0.5H, m), 2.12-2.05 (1.5H, m), 2.05 (1.5H, s), 1.92-1.80 (2H, m), 1.53-1.44 (2H, m), 1.26 (6H, d, J=6.2 Hz), 1.00 (3H, t, J=7.3 Hz). MS:ESI 607 (M+1)

### Example 166

### Isopropyl 2-{3-[(N-{3-[4-amino-2-(ethoxymethyl)-1H-imidazo[4,5-c]quinolin-1-yl]propyl}-2-{diethylamino}acetamido)methyl]-2-fluorophenoxy}acetate

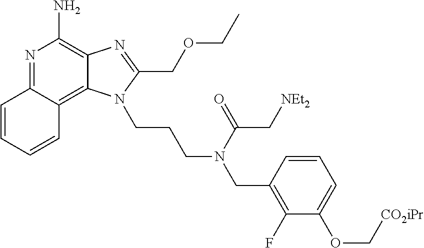

### (i) Isopropyl 2-[3-({3-[4-amino-2-(ethoxymethyl)-1H-imidazo[4,5-c]quinolin-1-yl]propylamino}methyl)-2-fluorophenoxy]acetate

By the method of example 1 step (viii) using the product from example 164 step (i) (0.30 g) and isopropyl 2-(2-fluoro-3-formylphenoxy)acetate (0.24 g) there was obtained the title compound, 0.51 g (97%) as a white solid.

¹H NMR δ (CDCl₃) 8.09 (1H, d, J=7.5 Hz), 7.81 (1H, dd, J=8.3 Hz, 0.8 Hz), 7.54-7.49 (1H, m), 7.33-7.26 (1H, m), 7.03-6.97 (2H, m), 6.90-6.78 (1H, m), 5.50 (2H, brs), 5.18-5.10 (1H, m), 4.84 (2H, s), 4.72 (2H, t, J=7.6 Hz), 4.66 (2H, s), 3.89 (2H, s), 3.60 (2H, q, J=7.0 Hz), 2.76 (2H, t, J=6.3 Hz), 2.18-2.10 (2H, m), 1.27 (6H, d, J=6.3 Hz), 1.26-1.21 (3H, m). MS:ESI 524 (M+1)

### (ii) Isopropyl 2-{3-[(N-{3-[4-amino-2-(ethoxymethyl)-1H-imidazo[4,5-c]quinolin-1-yl]propyl}-2-chloroacetamido)methyl]-2-fluorophenoxy}acetate

By the method of example 2 using the product of step (i) (0.50 g), there was obtained the title compound, 0.54 g (90%) as colorless gum.

¹H NMR δ (CDCl₃) 8.01-7.97 (2H, m), 7.69-7.63 (1H, m), 7.58-7.53 (1H, m), 7.03-6.96 (1H, m), 6.85-6.79 (1H, m), 6.75-6.71 (1H, m), 5.15-5.09 (1H, m), 4.80 (0.5H, s), 4.77 (1.5H, s), 4.70 (2H, s), 4.66-4.57 (4H, m), 4.21 (1.5H, s), 4.11 (0.5H, s), 3.67-3.57 (4H, m), 2.45-2.35 (0.5H, m), 2.25-2.16 (1.5H, m), 1.29-1.19 (9H, m). MS:ESI 601 (M+1)

### (iii) Isopropyl 2-{3-[(N-{3-[4-amino-2-(ethoxymethyl)-1H-imidazo[4,5-c]quinolin-1-yl]propyl}-2-{diethylamino}acetamido)methyl]-2-fluorophenoxy}acetate

The title compound was prepared by the method of example 5 using the product from step (ii) (0.27 g) to give a colorless gum (0.21 g). Yield 79%.

¹H NMR δ (CDCl₃) 7.93 (1H, d, J=8.1 Hz), 7.84-7.80 (1H, m), 7.56-7.52 (1H, m), 7.38-7.34 (1H, m), 7.00-6.88 (1.5H, m), 6.80-6.67 (1.5H, m), 5.43 (2H, brs), 5.17-5.08 (1H, m), 4.87 (1.5H, s), 4.78 (0.5H, s), 4.77 (1.5H, s), 4.71 (0.5H, s), 4.64 (0.5H, s), 4.61 (1.5H, s), 4.59-4.54 (2H, m), 3.65-3.52 (4H, m), 3.33 (1.5H, s), 3.21 (0.5H, s), 2.59 (3H, q, J=7.1 Hz), 2.48 (1H, q, J=7.1 Hz), 2.35-2.28 (0.5H, m), 2.20-2.15 (1.5H, m), 1.28-1.19 (9H, m), 1.02-0.90 (6H, m). MS:ESI 637 (M+1)

### Example 167

### Isopropyl 2-{3-[(N-{3-[4-amino-2-(ethoxymethyl)-1H-imidazo[4,5-c]quinolin-1-yl]propyl}-2-{dimethylamino}acetamido)methyl]-2-fluorophenoxy}acetate

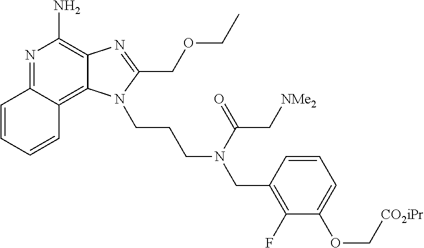

The title compound was prepared by the method of example 5 using the product from example 164 step (ii) (0.27 g) to give a colorless gum (0.23 g). Yield 89%.

¹H NMR δ (CDCl₃) 7.93 (1H, d, J=8.1 Hz), 7.82 (1H, d, J=8.3 Hz), 7.56-7.52 (1H, m), 7.38-7.34 (1H, m), 6.98-6.90 (1.5H, m), 6.80-6.64 (1.5H, m), 5.47 (2H, brs), 5.17-5.08 (1H, m), 4.81 (1.5H, s), 4.78 (0.5H, s), 4.77 (1.5H, s), 4.70 (0.5H, s), 4.65 (0.5H, s), 4.60 (1.5H, s), 4.59-4.54 (2H, m), 3.65-3.52 (4H, m), 3.19 (1.5H, s), 3.00 (0.5H, s), 2.38-2.30 (0.5H, m), 2.31 (4.5H, s), 2.20-2.15 (1.5H, m), 2.07 (1.5H, s), 1.28-1.19 (9H, m). MS:ESI 609 (M+1)

### Example 168

### Ethyl 2-[3-({N-[3-(4-amino-2-butyl-1H-imidazo[4,5-c]quinolin-1-yl)propyl]-2-(pyrrolidin-1-yl)acetamido}methyl)phenoxy]-2-methylpropanoate

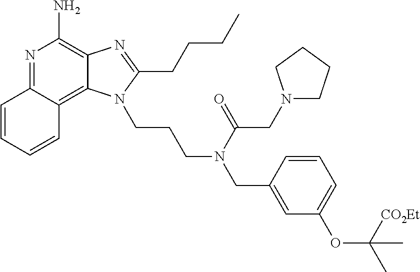

The title compound was prepared by the method of example 5 using the product from example 153 step (ii) (0.32 g) to give a colorless gum (0.28 g). Yield 87%.

¹H NMR δ (CDCl₃) 7.89 (0.75H, d, J=8.0 Hz), 7.85-7.81 (1.25H, m), 7.53-7.48 (1H, m), 7.35-7.31 (1H, m), 7.20-7.14 (1H, m), 6.80-6.68 (3H, m), 5.49 (2H, brs), 4.69 (1.5H, s), 4.55 (0.5H, s), 4.48-4.38 (2H, m), 4.21 (2H, q, J=7.1 Hz), 3.56 (1.5H, t, J=6.6 Hz), 3.52-3.46 (0.5H, m), 3.36 (1.5H, s), 3.19 (0.5H, s), 2.89-2.75 (2H, m), 2.63-2.57 (3H, m), 2.48-2.40 (1H, m), 2.25-2.18 (0.5H, m), 2.15-2.03 (1.5H, m), 1.90-1.80 (2H, m), 1.80-1.75 (3H, m), 1.68-1.62 (1H, m), 1.57 (6H, s), 1.55-1.43 (2H, m), 1.23 (3H, t, J=7.0 Hz), 0.98 (3H, t, J=7.4 Hz). MS:ESI 629 (M+1)

### Example 169

### Methyl 2-[3-({N-[3-(4-amino-2-butyl-1H-imidazo[4,5-c]quinolin-1-yl)propyl]-2-(pyrrolidin-1-yl)acetamido}methyl)phenoxy]-2-methylpropanoate

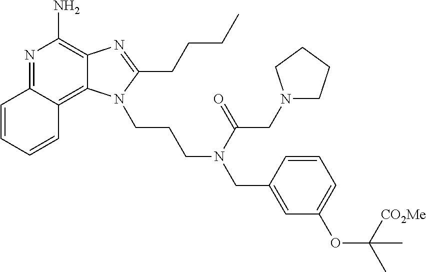

### (i) 2-[3-({N-[3-(4-Amino-2-butyl-1H-imidazo[4,5-c]quinolin-1-yl)propyl]-2-(pyrrolidin-1-yl)acetamido}methyl)phenoxy]-2-methylpropanoic acid

The title compound was prepared by the method of example 26 step (i) using the product from example 168 (0.25 g), to give a white solid (0.23 g). Yield 97%.

¹H NMR δ (DMSO-d₆) 7.93-7.85 (1H, m), 7.62-7.59 (1H, m), 7.47-7.42 (1H, m), 7.38-7.12 (4H, m), 6.78-6.66 (3H, m), 4.56 (1H, s), 4.46-4.42 (2H, m), 4.36-4.32 (1H, m), 3.68-3.58 (2H, m), 3.45-3.40 (2H, m), 2.90-2.79 (4H, m), 2.73-2.70 (2H, m), 2.12-2.05 (1H, m), 1.90-1.85 (1H, m), 1.79-1.64 (6H, m), 1.45-1.35 (8H, m), 0.97-0.91 (3H, m). MS:ESI 601 (M+1)

### (ii) Methyl 2-[3-({N-[3-(4-amino-2-butyl-1H-imidazo[4,5-c]quinolin-1-yl)propyl]-2-(pyrrolidin-1-yl)acetamido}methyl)phenoxy]-2-methylpropanoate

The title compound was prepared by the method of example 26 step (ii) using the product from step (i) (0.16 g) and methanol, to give a colorless gum (0.15 g). Yield 90%.

¹H NMR δ (CDCl₃) 7.89 (0.75H, d, J=8.1 Hz), 7.83 (1.25H, d, J=8.4 Hz), 7.54-7.49 (1H, m), 7.36-7.31 (1H, m), 7.21-7.15 (1H, m), 6.80-6.65 (3H, m), 5.40 (2H, brs), 4.70 (1.5H, s), 4.56 (0.5H, s), 4.49-4.40 (2H, m), 3.76 (3H, s), 3.57 (1.5H, t, J=6.6 Hz), 3.51-3.46 (0.5H, m), 3.36 (1.5H, s), 3.20 (0.5H, s), 2.90-2.79 (2H, m), 2.63-2.57 (3H, m), 2.47-2.40 (1H, m), 2.20-2.12 (0.5H, m), 2.12-2.03 (1.5H, m), 1.92-1.75 (5H, m), 1.68-1.64 (1H, m), 1.58 (6H, s), 1.56-1.47 (2H, m), 1.00 (3H, t, J=7.3 Hz). MS:ESI 615 (M+1)

### Example 170

### Ethyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}-2-methylpropanoate

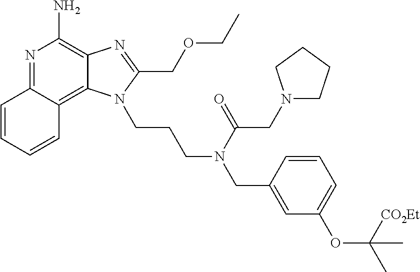

The title compound was prepared by the method of example 5 using the product from example 162 step (ii) (0.32 g) to give a colorless gum (0.28 g). Yield 89%.

¹H NMR δ (CDCl₃) 7.94 (0.75H, d, J=8.2 Hz), 7.87 (0.25H, d, J=8.2 Hz), 7.82 (1H, d, J=8.3 Hz), 7.56-7.50 (1H, m), 7.39-7.33 (1H, m), 7.21-7.12 (1H, m), 6.81-6.68 (3H, m), 5.44 (2H, brs), 4.77 (1.5H, s), 4.76 (0.5H, s), 4.71 (1.5H, s), 4.60-4.54 (2.5H, m), 4.21 (2H, q, J=7.1 Hz), 3.64-3.57 (3.5H, m), 3.50 (0.5H, t, J=7.5 Hz), 3.36 (1.5H, s), 3.23 (0.5H, s), 2.62-2.58 (3H, m), 2.49-2.43 (1H, m), 2.29-2.22 (0.5H, m), 2.19-2.12 (1.5H, m), 1.78-1.74 (3H, m), 1.69-1.65 (1H, m), 1.58 (6H, s), 1.24 (6H, t, J=7.1 Hz). MS:ESI 631 (M+1)

### Example 171

### Methyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}-2-methylpropanoate

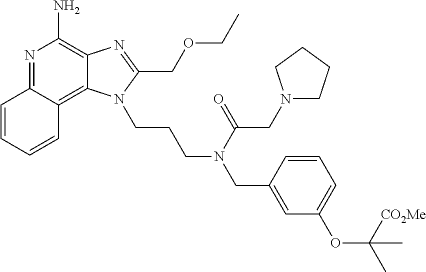

### (i) 2-{3-[(N-{3-[4-Amino-2-(ethoxymethyl)-1H-imidazo[4,5-c]quinolin-1-yl]propyl}-2-{pyrrolidin-1-yl}acetamido)methyl]phenoxy}-2-methylpropanoic acid

The title compound was prepared by the method of example 26 step (i) using the product from example 170 (0.24 g), to give a white solid (0.22 g). Yield 95%.

¹H NMR δ (DMSO-d₆) 8.01-7.93 (1H, m), 7.61 (1H, d, J=8.3 Hz), 7.48-7.44 (1H, m), 7.31-7.12 (2H, m), 6.94 (2H, brs), 6.74-6.67 (3H, m), 4.74 (0.5H, s), 4.73 (0.5H, s), 4.63 (1H, s), 4.60-4.55 (1H, m), 4.52-4.48 (2H, m), 3.57-3.44 (6H, m), 3.30-3.26 (1H, m), 2.72-2.64 (2H, m), 2.52-2.47 (2H, m), 2.14-2.10 (1H, m), 2.04-2.00 (1H, m), 1.69-1.64 (2H, m), 1.57-1.53 (2H, m), 1.43 (6H, s), 1.15-1.09 (3H, m). MS:ESI 603 (M+1)

### (ii) Methyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}-2-methylpropanoate

The title compound was prepared by the method of example 26 step (ii) using the product from step (i) (0.16 g) and methanol, to give a colorless gum (0.15 g). Yield 92%.

¹H NMR δ (CDCl₃) 7.94 (0.75H, d, J=7.9 Hz), 7.86-7.81 (1.25H, m), 7.56-7.51 (1H, m), 7.38-7.32 (1H, m), 7.21-7.14 (1H, m), 6.81-6.65 (3H, m), 5.49 (2H, brs), 4.77 (1.5H, s), 4.75 (0.5H, s), 4.71 (1.5H, s), 4.60-4.54 (2.5H, m), 3.75 (3H, s), 3.64-3.56 (3.5H, m), 3.51-3.46 (0.5H, m), 3.36 (1.5H, s), 3.23 (0.5H, s), 2.62-2.56 (3H, m), 2.48-2.44 (1H, m), 2.30-2.10 (2H, m), 1.78-1.72 (3H, m), 1.69-1.64 (1H, m), 1.58 (4.5H, s), 1.55 (1.5H, s), 1.21 (3H, t, J=7.1 Hz). MS:ESI 615 (M+1)

### Example 172

### Isopropyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}acetate

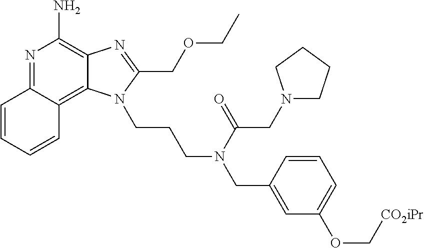

The title compound was prepared by the method of example 5 using the product from example 161 step (v) (0.32 g) to give a colorless gum (0.25 g). Yield 81%.

¹H NMR δ (CDCl₃) 7.93 (0.75H, d, J=8.0 Hz), 7.87 (0.25H, d, J=8.4 Hz), 7.82 (1H, d, J=8.3 Hz), 7.57-7.50 (1H, m), 7.38-7.32 (1H, m), 7.24-7.18 (1H, m), 6.80-6.73 (3H, m), 5.43 (2H, brs), 5.18-5.08 (1H, m), 4.77 (1.5H, s), 4.76 (0.5H, s), 4.72 (1.5H, s), 4.60-4.53 (4.5H, m), 3.64-3.55 (3.5H, m), 3.51 (0.5H, t, J=7.4 Hz), 3.37 (1.5H, s), 3.23 (0.5H, s), 2.62-2.58 (3H, m), 2.49-2.43 (1H, m), 2.29-2.22 (0.5H, m), 2.21-2.12 (1.5H, m), 1.78-1.74 (3H, m), 1.70-1.65 (1H, m), 1.27 (6H, d, J=6.3 Hz), 1.25-1.19 (3H, m). MS:ESI 617 (M+1)

### Example 173

### Ethyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate

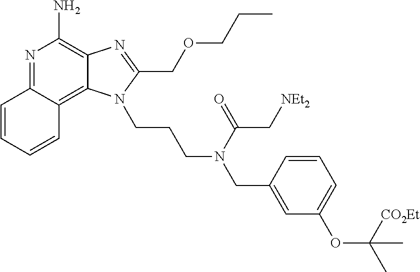

### (i) tert-Butyl 3-[2-(propoxymethyl)-1H-imidazo[4,5-c]quinolin-1-yl]propylcarbamate

The title compound was prepared by the method of example 15 step (i) using the product from example 1 step (iii) (3.00 g), to give the title compound (3.44 g). Yield 91%.

¹H NMR δ (CDCl₃) 9.30 (1H, s), 8.31-8.27 (1H, m), 8.24-8.21 (1H, m), 7.74-7.63 (2H, m), 4.99-4.95 (1H, m), 4.89 (2H, s), 4.75-4.71 (2H, m), 3.54-3.36 (2H, m), 3.34-3.29 (2H, m), 2.25-2.18 (2H, m), 1.69-1.62 (2H, m), 1.46 (9H, s), 0.94 (3H, t, J=7.4 Hz).

MS: ESI 399 (M+1)

### (ii) 1-(3-Aminopropyl)-2-(propoxymethyl)-1H-imidazo[4,5-c]quinolin-4-amine

The title compound was prepared by the method of example 15 step (ii-iv) using the product from previous step (3.44 g), to give the title compound (1.10 g). Yield 37%.

¹H NMR δ (CDCl₃) 8.10 (1H, d, J=8.2 Hz), 7.83 (1H, d, J=8.3 Hz), 7.54 (1H, dd, J=8.2 Hz, 7.2 Hz), 7.35 (1H, dd, J=8.1 Hz, 7.2 Hz), 5.42 (2H, brs), 4.85 (2H, s), 4.73 (2H, t, J=7.7 Hz), 3.52 (2H, t, J=6.7 Hz), 2.90 (2H, t, J=6.6 Hz), 2.16-2.07 (2H, m), 1.70-1.40 (4H, m), 0.94 (3H, t, J=7.4 Hz).

MS: ESI 314 (M+1)

### (iii) Ethyl 2-[3-({3-[4-amino-2-(propoxymethyl)-1H-imidazo[4,5-c]quinolin-1-yl]propylamino}methyl)phenoxy]-2-methylpropanoate

By the method of example 1 step (viii) using the product from step (ii) (0.23 g) and ethyl 2-(3-formylphenoxy)-2-methylpropanoate (0.17 g) there was obtained the title compound, 0.28 g (74%) as a white solid

¹H NMR δ (CDCl₃) 8.12 (1H, d, J=7.4 Hz), 7.83 (1H, d, J=8.4 Hz), 7.55-7.49 (1H, m), 7.32-7.26 (1H, m), 7.21 (1H, dd, J=7.9 Hz, 7.8 Hz), 7.01-6.97 (1H, m), 6.91-6.88 (1H, m), 6.72 (1H, dd, J=7.9 Hz, 2.2 Hz), 5.48 (2H, brs), 4.84 (2H, s), 4.74 (2H, t, J=7.6 Hz), 4.22 (2H, q, J=7.1 Hz), 3.78 (2H, s), 3.50 (2H, t, J=6.7 Hz), 2.77 (2H, t, J=6.3 Hz), 2.18-2.09 (2H, m), 1.68-1.60 (2H, m), 1.62 (6H, s), 1.24 (3H, t, J=7.1 Hz), 0.92 (3H, t, J=7.4 Hz). MS:ESI 534 (M+1)

### (iv) Ethyl 2-[3-[(N-{3-[4-amino-2-(propoxymethyl)-1H-imidazo[4,5-c]quinolin-1-yl]propyl}-2-chloroacetamido)methyl]phenoxy]-2-methylpropanoate hydrochloride

By the method of example 2 using the product of step (iii) (0.28 g), there was obtained the title compound, 0.33 g (98%) as colorless gum.

¹H NMR δ (CDCl₃) 7.97 (1H, d, J=8.1 Hz), 7.90-7.81 (1H, m), 7.59 (1H, dd, J=7.4 Hz, 7.4 Hz), 7.46-7.38 (1H, m), 7.27-7.10 (1H, m), 6.78-6.68 (3H, m), 6.30 (2H, brs), 4.78 (2H, s), 4.62 (2H, s), 4.62-4.51 (2H, m), 4.20 (2H, q, J=7.1 Hz), 4.09 (2H, s), 3.63 (1.5H, t, J=6.6 Hz), 3.54-3.40 (2.5H, m), 2.30-2.16 (2H, m), 1.67-1.58 (2H, m), 1.59 (4.5H, s), 1.54 (1.5H, s), 1.26-1.22 (3H, m), 0.93 (3H, t, J=7.4 Hz). MS:ESI 611 (M+1)

### (v) Ethyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate

The title compound was prepared by the method of example 5 using the product from step (iv) (0.33 g) to give a colorless gum (0.31 g). Yield 93%.

¹H NMR δ (CDCl₃) 7.95 (0.75H, d, J=7.7 Hz), 7.88-7.82 (1.25H, m), 7.57-7.52 (1H, m), 7.29-7.31 (1H, m), 7.21-7.14 (1H, m), 6.79-6.67 (3H, m), 5.40 (2H, brs), 4.78 (1.5H, s), 4.77-4.75 (2H, m), 4.60-4.55 (2.5H, m), 4.21 (2H, q, J=7.1 Hz), 3.59 (2H, t, J=6.9 Hz), 3.52-3.47 (2H, m), 3.29 (1.5H, s), 3.25 (0.5H, s), 2.60 (3H, q, J=7.1 Hz), 2.52 (1H, q, J=7.2 Hz), 2.30-2.22 (0.5H, m), 2.19-2.10 (1.5H, m), 1.64-1.57 (2H, m), 1.57 (6H, s), 1.28-1.20 (3H, m), 0.99 (6H, t, J=7.1 Hz), 0.92 (3H, t, J=7.4 Hz). MS:ESI 647 (M+1)

### Example 174

### Methyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate

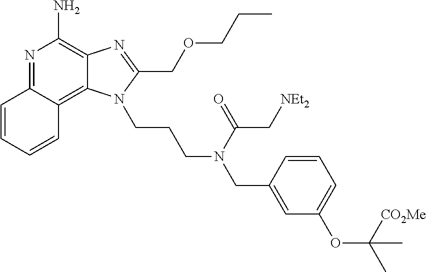

### (i) 2-{3-[(N-{3-[4-Amino-2-(propoxymethyl)-1H-imidazo[4,5-c]quinolin-1-yl]propyl}-2-{diethylamino}acetamido)methyl]phenoxy}-2-methylpropanoic acid

The title compound was prepared by the method of example 26 step (i) using the product from example 173 (0.30 g), to give a white solid (0.26 g). Yield 92%.

¹H NMR δ (DMSO-d₆) 8.01-7.93 (1H, m), 7.61 (1H, d, J=8.3 Hz), 7.46 (1H, dd, J=7.8 Hz, 7.4 Hz), 7.30-7.20 (1H, m), 7.18-7.07 (1H, m), 6.92 (2H, brs), 6.75-6.67 (3H, m), 4.74-4.73 (2H, m), 4.70 (1H, s), 4.60-4.55 (1H, m), 4.50-4.45 (1H, m), 4.43 (1H, s), 3.55-3.48 (1H, m), 3.45-3.42 (1H, m), 3.42 (2H, t, J=6.6 Hz), 3.20 (1H, s), 3.18 (1H, s), 2.55-2.46 (2H, m), 2.40 (2H, q, J=6.9 Hz), 2.20-2.10 (1H, m), 2.08-1.99 (1H, m), 1.60-1.48 (2H, m), 1.42 (6H, s), 0.89-0.80 (9H, m). MS:ESI 619 (M+1)

### (ii) Methyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate

The title compound was prepared by the method of example 26 step (ii) using the product from step (i) (0.20 g) and methanol, to give a white solid (0.19 g). Yield 92%.

¹H NMR δ (CDCl₃) 7.95 (0.75H, d, J=7.7 Hz), 7.90-7.80 (1.25H, m), 7.57-7.52 (1H, m), 7.39-7.33 (1H, m), 7.22-7.14 (1H, m), 6.79-6.66 (3H, m), 5.43 (2H, brs), 4.78-4.75 (3.5H, 3), 4.60-4.55 (2.5H, m), 3.76 (3H, s), 3.58 (2H, t, J=6.8 Hz), 3.52-3.44 (2H, m), 3.29 (1.5H, s), 3.26 (0.5H, s), 2.60 (3H, q, J=7.1 Hz), 2.52 (1H, q, J=7.2 Hz), 2.32-2.22 (0.5H, m), 2.20-2.10 (1.5H, m), 1.68-1.58 (2H, m), 1.58 (6H, s), 0.99 (6H, t, J=7.1 Hz), 0.92-0.85 (3H, m). MS:ESI 633 (M+1)

### Biological Assay

### Human TLR7 Assay

The most common variant sequence of human TLR7 (represented by the EMBL sequence AF240467) was cloned into the mammalian cell expression vector pUNO and transfected into a HEK293 cell line already stably expressing the pNiFty2-SEAP reporter plasmid; integration of the reporter gene was maintained by selection with the antibiotic zeocin. Transfectants with stable TLR7 expression were selected using the antibiotic blasticidin. In this reporter cell-line, expression of secreted alkaline phosphatase (SEAP) is controlled by an NFkB/ELAM-1 composite promoter comprising five NFkB sites combined with the proximal ELAM-1 promoter. TLR signaling leads to the translocation of NFkB and activation of the promoter results in expression of the SEAP gene. TLR7-specific activation was assessed by determining the level of SEAP produced following overnight incubation of the cells at 37° C. with the standard compound in the presence of 0.1% (v/v) dimethylsulfoxide (DMSO). Concentration dependent induction of SEAP production by compounds was expressed as the concentration of compound which produced half of the maximal level of SEAP induction for that compound (EC₅₀).

| Compound | hTLR7 EC50 (nM) |
|---|---|
| Example 1 | Not tested. |
| Example 2 | Not tested. |
| Example 3 | Not tested. |
| Example 4 | Not tested. |
| Example 5 | 247.1 |
| Example 6 | 168.9 |
| Example 7 | 633.1 |
| Example 8 | 904.0 |
| Example 9 | 191.6 |
| Example 10 | 135.1 |
| Example 11 | 323.1 |
| Example 12 | 432.5 |
| Example 13 | 136.2 |
| Example 14 | 73.6 |
| Example 15 | 181.3 |
| Example 16 | Not tested. |
| Example 17 | 348.7 |
| Example 18 | 1274.2 |
| Example 19 | 178.4 |
| Example 20 | 31.7 |
| Example 21 | 786.6 |
| Example 22 | 188.3 |
| Example 23 | Not tested. |
| Example 24 | Not tested. |
| Example 25 | 58.7 |
| Example 26 | 54.6 |
| Example 27 | 66.1 |
| Example 28 | 49.9 |
| Example 29 | 174.3 |
| Example 30 | 1461.7 |
| Example 31 | 149.6 |
| Example 32 | 177.3 |
| Example 33 | 177.2 |
| Example 34 | Not tested. |
| Example 35 | Not tested. |
| Example 36 | 84.5 |
| Example 37 | 38.3 |
| Example 38 | Not tested. |
| Example 39 | Not tested. |
| Example 40 | 243.4 |
| Example 41 | 136.6 |
| Example 42 | Not tested. |
| Example 43 | Not tested. |
| Example 44 | 22.7 |
| Example 45 | 20.4 |
| Example 46 | Not tested. |
| Example 47 | Not tested. |
| Example 48 | 24.7 |
| Example 49 | Not tested. |
| Example 50 | Not tested. |
| Example 51 | 42.6 |
| Example 52 | 24.2 |
| Example 53 | Not tested. |
| Example 54 | Not tested. |
| Example 55 | 46.4 |
| Example 56 | 33.1 |
| Example 57 | Not tested. |
| Example 58 | Not tested. |
| Example 59 | 69 |
| Example 60 | Not tested. |
| Example 61 | Not tested. |
| Example 62 | 84.2 |
| Example 63 | 211.9 |
| Example 64 | Not tested. |
| Example 65 | Not tested. |
| Example 66 | 27.3 |
| Example 67 | 45.8 |
| Example 68 | Not tested. |
| Example 69 | Not tested. |
| Example 70 | 25.9 |
| Example 71 | 23 |
| Example 72 | 55.8 |
| Example 73 | Not tested. |
| Example 74 | Not tested. |
| Example 75 | 404.7 |
| Example 76 | 210.7 |
| Example 77 | Not tested. |
| Example 78 | Not tested. |
| Example 79 | 51.8 |
| Example 80 | 56.6 |
| Example 81 | 56.1 |
| Example 82 | 114.7 |
| Example 83 | 61.3 |
| Example 84 | Not tested. |
| Example 85 | Not tested. |
| Example 86 | 52.5 |
| Example 87 | Not tested. |
| Example 88 | Not tested. |
| Example 89 | 95.7 |
| Example 90 | 81 |
| Example 91 | Not tested. |
| Example 92 | Not tested. |
| Example 93 | 73.9 |
| Example 94 | 27.9 |
| Example 95 | 447.4 |
| Example 96 | 303.8 |
| Example 97 | 256.5 |
| Example 98 | 227.5 |
| Example 99 | Not tested. |
| Example 100 | Not tested. |
| Example 101 | >10000 |
| Example 102 | >10000 |
| Example 103 | >10000 |
| Example 104 | Not tested. |
| Example 105 | Not tested. |
| Example 106 | >10000 |
| Example 107 | >10000 |
| Example 108 | Not tested. |
| Example 109 | Not tested. |
| Example 110 | 128 |
| Example 111 | 122.9 |
| Example 112 | 756.3 |
| Example 113 | 83.4 |
| Example 114 | 67.8 |
| Example 115 | 172.9 |
| Example 116 | Not tested. |
| Example 117 | Not tested. |
| Example 118 | 77.4 |
| Example 119 | 93.2 |
| Example 120 | 278.2 |
| Example 121 | Not tested. |
| Example 122 | Not tested. |
| Example 123 | 50.2 |
| Example 124 | 40.3 |
| Example 125 | 11.2 |
| Example 126 | 47 |
| Example 127 | 42.9 |
| Example 128 | 130.1 |
| Example 129 | 203.7 |
| Example 130 | 24.4 |
| Example 131 | 37.5 |
| Example 132 | 56.4 |
| Example 133 | 114.5 |
| Example 134 | 20.9 |
| Example 135 | 16.6 |
| Example 136 | 111.5 |
| Example 137 | 87.7 |
| Example 138 | Not tested. |
| Example 139 | 57 |
| Example 140 | 79.1 |
| Example 141 | 201.3 |
| Example 142 | 57.5 |
| Example 143 | 49.4 |
| Example 144 | 79.2 |
| Example 145 | Not tested. |
| Example 146 | Not tested. |
| Example 147 | 183 |
| Example 148 | 114.9 |
| Example 149 | 181.3 |
| Example 150 | 84.6 |
| Example 151 | 82 |
| Example 152 | 266 |
| Example 153 | 86.1 |
| Example 154 | 67.7 |
| Example 155 | 235.2 |
| Example 156 | 168.1 |
| Example 157 | 100.3 |
| Example 158 | 105.0 |
| Example 159 | 139.2 |
| Example 160 | 259.9 |
| Example 161 | 262.1 |
| Example 162 | 96.4 |
| Example 163 | 173.7 |
| Example 164 | 158.0 |
| Example 165 | 296.6 |
| Example 166 | 474.9 |
| Example 167 | 715.7 |
| Example 168 | 165.9 |
| Example 169 | 155.5 |
| Example 170 | 521.0 |
| Example 171 | 559.8 |
| Example 172 | 433.8 |
| Example 173 | Not tested |
| Example 174 | 117.5 |

Certain of the compounds exemplified exhibited low activity on the Human TLR7 assay described above. Accordingly, in one embodiment according to the invention there is provided a compound of the formula (I), or a pharmaceutically acceptable salt as described herein other than the compounds:
Ethyl 2-(31(N-(2-(4-amino-2-(2-methoxyethyl)-1H-imidazo[4,5-c]quinolin-1-yl)ethyl)-2-(diethylamino)acetamido)methyl)phenoxy)acetate;
Methyl 2-(31(N-(2-(4-amino-2-(2-methoxyethyl)-1H-imidazo[4,5-c]quinolin-1-yl)ethyl)-2-(diethylamino)acetamido)methyl)phenoxy)acetate;
Isopropyl 2-(3-((N-(2-(4-amino-2-(2-methoxyethyl)-1H-imidazo[4,5-c]quinolin-1-yl)ethyl)-2-(diethylamino)acetamido)methyl)phenoxy)acetate;
Ethyl 2-(3-((N-(2-(4-amino-2-(2-methoxyethyl)-1H-imidazo[4,5-c]quinolin-1-yl)ethyl)-2-(diethylamino)acetamido)methyl)phenoxy)-2-methylpropanoate; and Methyl 2-(3-((N-(2-(4-amino-2-(2-methoxyethyl)-1H-imidazo[4,5-c]quinolin-1-yl)ethyl)-2-(diethylamino)acetamido)methyl)phenoxy)-2-methylpropanoate.

### Effect of the Compounds on Antigen-Induced Pulmonary Inflammation in a Rat Asthma Model

Rats were sensitized and challenged to produce allergic airway inflammation in a similar manner to that described by Underwood et al (British Journal of Pharmacology 2002; 137: 263-275, 2002). Male Brown Norway rats were sensitized subcutaneously with ovalbumin (OVA) and aluminum hydroxide on day 0, and challenged with aerosolized OVA solution on day 14. The test compound was administered twice intratracheally 24 hours before and 24 hours after the OVA-challenge and bronchoalveolar lavage fluid (BALF) was collected 48 hours after the OVA-challenge. Then eosinophils and Th2 cytokines (IL-5 and IL-13) in the BALF were measured to evaluate efficacy of the test compounds of this invention. The results obtained are shown in the following table.

Eosinophils and Th2 cytokines in BALF

| Compounds | Dose(mg/kg) | | Eosinophiles | IL-5 | IL-13 |
|---|---|---|---|---|---|
| Example 9 | 0.1 | (n = 6) | 53% | 66% | 89% |
| | 1.0 | (n = 6) | 77% | 72% | 85% |
| Example 20 | 0.01 | (n = 6) | 14% | No effect | No effect |
| | 0.1 | (n = 5) | 56% | No effect | 22% |
| Example 27 | 0.1 | (n = 5) | 64% | 41% | 72% |
| | 1 | (n = 5) | 89% | 90% | 88% |
| Example 56 | 0.1 | (n = 4) | 78% | 86% | 89% |
| | 1.0 | (n = 6) | 94% | 99% | 98% |
| Example 80 | 0.1 | (n = 5) | 15% | No effect | No effect |
| | 1 | (n = 5) | 89% | 91% | 83% |
| Example 94 | 0.1 | (n = 6) | 68% | 34% | 50% |
| | 1 | (n = 6) | 91% | 90% | 82% |
| Example 161 | 0.03 | (n = 8) | 54% | NT | No effect |
| | 0.3 | (n = 8) | 66% | NT | 64% |
| Example 163 | 0.03 | (n = 8) | 45% | NT | 50% |
| | 0.3 | (n = 8) | 65% | NT | 80% |

* Eosinophil(Cells/BALF), IL-5(pg/mL BALF) and IL-13(pg/mL BALF): data shows inhibition (%) to OVA-Challenge control.
“No effect” in the Table means that the test compound showed the almost same level of IL-5/IL-13 induction as the OVA-challenge control.

What is claimed is:

1. A compound of formula (I): 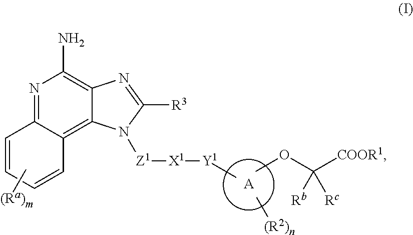
wherein
R¹ represents C₁-C₈ alkyl, C₃-C₈ cycloalkyl, or a 3- to 8-membered saturated heterocyclic ring group comprising a O atom, wherein R¹ is optionally substituted by one or more substituents independently selected from halogen, cyano, hydroxyl and C₁-C₃ alkoxy;
Z¹ represents a C₂-C₆ alkylene group, wherein a carbon atom in Z¹ which is not adjacent to a nitrogen atom may be replaced with an oxygen atom;
X¹ represents NR⁵, >N—COR⁵, >N—CONR⁵R^{5a}, CONR⁵, NR⁵CO, NR⁵CONR⁶ or NR⁶CONR⁵;
Y¹ represents a single bond or C₁-C₆ alkylene;
each R² is independently selected from halogen, cyano, hydroxy, thiol, C₁-C₃ alkyl, C₁-C₃ hydroxyalkyl, C₁-C₃ haloalkyl, C₁-C₃ alkoxy, C₁-C₃ haloalkoxy, C₁₋₃ alkylthio, C₁₋₃ alkylsulfonyl and C₁₋₃ alkylsulfinyl;
R³ represents C₁₋₆ alkyl optionally substituted by C₁₋₆ alkoxy;
each R^{a} is independently selected from halogen, cyano, hydroxy, thiol, C₁-C₃ alkyl, C₁-C₃ hydroxyalkyl, C₁-C₃ haloalkyl, C₁-C₃ alkoxy, C₁-C₃ haloalkoxy, C₁₋₃alkylthio, C₁₋₃alkylsulfonyl and C₁₋₃alkylsulfinyl;
R⁵ and R^{5a} each independently represents hydrogen, a 3- to 8-membered saturated heterocyclic ring comprising a ring group O, S(O)ₚ or NR¹⁰, a C₁-C₆ alkyl group or C₃-C₆ cycloalkyl group, the latter two groups being optionally substituted by one or more substituents independently selected from NR⁷R⁸ or R⁹,
R⁷ and R⁸ each independently represent hydrogen, a 3- to 8-membered saturated heterocyclic ring comprising a ring group O, S(O)ₚ or NR^{10a}, C₁-C₆ alkyl or C₃-C₆ cycloalkyl, the latter two groups being optionally substituted by one or more groups independently selected from halogen, cyano, S(O)_{q}R¹¹, OR¹², CO₂R¹², OC(O)R¹², SO₂NR¹²R¹³, CONR¹²R¹³, NR¹²R¹³, NR¹²SO₂R¹⁴, NR¹²COR¹³, or a 3- to 8-membered saturated heterocyclic ring comprising a ring group O, S(O)ₚ or NR^{10b},
or R⁷ and R⁸ together with the nitrogen atom to which they are attached form a 3- to 8-membered saturated heterocyclic ring comprising a ring nitrogen atom and optionally one or more further heteroatoms independently selected from nitrogen, oxygen, sulphur and sulphonyl, the heterocyclic ring being optionally substituted by one or more substituents independently selected from halogen, cyano, S(O)_{q}R¹⁵, OR¹⁵, CO₂R¹⁵, COR¹⁵, OC(O)R¹⁵, SO₂NR¹⁵R¹⁶, CONR¹⁵R¹⁶, NR¹⁵R¹⁶, NR¹⁵SO₂R¹⁷, NR¹⁵COR¹⁶, NR¹⁵CO₂R¹⁶, heteroaryl, C₁-C₆ haloalkyl, C₃-C₈ cycloalkyl and C₁-C₆ alkyl, the latter two groups being optionally substituted by one or more groups independently selected from cyano, S(O)_{q}R¹⁸, OR¹⁸, CO₂R¹⁸, SO₂NR¹⁸R¹⁹, CONR¹⁸R¹⁹ or NR¹⁸R¹⁹;
R⁹ represents halogen, cyano, CO₂R²⁰, S(O)_{q}R²⁰, OR²⁰, SO₂NR²OR²², CONR²OR²², NR²⁰SO₂R²¹, NR²⁰CO₂R²¹, NR²⁰COR²² or a 3- to 8-membered saturated heterocyclic ring comprising a ring group NR^{10c};
R¹⁰, R^{10a}, R^{10b} and R^{10c} independently represent hydrogen, CO₂R²³, S(O)_{q}R²³, COR²⁴, or a C₁-C₆ alkyl, C₂-C₆ alkenyl, C₂-C₆ alkynyl or C₃-C₈ cycloalkyl group, each of which may be optionally substituted by one or more substituents independently selected from halogen, cyano, OR²⁵ or NR²⁵R²⁶;
R⁶, R¹¹, R¹², R¹³, R¹⁵, R¹⁶, R¹⁸, R¹⁹, R²⁰, R²², R²⁴, R²⁵ and R²⁶ each independently represent hydrogen, C₁-C₆ alkyl or C₃-C₆ cycloalkyl;
R¹⁴, R¹⁷, R²¹ and R²³ each independently represent C₁-C₆ alkyl or C₃-C₆ cycloalkyl;
m, n, p and q each independently represent an integer 0, 1 or 2; and
A represents a monocyclic or bicyclic C₆-C₁₀ aryl or a monocyclic or bicyclic C₅-C₁₂ heteroaryl group containing 1-3 heteroatoms; and
R^{b} and R^{c} independently represent hydrogen or C₁-C₆ alkyl, or R^{b} and R^{c} combine together to form C₃-C₈ cycloalkyl.
or a pharmaceutically acceptable salt thereof.

2. A compound according to claim 1 wherein R¹ is straight chain C₁₋₄ alkyl.

3. A compound according to claim 2, wherein R¹ is methyl or ethyl.

4. A compound according to claim 2 or 3, wherein at least one of R^{b} and R^{c} is C₁₋₃ alkyl, or R^{b} and R^{c} combine together to form C₃-C₆ cycloalkyl.

5. A compound according to claim 4, wherein R¹, R^{b} and R^{c} are methyl.

6. A compound according to claim 4, wherein R¹ is ethyl, R^{b} is methyl and R^{c} is hydrogen.

7. A compound according to claim 1 wherein R¹ is a branched chain C₃₋₆ alkyl, a C₃₋₆ cycloalkyl or a tetrahydropyranyl.

8. A compound according to claim 7, wherein R¹ is isopropyl.

9. A compound according to claim 7 or 8, wherein R^{b} and R^{c} are hydrogen.

10. A compound according to any one of the preceding claims wherein Z¹ is n-propylene.

11. A compound according to any one of the preceding claims wherein X¹ is a group NR⁵, >NCOR⁵, or >NCONR⁵R^{5a}.

12. A compound according to any one of the preceding claims wherein X¹ is a group >NCOR⁵.

13. A compound according to any one of the preceding claims wherein R⁵ is hydrogen or a C₁-C₆ alkyl optionally substituted by one or more groups NR⁷R⁸ or R⁹ where R⁷, R⁸ and R⁹ are as defined in claim 1.

14. A compound according to any one of the preceding claims wherein Y¹ represents C₁-C₆ alkylene.

15. A compound according to any one of the preceding claims wherein A is phenyl.

16. A compound according to any one of the preceding claims where n is 0.

17. A compound according to any one of the preceding claims where R³ is n-propyl, n-butyl, methoxyethyl or ethoxymethyl.

18. A compound according to any one of the preceding claims where m is 0.

19. A compound according to claim 1 selected from the group consisting of following compounds or a pharmaceutically acceptable salt thereof:
Methyl 2-(3-{[3-(4-amino-2-butyl-1H-imidazo[4,5-c]quinolin-1-yl)propylamino]methyl}phenoxy)acetate,
Methyl (3-{[[3-(4-amino-2-butyl-1H-imidazo[4,5-c]quinolin-1-yl)propyl](chloroacetyl)amino]methyl}phenoxy)acetate,
Methyl (4-{[[3-(4-amino-2-butyl-1H-imidazo[4,5-c]quinolin-1-yl)propyl]amino]methyl}phenoxy)acetate,
Methyl (4-{[[3-(4-amino-2-butyl-1H-imidazo[4,5-c]quinolin-1-yl)propyl](chloroacetyl)amino]methyl}phenoxy)acetate,
Methyl (4-{[[3-(4-amino-2-butyl-1H-imidazo[4,5-c]quinolin-1-yl)propyl](N,N-dimethylglycyl)amino]methyl}phenoxy)acetate,
Methyl (4-{[[3-(4-amino-2-butyl-1H-imidazo[4,5-c]quinolin-1-yl)propyl](piperidin-1-ylacetyl)amino]methyl}phenoxy)acetate,
Methyl [4-({[3-(4-amino-2-butyl-1H-imidazo[4,5-c]quinolin-1-yl)propyl][(4-methylpiperazin-1-yl)acetyl]amino}methyl)phenoxy]acetate,
Methyl {4-[([3-(4-amino-2-butyl-1H-imidazo[4,5-c]quinolin-1-yl)propyl]{[4-(2-methoxyethyl)piperazin-1-yl]acetyl}amino)methyl]phenoxy}acetate,
Methyl (3-{[[3-(4-amino-2-butyl-1H-imidazo[4,5-c]quinolin-1-yl)propyl](N,N-dimethylglycyl)amino]methyl}phenoxy)acetate,
Methyl (3-{[[3-(4-amino-2-butyl-1H-imidazo[4,5-c]quinolin-1-yl)propyl](piperidin-1-ylacetyl)amino]methyl}phenoxy)acetate,
Methyl [3-({[3-(4-amino-2-butyl-1H-imidazo[4,5-c]quinolin-1-yl)propyl][(4-methylpiperazin-1-yl)acetyl]amino}methyl)phenoxy]acetate,
Methyl {3-[([3-(4-amino-2-butyl-1H-imidazo[4,5-c]quinolin-1-yl)propyl]{[4-(2-methoxyethyl)piperazin-1-yl]acetyl}amino)methyl]phenoxy}acetate,
Methyl (3-{[[3-(4-amino-2-butyl-1H-imidazo[4,5-c]quinolin-1-yl)propyl](pyrrolidin-1-ylacetyl)amino]methyl}phenoxy)acetate,
Methyl (3-{[[3-(4-amino-2-butyl-1H-imidazo[4,5-c]quinolin-1-yl)propyl](N,N-diethylglycyl)amino]methyl}phenoxy)acetate,
Methyl (3-{[[3-(4-amino-2-(2-methoxyethyl)-1H-imidazo[4,5-c]quinolin-1-yl)propyl]amino]methyl}phenoxy)acetate,
Methyl (3-{[[3-(4-amino-2-(2-methoxyethyl)-1H-imidazo[4,5-c]quinolin-1-yl)propyl](chloroacetyl)amino]methyl}phenoxy)acetate,
Methyl (3-{[[3-(4-amino-2-(2-methoxyethyl)-1H-imidazo[4,5-c]quinolin-1-yl)propyl](N,N-dimethylglycyl)amino]methyl}phenoxy)acetate,
Methyl (3-{[[3-(4-amino-2-(2-methoxyethyl)-1H-imidazo[4,5-c]quinolin-1-yl)propyl][(4-methylpiperazin-1-yl)acetyl]amino]methyl}phenoxy)acetate,
Methyl (3-{[[3-(4-amino-2-(2-methoxyethyl)-1H-imidazo[4,5-c]quinolin-1-yl)propyl](piperidin-1-ylacetyl)amino]methyl}phenoxy)acetate,
Methyl (3-{[[3-(4-amino-2-(2-methoxyethyl)-1H-imidazo[4,5-c]quinolin-1-yl)propyl](N,N-diethylglycyl)amino]methyl}phenoxy)acetate,
Methyl (3-{[[3-(4-amino-2-butyl-1H-imidazo[4,5-c]quinolin-1-yl)propyl](3-morpholinopropyl)amino]methyl}phenoxy)acetate,
Methyl [4-({[({3-[4-amino-2-(2-methoxyethyl)-1H-imidazo[4,5-c]quinolin-1-yl]propyl}amino)carbonyl][3-(dimethylamino)propyl]amino}methyl)phenoxy]acetate,
Ethyl 2-[3-({3-[4-amino-2-(2-methoxyethyl)-1H-imidazo[4,5-c]quinolin-1-yl]propylamino}methyl)phenoxy]acetate,
Ethyl 2-{3-[(N-{3-[4-amino-2-(2-methoxyethyl)-1H-imidazo[4,5-c]quinolin-1-yl]propyl}-2-chloroacetamido)methyl)phenoxy]acetate,
Ethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate,
Propyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate,
Isopropyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate,
Isobutyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate,
2-Methoxyethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate,
2-Hydroxyethyl 2-{3-[(N-{3-[4-amino-2-(2-methoxyethyl)-1H-imidazo[4,5-c]quinolin-1-yl]propyl}-2-(diethylamino)acetamido)methyl]phenoxy}acetate,
Ethyl 2-{3-[(N-{3-[4-amino-2-(2-methoxyethyl)-1H-imidazo[4,5-c]quinolin-1-yl]propyl}-2-(pyrrolidin-1-yl)acetamido)methyl]phenoxy}acetate,
Ethyl 2-{3-[(N-{3-[4-amino-2-(2-methoxyethyl)-1H-imidazo[4,5-c]quinolin-1-yl]propyl}-2-(piperidin-1-yl)acetamido)methyl]phenoxy}acetate,
Ethyl 2-{3-[(N-{3-[4-amino-2-(2-methoxyethyl)-1H-imidazo[4,5-c]quinolin-1-yl]propyl}-2-(dimethylamino) acetamido)methyl]phenoxy}acetate,
Methyl 2-[4-({3-[4-amino-2-(2-methoxyethyl)-1H-imidazo[4,5-c]quinolin-1-yl]propylamino}methyl)phenoxy]acetate,
Methyl 2-{4-[(N-{3-[4-amino-2-(2-methoxyethyl)-1H-imidazo[4,5-c]quinolin-1-yl]propyl}-2-chloroacetamido)methyl]phenoxy}acetate,
Methyl 2-{4-[(N-{3-[4-amino-2-(2-methoxyethyl)-1H-imidazo[4,5-c]quinolin-1-yl]propyl}-2-(diethylamino)acetamido)methyl]phenoxy}acetate,
Ethyl 2-{4-[(N-{3-[4-amino-2-(2-methoxyethyl)-1H-imidazo[4,5-c]quinolin-1-yl]propyl}-2-(diethylamino)acetamido)methyl]phenoxy}acetate,
Methyl 2-[2-({3-[4-amino-2-(2-methoxyethyl)-1H-imidazo[4,5-c]quinolin-1-yl]propylamino}methyl)phenoxy]acetate,
Methyl 2-{2-[(N-{3-[4-amino-2-(2-methoxyethyl)-1H-imidazo[4,5-c]quinolin-1-yl]propyl}-2-chloroacetamido)methyl]phenoxy}acetate,
Methyl 2-{2-[(N-{3-[4-amino-2-(2-methoxyethyl)-1H-imidazo[4,5-c]quinolin-1-yl]propyl}-2-(diethylamino)acetamido)methyl]phenoxy}acetate,
Ethyl 2-{2-[(N-{3-[4-amino-2-(2-methoxyethyl)-1H-imidazo[4,5-c]quinolin-1-yl]propyl}-2-(diethylamino)acetamido)methyl]phenoxy}acetate,
Ethyl 2-[3-({4-[4-amino-2-(2-methoxyethyl)-1H-imidazo[4,5-c]quinolin-1-yl]butylamino}methyl)phenoxy]acetate,
Ethyl 2-{3-[(N-{4-[4-amino-2-(2-methoxyethyl)-1H-imidazo[4,5-c]quinolin-1-yl]butyl}-2-chloroacetamido)methyl]phenoxy}acetate,
Ethyl 2-{3-[(N-{4-[4-amino-2-(2-methoxyethyl)-1H-imidazo[4,5-c]quinolin-1-yl]butyl}-2-(diethylamino)acetamido)methyl]phenoxy}acetate,
Isopropyl 2-{3-[(N-{4-[4-amino-2-(2-methoxyethyl)-1H-imidazo[4,5-c]quinolin-1-yl]butyl}-2-{diethylamino}acetamido)methyl]phenoxy}acetate,
tert-Butyl 2-[3-({4-[4-amino-2-(2-methoxyethyl)-1H-imidazo[4,5-c]quinolin-1-yl]butylamino}methyl)phenoxy]acetate,
tert-Butyl 2-{3-[(N-{4-[4-amino-2-(2-methoxyethyl)-1H-imidazo[4,5-c]quinolin-1-yl]butyl}-2-chloro acetamido)methyl]phenoxy}acetate,
tert-Butyl 2-{3-[(N-{4-[4-amino-2-(2-methoxyethyl)-1H-imidazo[4,5-c]quinolin-1-yl]butyl}-2-{diethylamino}acetamido)methyl]phenoxy}acetate,
Methyl 2-[3-({3-[4-amino-2-(2-methoxyethyl)-1H-imidazo[4,5-c]quinolin-1-yl]propylamino}methyl)phenoxy]propanoate,
Methyl 2-{3-[(N-{3-[4-amino-2-(2-methoxyethyl)-1H-imidazo[4,5-c]quinolin-1-yl]propyl}-2-chloroacetamido)methyl]phenoxy}propanoate,
Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}propanoate,
Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}propanoate,
Ethyl 2-[3-({3-[4-amino-2-(2-methoxyethyl)-1H-imidazo[4,5-c]quinolin-1-yl]propylamino}methyl)phenoxy]-2-methylpropanoate,
Ethyl 2-{3-[(N-{3-[4-amino-2-(2-methoxyethyl)-1H-imidazo[4,5-c]quinolin-1-yl]propyl}-2-chloroacetamido)methyl]phenoxy}-2-methylpropanoate,
Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}-2-methylpropanoate,
Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}-2-methylpropanoate,
Ethyl 1-[3-({3-[4-amino-2-(2-methoxyethyl)-1H-imidazo[4,5-c]quinolin-1-yl]propylamino}methyl)phenoxy]cyclobutanecarboxylate,
Ethyl 1-{3-[(N-{3-[4-amino-2-(2-methoxyethyl)-1H-imidazo[4,5-c]quinolin-1-yl]propyl}-2-chloroacetamido)methyl]phenoxy}cyclobutanecarboxylate,
Ethyl 1-{3-[(N-{3-[4-amino-2-(2-methoxyethyl)-1H-imidazo[4,5-c]quinolin-1-yl]propyl}-2-{diethylamino}acetamido)methyl]phenoxy}cyclobutanecarboxylate,
Ethyl 2-[5-({3-[4-amino-2-(2-methoxyethyl)-1H-imidazo[4,5-c]quinolin-1-yl]propylamino}methyl)-2-methoxyphenoxy]acetate,
Ethyl 2-{5-[(N-{3-[4-amino-2-(2-methoxyethyl)-1H-imidazo[4,5-c]quinolin-1-yl]propyl}2-chloroacetamido)methyl]-2-methoxyphenoxy}acetate,
Ethyl 2-{5-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate,
Methyl 2-{5-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate,
Ethyl 2-[5-({3-[4-amino-2-(2-methoxyethyl)-1H-imidazo[4,5-c]quinolin-1-yl]propylamino}methyl)-2-methylphenoxy]acetate,
Ethyl 2-{5-[(N-{3-[4-amino-2-(2-methoxyethyl)-1H-imidazo[4,5-c]quinolin-1-yl]propyl}-2-chloroacetamido)methyl]-2-methylphenoxy}acetate,
Ethyl 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetate,
Isopropyl 2-{5-[(N-{3-[4-amino-2-(2-methoxyethyl)-1H-imidazo[4,5-c]quinolin-1-yl]propyl}-2-{diethylamino}acetamido)methyl]-2-methylphenoxy}acetate,
Methyl 2-[3-({3-[4-amino-2-(2-methoxyethyl)-1H-imidazo[4,5-c]quinolin-1-yl]propylamino}methyl)phenoxy]butanoate,
Methyl 2-{3-[(N-{3-[4-amino-2-(2-methoxyethyl)-1H-imidazo[4,5-c]quinolin-1-yl]propyl}-2-chloroacetamido)methyl]phenoxy}butanoate,
Methyl 2-{3-[(N-{3-[4-amino-2-(2-methoxyethyl)-1H-imidazo[4,5-c]quinolin-1-yl]propyl}-2-{diethylamino}acetamido)methyl]phenoxy}butanoate,
Ethyl 2-{3-[(N-{3-[4-amino-2-(2-methoxyethyl)-1H-imidazo[4,5-c]quinolin-1-yl]propyl}-2-{diethylamino}acetamido)methyl]phenoxy}butanoate,
Isopropyl 2-{5-[(N-{3-[4-amino-2-(2-methoxyethyl)-1H-imidazo[4,5-c]quinolin-1-yl]propyl}-2-{diethylamino}acetamido)methyl]-2-methoylphenoxy}acetate,
Isopropyl 2-[5-({3-[4-amino-2-(2-methoxyethyl)-1H-imidazo[4,5-c]quinolin-1-yl]propylamino}methyl)-2-methoxyphenoxy]acetate,
Isopropyl 2-{5-[(N-{3-[4-amino-2-(2-methoxyethyl)-1H-imidazo[4,5-c]quinolin-1-yl]propyl}-2-chloroacetamido)methyl]-2-methoxyphenoxy}acetate,
Isopropyl 2-{5-[(N-{3-[4-amino-2-(2-methoxyethyl)-1H-imidazo[4,5-c]quinolin-1-yl]propyl}-2-{dimethylamino}acetamido)methyl]-2-methoxyphenoxy}acetate,
Isopropyl 2-{5-[(N-{3-[4-amino-2-(2-methoxyethyl)-1H-imidazo[4,5-c]quinolin-1-yl]propyl}-2-{ethyl(methyl)amino}acetamido)methyl]-2-methoxyphenoxy}acetate,
Methyl 1-[3-({3-[4-amino-2-(2-methoxyethyl)-1H-imidazo[4,5-c]quinolin-1-yl]propylamino}methyl)phenoxy]cyclopropanecarboxylate,
Methyl 1-{3-[(N-{3-[4-amino-2-(2-methoxyethyl)-1H-imidazo[4,5-c]quinolin-1-yl]propyl}-2-chloroacetamido)methyl]phenoxy}cyclopropanecarboxylate,
Methyl 1-{3-[(N-{3-[4-amino-2-(2-methoxyethyl)-1H-imidazo[4,5-c]quinolin-1-yl]propyl}-2-{diethylamino}acetamido)methyl]phenoxy}cyclopropanecarboxylate,
Cyclopentyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate,
Cyclobutyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate,
Tetrahydro-2H-pyran-4-yl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate,
Butyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate,
tert-Butyl 2-[3-({3-[4-amino-2-(2-methoxyethyl)-1H-imidazo[4,5-c]quinolin-1-yl]propylamino}methyl)phenoxy]acetate,
tert-Butyl 2-{3-[(N-{3-[4-amino-2-(2-methoxyethyl)-1H-imidazo[4,5-c]quinolin-1-yl]propyl}-2-chloroacetamido)methyl]phenoxy}acetate,
tert-Butyl 2-{3-[(N-{3-[4-amino-2-(2-methoxyethyl)-1H-imidazo[4,5-c]quinolin-1-yl]propyl}-2-{diethylamino}acetamido)methyl]phenoxy}acetate,
Ethyl 2-[3-({3-[4-amino-2-(2-methoxyethyl)-1H-imidazo[4,5-c]quinolin-1-yl]propylamino}methyl)-2-methoxyphenoxy]acetate,
Ethyl 2-{3-[(N-{3-[4-amino-2-(2-methoxyethyl)-1H-imidazo[4,5-c]quinolin-1-yl]propyl}-2-chloroacetamido)methyl]-2-methoxyphenoxy}acetate,
Ethyl 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate,
Isopropyl 2-{3-[(N-{3-[4-amino-2-(2-methoxyethyl)-1H-imidazo[4,5-c]quinolin-1-yl]propyl}-2-{diethylamino}acetamido)methyl]-2-methoxyphenoxy}acetate,
Ethyl 2-[3-({3-[4-amino-2-(2-methoxyethyl)-1H-imidazo[4,5-c]quinolin-1-yl]propylamino}methyl)-2-fluorophenoxy]acetate,
Ethyl 2-{3-[(N-{3-[4-amino-2-(2-methoxyethyl)-1H-imidazo[4,5-c]quinolin-1-yl]propyl}-2-chloroacetamido)methyl]-2-fluorophenoxy}acetate,
Ethyl 2-{3-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate,
Isopropyl 2-{3-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate,
Ethyl 2-{3-[(N-{3-[4-amino-2-(2-methoxyethyl)-1H-imidazo[4,5-c]quinolin-1-yl]propyl}-2-{dimethylamino}acetamido)methyl]-2-fluorophenoxy}acetate,
Isopropyl 2-{3-[(N-{3-[4-amino-2-(2-methoxyethyl)-1H-imidazo[4,5-c]quinolin-1-yl]propyl}-2-{dimethylamino}acetamido)methyl]-2-fluorophenoxy}acetate,
Ethyl 2-{3-[(N-{3-[4-amino-2-(2-methoxyethyl)-1H-imidazo[4,5-c]quinolin-1-yl]propyl}-2-{ethyl(methyl)amino}acetamido)methyl]-2-fluorophenoxy}acetate,
Isopropyl 2-{3-[(N-{3-[4-amino-2-(2-methoxyethyl)-1H-imidazo[4,5-c]quinolin-1-yl]propyl}-2-{ethyl(methyl)amino}acetamido)methyl]-2-fluorophenoxy}acetate,
Ethyl 2-[3-({2-[4-amino-2-(2-methoxyethyl)-1H-imidazo[4,5-c]quinolin-1-yl]ethylamino}methyl)phenoxy]acetate,
Ethyl 2-{3-[(N-{2-[4-amino-2-(2-methoxyethyl)-1H-imidazo[4,5-c]quinolin-1-yl]ethyl}-2-chloro acetamido)methyl]phenoxy}acetate,
Ethyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate,
Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate,
Isopropyl 2-{3-[(N-{2-[4-amino-2-(2-methoxyethyl)-1H-imidazo[4,5-c]quinolin-1-yl]ethyl}-2-{diethylamino}acetamido)methyl]phenoxy}acetate,
Ethyl 2-[3-({2-[4-amino-2-(2-methoxyethyl)-1H-imidazo[4,5-c]quinolin-1-yl]ethylamino}methyl)phenoxy]-2-methylpropanoate,
Ethyl 2-{3-[(N-{2-[4-amino-2-(2-methoxyethyl)-1H-imidazo[4,5-c]quinolin-1-yl]ethyl}-2-chloroacetamido)methyl]phenoxy}-2-methylpropanoate,
Ethyl 2-{3-[(N-{2-[4-amino-2-(2-methoxyethyl)-1H-imidazo[4,5-c]quinolin-1-yl]ethyl}-2-{diethylamino}acetamido)methyl]phenoxy}-2-methylpropanoate,
Methyl 2-{3-[(N-{2-[4-amino-2-(2-methoxyethyl)-1H-imidazo[4,5-c]quinolin-1-yl]ethyl}-2-{diethylamino}acetamido)methyl]phenoxy}-2-methylpropanoate,
Cyclopentyl 2-[3-({3-[4-amino-2-(2-methoxyethyl)-1H-imidazo[4,5-c]quinolin-1-yl]propylamino}methyl)phenoxy]acetate,
Cyclopentyl 2-{3-[(N-{3-[4-amino-2-(2-methoxyethyl)-1H-imidazo[4,5-c]quinolin-1-yl]propyl}-2-chloroacetamido)methyl]phenoxy}acetate,
Cyclopentyl 2-{3-[(N-{3-[4-amino-2-(2-methoxyethyl)-1H-imidazo[4,5-c]quinolin-1-yl]propyl}-2-{dimethylamino}acetamido)methyl]phenoxy}acetate,
Cyclopentyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}acetate,
Isopropyl 2-{3-[(N-{3-[4-amino-2-(2-methoxyethyl)-1H-imidazo[4,5-c]quinolin-1-yl]propyl}-2-morpholinoacetamido)methyl]phenoxy}acetate,
Isopropyl 2-{3-[(N-{3-[4-amino-2-(2-methoxyethyl)-1H-imidazo[4,5-c]quinolin-1-yl]propyl}-2-{dimethylamino}acetamido)methyl]phenoxy}acetate,
Isopropyl 2-{3-[(N-{3-[4-amino-2-(2-methoxyethyl)-1H-imidazo[4,5-c]quinolin-1-yl]propyl}-2-{ethyl(methyl)amino}acetamido]methyl)phenoxy}acetate,
Isopropyl 2-{3-[(N-{3-[4-amino-2-(2-methoxyethyl)-1H-imidazo[4,5-c]quinolin-1-yl]propyl}-2-{(2-methoxyethyl)(methyl)amino}acetamido)methyl]phenoxy}acetate,
Isopropyl 2-[5-({3-[4-amino-2-(2-methoxyethyl)-1H-imidazo[4,5-c]quinolin-1-yl]propylamino}methyl)-2-fluorophenoxy]acetate,
Isopropyl 2-{5-[(N-{3-[4-amino-2-(2-methoxyethyl)-1H-imidazo[4,5-c]quinolin-1-yl]propyl}-2-chloroacetamido)methyl]-2-fluorophenoxy}acetate,
Isopropyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate,
Ethyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate,
Methyl 2-{5-[(N-{3-[4-amino-2-(2-methoxyethyl)-1H-imidazo[4,5-c]quinolin-1-yl]propyl}-2-{diethylamino}acetamido)methyl]-2-fluorophenoxy}acetate,
Isopropyl 2-[3-({3-[4-amino-2-(2-methoxyethyl)-1H-imidazo[4,5-c]quinolin-1-yl]propylamino}methyl)-5-fluorophenoxy]acetate,
Isopropyl 2-{3-[(N-{3-[4-amino-2-(2-methoxyethyl)-1H-imidazo[4,5-c]quinolin-1-yl]propyl}-2-chloroacetamido)methyl]-5-fluorophenoxy}acetate,
Isopropyl 2-{3-[(N-{3-[4-amino-2-(2-methoxyethyl)-1H-imidazo[4,5-c]quinolin-1-yl]propyl}-2-{diethylamino}acetamido)methyl]-5-fluorophenoxy]acetate,
Ethyl 2-{3-[(N-{3-[4-amino-2-(2-methoxyethyl)-1H-imidazo[4,5-c]quinolin-1-yl]propyl}-2-{diethylamino}acetamido)methyl]-5-fluorophenoxy}acetate,
Ethyl 2-{4-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate,
Ethyl 2-{3-[(1-{4-[4-amino-2-(2-methoxyethyl)-1H-imidazo[4,5-c]quinolin-1-yl]butyl}-3-{2-(piperidin-1-yl)ethyl}ureido)methyl]phenoxy}acetate,
Ethyl 2-{3-[(1-{4-[4-amino-2-(2-methoxyethyl)-1H-imidazo[4,5-c]quinolin-1-yl]butyl}-3-{2-(dimethylamino) ethyl}ureido)methyl]phenoxy}acetate,
Ethyl 2-{3-[(1-{4-[4-amino-2-(2-methoxyethyl)-1H-imidazo[4,5-c]quinolin-1-yl]butyl}-3-{3-(piperidin-1-yl)propyl}ureido)methyl]phenoxy}acetate,
Ethyl 2-{3-[(1-{4-[4-amino-2-(2-methoxyethyl)-1H-imidazo[4,5-c]quinolin-1-yl]butyl}-3-{3-(dimethylamino)propyl}ureido)methyl]phenoxy}acetate,
Ethyl 2-{3-[(3-{4-[4-amino-2-(2-methoxyethyl)-1H-imidazo[4,5-c]quinolin-1-yl]butyl}-1-{2-(piperidin-1-yl)ethyl}ureido)methyl]phenoxy}acetate,
Ethyl 2-{4-[(3-{4-[4-amino-2-(2-methoxyethyl)-1H-imidazo[4,5-c]quinolin-1-yl]butyl}-1-{2-(piperidin-1-yl)ethyl}ureido)methyl]phenoxy}acetate,
Isopropyl 2-{3-[({4-[4-amino-2-(2-methoxyethyl)-1H-imidazo[4,5-c]quinolin-1-yl]butyl}{2-[dimethylamino]ethyl}amino)methyl]phenoxy}acetate,
Isopropyl 2-{3-[({4-[4-amino-2-(2-methoxyethyl)-1H-imidazo[4,5-c]quinolin-1-yl]butyl}{3-morpholinopropyl}amino)methyl]phenoxy}acetate,
Ethyl 2-{3-[({4-[4-amino-2-(2-methoxyethyl)-1H-imidazo[4,5-c]quinolin-1-yl]butyl}{2-(dimethylamino)ethyl}amino)methyl]phenoxy}-2-methylpropanoate,
Methyl 2-{3-[({4-[4-amino-2-(2-methoxyethyl)-1H-imidazo[4,5-c]quinolin-1-yl]butyl}{2-(dimethylamino)ethyl}amino)methyl]phenoxy}-2-methylpropanoate,
Ethyl 2-{3-[({4-[4-amino-2-(2-methoxyethyl)-1H-imidazo[4,5-c]quinolin-1-yl]butyl}{3-morpholinopropyl}amino)methyl]phenoxy}-2-methylpropanoate,
Methyl 2-{3-[({4-[4-amino-2-(2-methoxyethyl)-1H-imidazo[4,5-c]quinolin-1-yl]butyl}{3-morpholinopropyl}amino)methyl]phenoxy}-2-methylpropanoate,
Isopropyl 2-[5-({4-[4-amino-2-(2-methoxyethyl)-1H-imidazo[4,5-c]quinolin-1-yl]butylamino}methyl)-2-fluorophenoxy]acetate,
Isopropyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate,
Ethyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate,
Methyl 2-{5-[(1-{4-[4-amino-2-(2-methoxyethyl)-1H-imidazo[4,5-c]quinolin-1-yl]butyl}-3-{2-(piperidin-1-yl)ethyl}ureido)methyl]-2-fluorophenoxy}acetate,
Isopropyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate,
Ethyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate,
Methyl 2-{5-[(3-{4-[4-amino-2-(2-methoxyethyl)-1H-imidazo[4,5-c]quinolin-1-yl]butyl}-1-{2-(piperidin-1-yl)ethyl}ureido)methyl]-2-fluorophenoxy}acetate,
Isopropyl 2-[3-({3-[4-amino-2-(2-methoxyethyl)-1H-imidazo[4,5-c]quinolin-1-yl]propylamino}methyl)-2-methylphenoxy]acetate,
Isopropyl 2-{3-[(N-{3-[4-amino-2-(2-methoxyethyl)-1H-imidazo[4,5-c]quinolin-1-yl]propyl}-2-chloroacetamido)methyl]-2-methylphenoxy}acetate,
Isopropyl 2-{3-[(N-{3-[4-amino-2-(2-methoxyethyl)-1H-imidazo[4,5-c]quinolin-1-yl]propyl}-2-{diethylamino}acetamido)methyl]-2-methylphenoxy}acetate,
Ethyl 2-{3-[(N-{3-[4-amino-2-(2-methoxyethyl)-1H-imidazo[4,5-c]quinolin-1-yl]propyl}-2-{dimethylamino}acetamido)methyl]phenoxy}-2-methylpropanoate,
Methyl 2-{3-[(N-{3-[4-amino-2-(2-methoxyethyl)-1H-imidazo[4,5-c]quinolin-1-yl]propyl}-2-{dimethylamino}acetamido)methyl]phenoxy}-2-methylpropanoate,
Ethyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}-2-methylpropanoate,
Methyl 2-{3-[(N-{3-[4-amino-2-(2-methoxyethyl)-1H-imidazo[4,5-c]quinolin-1-yl]propyl}-2-{ethyl(methyl)amino}acetamido)methyl]phenoxy}-2-methylpropanoate,
Isopropyl 2-(3-{[1-(2-{2-[4-amino-2-(2-methoxyethyl)-1H-imidazo[4,5-c]quinolin-1-yl]ethoxy}ethyl)-3-{2-(piperidin-1-yl)ethyl}ureido]methyl}phenoxy)acetate,
Ethyl 2-[3-({N-[3-(4-amino-2-butyl-1H-imidazo[4,5-c]quinolin-1-yl)propyl]-2-(diethylamino)acetamido}methyl)phenoxy]-2-methylpropanoate,
Methyl 2-[3-({N-[3-(4-amino-2-butyl-1H-imidazo[4,5-c]quinolin-1-yl)propyl]-2-(diethylamino)acetamido}methyl)phenoxy]-2-methylpropanoate,
Ethyl 2-[3-({N-[3-(4-amino-2-butyl-1H-imidazo[4,5-c]quinolin-1-yl)propyl]-2-(dimethylamino)acetamido}methyl)phenoxy]-2-methylpropanoate,
Methyl 2-[3-({N-[3-(4-amino-2-butyl-1H-imidazo[4,5-c]quinolin-1-yl)propyl]-2-(dimethylamino)acetamido}methyl)phenoxy]-2-methylpropanoate,
Isopropyl 2-[3-({N-[3-(4-amino-2-butyl-1H-imidazo[4,5-c]quinolin-1-yl)propyl]-2-(diethylamino)acetamido}methyl)phenoxy]acetate,
Isopropyl 2-[3-({N-[3-(4-amino-2-butyl-1H-imidazo[4,5-c]quinolin-1-yl)propyl]-2-[ethyl(methyl)amino]acetamido}methyl)phenoxy]acetate,
Isopropyl 2-[3-({N-[3-(4-amino-2-butyl-1H-imidazo[4,5-c]quinolin-1-yl)propyl]-2-(dimethylamino)acetamido}methyl)phenoxy]acetate,
Isopropyl 2-[3-({N-[3-(4-amino-2-propyl-1H-imidazo[4,5-c]quinolin-1-yl)propyl]-2-(diethylamino)acetamido}methyl)phenoxy]acetate,
Isopropyl 2-{3-[(N-{3-[4-amino-2-(ethoxymethyl)-1H-imidazo[4,5-c]quinolin-1-yl]propyl}-2-{diethylamino}acetamido)methyl]phenoxy}acetate,
Ethyl 2-{3-[(N-{3-[4-amino-2-(ethoxymethyl)-1H-imidazo[4,5-c]quinolin-1-yl]propyl}-2-{diethylamino}acetamido)methyl]phenoxy}-2-methylpropanoate,
Methyl 2-{3-[(N-{3-[4-amino-2-(ethoxymethyl)-1H-imidazo[4,5-c]quinolin-1-yl]propyl}-2-{diethylamino}acetamido)methyl]phenoxy}-2-methylpropanoate,
Isopropyl 2-[3-({N-[3-(4-amino-2-butyl-1H-imidazo[4,5-c]quinolin-1-yl)propyl]-2-(diethylamino)acetamido}methyl)-2-fluorophenoxy]acetate,
Isopropyl 2-[3-({N-[3-(4-amino-2-butyl-1H-imidazo[4,5-c]quinolin-1-yl)propyl]-2-(dimethylamino)acetamido}methyl)-2-fluorophenoxy]acetate,
Isopropyl 2-{3-[(N-{3-[4-amino-2-(ethoxymethyl)-1H-imidazo[4,5-c]quinolin-1-yl]propyl}-2-{diethylamino}acetamido)methyl]-2-fluorophenoxy}acetate,
Isopropyl 2-{3-[(N-{3-[4-amino-2-(ethoxymethyl)-1H-imidazo[4,5-c]quinolin-1-yl]propyl}-2-{dimethylamino}acetamido)methyl]-2-fluorophenoxy}acetate,
Ethyl 2-[3-({N-[3-(4-amino-2-butyl-1H-imidazo[4,5-c]quinolin-1-yl)propyl]-2-(pyrrolidin-1-yl)acetamido}methyl)phenoxy]-2-methylpropanoate,
Methyl 2-[3-({N-[3-(4-amino-2-butyl-1H-imidazo[4,5-c]quinolin-1-yl)propyl]-2-(pyrrolidin-1-yl)acetamido}methyl)phenoxy]-2-methylpropanoate,
Ethyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}-2-methylpropanoate,
Methyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}-2-methylpropanoate,
Isopropyl 2-{3-[(N-{3-[4-amino-2-(ethoxymethyl)-1H-imidazo[4,5-c]quinolin-1-yl]propyl}-2-{pyrrolidin-1-yl}acetamido)methyl]phenoxy}acetate,
Ethyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate and Methyl 2-{3-[(N-{3-[4-amino-2-(propoxymethyl)-1H-imidazo[4,5-c]quinolin-1-yl]propyl}-2-{diethylamino}acetamido)methyl]phenoxy}-2-methylpropanoate.

20. A pharmaceutical composition comprising a compound of formula (I) or a pharmaceutically acceptable salt thereof as claimed in any one of claims 1 to 19 in association with a pharmaceutically acceptable adjuvant, diluent or carrier.

21. A compound of formula (I) or a pharmaceutically acceptable salt thereof as claimed in any one of claims 1 to 19 for use in the treatment of allergic or viral diseases or cancers or for use in treating asthma, COPD, allergic rhinitis, allergic conjunctivitis, atopic dermatitis, cancer, hepatitis B, hepatitis C, HIV, HPV, bacterial infections and dermatosis.

22. A method of treating, or reducing the risk of, a disease or condition in which modulation of TLR7 activity is beneficial which comprises administering to a patient in need thereof a therapeutically effective amount of a compound of formula (I) or a pharmaceutically acceptable salt thereof as claimed in any one of claims 1 to 19.

23. A compound of formula (I′): 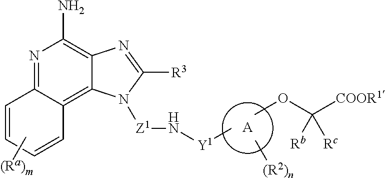
wherein Z¹, Y¹, R^{a}, R^{b}, R^{c}, R², R³, m and n are as defined in claim 1; and R^{1′} represents hydrogen, C₁-C₈ alkyl, C₃-C₈ cycloalkyl, or a 3- to 8-membered saturated heterocyclic ring group comprising a O atom, wherein R^{1′} is optionally substituted by one or more substituents independently selected from halogen, cyano, hydroxyl and C₁-C₃ alkoxy;
or a salt thereof.
